(19) United States
(12) Patent Application Publication
(10) Pub. No.: US 2007/0167452 A1
(43) Pub. Date: Jul. 19, 2007

(54) Therapeutic amide derivatives

(75) Inventors: Makoto Kawai, Aichi-ken (JP); Mitsuhiro Kawamura, Aichi-ken (JP); Asato Morita, Aichi-ken (JP); Isao Sakurada, Aichi-ken (JP)

Correspondence Address: WARNER-LAMBERT COMPANY, 2800 PLYMOUTH RD, ANN ARBOR, MI, 48105, US

(73) Assignee: PFIZER, INC.

(21) Appl. No.: 10/597,868

(22) PCT Filed: Feb. 1, 2005
(86) PCT No.: PCT/IB05/00258

§ 371 Date: Aug. 10, 2006

Related U.S. Application Data
(60) Provisional application No. 60/544,258, filed on Feb. 11, 2004.

(51) Int. Cl. A61K 31/53; A61K 31/513; A61K 31/452; A61K 31/4433; A61K 31/428; A61K 31/423; A61K 31/165

(52) U.S. Cl. 514/242; 514/269; 514/326; 514/312; 514/336; 514/375; 514/367; 514/617; 544/183; 544/314; 546/158; 546/282.1; 564/170; 548/165; 548/221

(57) ABSTRACT

The present invention relates to compounds of the formula (I): 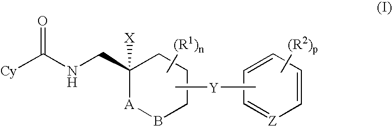
or a pharmaceutically acceptable salt or solvate thereof, 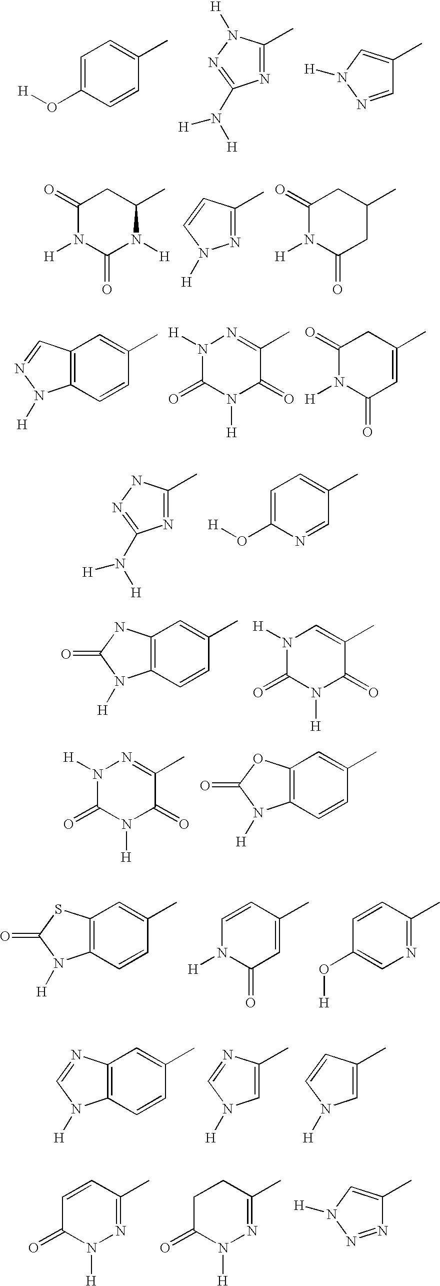 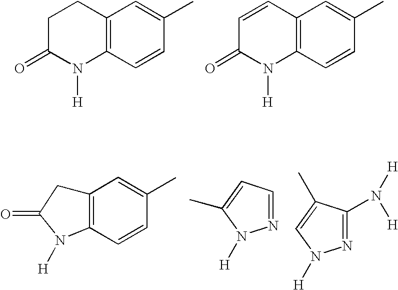 to processes for the preparation of, intermediates used in the preparation of, compositions containing such compounds and the uses of such compounds as antagonists of the NMDA NR2B receptor.

### TECHNICAL FIELD

This invention relates to amide derivatives and to processes for the preparation of, intermediates used in the preparation of, compositions containing and the uses of, such derivatives.

### BACKGROUND ART

The amide derivatives of the present invention are antagonists of NMDA (N-methyl-D-aspartate) NR2B receptor, and have a number of therapeutic applications, particularly in the treatment of pain, stroke, traumatic brain injury, Parkinson's disease, Alzheimer's disease, depression, anxiety, migraine, or the like.

Glutamate plays a dual role in the central nervous system (CNS) as essential amino acid and the principal excitatory neurotransmitters. There are two major class of receptors, ionotoropic and metabotropic. Ionotropic receptors are classified into three major subclass, N-methyl-asparatate(NMDA), 2-amino-3(methyl-3-hydroxyisoxazol-4-yl)propionic acid (AMPA) and kainate. There is considerable preclinical evidence that hyperalgesia and allodynia following peripheral tissue or nerve injury is not only due to an increase in the sensitivity of primary afferent nociceptors at the site of injury but also depends on NMDA receptor-mediated central changes in synaptic excitability. In humans, NMDA receptor antagonists have also been found to decrease both pain perception and sensitization. Also, overactivation of the NMDA receptor is a key event for triggering neuronal cell death under pathological conditions of acute and chronic forms of neurodegeneration. However, while NMDA receptor inhibition has therapeutic utility in the treatment of pain and neurodegenerative diseases, there are significant liabilities to many available NMDA receptor antagonists that can cause potentially serious side effects. NMDA subunits are differentially distributed in the CNS. Especially, NR2B is believed to be restricted to the forebrain and laminas I and II of the dosal horn. The more discrete distribution of NR2B subunit in the CNS may support a reduced side-effect profile of agents that act selectively at this site. For example, NMDA NR2B selective antagonists may have clinical utility for the treatment of neuropathic and other pain conditions in human with a reduced side-effect profile than existing NMDA antagonists (S. Boyce, et al., Neuropharmacology, 38, pp. 611-623 (1999)).

International Patent Application Number (WO) 0208928 discloses a variety of benzamide compounds, which are NMDA NR2B antagonists, for example, compound (i) below:

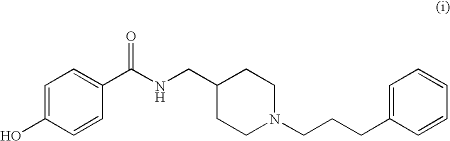

Compound (i) shows an IC50 of <3mcM at HERG potassium channel.

WO09967203 describes cyclohexyl derivatives which are claimed to be useful in the treatment of pain.

There is a need to provide new NMDA NR2B antagonists that are good drug candidates. In particular, preferred compounds should bind potently to the NR2B receptor and show functional activity as antagonists whilst showing little affinity for other receptors. They should be well absorbed from the gastrointestinal tract, be metabolically stable and possess favourable pharmacokinetic properties. They should be non-toxic and demonstrate few side-effects.

Furthermore, the ideal drug candidate will exist in a physical form that is stable, non-hygroscopic and easily formulated.

In particular, it would be desirable to provide a NMDA NR2B selective antagonist with reduced inhibitory activity at HERG potassium channel.

### DETAILED DESCRIPTION OF THE INVENTION

The invention, therefore, provides a compound of the formula (I): 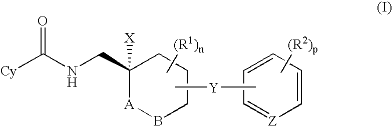
or a pharmaceutically acceptable salt or solvate thereof, wherein:
A and B independently represent CH₂ or O, with the proviso that A and B are not simultaneously O;
Cy represents one of the following 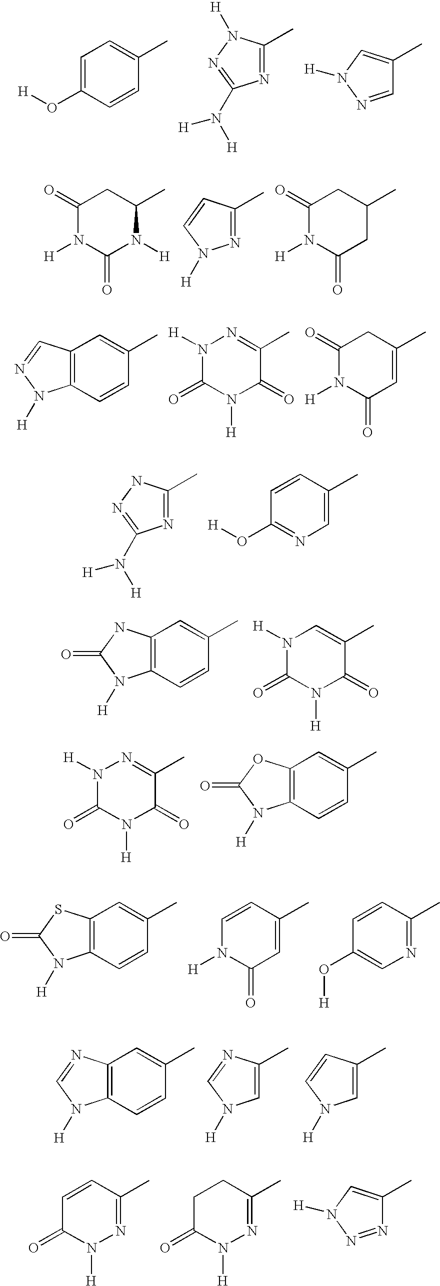 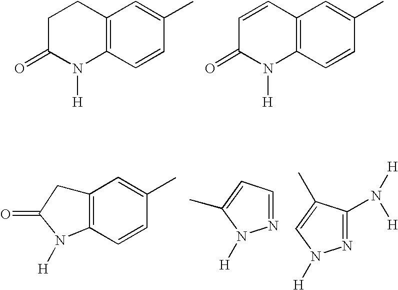
optionally substituted by one to three groups selected from hydroxy, halogen, C₁₋₆alkyl, C₁₋₆alkoxy, C₁₋₆haloalkyl, C₁₋₆alkylamino and amino;
R¹ and R² are independently selected from hydroxy, halogen, C₁₋₆alkyl, C₁₋₆alkoxy, C₁₋₆haloalkyl and C₃₋₈ cycloalkyl;
n represents an integer from 0-4;
X is hydrogen, hydroxy, halogen or C₁₋₆ alkoxy;
Y is oxy, thio, a 1-4 membered alkylene, a 2-4 membered alkylene ether, 2-4 membered alkylene thioether or an oxyethyleneoxy group, optionally substituted by 1 to 4 groups independently selected from hydroxy, halogen, C₁₋₆alkyl, C₁₋₆alkoxy and C₁₋₆haloalkyl;
Z is CH or N; and
p represents an integer from 0-5 when Z is CH or 0-4 when Z is N, when p represents 2 or more, two of R²s may be taken together with the carbon atoms to which they are attached to form a 5-8 membered cycloalkyl ring.

In the above definitions, halo means fluoro, chloro, bromo or iodo. Alkyl, alkylene, and alkoxy groups, containing the requisite number of carbon atoms, can be unbranched or branched. Examples of alkyl include methyl, ethyl, n-propyl, i-propyl, n-butyl, i-butyl, sec-butyl and t-butyl. Examples of cycloalkyl include cyclopropyl, cyclobutyl, cyclopentyl, cyclohexyl, cycloheptyl and cyclooctyl. Examples of alkylene include methylene, ethylene, n-propylene, 1-methylethylene, n-butylene, 1-methylpropylene, 2-methylpropylene and 1,1-dimethylethylene. Examples of alkoxy include methoxy, ethoxy, n-propoxy, i-propoxy, n-butoxy, i-butoxy, sec-butoxy and t-butoxy. Haloalkyl defines an alkyl group substituted by one or more halogen groups. Examples of haloalkyl include difluoromethyl, trifluoromethyl and pentafluoroethyl. 2-4 membered alkylene ether difines a 2 to 4 membered chain wherein one member is oxygen and at least one ther member is C₁-C₃ alkylene. Examples of 2-4 membered alkylene ether groups include oxymethylene, methyleneoxy, ethyleneoxy, oxyethylene and methyleneoxymethylene. Examples of 2-4 membered alkylene thioether groups include thiomethylene, methylenethio, ethylenethio and thioethylene. Examples of 5-8 membered cycloalkyl rings include cyclopropyl, cyclobutyl, cyclopentyl, cyclohexyl, cycloheptyl and cyclooctyl.

In a preferred aspect (A), the invention provides a compound of the formula (I), or a pharmaceutically acceptable salt or solvate thereof, wherein Cy is selected from 4-hydroxyphenyl, 1H-pyrazol-4-yl, 2-oxo-2,3-dihydro-1,3-benzoxazole-6-yl, 2-hydroxy-4-pyridyl, 5-pyrazolyl, 3-pyrazolyl, 4-pyrazolyl, 2-oxoindoline, 3-amino-4-pyrazolyl and 2-hydroxy-5-pyridyl, unsubstituted or substititued by halogen, e.g. fluoro or C₁₋₆alkyl, e.g methyl, more preferably 4-hydroxyphenyl unsubstituted or substititued by fluoro, most preferably substituted by fluoro ortho to the phenolic hydroxy group, and A, B, R¹, R², n, p, X, Y and Z are as defined above.

In a further preferred aspect (B), the invention provides a compound of the formula (I), or a pharmaceutically acceptable salt or solvate thereof, wherein Cy is defined above, either in its broadest aspect or in a preferred, more or most preferred aspect under (A), n is 0 and A, B, R¹, R², p, X, Y and Z are as defined above.

In a further preferred aspect (C), the invention provides a compound of the formula (I), or a pharmaceutically acceptable salt or solvate thereof, wherein Cy is defined above, either in its broadest aspect or in a preferred, more or most preferred aspect under (A) or (B), n is defined above, either in the broadest aspect or in a preferred aspect under (B), p is 0-2 and R² is selected from fluoro, chloro, C₁₋₆alkyl, e.g. methyl, ethyl, isopropyl or n-propyl, methoxy or trifluoromethyl, more preferably methoxy, chloro, fluoro and methyl, and A, B, R¹, X, Y and Z are as defined above.

In a further preferred aspect (D), the invention provides a compound of the formula (I), or a pharmaceutically acceptable salt or solvate thereof, wherein Cy is defined above, either in its broadest aspect or in a preferred, more or most preferred aspect under (A), (B) or (C), n is defined above, either in the broadest aspect or in a preferred aspect under (B) or (C), p and R² are defined above, either in the broadest aspect or in a preferred or more preferred aspect under (C), X is hydrogen, fluoro, hydroxy or methoxy, more preferably hydrogen or hydroxy, and A, B, R¹, Y and Z are as defined above.

In a further preferred aspect (E), the invention provides a compound of the formula (I), or a pharmaceutically acceptable salt or solvate thereof, wherein Cy is defined above, either in its broadest aspect or in a preferred, more or most preferred aspect under (A), (B) or (C) or (D), n is defined above, either in the broadest aspect or in a preferred aspect under (B), (C) or (D), p and R² are defined above, either in the broadest aspect or in a preferred or more preferred aspect under (C) or (D), X is defined above, either in its broadest aspect or in a preferred or more preferred aspect under (D), Y is methylene, oxyethyleneoxy, oxymethylene, methyleneoxy, methyleneoxymethylene, ethyleneoxy, oxyethylene or oxy, more preferably methyleneoxy, and A, B, R¹, and Z are as defined above.

In a further preferred aspect (F), the invention provides a compound of the formula (I), or a pharmaceutically acceptable salt or solvate thereof, wherein Cy is defined above, either in its broadest aspect or in a preferred, more or most preferred aspect under (A), (B), (C), (D) or (E), n is defined above, either in the broadest aspect or in a preferred aspect under (B), (C), (D) or (E), p and R² are defined above, either in the broadest aspect or in a preferred or more preferred aspect under (C), (D) or (E), X is defined above, either in its broadest aspect or in a preferred or more preferred aspect under (D) or (E), Y is defined above, either in its broadest aspect or in a preferred or more preferred aspect under (E), Z is C and A, B and R¹ are as defined above.

In a further preferred aspect (G), the invention provides a compound of the formula (I), or a pharmaceutically acceptable salt or solvate thereof, wherein Cy is defined above, either in its broadest aspect or in a preferred, more or most preferred aspect under (A), (B), (C), (D), (E) or (F), n is defined above, either in the broadest aspect or in a preferred aspect under (B), (C), (D), (E) or (F), p and R² are defined above, either in the broadest aspect or in a preferred or more preferred aspect under (C), (D), (E) or (F), X is defined above, either in its broadest aspect or in a preferred or more preferred aspect under (D), (E) or (F), Y is defined above, either in its broadest aspect or in a preferred or more preferred aspect under (E) or (F), Z is defined above, either in its broadest aspect or in a preferred aspect under (F), the group Y is para located to and in a trans configuration to X, and A, B and R¹ are as defined above.

Individual preferred A, B, Cy, R¹, R², n, p, X, Y and Z groups are those defined by the A, B, Cy, R¹, R², n, p, X, Y and Z groups in the Examples section below.

Particularly preferred compounds of the invention include those in which each variable in Formula (I) is selected from the preferred groups for each variable. Even more preferable compounds of the invention include those where each variable in Formula (I) is selected from the more or most preferred groups for each variable.

A specific compound according to the invention is selected from the list consisting of:
4-Hydroxy-N-{[cis-4-(phenoxymethyl)cyclohexyl]methyl}benzamide;
4-Hydroxy-N-({cis-4-[(4-methoxyphenoxy)methyl]cyclohexyl}methyl)benzamide;
N-{[cis-4-(Benzyloxy)cyclohexyl]methyl}-4-hydroxybenzamide;
N-({cis-4-[(4-Chlorobenzyl)oxy]cyclohexyl}methyl)-4-hydroxybenzamide;
N-({cis-4-[(3-Chlorobenzyl)oxy]cyclohexyl}methyl)-4-hydroxybenzamide;
4-Hydroxy-N-{[cis-4-(4-methoxyphenoxy)cyclohexyl]methyl}benzamide;
N-{[cis-4-(4-Chlorophenoxy)cyclohexyl]methyl}-4-hydroxybenzamide;
4-Hydroxy-N-{[1-hydroxy-4-(phenoxymethyl)cyclohexyl]methyl}benzamide;
N-({trans-4-[(4-Fluorophenoxy)methyl]-1-hydroxycyclohexyl}methyl)-4-hydroxybenzamide;
N-({trans-4-[(3-Fluorophenoxy)methyl]-1-hydroxycyclohexyl}methyl)-4-hydroxybenzamide;
N-({trans-4-[(2-Fluorophenoxy)methyl]-1-hydroxycyclohexyl}methyl)-4-hydroxybenzamide;
N-({trans-4-[(2,6-Difluorophenoxy)methyl]-1-hydroxycyclohexyl}methyl)-4-hydroxybenzamide;
N-({trans-4-[(3,5-Difluorophenoxy)methyl]-1-hydroxycyclohexyl}methyl)-4-hydroxybenzamide;
N-({trans-4-[(3-Chlorophenoxy)methyl]-1-hydroxycyclohexyl}methyl)-4-hydroxybenzamide;
N-({trans-4-[(4-Chlorophenoxy)methyl]-1-hydroxycyclohexyl}methyl)-4-hydroxybenzamide;
4-Hydroxy-N-({trans-1-hydroxy-4-[(2-methylphenoxy)methyl]cyclohexyl}methyl)benzamide;
4-Hydroxy-N-({trans-1-hydroxy-4-[(3-methylphenoxy)methyl]cyclohexyl}methyl)benzamide;
4-Hydroxy-N-({trans-1-hydroxy-4-[(4-methylphenoxy)methyl]cyclohexyl}methyl)benzamide;
N-({trans-4-[(Benzyloxy)methyl]-1-hydroxycyclohexyl}methyl)-4-hydroxybenzamide;
N-[(trans-4-{[(2-Fluorobenzyl)oxy]methyl}-1-hydroxycyclohexyl)methyl]-4-hydroxybenzamide;
N-[(trans-4-{[(4-Fluorobenzyl)oxy]methyl}-1-hydroxycyclohexyl)methyl]-4-hydroxybenzamide;
4-Hydroxy-N-{[trans-1-hydroxy-4-(2-phenoxyethyl)cyclohexyl]methyl}benzamide;
N-({trans-4-[2-(2-Fluorophenoxy)ethyl]-1-hydroxycyclohexyl}methyl)-4-hydroxybenzamide;
N-({trans-4-[2-(3-Fluorophenoxy)ethyl]-1-hydroxycyclohexyl}methyl)-4-hydroxybenzamide;
N-({trans-4-[2-(4-Fluorophenoxy)ethyl]-1-hydroxycyclohexyl}methyl)-4-hydroxybenzamide;
N-{[trans-4-(Benzyloxy)-1-hydroxycyclohexyl]methyl}-4-hydroxybenzamide;
N-{[trans-4-(4-Chlorophenoxy)-1-hydroxycyclohexyl]methyl}-4-hydroxybenzamide;
N-{[cis-4-(4-Chlorophenoxy)-1-hydroxycyclohexyl]methyl}-4-hydroxybenzamide;
N-{[trans-4-(4-Chlorophenoxy)-1-hydroxycyclohexyl]methyl}-3-fluoro-4-hydroxybenzamide;
N-{[cis-4-(4-Chlorophenoxy)-1-hydroxycyclohexyl]methyl}-3-fluoro-4-hydroxybenzamide;
(+)-4-hydroxy-N-{[5S-(phenoxymethyl)tetrahydro-2H-pyran-2S-yl]methyl}benzamide;
(−)-4-hydroxy-N-{[5R-(phenoxymethyl)tetrahydro-2H-pyran-2R-yl]methyl}benzamide;
4-hydroxy-N-{[5S-(benzyloxymethyl)tetrahydro-2H-pyran-2S-yl]methyl}benzamide;
4-hydroxy-N-{[5R-(benzyloxymethyl)tetrahydro-2H-pyran-2R-yl]methyl}benzamide;
(−)-4-Hydroxy-N-{[(3R,6S)-6-(phenoxymethyl)tetrahydro-2H-pyran-3-yl]methyl}benzamide;
(+)-4-Hydroxy-N-{[(3S,6R)-6-(phenoxymethyl)tetrahydro-2H-pyran-3-yl]methyl}benzamide;
N-({trans-4-[(2-Fluorobenzyl)oxy]-1-hydroxycyclohexyl}methyl)-4-hydroxybenzamide;
3-Fluoro-N-({trans-4-[2-(2-fluorophenoxy)ethyl]-1-hydroxycyclohexyl}methyl)-4-hydroxybenzamide;
trans-N-{[4-(4-Chlorophenoxy)cyclohexyl]methyl}-3-fluoro-4-hydroxybenzamide;
cis-N-{[4-(4-Chlorophenoxy)cyclohexyl]methyl}-3-fluoro-4-hydroxybenzamide;
N-{[cis-4-(4-Fluorophenoxy)cyclohexyl]methyl}-4-hydroxybenzamide;
3-Fluoro-N-{[cis-4-(4-fluorophenoxy)cyclohexyl]methyl}-4-hydroxybenzamide;
N-({trans-4-[2-(2-Fluorophenoxy)ethyl]-1-hydroxycyclohexyl}methyl)-1H-pyrazole-4-carboxamide;
4-Hydroxy-N-{[cis-4-(2-phenylethoxy)cyclohexyl]methyl}benzamide;
2-Fluoro-4-hydroxy-N-{[trans-1-hydroxy-4-(phenoxymethyl)cyclohexyl]methyl}benzamide;
N-({trans-4-[(Benzyloxy)methyl]-1-hydroxycyclohexyl}methyl)-3-fluoro-4-hydoxybenzamide;
N-({cis-4-[(Benzyloxy)methyl]cyclohexyl}methyl)-4-hydroxybenzamide 3-Fluoro-4-hydroxy-N-{[trans-1-hydroxy-4-(phenoxymethyl)cyclohexyl]methyl}benzamide;
3-Fluoro-4-hydroxy-N-{[trans-1-hydroxy-4-(2-phenoxyethyl)cyclohexyl]methyl}benzamide;
3-Fluoro-N-[(trans-4-{[(4-fluorobenzyl)oxy]methyl}-1-hydroxycyclohexyl)methyl]-4-hydroxybenzamide;
3-Fluoro-N-({trans-4-[(2-fluorophenoxy)methyl]-1-hydroxycyclohexyl}methyl)-4-hydroxybenzamide;
3-Fluoro-N-({trans-4-[(4-fluorophenoxy)methyl]-1-hydroxycyclohexyl}methyl)-4-hydroxybenzamide;
4-Hydroxy-N-[(trans-1-hydroxy-4-{[(5-methylpyridin-2-yl)oxy]methyl}cyclohexyl)methyl]benzamide;
N-[(trans-4-Benzyl-1-hydroxycyclohexyl)methyl]-4-hydroxybenzamide;
3-Fluoro-N-[(trans-4-{[(2-fluorobenzyl)oxy]methyl}-1-hydroxycyclohexyl)methyl]-4-hydroxybenzamide;
6-Hydroxy-N-{[cis-4-(2-phenethoxy)cyclohexyl]methyl}nicotinamide;
N-{[cis-4-(2-Phenylethoxy)cyclohexyl]methyl}-1H-pyrazole-4-carboxamide;
N-{[cis-4-(Phenoxymethyl)cyclohexyl]methyl}-1H-pyrazole-4-carboxamide;
N-{[cis-4-(2-Phenoxyethyl)cyclohexyl]methyl}-1H-pyrazole-4-carboxamide;
N-({cis-4-[(3-Fluorophenoxy)methyl]cyclohexyl}methyl)-1H-pyrazole-4-carboxamide;
N-({cis-4-[(4-Fluorophenoxy)methyl]cyclohexyl}methyl)-1H-pyrazole-4-carboxamide;
N-({(2R,5R)-5-[(4-Fluorophenoxy)methyl]tetrahydro-2H-pyran-2-yl}methyl)-1H-pyrazole-4-carboxamide;
N-{[cis-4-(4-Methoxybenzyl)cyclohexyl]methyl}-1H-pyrazole-4-carboxamide;
3-Amino-N-[(cis-4-benzylcyclohexyl)methyl]-1H-pyrazole-4-carboxamide;
N-({(2R,5R)-5-[(4-Chlorophenoxy)methyl]tetrahydro-2H-pyran-2-yl}methyl)-1H-pyrazole-4-carboxamide;
3-Amino-N-({(2R,5R)-5-[(4-fluorophenoxy)methyl]tetrahydro-2H-pyran-2-yl}methyl)-1H-pyrazole-4-carboxamide;
3-Amino-N-({(2R,5R)-5-[(4-chlorophenoxy)methyl]tetrahydro-2H-pyran-2-yl}methyl)-1H-pyrazole-4-carboxamide; and
3-Amino-N-({(2R,5R)-5-[(4-ethylphenoxy)methyl]tetrahydro-2H-pyran-2-yl}methyl)-1H-pyrazole-4-carboxamide;
or a pharmaceutically acceptable salt or solvate thereof.

A suitable sub-formula of compounds of formula (I) may be represented by formula (Ia) 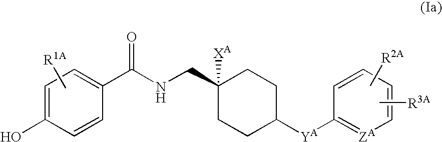
or a pharmaceutically acceptable salt or solvate thereof, wherein:
R^{1A}, R^{2A} or R^{3A} are independently selected from hydrogen, halogen, C₁₋₆alkyl, C₁₋₆alkoxy, C₁₋₆haloalkyl or C₃₋₈ cycloalkyl;
X^{A} is hydrogen or hydroxy;
Y^{A} is oxy, a 1-4 membered alkylene group, a 2-4 membered alkylene ether group or an oxyethyleneoxy group; and
Z^{A} is C or N.

Pharmaceutically acceptable salts of the compounds of formula (I) include the base salts thereof. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. For a review on suitable salts, see “Handbook of Pharmaceutical Salts: Properties, Selection, and Use” by Stahl and Wermuth (Wiley-VCH, Weinheim, Germany, 2002).

A pharmaceutically acceptable salt of a compound of formula (I) may be readily prepared by mixing together solutions of the compound of formula (I) and the desired base, as appropriate. The salt may precipitate from solution and be collected by filtration or may be recovered by evaporation of the solvent. The degree of ionisation in the salt may vary from completely ionised to almost non-ionised.

The compounds of the invention may exist in both unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

Included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components, which may be in stoichiometric or non-stoichiometric amounts. The resulting complexes may be ionised, partially ionised, or non-ionised. For a review of such complexes, see J Pharm Sci, 64 (8), 1269-1288 by Haleblian (August 1975).

Hereinafter all references to compounds of formula (I) include references to salts, solvates and complexes thereof and to solvates and complexes of salts thereof.

The compounds of the invention include compounds of formula (I) as hereinbefore defined, polymorphs, prodrugs, and isomers thereof (including optical, geometric and tautomeric isomers) as hereinafter defined and isotopically-labeled compounds of formula (I).

As stated, the invention includes all polymorphs of the compounds of formula (I) as hereinbefore defined.

Also within the scope of the invention are so-called ‘prodrugs’ of the compounds of formula (I). Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in ‘Pro-drugs as Novel Delivery Systems, Vol. 14, ACS Symposium Series (T Higuchi and W Stella) and ‘Bioreversible Carriers in Drug Design’, Pergamon Press, 1987 (ed. E B Roche, American Pharmaceutical Association).

Prodrugs in accordance with the invention can, for example, be produced by replacing appropriate functionalities present in the compounds of formula (I) with certain moieties known to those skilled in the art as ‘pro-moieties’ as described, for example, in “Design of Prodrugs” by H Bundgaard (Elsevier, 1985).

Some examples of prodrugs in accordance with the invention include:
(i) where the compound of formula (I) contains an alcohol functionality (—OH), an ether thereof, for example, replacement of the hydrogen with (C₁-C₆)alkanoyloxymethyl; and
(ii) where the compound of formula (I) contains a primary or a secondary amino functionality (NHR where R≠H), an amide thereof, for example, replacement of one or both hydrogens with (C₁-C₁₀)alkanoyl.

Further examples of replacement groups in accordance with the foregoing examples and examples of other prodrug types may be found in the aforementioned references.

Compounds of formula (I) containing one or more asymmetric carbon atoms can exist as two or more stereoisomers. Where a compound of formula (I) contains an alkenyl or alkenylene group, geometric cis/trans (or Z/E) isomers are possible. Where the compound contains, for example, a keto or oxime group or an aromatic moiety, tautomeric isomerism (‘tautomerism’) can occur. It follows that a single compound may exhibit more than one type of isomerism.

Included within the scope of the present invention are all stereoisomers, geometric isomers and tautomeric forms of the compounds of formula (I), including compounds exhibiting more than one type of isomerism, and mixtures of one or more thereof.

Cis/trans isomers may be separated by conventional techniques well known to those skilled in the art, for example, chromatography and fractional crystallisation. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic moiety, a suitable base. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Stereoisomeric conglomerates may be separated by conventional techniques known to those skilled in the art—see, for example, “Stereochemistry of Organic Compounds” by E L Eliel (Wiley, New York, 1994).

The present invention includes all pharmaceutically acceptable isotopically-labelled compounds of formula (I) wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

The compounds of the present invention are antagonists of NMDA (N-methyl-D-aspartate) NR2B receptor, and have a number of therapeutic applications, particularly in the treatment of pain, stroke, traumatic brain injury, Parkinson's disease, Alzheimer's disease, depression, anxiety, migraine, or the like.

The compounds of the present invention are useful for the general treatment of pain, particularly neuropathic pain. Physiological pain is an important protective mechanism designed to warn of danger from potentially injurious stimuli from the external environment. The system operates through a specific set of primary sensory neurones and is exclusively activated by noxious stimuli via peripheral transducing mechanisms (Millan 1999 Prog. Neurobio. 57: 1-164 for an integrative Review). These sensory fibres are known as nociceptors and are characterised by small diameter axons with slow conduction velocities. Nociceptors encode the intensity, duration and quality of noxious stimulus and by virtue of their topographically organised projection to the spinal cord, the location of the stimulus. The nociceptors are found on nociceptive nerve fibres of which there are two main types, A-delta fibres (myelinated) and C fibres (non-myelinated). The activity generated by nociceptor input is transferred after complex processing in the dorsal horn, either directly or via brain stem relay nuclei to the ventrobasal thalamus and then on to the cortex, where the sensation of pain is generated.

Intense acute pain and chronic pain may involve the same pathways driven by pathophysiological processes and as such cease to provide a protective mechanism and instead contribute to debilitating symptoms associated with a wide range of disease states. Pain is a feature of many trauma and disease states. When a substantial injury, via disease or trauma, to body tissue occurs the characteristics of nociceptor activation are altered. There is sensitisation in the periphery, locally around the injury and centrally where the nociceptors terminate. This leads to hypersensitivity at the site of damage and in nearby normal tissue. In acute pain these mechanisms can be useful and allow for the repair processes to take place and the hypersensitivity returns to normal once the injury has healed. However, in many chronic pain states, the hypersensitivity far outlasts the healing process and is normally due to nervous system injury. This injury often leads to maladaptation of the afferent fibres (Woolf & Salter 2000 Science 288: 1765-1768). Clinical pain is present when discomfort and abnormal sensitivity feature among the patient's symptoms. Patients tend to be quite heterogeneous and may present with various pain symptoms. There are a number of typical pain subtypes: 1) spontaneous pain which may be dull, burning, or stabbing; 2) pain responses to noxious stimuli are exaggerated (hyperalgesia); 3) pain is produced by normally innocuous stimuli (allodynia) (Meyer et al., 1994 Textbook of Pain 13-44). Although patients with back pain, arthritis pain, CNS trauma, or neuropathic pain may have similar symptoms, the underlying mechanisms are different and, therefore, may require different treatment strategies. Therefore pain can be divided into a number of different areas because of differing pathophysiology, these include nociceptive, inflammatory, neuropathic pain etc. It should be noted that some types of pain have multiple aetiologies and thus can be classified in more than one area, e.g. Back pain, Cancer pain have both nociceptive and neuropathic components.

Nociceptive pain is induced by tissue injury or by intense stimuli with the potential to cause injury. Pain afferents are activated by transduction of stimuli by nociceptors at the site of injury and sensitise the spinal cord at the level of their termination. This is then relayed up the spinal tracts to the brain where pain is perceived (Meyer et al., 1994 Textbook of Pain 13-44). The activation of nociceptors activates two types of afferent nerve fibres. Myelinated A-delta fibres transmitted rapidly and are responsible for the sharp and stabbing pain sensations, whilst unmyelinated C fibres transmit at a slower rate and convey the dull or aching pain. Moderate to severe acute nociceptive pain is a prominent feature of, but is not limited to pain from strains/sprains, post-operative pain (pain following any type of surgical procedure), posttraumatic pain, burns, myocardial infarction, acute pancreatitis, and renal colic. Also cancer related acute pain syndromes commonly due to therapeutic interactions such as chemotherapy toxicity, immunotherapy, hormonal therapy and radiotherapy. Moderate to severe acute nociceptive pain is a prominent feature of, but is not limited to, cancer pain which may be tumour related pain, (e.g. bone pain, headache and facial pain, viscera pain) or associated with cancer therapy (e.g. postchemotherapy syndromes, chronic postsurgical pain syndromes, post radiation syndromes), back pain which may be due to herniated or ruptured intervertabral discs or abnormalities of the lumber facet joints, sacroiliac joints, paraspinal muscles or the posterior longitudinal ligament

Neuropathic pain is defined as pain initiated or caused by a primary lesion or dysfunction in the nervous system (IASP definition). Nerve damage can be caused by trauma and disease and thus the term ‘neuropathic pain’ encompasses many disorders with diverse aetiologies. These include but are not limited to, diabetic neuropathy, post herpetic neuralgia, back pain, cancer neuropathy, HIV neuropathy, Phantom limb pain, Carpal Tunnel Syndrome, chronic alcoholism, hypothyroidism, trigeminal neuralgia, uremia, or vitamin deficiencies. Neuropathic pain is pathological as it has no protective role. It is often present well after the original cause has dissipated, commonly lasting for years, significantly decreasing a patients quality of life (Woolf and Mannion 1999 Lancet 353: 1959-1964). The symptoms of neuropathic pain are difficult to treat, as they are often heterogeneous even between patients with the same disease (Woolf & Decosterd 1999 Pain Supp. 6: S141-S147; Woolf and Mannion 1999 Lancet 353: 1959-1964). They include spontaneous pain, which can be continuous, or paroxysmal and abnormal evoked pain, such as hyperalgesia (increased sensitivity to a noxious stimulus) and allodynia (sensitivity to a normally innocuous stimulus).

The inflammatory process is a complex series of biochemical and cellular events activated in response to tissue injury or the presence of foreign substances, which result in swelling and pain (Levine and Taiwo 1994: Textbook of Pain 45-56). Arthritic pain makes up the majority of the inflammatory pain population. Rheumatoid disease is one of the commonest chronic inflammatory conditions in developed countries and rheumatoid arthritis is a common cause of disability. The exact aetiology of RA is unknown, but current hypotheses suggest that both genetic and microbiological factors may be important (Grennan & Jayson 1994 Textbook of Pain 397-407). It has been estimated that almost 16 million Americans have symptomatic osteoarthritis (OA) or degenerative joint disease, most of whom are over 60 years of age, and this is expected to increase to 40 million as the age of the population increases, making this a public health problem of enormous magnitude (Houge & Mersfelder 2002 Ann Pharmacother. 36: 679-686; McCarthy et al., 1994 Textbook of Pain 387-395). Most patients with OA seek medical attention because of pain. Arthritis has a significant impact on psychosocial and physical function and is known to be the leading cause of disability in later life. Other types of inflammatory pain include but are not limited to inflammatory bowel diseases (IBD),

Other Types of Pain Include but are not Limited to;

Musculo-skeletal disorders including but not limited to myalgia, fibromyalgia, spondylitis, sero-negative (non-rheumatoid) arthropathies, non-articular rheumatism, dystrophinopathy, Glycogenolysis, polymyositis, pyomyositis.
Central pain or ‘thalamic pain’ as defined by pain caused by lesion or dysfunction of the nervous system including but not limited to central post-stroke pain, multiple sclerosis, spinal cord injury, Parkinson's disease and epilepsy.
Heart and vascular pain including but not limited to angina, myocardical infarction, mitral stenosis, pericarditis, Raynaud's phenomenon, scleredoma, scleredoma, skeletal muscle ischemia.
Visceral pain, and gastrointestinal disorders. The viscera encompasses the organs of the abdominal cavity. These organs include the sex organs, spleen and part of the digestive system. Pain associated with the viscera can be divided into digestive visceral pain and non-digestive visceral pain. Commonly encountered gastrointestinal (GI) disorders include the functional bowel disorders (FBD) and the inflammatory bowel diseases (IBD). These GI disorders include a wide range of disease states that are currently only moderately controlled, including—for FBD, gastro-esophageal reflux, dyspepsia, the irritable bowel syndrome (IBS) and functional abdominal pain syndrome (FAPS), and—for IBD, Crohn's disease, ileitis, and ulcerative colitis, which all regularly produce visceral pain. Other types of visceral pain include the pain associated with dysmenorrhea, pelvic pain, cystitis and pancreatitis.
Head pain including but not limited to migraine, migraine with aura, migraine without aura cluster headache, tension-type headache.
Orofacial pain including but not limited to dental pain, temporomandibular myofascial pain.

Thus, as a yet further aspect of the present invention, there is provided the use of a compound of formula (I), or a pharmaceutically acceptable salt or solvate thereof, in the manufacture of a medicament for the treatment of pain, particularly neuropathic pain.

As an alternative aspect, there is provided a method for the treatment of pain, particularly neuropathic pain, comprising administration of a therapeutically effective amount of a compound of formula (I), or a pharmaceutically acceptable salt or solvate thereof, to a mammal in need of said treatment.

General Synthesis

All of the compounds of the formula (I) can be prepared by the procedures described in the general methods presented below or by the specific methods described in the Examples section and the Preparations section, or by routine modifications thereof. The present invention also encompasses any one or more of these processes for preparing the compounds of formula (I), in addition to any novel intermediates used therein.

In the following general methods, Cy, R¹, R², R³, A, B, n, p, X, Y and Z are as previously defined for a compound of the formula (I), unless otherwise stated. The methods exemplify preparation of compounds of formula (I) where Y and X are in the trans configuration. It will be appreciated by those skilled in the art that compounds having a cis configuration may be prepared from the appropriate regiospecific starting materials or by separation of the alternative regioisomer from a mixture of cis and trains intermediates or final compounds.

According to process (A), a compound of the formula (I), where Y is —O— or —(CH₂)_{q}O— and q is 1-3, may be prepared by the reaction of a compound of the formula (IIa) or (IIb) with a compound of the formula (III) 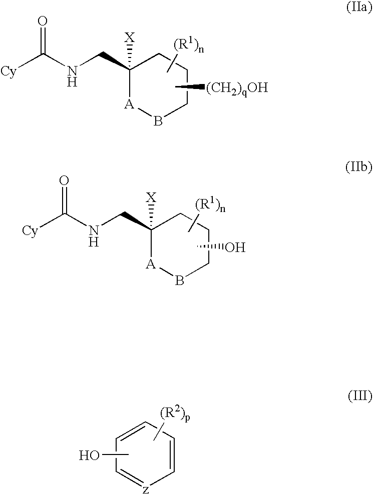
under standard Mitsunobu-type conditions, e.g. diisopropyl azodicarboxylate and Ph₃P, in a suitable solvent such as tetrahydrofuran.

According to process (B), a compound of the formula (I), where Y is —(CH₂)ᵣOCH₂— and r is 0-2, may be prepared by the reaction of a compound of the formula (IIc), with a compound of the formula (IV) 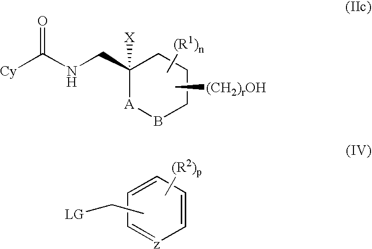
where LG is a suitable leaving group, such as bromide, using a suitable base such as sodium hydride, in a suitable solvent such as dimethyl formamide.

According to process (C), a compound of the formula (I), where Z is N, Y is —(CH₂)ᵣO— and r is 0-3, may be prepared by the reaction of a compound of the formula (IIc), where r is 0-3, with a compound of the formula (IVa) 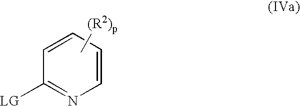
where LG is a suitable leaving group, e.g. halogen, under suitable alkylating conditions, e.g. sodium hydride in a suitable solvent, such as DMF, at elevated temperature and in the presence of microwaves.

A compound of formula (IIa-c) may be prepared by reaction of a compound of formula (Va) or (Vb) with a compound of formula (VI) 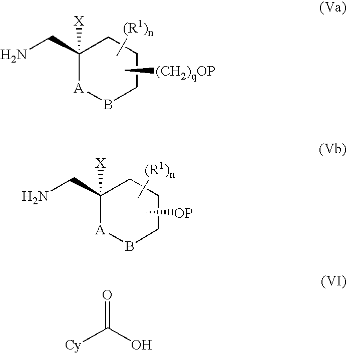
where q is 1-3, P is a suitable hydroxy protecting group, e.g. benzyl, under standard acid/amine coupling conditions, e.g. using N-ethyl-N′-(3-dimethylaminopropyl)carbodiimide and 1-hydroxybenzotriazole (HOBT), in a suitable solvent such as dimethyl formamide, followed by removal of the P group under standard conditions, e.g. by hydrogenation.

A compound of formula (Va) or (Vb), where X is H, may be prepared from a compound of formula (VIIa) or (VIIb) 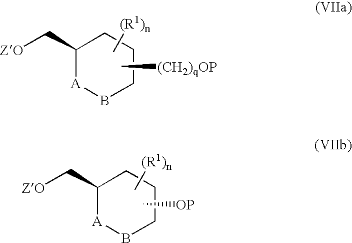
where P is as defined above and Z′O is a suitable leaving group such as mesylate or tosylate, by treatment with a suitable azide, such as sodium azide, in a suitable solvent such as dimethyl formamide, at elevated temperature, followed by reduction of the azide to amino under standard conditions, such as hydrogenation.

A compound of formula (Va) or (Vb), where A=B=C and X is OH, may be prepared from a compound of formula (VIII) 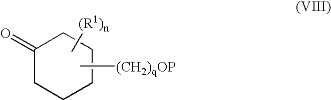
by treatment with a suitable cyanide, such as trimethylsilyl cyanide, with zinc iodide in toluene at reduced temperature, followed by reduction of the resulting cyano group with a suitable reducing agent, such as lithium aluminium hydride, and separation of the desired cis or trans-isomer.

Compounds of formula (VIIa) and (VIIb) may be prepared from a compound of formula (IXa) or (IXb) 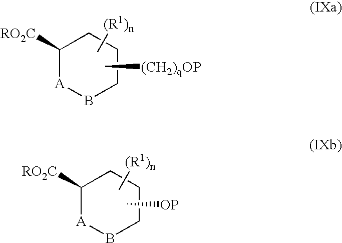
where R is a suitable ester group, e.g. methyl, by reduction with a suitable agent, e.g. lithium aluminium hydride, follwed by activation of the hydroxy group with Z′ under suitable conditions.

According to process (D), a compound of the formula (I) may be prepared by the reaction of a compound of the formula (VI), with a compound of the formula (X) 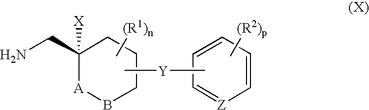
under standard acid/amine coupling conditions, such as N-ethyl-N′-(3-dimethylaminopropyl)carbodiimide and 1-hydroxybenzotriazole (HOBT) in a suitable solvent, such as dimethyl formamide.

A compound of formula (X), where A=B=C and X is OH, may be prepared from a compound of formula (XI) 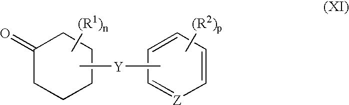
by reaction with a suitable cyanide compound, such as trimethylsilyl cyanide, with zinc iodide in a suitable solvent, such as toluene, at reduced temperature, followed by reduction with a suitable reducing agent, such as lithium aluminium hydride, and separation of the desired isomer.

A compound of formula (X), where X is H, may be prepared from a compound of formula (XII) 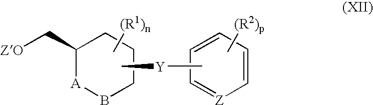
where Z′O is defined above, by treatment with a suitable azide, such as sodium azide, in a suitable solvent such as dimethyl formamide, at elevated temperature, followed by reduction of the azide to amino under standard conditions, such as hydrogenation.

A compound of formula (XII) may be prepared from a compound of formula (XIIa) 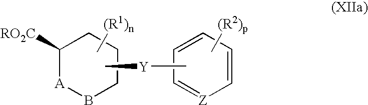
where R is a suitable ester group, e.g. methyl, by reduction with a suitable agent, e.g. lithium aluminium hydride, follwed by activation of the hydroxy group with Z′ under suitable conditions.

A compound of formula (X) where Y is —(CH₂)_{q}O— and q is 1-3, may be prepared by reaction of a compound of formula (IV) with a compound of formula (XIIIa) or (XIIIb) 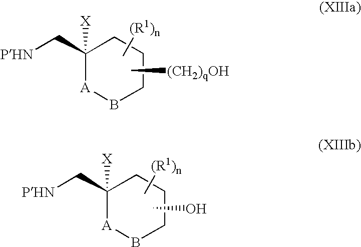
where P′ is a suitable N-protecting group, such as Boc, under Mitsunobu type conditions, as described above, followed by deprotection of the P′ group under standard conditions.

A compound of formula (X) where Y is —(CH₂)ᵣOCH₂— and r is 0-2, may be prepared by reaction of a compound of formula (IV) with a compound of formula (XIIIc) 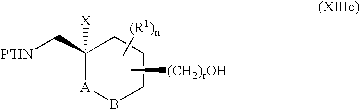
where P′ is defined above, under standard nucleophilic displacement conditions, as described above, followed by deprotection of the P′ group under standard conditions.

Compounds of formula (XIIIa-c), where A=B=C and X is OH, may be prepared from a compound of formula (XIV) 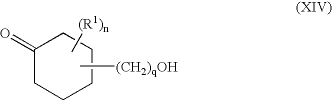
where q is 0-3, by reaction with a suitable cyanide compound, such as trimethylsilyl cyanide, with zinc iodide in a suitable solvent, such as toluene, at reduced temperature, followed by reduction with a suitable reducing agent, such as lithium aluminium hydride, and separation of the desired cis or trans-isomer.

A compound of formula (XIIIa-c), where X is H, may be prepared from a compound of formula (Va) or (Vb) by selective protection of the amino group with a suitable protecting group P′ followed by selective deprotection of the protecting group P.

A compound of formula (X), where X is H, may be prepared from a compound of formula (XI) by nitromethylation using nitromethane with a catalytic amount of ethylenediamine at elevated temperature followed by sequential reduction of the resulting nitro group and double bond under standard conditions.

A compound of formula (XI), where Y is —O— or —(CH₂)_{q}O— and q is 1-3, or Y is oxyethyleneoxy, may be prepared by reaction of a compound of formula (III) with a compound of formula (XVa) or (XVb), as appropriate 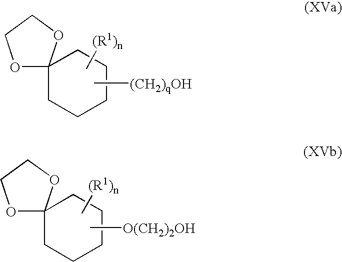
under Mitsunobu type conditions, as described above, followed by deprotecton of the ketone group under standard conditions.

A compound of formula (XI), where Y is —(CH₂)ᵣOCH₂— and r is 0-2 or Y is oxyethyleneoxy, may be prepared by reaction of a compound of formula (XVc) with a compound of formula (IV) or (IVa), as appropriate 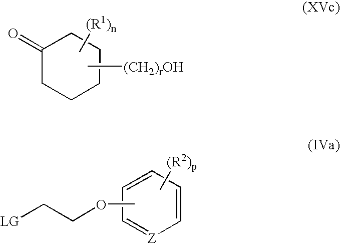
under standard nucleophilic displacement conditions, as described above.

A compound of formula (XI) where Y is a 1-4 membered alkylene may be prepared by reaction of a compound of formula (XVd) with a compound of formula (XVe) 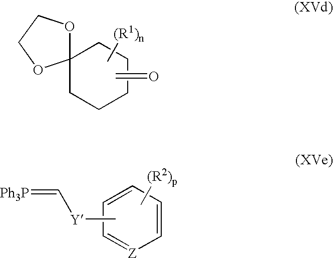
where Y′ is a covalent bond or a 1-3 membered alkylene, under Wittig reaction conditions, followed by hydrogenation of the resulting double bond using a suitable metal catalyst, e.g. PD(OH)₂ on carbon in a suitable solvent such as methanol, followed by deprotection of the keto group under suitable conditions.

A compound of formula (XI) where Y is —(CH₂)_{q}OCH₂CH₂— and q is 0-1, may be prepared by reaction of a compound of formula (XVa), where q is 0-1, with a compound of formula (XVI) 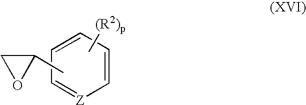
using a suitable base, such as sodium hydride, in a suitable solvent, such as dimethyl formamide, followed by acetylation then reduction of the OH group under standard conditions, followed by deprotection of the ketone group.

A compound of formula (XII), where A=B=C and Y is —O(CH₂)₂— may be prepared by reaction of compound of formula (XVII) with a compound of formula (XVIII) 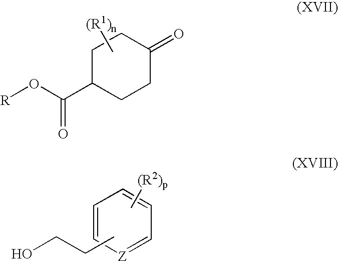
where R is a suitable carboxylic acid ester protecting group, e.g. methyl, by treatment with p-toluenesulfonic acid in benzene followed by removal of one of the ether groups using, e.g. triethylsilane and trimethylsilyl triflate, followed by reduction of the ester under standard conditions, e.g. with lithium aluminium hydride, then activation of the hydroxy group with Z′ under standard conditions.

A compound of formula (XII) where A=O and B=C, may be prepared from a compound of formula (XIX) 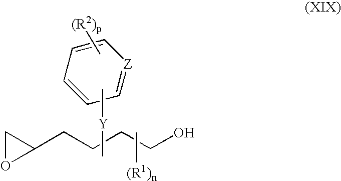
by treatment with a suitable agent, such as p-toluenesulfonic acid, in a suitable solvent such as dichloromethane.

A compound of formula (VIIa) where A=O and B=C, may be prepared from a compound of formula (XX) 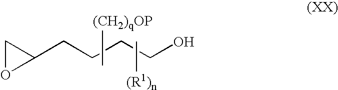
where P is defined above, by treatment with a suitable agent, such as p-toluenesulfonic acid, in a suitable solvent such as dichloromethane, followed by deprotection of the protecting group.

According to a fifth process (E), a compound of formula (I), where A=B=C and Y represents a 1-4 membered alkylene, may be prepared by reaction of a compound of formula (XXI): 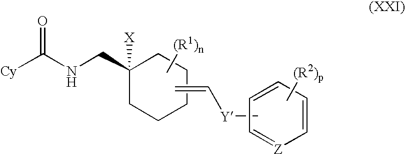
where Y′ represents a covalent bond or a 1-3membered alkylene, by hydrogenation of the double bond using a suitable metal catalyst, e.g. Pd(OH)₂ on carbon in a suitable solvent such as methanol.

A compound of formula (XXI) may be prepared by reaction of a compound of formula (XXII) with a compound of formula (XVe) as described above 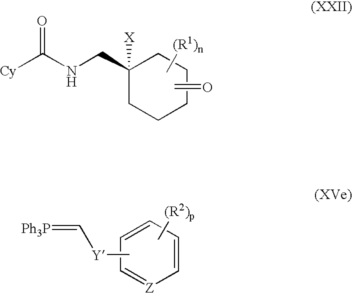
under Wittig reaction conditions.

A compound of formula (XXII) may be prepared by reaction of a compound of formula (XXIII) with a compound of formula (VI): 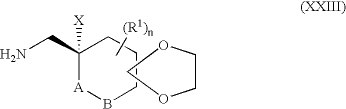
under suitable acid amine coupling conditions as described above, followed by deprotection of the ketone group under suitable conditions.

Compounds of formulae (III), (IV), (VI), (VIII), (IX), (XIIc), (XIV), (XV), (XVI), (XVII), (XVIII), (XIX), (XX) and (XXIII) are known in the art or may be prepared by well-known methods.

The use of protecting groups as described is well-known in the art. Suitable protecting groups for use in the afore-mentioned processes may be referenced in ‘Protecting Groups in Organic Synthesis’, Greene and Wuts, 3^{rd} Edition, John Wiley and Sons, Inc.

The various general methods described above may be useful for the introduction of the desired groups at any stage in the stepwise formation of the required compound, and it will be appreciated that these general methods can be combined in different ways in such multi-stage processes. The sequence of the reactions in multi-stage processes should of course be chosen so that the reaction conditions used do not affect groups in the molecule which are desired in the final product.

Compounds of the invention intended for pharmaceutical use may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

They may be administered alone or in combination with one or more other compounds of the invention or in combination with one or more other drugs (or as any combination thereof). Generally, they will be administered as a formulation in association with one or more pharmaceutically acceptable excipients. The term “excipient” is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in ‘Remington's Pharmaceutical Sciences’, 19th Edition (Mack Publishing Company, 1995).

Oral Administration

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986 by Liang and Chen (2001).

For tablet dosage forms, depending on dose, the drug may make up from 1 wt % to 80 wt % of the dosage form, more typically from 5 wt % to 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 wt % to 5 wt % of the tablet, and glidants may comprise from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 wt % to 10 wt %, preferably from 0.5 wt % to 3 wt % of the tablet.

Other possible ingredients include anti-oxidants, colourants, flavouring agents, preservatives and taste-masking agents.

Exemplary tablets contain up to about 80% drug, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in “Pharmaceutical Dosage Forms: Tablets, Vol. 1”, by H. Lieberman and L. Lachman, Marcel Dekker, N.Y., N.Y., 1980 (ISBN 0-8247-6918-X).

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Verma et al, Pharmaceutical Technology On-line, 25(2), 1-14 (2001). The use of chewing gum to achieve controlled release is described in WO 00/35298.

Parenteral Administration

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and PGLA microspheres.

Topical Administration

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958 by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

Inhaled/Intranasal Administration

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or HPMC), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula (I), propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, poly(DL-lactic-coglycolic acid (PGLA). Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a suitable metered dose or “puff” containing the compound of formula (I), which may be administered in a single dose or, more usually, as divided doses throughout the day.

Rectal/Intravaginal Administration

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

Ocular/Aural Administration

The compounds of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

Other Technologies

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

Thus, as a yet further or alternative aspect, the invention provides a pharmaceutical composition including a compound of formula (I), or a pharmaceutically acceptable salt or solvate thereof, together with a suitable excipient. The composition is useful in the treatment of a disease for which an NMDA NR²B antagonist is indicated, particularly pain, stroke, traumatic brain injury, Parkinson's disease, Alzheimer's disease, depression, anxiety and migraine.

Kit-of-Parts

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions.

Thus, the kit of the invention comprises two or more separate pharmaceutical compositions, at least one of which contains a compound of formula (I) in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

Dosage

For administration to human patients, the total daily dose of the compounds of the invention is typically in the range 0.1 mg to 1000 mg depending, of course, on the mode of administration. The quantity of active component in a unit dose preparation may be varied or adjusted from 0.1 mg to 1 g according to the particular application and the potency of the active components. In medical use the drug may be administered one to three times daily as, for example, capsules of 100 or 300 mg. In therapeutic use, the compounds utilized in the pharmaceutical method of this invention are administered at the initial dosage of about 0.01 mg to about 100 mg/kg daily. A daily dose range of about 0.01 mg to about 100 mg/kg is preferred.

These dosages are based on an average human subject having a weight of about 65 kg to 70 kg. The physician will readily be able to determine doses for subjects whose weight falls outside this range, such as infants and the elderly.

For the avoidance of doubt, references herein to “treatment” include references to curative, palliative and prophylactic treatment.

The biological activity and safety profile of the compounds of the formula (I) to may be measured using the assays described below.

NR2B Binding Assay

The activity of the cycloalkylene amide compounds of the present invention, as NR2B antagonists, is determined by their ability to inhibit the binding of NR2B subunit at its receptor sites employing radioactive ligands.

The NR²B antagonist activity of the cycloalkylene amide compounds is evaluated by using the standard assay procedure described in, for example, J. Pharmacol., 331, pp 117-126, 1997. This method essentially involves determining the concentration of the individual compound required to reduce the amount of radiolabelled NR2B ligands by 50% at their receptor sites, thereby affording characteristic IC₅₀ values for each compound tested. More specifically, the assay is carried out as follows.

Membranes were prepared by homogenization of forebrain of male CD rats weighing between 170˜190 g by using glass-Teflon homogenizer in 0.32 M sucrose at 4° C. The crude nuclear pellet was removed by centrifugation at 1000×g for 10 min, and the supernatant centrifuged at 17000×g for 25 min. The resulting pellet was resuspended in 5 mM Tris acetate pH 7.4 at 4° C. for 10 min to lyse cellular particles and again centrifuged at 17000×g. The resulting pellet (P2 membrane) was washed twice in Tris acetate, resuspended at 5.5 mg protein/ml and stored at −20° C. until use. All the manipulation was done on ice, and stock solution and equipment were kept on ice at all time.

For the saturation assay, receptor saturation was determined by incubating [³H]-1-[(1S*,2S*)-2-hydroxy-2-(4-hydroxyphenyl)-1-methylethyl]-4-phenylpiperidin-4-ol and 50 μg protein of P2 membrane for 60 minutes at room temperature in a final 100 μl of incubation buffer (50 mM Tris HCl, pH7.4). Total and non-specific bindings (in the presence of 10 μM of unlabeled 1-[(1S*,2S*)-2-hydroxy-2-(4-hydroxyphenyl)-1-methylethyl]-4-phenylpiperidin-4-ol) were determined in a range of [³H]-1-[(1S*,2S*)-2-hydroxy-2-(4-hydroxyphenyl)-1-methylethyl]-4-phenylpiperidin-4-ol concentrations (0.625 nM to 60 nM).

For the competition assay, test compounds were incubated in duplicate with 5 nM [³H]-1-[(1S*,2S*)-2-hydroxy-2-(4-hydroxyphenyl)-1-methylethyl]-4-phenylpiperidin-4-ol and 50 μg protein of P2 membrane for 60 minutes at room temperature in a final 100 μl of 50 mM Tris HCl buffer (pH7.4). Nonspecific binding was determined by 10 μM of unlabeled 1-[(1S*,2S*)-2-hydroxy-2-(4-hydroxyphenyl)-1-methylethyl]-4-phenylpiperidin-4-ol (25 μl). The saturation derived K_{D} gained in saturation assay was used for all Ki calculations.

All incubations were terminated by rapid vacuum filtration over 0.2% polyethyleneimine soaked Whatman GF/B glass fibre filter paper using a SKATRON cell harvester followed by three washes with ice-cold filtration buffer (5 mM Tris HCl, pH 7.4.). Receptor-bound radioactivity was quantified by liquid scintillation counting using Packard LS counter. Competition assays were performed by counting Wallac GF/B filters on Betaplate scintillation counter (Wallac).

The compound prepared in the working example 11 as described below was tested by this method, and showed a Ki value of 6.2 nM with respect to binding affinity for the NR2B receptor. In this test, the compounds of the present invention exhibited excellent binding activity for the NR2B receptor.

Human NR2B Cell Functional Assay

HEK293 cells stably expressing human NR1b/2B receptor were used for cell functional assay. Cells were grown in 75-cm² culture flasks, using Dulbecco's modified Eagle's medium (DMEM, high glucose) supplemented with 10% fetal bovine, 52 μg/ml Zeocin, 530 μg/ml Geneticin, 100 units/ml penicillin and 100 μg/ml streptomycin. Cells were maintained in a humidified atmosphere in 5% CO₂ at 37° C., and 50-60% confluent cells were harvested by 0.05% trypsin containing 0.53 mM EDTA. The day before the experiment, expression of NR1b/2B receptor was induced by 5 μM ponasteron A in DMEM (40 ml) in the presence of 400 μM ketamine to prevent excitotoxicity. The induction was performed for 19-24 hours, using 50-60% confluent cells.

Cells were washed with 10 ml of Ca²⁺-free Krebs-Ringer Hepes buffer (KRH) containing 400 μM ketamine, and the loading of 5 μM fura-2 acetoxymethyl ester was made for 2 hrs at room temperature in the presence of 400 μM ketamine in Ca²⁺-free KRH (10 ml). Subsequently, cells were collected in 50 ml tube by pipetting manipulation and centrifuged at 850 rpm for 2 min. Supernatant was removed, and cells were washed with 10 ml of Ca²⁺-free KRH buffer, followed by centrifugation again. This manipulation was repeated 4 times to remove ketamine, glutamate and glycine. Cells were re-suspended in Ca²⁺-free KRH buffer, and 50 μl of cell suspension was addesud to each well of 96-well plates at a density of 100,000 cells/well, followed by adding test compounds dissolved in 50 μl of Ca²⁺-free KRH. After pre-incubation for 30 min, agonists (final 100 μM glutamic acid and 10 □M glycine) dissolved in 25 μl of KRH containing 9 mM Ca²⁺ (final 1.8 mM) were added. Fura-2 fluorescence (excitation wavelengths: 340 nm and 380 nm; emission wavelengths 510-520 nm) was monitored with a fluorescence imaging system, FDSS6000. The □ fluorescence ratio F340/F380 (i.e., the fluorescence ratio immediately post-agonist—the basal fluorescence ratio; calculated as AUC) was used for evaluation of drug effects on agonists-induced changes in intracellular Ca²⁺. The basal fluorescence ratio was determined in the presence of 10 μM MK-801.

Rat Haloperidol-Induced Catalepsy Assay

Fasted male CD rats were used (7-8 weeks old). Test compound or vehicle was given subcutaneously then haloperidol 0.5 mg/kg s.c. Sixty minutes after haloperidol-injection, the duration of catalepsy was quantified by placing the animals forepaws on an elevated bar and determining the latency to remove both forepaws from the bar. The cutoff latency was 60 seconds. The experimenter was blind to treatments during testing.

Human Dofetilide Binding

Human HERG transfected HEK293S cells were prepared and grown in-house. The collected cells were suspended in 50 mM Tris-HCl (pH 7.4 at 4° C.) and homogenized using a hand held Polytron PT 1200 disruptor set at full power for 20 sec on ice. The homogenates were centrifuged at 48,000×g at 4° C. for 20 min. The pellets were then resuspended, homogenized, and centrifuged once more in the same manner. The final pellets were resuspended in an appropriate volume of 50 mM Tris-HCl, 10 mM KCl, 1 mM MgCl₂ (pH 7.4 at 4° C.), homogenized, aliquoted and stored at −80° C. until use. An aliquot of membrane fractions was used for protein concentration determination using BCA protein assay kit (PIERCE) and ARVOsx plate reader (Wallac).

Binding assays were conducted in a total volume of 200 μl in 96-well plates. Twenty μl of test compounds were incubated with 20 μl of [³H]-dofetilide (Amersham, final 5 nM) and 160 μl of membrane homogenate (25 μg protein) for 60 minutes at room temperature. Nonspecific binding was determined by 10 μM dofetilide at the final concentration. Incubation was terminated by rapid vacuum filtration over 0.5% presoaked GF/B Betaplate filter using Skatron cell harvester with 50 mM Tris-HCl, 10 mM KCl, 1 mM MgCl₂, pH 7.4 at 4° C. The filters were dried, put into sample bags and filled with Betaplate Scint. Radioactivity bound to filter was counted with Wallac Betaplate counter.

I_{HERG} Assay

HEK 293 cells which stably express the HERG potassium channel were used for electrophysiological study. The methodology for stable transfection of this channel in HEK cells can be found elsewhere (Z. Zhou et al., 1998, Biophysical journal, 74, pp 230-241). Before the day of experimentation, the cells were harvested from culture flasks and plated onto glass coverslips in a standard MEM medium with 10% FCS. The plated cells were stored in an incubator at 37° C. maintained in an atmosphere of 95% O₂/5% CO₂. Cells were studied between 15-28 hrs after harvest.

HERG currents were studied using standard patch clamp techniques in the whole-cell mode. During the experiment the cells were superfused with a standard external solution of the following composition (mM); NaCl, 130; KCl, 4; CaCl₂, 2; MgCl₂, 1; Glucose, 10; HEPES, 5; pH 7.4 with NaOH. Whole-cell recordings was made using a patch clamp amplifier and patch pipettes which have a resistance of 1-3MOhm when filled with the standard internal solution of the following composition (mM); KCl, 130; MgATP, 5; MgCl₂, 1.0; HEPES, 10; EGTA 5, pH 7.2 with KOH. Only those cells with access resistances below 15MΩ and seal resistances >1GΩ was accepted for further experimentation. Series resistance compensation was applied up to a maximum of 80%. No leak subtraction was done. However, acceptable access resistance depended on the size of the recorded currents and the level of series resistance compensation that can safely be used. Following the achievement of whole cell configuration and sufficient for cell dialysis with pipette solution (>5 min), a standard voltage protocol was applied to the cell to evoke membrane currents. The voltage protocol is as follows. The membrane was depolarized from a holding potential of −80 mV to +20 mV for 1000 ms. This was followed by a descending voltage ramp (rate 0.5 mV msec⁻¹) back to the holding potential. The voltage protocol was applied to a cell continuously throughout the experiment every 4 seconds (0.25 Hz). The amplitude of the peak current elicited around −40 mV during the ramp was measured. Once stable evoked current responses were obtained in the external solution, vehicle (0.5% DMSO in the standard external solution) was applied for 10-20 min by a peristalic pump. Provided there were minimal changes in the amplitude of the evoked current response in the vehicle control condition, the test compound of either 0.3, 1, 3, 10 μM was applied for a 10 min period. The 10 min period included the time which supplying solution was passing through the tube from solution reservoir to the recording chamber via the pump. Exposing time of cells to the compound solution was more than 5 min after the drug concentration in the chamber well reached the attempting concentration. There reversibility. Finally, the cells was exposed to high dose of dofetilide (5 μM), a specific IKr blocker, to evaluate the insensitive endogenous current.

All experiments were performed at room temperature (23±1° C.). Evoked membrane currents were recorded on-line on a computer, filtered at 500-1 KHz (Bessel −3 dB) and sampled at 1-2 KHz using the patch clamp amplifier and a specific data analyzing software. Peak current amplitude, which occurred at around −40 mV, was measured off line on the computer.

The arithmetic mean of the ten values of amplitude was calculated under control conditions and in the presence of drug. Percent decrease of I_{N} in each experiment was obtained by the normalized current value using the following formula: I_{N}=(1−I_{D}/I_{C})×100, where I_{D} is the mean current value in the presence of drug and I_{C} is the mean current value under control conditions. Separate experiments were performed for each drug concentration or time-matched control, and arithmetic mean in each experiment is defined as the result of the study.

Mice PSL Method

Surgery of partial sciatic nerve ligation (PSL) was made according to Seltzer et al. (Pain 43, 1990, 205-218). Von Fray hair test was applied slowly to the plantar surface of the hind operated paw until the hairs bent. Each hair was tested 10 times in ascending order of force to different loci of the paw with one to two second intervals between each application. Once a withdrawal response was established, the paw was re-tested with the same hair. The lowest amount of force required to elicit a response was recorded as the paw-withdrawal threshold, measured in grams.

In Vitro Micronucleus Assay

In vitro micronucleus assay detects chemically induced micronucleus formation (chromosome breakage and/or whole chromosome loss) in vitro, by evaluating treated cultures of Chinese Hamster Ovary (CHO-WBL) cells. The growth medium is McCoy's 5A mediasupplemented with fetal bovine serum (FBS). Cells are incubated at approximately 37° C., 95% air/5% CO₂ in a humidified chamber. Compound is dissolved in DMSO (dimethylsulfoxide). The final volume of compound in the medium is 1%. The maximum concentration of compound should be at or near a cytotoxic level. With non-toxic compound a maximum of 5 mg/mL or the lowest precipitating concentration is used. Assay conditions include both direct assay and metabolic activation assay where the compound is tested in the presence of Aroclor 1254-induced rat liver S9 fraction. Cultures are initiated by seeding approximately 1×10⁴ exponentially growing CHO-WBL in McCoy's 5A medium into 8 well slide chamber. Twenty-four hours after the seeding, cells are treated with compounds. In direct assay, cells are treated with compound and Cytochalasin B for 24 hours. In metabolic activation assay, cells are treated with compound in the presence of rat liver S9 fraction for 3 hours, and then cells are incubated with a fresh medium including and Cytochalasin B for 21 hours. Approximately 24 hours from the initiation of treatment the cells are incubated in hypotonic buffer (75 mM KCl) for 5 min. After the hypotonic treatment, the cells are fixed in the fixative solution (MeOH:acetic acid=3:1 v/v) and stained with Acridine Orange. One hundred consecutive cells per concentration for the proportion of those with 1, 2 or ≧3 nuclei per cell and 1000 binucleated cells for the presence of micronuclei are analyzed (minimum 500 binucleated cells should be analyzed). A dose-dependent, two-fold or greater increase over negative control value is considered a positive response.

Serum Protein Binding

Serum protein binding of NR2B topic compounds (1 μM) in humans and ddY mice were measured in method of equilibrium dialysis using 96-well plate type equipment. Spectra-Por® regenerated cellulose membranes (molecular weight cut-off 12,000-14,000, 12 mm×120 mm) was soaked for over night in distilled water, then for 20 minutes in 30% ethanol, and finally for 15 minutes in dialysis buffer (0.10 M PBS: phosphate buffered saline, pH 7.4). Fresh humans and ddY mice serum (20 ml each) was prepared. The dialysis was assembled with being careful not to puncture or tear the membranes and added 150 μl of serum to one side of each well and 150 μl of dialysis buffer to the other side of each well. After 4 hours incubation at 37° C. for 60 r.p.m, remove the serum and buffer samples and an aliquot of collected serum and buffer samples were mixed for buffer and serum at following rates:
1) 40 μl serum samples were mixed with 120 μl buffer
2) 120 μl buffer samples were mixed with 40 μl serum
Then, mixed samples were extracted with 600 μl acetonitrile containing (2R,3R)-2-(diphenylmethyl)-N-(2-methoxybenzyl)quinuclidin-3-amine at 25 ng/ml (as HPLC-MS-MS internal standard) and measured in LC/MS/MS analysis.
   Calculations:
The fraction of substrate unbound, fᵤ=1−{([plasma]_{eq}−[buffer]_{eq})/([plasma]_{eq})} where [plasma]_{eq} and [buffer]_{eq} are the concentrations of substrate in plasma and buffer, respectively.
   Aqueous Solubility

Aqueous solubility in the mediums (a)-(c) was determined by method (1) or (2). (1) Vials containing approx. 1 mg of compound and 1 mL of each medium were agitated for 24 hours at room temperature. Insoluble materials were removed by centrifugation at 10,000 rpm for 10 minutes twice. The supernatants were assayed by HPLC. (2) Whatman Mini-UniPrep chambers (Clifton, N.J., USA) containing more than 0.5 mg of compound and 0.5 mL of each medium were shaken overnight (over 8 hours) at room temperature. All samples were filtered through a 0.45 μm PVDF membrane into a Whatman Mini-UniPrep plunger before analysis. The filtrates were assayed by HPLC.

<Mediums>:

(a) Simulated gastric fluid with no enzyme (SGN) at pH 1.2: Dissolve 2.0 g of NaCl in 7.0 mL of 10N HCl and sufficient water to make 1000 mL.
(b) Phosphate buffered saline (PBS) at pH 6.5: Dissolve 6.35 g of KH₂PO₄, 2.84 g of Na₂HPO₄ and 5.50 g of NaCl in sufficient water to make 1000 mL, adjusting the pH of this solution to 6.5.
(c) Water for injection (WFI).
   Human V1A Binding Assay

Cell paste of CHO cells expressing human V1a receptor was suspended in 3-fold volume of ice-cold wash buffer (50 mM Tris-HCl, 5 mM MgCl₂, protease inhibitors, adjusted pH 7.4). The cells were homogenized and centrifuged at 25,000 g for 30 minutes at 4° C. The pellet was re-suspended by homogenization in freezing buffer (50 mM Tris-HCl, 5 mM MgCl₂, 20% glycerol, adjusted pH 7.4). The membrane homogenate was stored at −80° C. until use. All the manipulation was done on ice, and stock solution and equipment were kept on ice at all time.

For the saturation assay, receptor saturation was determined by incubating 8-Arg[phenylalanyl-3,4,5-³H]-vasopressin (³H-AVP) and 20 μg protein of cell membrane for 60 minutes at 25° C. in a final 250 μl of incubation buffer (50 mM Tris-HCl, 5 mM MgCl₂, 0.05% BSA, adjusted pH 7.4). Total and non-specific bindings (in the presence of 1 μM of d(CH₂)₅Tyr(Me)AVP [β-mercapto-β,β-cyclopentamethylene propionyl,O-Me-Tyr²,Arg⁸]-vasopressin (βMCPVP)) were determined in a range of ³H-AVP concentrations (0.05 nM to 100 nM).

For the competition assay, test compounds were incubated with 0.5 nM ³H-AVP and 20 μg protein of cell membrane for 60 minutes at 25° C. in a final 250 μl of incubation buffer (50 mM Tris-HCl, 5 mM MgCl₂, 0.05% BSA, adjusted pH 7.4). Nonspecific binding was determined by 1 μM of pMCPVP. The saturation derived K_{D} gained in saturation assay was used for all Ki calculations.

All incubations were terminated by filtration through Packard GF/C Unfilter plates pre-soaked in 0.5% polyethyleneimine followed by three washes with ice-cold filtration buffer (50 mM Tris-HCl, 5 mM MgCl₂, adjusted pH 7.4). The plates were then placed back into the incubator at 50° C. to dry. The bottom of the Unifilter plates were sealed using Packard plate seals and 50 μl of Microscint 0 was added to each well. The plates were then sealed with Packard Topseal A, and receptor-bound radioactivity was counted by Packard Topcount NXT.

An NMDA NR2B antagonist of the present invention may be usefully combined with another pharmacologically active compound, or with two or more other pharmacologically active compounds, particularly in the treatment of pain. For example, an NMDA NR2B antagonist, particularly a compound of the formula (I), or a pharmaceutically acceptable salt or solvate thereof, as defined above, may be administered simultaneously, sequentially or separately in combination with one or more agents selected from:
(i) opioid analgesics, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine and pentazocine;
(ii) nonsteroidal antiinflammatory drugs (NSAIDs), e.g. aspirin, diclofenac, diflusinal, etodolac, fenbufen, fenoprofen, flufenisal, flurbiprofen,ibuprofen, indomethacin, ketoprofen, ketorolac, meclofenamic acid, mefenamic acid, nabumetone, naproxen, oxaprozin, phenylbutazone, piroxicam, sulindac, tolmetin, zomepirac, and their pharmaceutically acceptable salts;
(iii) barbiturate sedatives, e.g. amobarbital, aprobarbital, butabarbital, butabital, mephobarbital, metharbital, methohexital, pentobarbital, phenobartital, secobarbital, talbutal, theamylal, thiopental and their pharmaceutically acceptable salts;
(iv) benzodiazepines having a sedative action, e.g. chlordiazepoxide, clorazepate, diazepam, flurazepam, lorazepam, oxazepam, temazepam, triazolam and their pharmaceutically acceptable salts,
(v) H₁ antagonists having a sedative action, e.g. diphenhydramine, pyrilamine, promethazine, chlorpheniramine, chlorcyclizine and their pharmaceutically acceptable salts;
(vi) miscellaneous sedatives such as glutethimide, meprobamate, methaqualone, dichloralphenazone and their pharmaceutically acceptable salts;
(vii) skeletal muscle relaxants, e.g. baclofen, carisoprodol, chlorzoxazone, cyclobenzaprine, methocarbamol, orphrenadine and their pharmaceutically acceptable salts,
(viii) NMDA receptor antagonists, e.g. dextromethorphan ((+)-3-hydroxy-N-methylmorphinan) and its metabolite dextrorphan ((+)-3-hydroxy-N-methylmorphinan), ketamine, memantine, pyrroloquinoline quinone and cis-4-(phosphonomethyl)-2-piperidinecarboxylic acid and their pharmaceutically acceptable salts;
(ix) alpha-adrenergic active compounds, e.g. doxazosin, tamsulosin, clonidine and 4-amino-6,7-dimethoxy-2-(5-methanesulfonamido-1,2,3,4-tetrahydroisoquinol-2-yl)-5-(2-pyridyl) quinazoline;
(x) tricyclic antidepressants, e.g. desipramine, imipramine, amytriptiline and nortriptiline;
(xi) anticonvulsants, e.g. carbamazepine and valproate;
(xii) Tachykinin (NK) antagonists, particularly Nk-3, NK-2 and NK-1 e.g. antagonists, (αR,9R)-7-[3,5-bis(trifluoromethyl)benzyl]-8,9,10,11-tetrahydro-9-methyl-5-(4-methylphenyl)-7H-[1,4]diazocino[2,1-g][1,7]naphthridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]methyl]-1,2-dihydro-3H-1,2,4-triazol-3-one (MK-869), lanepitant, dapitant and 3-[[2-methoxy-5-(trifluoromethoxy)phenyl]methylamino]-2-phenyl-piperidine (2S,3S)
(xiii) Muscarinic antagonists, e.g oxybutin, tolterodine, propiverine, tropsium chloride and darifenacin;
(xiv) COX-2 inhibitors, e.g. celecoxib, rofecoxib and valdecoxib;
(xv) Non-selective COX inhibitors (preferably with GI protection), e.g. nitroflurbiprofen (HCT-1026);
(xvi) coal-tar analgesics, in particular, paracetamol;
(xvii) neuroleptics, such as droperidol;
(xviii) Vanilloid receptor agonists, e.g. resinferatoxin;
(xix) Beta-adrenergic compounds such as propranolol;
(xx) Local anaesthetics, such as mexiletine;
(xxi) Corticosteriods, such as dexamethasone
(xxii) serotonin receptor agonists and antagonists;
(xxiii) cholinergic (nicotinic) analgesics;
(xxiv) miscellaneous agents such as Tramadol®;
(xxv) PDEV inhibitors, such as sildenafil, vardenafil or taladafil;
(xxvi) serotonin reuptake inhibitors, e.g. fluoxetine, paroxetine, citalopram and sertraline;
(xxvii) mixed serotonin-noradrenaline reuptake inhibitors, e.g. milnacipran, venlafaxine and duloxetine;
(xxviii) noradrenaline reuptake inhibitors , e.g. reboxetine;
(xxix) atypical anti-psychotics, e.g. ziprasidone, olanzapine, clozapine, risperidone, sertindole, quetiapine, aripiprazole and amisulpride.

### EXAMPLES

The following Examples and Preparations illustrate the preparation of compounds of the formula (I).

¹H Nuclear magnetic resonance (NMR) spectra were in all cases consistent with the proposed structures. Characteristic chemical shifts (δ) are given in parts-per-million downfield from tetramethylsilane using conventional abbreviations for designation of major peaks: e.g. s, singlet; d, doublet; t, triplet; q, quartet; m, multiplet; br, broad. The mass spectra (m/z) were recorded using either electrospray ionisation (ESI) or atmospheric pressure chemical ionisation (APCI). The following abbreviations have been used: CDCl₃, deuterochloroform; D₆-DMSO, deuterodimethylsulphoxide; CD₃OD, deuteromethanol; THF, tetrahydrofuran; MeOH, methanol; EtOH, ethanol; AcOEt, ethyl acetate; DMF, dimethyl formamide, EDCl, N-ethyl-N′-(3-dimethylaminopropyl)carbodiimide; HOBt, 1-hydroxybenzotriazole; DIAD, Diisopropyl azodicarboxylate; TBAF, tetrabutylammonium fluoride; TMSCN, trimethylsilylcyanide; PPh₃, Triphenylphosphine; SEMCl 2-(Trimethylsilyl)ethoxymethyl chloride; Pd—C, palladium carbon; mCPBA, m-Chloroperbenzoic acid. ‘Ammonia’ refers to a concentrated solution of ammonia in water possessing a specific gravity of 0.88. Where thin layer chromatography (TLC) has been used it refers to silica gel TLC using silica gel 60 F₂₅₄ plates, R_{f} is the distance travelled by a compound divided by the distance travelled by the solvent front on a TLC plate.

### Example 1

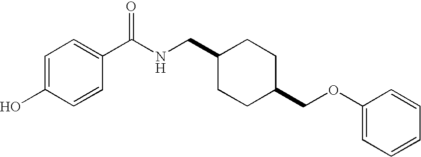

### 4-Hydroxy-N-{[cis-4-(phenoxymethyl)cyclohexyl]methyl}benzamide

DIAD (0.89 mL, 4.5 mmol) was added dropwise to a mixture of 4-(benzyloxy)-N-{[cis-4-(hydroxymethyl)cyclohexyl]methyl}benzamide (1.1 g, 3.0 mmol), phenol (0.42 g, 4.5 mmol) and triphenylphosphine (1.2 g, 4.5 mmol) in THF (10 mL) at 0° C. The mixture was stirred at room temperature for 8 hours and quenched with water and 2 N aq. NaOH. The whole was extracted with CH₂Cl₂. The extract was dried over MgSO₄ and concentrated in vacuum. The residue was purified by silica gel column chlomatography (hexane:AcOEt=3:1) to afford 4-(benzyloxy)-N-{[cis-4-(phenoxymethyl)cyclohexyl]methyl}benzamide. A mixture of 4-(benzyloxy)-N-{[cis-4-(phenoxymethyl)cyclohexyl]methyl}benzamide and 10% Pd—C (0.20 g) was hydrogenated at 1 atm for 3 hours. The mixture was filtered through a pad of celite and the filtrate was evaporated. The residue was purified by silica gel column chromatography (hexane:AcOEt=2:1) to give the titled compound (0.68 g).

¹H NMR (CDCl₃) δ: 7.66 (d, J=8.4 Hz, 2H), 7.31-7.24 (m, 2 H), 6.96-6.84 (m, 5H), 6.10-6.00 (m, 1H), 3.86 (d, J=6.9 Hz, 2H), 3.42 (t, J=6.4 Hz, 2H), 2.10-1.40 (m, 10H) ppm. (OH was not observed.) MS (ESI): 340.18 (M+H)⁺, 338.15 (M−H)⁻

### Example 1A

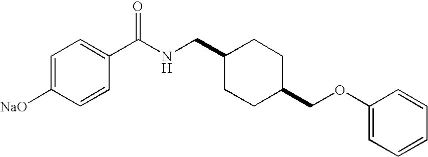

### 4-Hydroxy-N-{cis-4-(phenoxymethyl)cyclohexyl]methyl}benzamide sodium salt

To a solution of 4-hydroxy-N-{[cis-4-(phenoxymethyl)cyclohexyl]methyl}benzamide (0.68 g, 2.0 mmol) in EtOH (20 mL), 2 N aq. NaOH (0.95 mL) was added and the mixture was concentrated in vacuum. The solid was washed with CH₂Cl₂ and filtered to give the titled compound (0.53 g) as a white solid.

¹H NMR (DMSO-d₆) δ: 7.44 (t, J=5.7 Hz, 1H), 7.37-7.23 (m, 4 H), 6.96-6.88 (m, 3H), 5.97 (d, J=8.8 Hz, 2H), 3.86 (d, J=7.0 Hz, 2H), 3.13 (t, J=6.6 Hz, 2H), 1.96-1.30 (m, 10H) ppm. MS (ESI): 340.24 (M+H)⁺, 338.19 (M−H)⁻ IR (KBr)νₘₐₓ: 3350, 1599, 1547, 1497, 1296, 1246, 1175, 1035 cm⁻¹

### Example 2

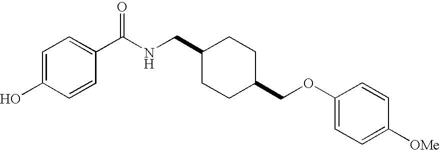

### 4-Hydroxy-N-({cis-4-[(4-methoxyphenoxy)methyl]cyclohexyl}methyl)benzamide

A mixture of 4-(benzyloxy)-N-({cis-4-[(4-methoxyphenoxy)methyl]cyclohexyl}methyl)benzamide (70 mg, 0.15 mmol) and 10% Pd—C (15 mg) in MeOH (10 mL) was hydrogenated at 1 atm for 2 hours. The mixture was filtered through a pad of celite and the filtrate was evaporated. The residue was purified by silica gel column chromatography (hexane-AcOEt 1:1) and crystallization from CH₂Cl₂-hexane to give the titled compound (41 mg).

¹H NMR (CDCl₃) δ: 7.65 (d, J=8.6 Hz, 2H), 6.90-6.81 (m, 6H), 6.62 (br, 1H), 6.13-6.05 (m, 1H), 3.81 (d, J=6.8 Hz, 2H), 3.77 (s, 3H), 3.46-3.39 (m, 2H), 2.05-1.40 (m, 10H) ppm. MS (ESI): 370.7 (M+H)⁺, 367.9 (M−H)⁻ IR (KBr)νₘₐₓ: 3119, 2926, 1601, 1501, 1450, 1281, 1227, 1177 cm⁻¹

### Example 3

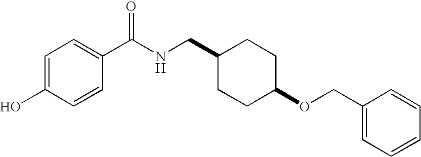

### N-{[cis-4-(Benzyloxy)cyclohexyl]methyl}-4-hydroxybenzamide

A mixture of 4-hydroxybenzoic acid (0.17 g, 1.2 mmol), [cis-4-(benzyloxy)cyclohexyl]methylamine (0.25 g, 1.2 mmol) HOBt.H₂O (0.21 g, 1.4 mmol), and EDCI (0.27 g, 1.4 mmol) in DMF (12 mL) was stirred at room temperature for 16 hours. 2 N aq. NaOH (10 mL) was added and the mixture was stirred for 1 hour. The mixture was neutralized with 2 N aq. HCl (10 mL) and extracted with AcOEt. The extract was washed with sat. aq. NaHCO₃ and water. The organic layer was dried over MgSO₄ and concentrated in vacuo. The residue was purified by silica gel column chromatography (hexane:AcOEt=1:1).

¹H NMR (CDCl₃) δ: 7.63 (d, J=8.6 Hz, 2H), 7.36-7.24 (m, 5H), 7.09 (s, 1H), 6.85 (d, J=8.6 Hz, 2H), 6.23-6.15 (m, 1H), 4.50 (s, 2H), 3.68-3.62 (m, 1H), 3.33 (t, J=6.2 Hz, 2H), 2.00-1.40 (m, 9H) ppm. MS (ESI): 340.21 (M+H)⁺, 338.18 (M−H)⁻ IR (KBr)νₘₐₓ: 3227, 2926, 1612, 1508, 1439, 1277, 1175, 1094, 1045 cm⁻¹

### Example 4

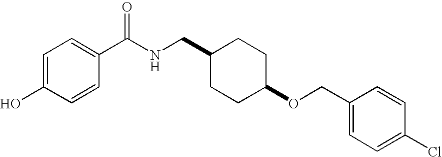

### N-({cis-4-[(4-Chlorobenzyl)oxy]cyclohexyl}methyl)-4-hydroxybenzamide

NaH (60%, 9.6 mg, 0.24 mmol) was added to a solution of N-[(cis-4-hydroxycyclohexyl)methyl]-4-(methoxymethoxy)benzamide (60 mg, 0.20 mmol) in DMF (1.0 mL) and the mixture was stirred at room temperature for 30 min. To the mixture, 4-chlorobenzylbromide (49 mg, 0.24 mmol) was added and the mixture was stirred at room temperature for 2 hours. To the mixture, 10% HCl-MeOH (2.0 mL) was added at room temperature and the mixture was stirred at 50° C. for 30 min. The mixture was diluted with AcOEt and washed with sat. aq. NaHCO₃ and water. The organic layer was dried over MgSO₄ and was evaporated. The residue was purified by prep.TLC (hexane:AcOEt=1:2) to give the titled compound (2.0 mg).

¹H NMR (CDCl₃) δ: 7.65 (d, J=8.7 Hz, 2H), 7.32-7.26 (m, 4H), 6.85 (d, J=8.6 Hz, 2H), 6.20-6.10 (m, 1H), 4.45 (s, 2H), 3.67-3.60 (m, 1H), 3.37-3.30 (m, 2H), 2.05-1.40 (m, 9H) ppm. (OH was not observed.) MS (ESI): 374.0 (M+H)⁺, 371.9 (M−H)⁻

### Example 5

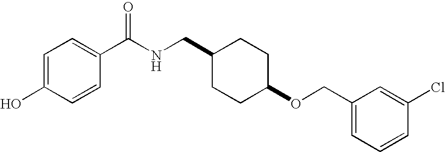

### N-({cis-4-[(3-Chlorobenzyl)oxy]cyclohexyl}methyl)-4-hydroxybenzamide

This compound was prepared with 3-chlorobenzylbromide by a procedure similar to that in Example 4.

¹H NMR (CDCl₃) δ: 7.64 (d, J=8.6 Hz, 2H), 7.36-7.20 (m, 4H), 6.85 (d, J=8.7 Hz, 2H), 6.20-6.10 (m, 1H), 4.46 (s, 2H), 3.67-3.60 (m, 1H), 3.38-3.30 (m, 2H), 2.05-1.40 (m, 9H) ppm. (—OH was not observed.) MS (ESI): 374.0 (M+H)⁺, 371.9 (M−H)⁻

### Example 6

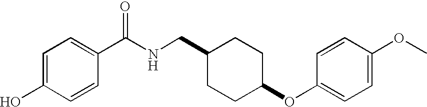

### 4-Hydroxy-N-{[cis-4-(4-methoxyphenoxy)cyclohexyl]methyl}benzamide

A mixture of 4-(methoxymethoxy)-N-{[cis-4-(4-methoxyphenoxy)cyclohexyl]methyl}benzamide (11 mg, 0.027 mmol) and 10% HCl-MeOH (1.0 mL) was stirred at 50° C. for 30 min. The mixture was evaporated. The residue was purified by silica gel column chromatography (hexane:AcOEt=1:2) to give the titled compound (10 mg).

¹H NMR (CDCl₃) δ: 7.65 (d, J=8.6 Hz, 2H), 7.19 (br, 1H), 6.90-6.79 (m, 6H), 6.28-6.18 (m, 1H), 4.45-4.38 (m, 1H), 3.77 (s, 3H), 3.36 (t, J=6.4 Hz, 2H), 2.10-1.45 (m, 9H) ppm. MS (ESI): 356.24 (M+H)⁺, 354.21 (M−H)⁻ IR (KBr)νₘₐₓ: 3327, 2930, 1609, 1506, 1443, 1281, 1229, 1177, 1038 cm⁻¹

### Example 7

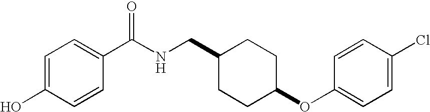

### N-{[cis-4-(4-Chlorophenoxy)cyclohexyl]methyl}-4-hydroxybenzamide

This compound was prepared with N-{[cis-4-(4-chlorophenoxy)cyclohexyl]methyl}-4-(methoxymethoxy)benzamide by a procedure similar to that in Example 6.

¹H NMR (CDCl₃) δ: 7.69 (s, 1H), 7.62 (d, J=8.7 Hz, 2H), 7.20 (d, J=8.9 Hz, 2H), 6.90-6.78 (m, 4 H), 6.32-6.22 (m, 1H), 4.52-4.45 (m, 1H), 3.35 (t, J=6.3 Hz, 2H), 2.10-1.96 (m, 2H), 1.78-1.40 (m, 7H) ppm. MS (ESI): 360.19 (M+H)⁺, 358.14 (M−H)⁻ IR (KBr)νₘₐₓ: 3358, 2928, 1609, 1508, 1489, 1443, 1281, 1242, 1173 cm⁻¹

### Example 8

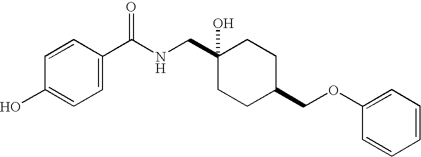

### 4-Hydroxy-N-{[trans-1-hydroxy-4-(phenoxymethyl)cyclohexyl]methyl}benzamide

A mixture of 1-(aminomethyl)-4-(phenoxymethyl)cyclohexanol hydrochloride (1.1 g, 4.0 mmol), 4-hydroxybenzoic acid (0.79 g, 4.4 mmol), HOBt-H₂O (0.12 g, 0.8 mmol), Et₃N (1.1 mL, 8.0 mmol), and EDCI (0.92 g, 4.8 mmol) in DMF (40 mL) was stirred at room temperature for 16 hours. 2 N aq. NaOH (15 mL) and MeOH (10 mL) were added and the mixture was stirred at room temperature for 4 hours. The mixture was neutrized with 2 N aq. HCl (15 mL) and extracted with AcOEt. The extract was washed with sat. aq. NaHCO₃ and water, dried over MgSO₄, and evaporated. The residue was purified by silica gel column chlomatography (CH₂Cl₂:MeOH=25:1) to give the titled compound (0.43 g).

¹H NMR (DMSO-d₆) δ: 7.92 (t, J=5.5 Hz, 1H), 7.73 (d, J=8.8 Hz, 2H), 7.32-7.22 (m, 2H), 6.96-6.76 (m, 5H), 4.73 (br, 1H), 3.82 (d, J=6.2 Hz, 2H), 3.40-3.34 (m, 2H), 1.84-1.20 (m, 9H) ppm. (—OH was not obserbed.) MS (ESI): 356.17 (M+H)⁺, 354.13 (M−H)⁻ IR (KBr)νₘₐₓ: 3190, 2931, 2864, 1608, 1541, 1512, 1247, 1174, 1224, 1043 cm⁻¹ m.p. 189.5° C.

### Example 9

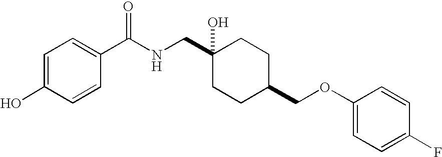

### N-({trans-4-[(4-Fluorophenoxy)methyl]-1-hydroxcyclohexyl}methyl)-4-hydroxybenzamide

A mixture of N-{[trans-1-hydroxy-4-(hydroxymethyl)cyclohexyl]methyl}-4-(methoxymethoxy)benzamide (97 mg, 0.30 mmol), 4-fluorophenol (50 mg, 0.45 moml) and cyanomethylenetributylphosphorane (0.12 g, 0.45 mmol) in toluene (1.5 mL) was stirred at 90° C. for 1 hour. After cooling to room temperature, the mixture was purified by silica gel column chromatography (hexane:AcOEt=2:1) to give N-({trans-4-[(4-fluorophenoxy)methyl]-1-hydroxycyclohexyl}methyl)-4-(methoxymethoxy)benzamide. N-({trans-4-[(4-Fluorophenoxy)methyl]-1-hydroxycyclohexyl}methyl)-4-(methoxymethoxy)benzamide was dissolved with 10% HCl-MeOH (2.0 mL) and the mixture was stirred at 50° C. for 30 min. After evaporation, the residue was purified by silica gel column chromatography (CH₂Cl₂:MeOH=30:1) to the titled compound (57 mg) as a white solid.

¹H NMR (DMSO-d₆) δ: 10.00 (br, 1H), 7.95-7.88 (m, 1H), 7.73 (d, J=8.6 Hz, 2H), 7.13-7.05 (m, 2 H), 6.96-6.90 (m, 2H), 6.79 (d, J=8.6 Hz, 2H), 4.71 (br, 1H), 3.80 (d, J=6.0 Hz, 2H), 3.37 (d, J=6.0 Hz, 2H), 1.82-1.60 (m, 5H), 1.35-1.14 (m, 4H) ppm. MS (ESI): 374.21 (M+H)⁺, 372.13 (M−H)⁻ IR (KBr)νₘₐₓ: 3379, 2937, 1630, 1611, 1555, 1508, 1248, 1207 cm⁻¹ m.p. 176.4° C.

### Example 10

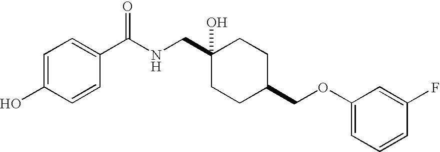

### N-({trans-4-[(3-Fluorophenoxy)methyl]-1-hydroxycyclohexyl}methyl)-4-hydroxybenzamide

This compound was prepared with 3-fluorophenol by a procedure similar to that in Example 9.

¹H NMR (DMSO-d₆) δ: 9.97 (br, 1H), 7.96-7.88 (m, 1H), 7.73 (d, J=8.6 Hz, 2H), 7.34-7.24 (m, 1H), 6.84-6.70 (m, 5H), 4.71 (br, 1H), 3.84 (d, J=6.1 Hz, 2H), 3.39-3.34 (m, 2H), 1.84-1.62 (m, 5H), 1.40-1.24 (m, 4H) ppm. MS (ESI): 374.18 (M+H)⁺, 372.13 (M−H)⁻ IR (KBr)νₘₐₓ: 3248,2937, 1630, 1611, 1593, 1508, 1277, 1136, 1119 cm⁻¹ m.p. 186.0° C.

### Example 11

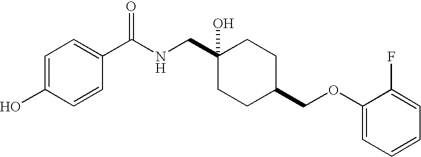

### N-({trans-4-[(2-Fluorophenoxy)methyl]-1-hydroxycyclohexyl}methyl)-4-hydroxybenzamide

This compound was prepared with 2-fluorophenol by a procedure similar to that in Example 9.

¹H NMR (DMSO-d₆) δ: 10.00 (br, 1H), 7.96-7.89 (m, 1H), 7.73 (d, J=8.6 Hz, 2H), 7.24-7.07 (m, 3H), 6.96-6.87 (m, 1H), 6.79 (d, J=8.6 Hz, 2H), 4.72 (br, 1H), 3.90 (d, J=6.4 Hz, 2H), 3.40-3.34 (m, 2H), 1.90-1.62 (m, 5H), 1.40-1.20 (m, 4H) ppm. MS (ESI): 374.22 (M+H)⁺, 372.16 (M−H)⁻ IR (KBr)νₘₐₓ: 3252, 2937, 1630, 1611, 1508, 1277, 1256, 1109 cm⁻¹ m.p. 185.0° C.

### Example 12

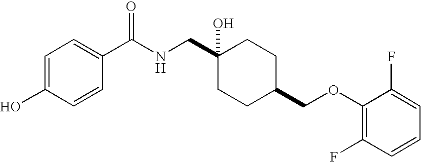

### N-({trans-4-[(2,6-Difluorophenoxy)methyl]-1-hydroxycyclohexyl}methyl)-4-hydroxybenzamide

This compound was prepared with 2,6-difluorophenol by a procedure similar to that in Example 9.

¹H NMR (DMSO-d₆) δ: 10.00 (br, 1H), 7.97-7.89 (m, 1H), 7.73 (d, J=8.8 Hz, 2H), 7.18-7.08 (m, 3H), 6.79 (d, J=8.6 Hz, 2H), 4.73 (br, 1H), 3.95 (d, J=6.0 Hz, 2H), 3.38-3.34 (m, 2H), 1.82-1.62 (m, 5H), 1.40-1.25 (m, 4H) ppm. MS (ESI): 392.18 (M+H)⁺, 390.14 (M−H)⁻ IR (KBr)νₘₐₓ: 3150, 2950, 1638, 1508, 1238 cm⁻¹ m.p. 153.7° C.

### Example 13

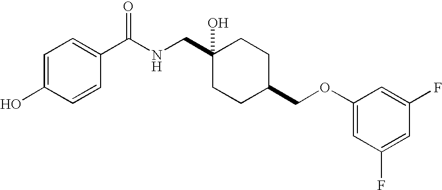

### N-({trans-4-[(3,5-Difluorophenoxy)methyl]-1-hydroxycyclohexyl}methyl)-4-hydroxybenzamide

This compound was prepared with 3,5-difluorophenol by a procedure similar to that in Example 9.

¹H NMR (DMSO-d₆) δ: 10.00 (br, 1H), 7.95-7.88 (m, 1H), 7.73 (d, J=8.6 Hz, 2H), 6.82-6.68 (m, 5H), 4.71 (br, 1H), 3.86 (d, J=6.4 Hz, 2H), 3.40-3.34 (m, 2H), 1.85-1.60 (m, 5H), 1.40-1.20 (m, 4H) ppm. MS (ESI): 392.15 (M+H)⁺, 390.09 (M−H)⁻ IR (KBr)νₘₐₓ: 3256, 2941, 1624, 1508, 1466, 1285, 1153, 1115 cm⁻¹ m.p. 102.4° C.

### Example 14

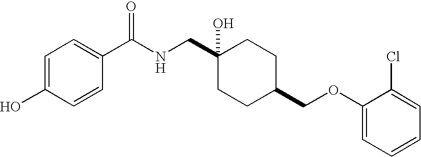

### N-({trans-4-[(2-Chlorophenoxy)methyl]-1-hydroxycyclohexyl}methyl)-4-hydroxybenzamide

This compound was prepared with 2-chlorophenol by a procedure similar to that in Example 9.

¹H NMR (DMSO-d₆) δ: 9.97 (br, 1H), 7.96-7.88 (m, 1H), 7.74 (d, J=8.6 Hz, 2H), 7.41 (dd, J=1.7, 8.9 Hz, 1H), 7.32-7.25 (m, 1H), 7.15 (dd, J=1.5, 8.2 Hz, 1H), 6.97-6.90 (m, 1H), 6.80 (d, J=8.6 Hz, 2H), 4.71 (br, 1H), 3.91 (d, J=6.4 Hz, 2H), 3.40-3.35 (m, 2H), 1.90-1.60 (m, 5H), 1.40-1.25 (m, 4H) ppm. MS (ESI): 390.17, 392.17 (M+H)⁺, 388.09, 389.98 (M−H)⁻ IR (KBr)νₘₐₓ: 3296, 2934, 1634, 1508, 1468, 1281, 1252 cm⁻¹ m.p. 159.0° C.

### Example 15

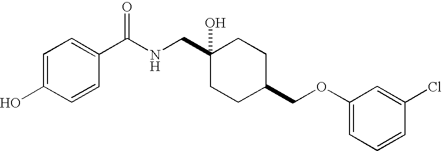

### N-({trans-4-[(3-Chlorophenoxy)methyl]-1-hydroxycyclohexyl}methyl)-4-hydroxybenzamide

This compound was prepared with 3-chlorophenol by a procedure similar to that in Example 9.

¹H NMR (DMSO-d₆) δ: 10.00 (br, 1H), 7.96-7.88 (m, 1H), 7.73 (d, J=8.8 Hz, 2H), 7.28 (t, J=8.1 Hz, 1H), 7.03-6.88 (m, 3H), 6.80 (d, J=8.6 Hz, 2H), 4.71 (br, 1H), 3.85 (d, J=6.1 Hz, 2H), 3.40-3.35 (m, 2H), 1.84-1.60 (m, 5H), 1.40-1.25 (m, 4H) ppm. MS (ESI): 390.15 (M+H)⁺, 388.06 (M−H)⁻ IR (KBr)νₘₐₓ: 3179, 2928, 1636, 1593, 1512, 1458, 1286, 1236, 1042 cm⁻¹ m.p. 164.9° C.

### Example 16

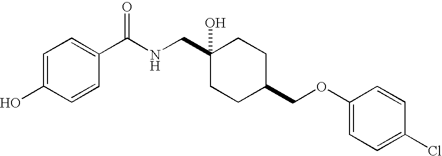

### N-({trans-4-[(4-Chlorophenoxy)methyl]-1-hydroxycyclohexyl}methyl)-4-hydroxybenzamide

This compound was prepared with 4-chlorophenol by a procedure similar to that in Example 9.

¹H NMR (DMSO-d₆) δ: 9.99 (br, 1H), 7.96-7.89 (m, 1H), 7.73 (d, J=8.6 Hz, 2H), 7.31 (d, J=9.0 Hz, 2H), 6.96 (d, J=9.0 Hz, 2H), 6.79 (d, J=8.6 Hz, 2H), 4.72 (br, 1H), 3.82 (d, J=6.4 Hz, 2H), 3.40-3.35 (m, 2H), 1.82-1.60 (m, 5H), 1.40-1.20 (m, 4H) ppm. MS (ESI): 390.13 (M+H)⁺, 388.08 (M−H)⁻ IR (KBr)νₘₐₓ: 3198, 2941, 1631, 1508, 1491, 1279, 1244, 1121 cm⁻¹ m.p. 208.2° C.

### Example 17

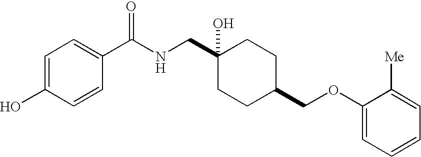

### 4-Hydroxy-N-({trans-1-hydroxy-4-[(2-methylphenoxy)methyl]cyclohexyl}methyl)benzamide

This compound was prepared with 2-methylphenol by a procedure similar to that in Example 9.

¹H NMR (DMSO-d₆) δ: 9.99 (br, 1H), 7.96-7.88 (m, 1H), 7.73 (d, J=8.8 Hz, 2H), 7.16-7.09 (m, 2H), 6.92-6.75 (m, 4H), 4.72 (br, 1H), 3.82 (d, J=6.0 Hz, 2H), 3.38 (d, J=5.9 Hz, 2H), 2.16 (s, 3H), 1.86-1.60 (m, 5H), 1.42-1.25 (m, 4H) ppm. MS (ESI): 370.18 (M+H)⁺, 368.12 (M−H)⁻ IR (KBr)νₘₐₓ: 3231, 2936, 1628, 1533, 1497, 1281, 1244, 1121 cm⁻¹ m.p. 189.1° C.

### Example 18

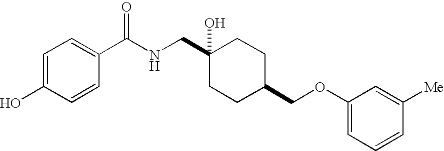

### 4-Hydroxy-N-({trans-1-hydroxy-4-[(3-methylphenoxy)methyl]cyclohexyl}methyl)benzamide

This compound was prepared with 3-methylphenol by a procedure similar to that in Example 9.

¹H NMR (DMSO-d₆) δ: 9.99 (br, 1H), 7.97-7.89 (m, 1H), 7.73 (d, J=8.6 Hz, 2H), 7.14 (t, J=7.9, 1H), 6.82-6.68 (m, 5H), 4.72 (br, 1H), 3.79 (d, J=6.0 Hz, 2H), 3.40-3.35 (m, 2H), 2.26 (s, 3H), 1.82-1.62 (m, 5H), 1.40-1.20 (m, 4H) ppm. MS (ESI): 370.21 (M+H)⁺, 368.13 (M−H)⁻ IR (KBr)νₘₐₓ: 3227, 2934, 1636, 1611, 1508, 1281, 1157 cm⁻¹ m.p. 201.40° C.

### Example 19

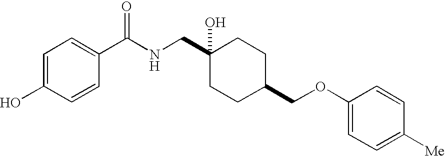

### 4-Hydroxy-N-({trans-1-hydroxy-4-[(4-methylphenoxy)methyl]cyclohexyl}methyl)benzamide

This compound was prepared with 4-methylphenol by a procedure similar to that in Example 9.

¹H NMR (DMSO-d₆) δ: 10.00 (br, 1H), 7.96-7.89 (m, 1H), 7.73 (d, J=8.6 Hz, 2H), 7.06 (d, J=8.3 Hz, 2H), 6.83-6.76 (m, 4H), 4.72 (br, 1H), 3.77 (d, J=6.2 Hz, 2H), 3.40-3.35 (m, 2H), 2.22 (s, 3H), 1.82-1.60 (m, 5H), 1.40-1.20 (m, 4H) ppm. MS (ESI): 370.21 (M+H)⁺, 368.16 (M−H)⁻ IR (KBr)νₘₐₓ: 3246, 2941, 1632, 1508, 1279, 1246, 1119 cm⁻¹ m.p. 203.1° C.

### Example 20

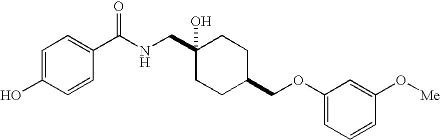

### 4-Hydroxy-N-({trans-1-hydroxy-4-[(3-methoxyphenoxy)methyl]cyclohexyl}methyl)benzamide

Diisopropyl azodicarboxylate (0.30 mL, 1.5 mmol) was added dropwise to a mixture of N-{[trans-1-hydroxy-4-(hydroxymethyl)cyclohexyl]methyl}-4-(methoxymethoxy)benzamide (0.32 g, 1.0 mmol), 3-methoxyphenol (0.19 g, 1.5 mmol) and triphenylphosphine (0.39 g, 1.5 mmol) in THF at 0° C. and the mixture was stirred at room temperature for 2 hours. The mixture was treated with 2N aq. NaOH and was extracted with CH₂Cl₂. The extract was washed with sat. aq. NaCl, dried over MgSO₄, and then concentrated in vacuo. The residue was purified by silica gel column chromatography (hexane:AcOEt=1:1) to give N-({trans-1-hydroxy-4-[(3-methoxyphenoxy)methyl]cyclohexyl}methyl)-4-(methoxymethoxy)benzamide. N-({trans-1-hydroxy-4-[(3-methoxyphenoxy)methyl]cyclohexyl}methyl)-4-(methoxymethoxy)benzamide was dissolved in 10% HCl-MeOH and the mixture was stirred at 50° C. for 30 min. The mixture was evaporated and the residue was purified by silica gel column chromatography (CH₂Cl₂:MeOH=15:1), followed by prep.TLC (CH₂Cl₂:MeOH=8:1) to give the titled compound (0.21 g) as a white solid.

¹H NMR (DMSO-d₆) δ: 9.96 (br, 1H), 7.90 (t, J=5.6, 1H), 7.73 (d, J=8.9 Hz, 2H), 7.20-7.11 (m, 1H), 6.80 (d, J=8.6 Hz, 2H), 6.54-6.46 (m, 3H), 4.70 (br, 1H), 3.80 (d, J=6.1 Hz, 2H), 3.72 (s, 3H), 3.37 (d, J=5.8 Hz, 2H), 1.85-1.60 (m, 5H), 1.42-1.20 (m, 4H) ppm. MS (ESI): 386.14 (M+H)⁺, 384.13 (M−H)⁻ IR (KBr)νₘₐₓ: 3229, 2941, 1636, 1587, 1508, 1279, 1155 cm⁻¹ m.p. 190.3° C.

### Example 21

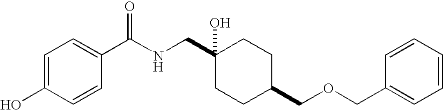

### N-({trans-4-[(Benzyloxy)methyl]-1-hydroxycyclohexyl}methyl)-4-hydroxybenzamide

A mixture of N-({trans-4-[(benzyloxy)methyl]-1-hydroxycyclohexyl}methyl)-4-(methoxymethoxy)benzamide (0.37 g, 1.0 mmol) and 10% HCl-MeOH (10 mL) was stirred at 50° C. for 1 hour. After evaporation, the residue was purified by silica gel column chromatography (hexane:AcOEt=2:3) to give the titled compound (0.21 g).

¹H NMR (DMSO-d₆) δ: 9.99 (br, 1H), 7.91 (t, J=5.7, 1H), 7.73 (d, J=8.6 Hz, 2H), 7.38-7.24 (m, 5H), 6.80 (d, J=8.6 Hz, 2H), 4.71 (br, 1H), 4.45 (s, 2H), 3.36-3.24 (m, 4H), 1.70-1.54 (m, 5H), 1.36-1.08 (m, 4H) ppm. MS (ESI): 368.11 (M−H)⁻ IR (KBr)νₘₐₓ: 3242, 2941, 1609, 1508, 1275, 1115 cm⁻¹ m.p. 165.7° C.

### Example 22

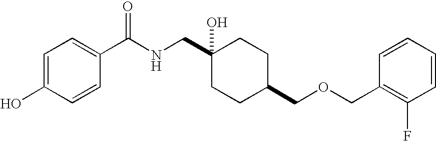

### N-[(trans-4-{[(2-Fluorobenzyl)oxy]methyl}-1-hydroxycyclohexyl)methyl]-4-hydroxybenzamide

NaH (60%, 20 mg, 0.5 mmol) was added to a solution of N-{[trans-1-hydroxy-4-(hydroxymethyl)cyclohexyl]methyl}-4-(methoxymethoxy)benzamide (0.16 g, 0.5 mmol) in DMF (2.5 mL) and the mixture was stirred at room temperature for 1 hour. To the mixture, 2-fluorobenzylbromide (95 mg, 0.5 mmol) was added at 0° C. and the mixture was stirred overnight at room temperature. The mixture was quenched with water and diluted with AcOEt. The organic layer was washed with water and dried over MgSO₄. After evaporation, the residue was purified by silica gel column chlomatography (hexane:AcOEt=2:1) to afford N-[(trans-4-{[(2-fluorobenzyl)oxy]methyl}-1-hydroxycyclohexyl)methyl]-4-(methoxymethoxy)benzamide. N-[(trans-4-{[(2-Fluorobenzyl)oxy]methyl}-1-hydroxycyclohexyl)methyl]-4-(methoxymethoxy)benzamide was dissolved in 10% HCl-MeOH (2 mL) and the mixture was stirred at 50° C. for 30 min. The mixture was evaporated. After evaporation, the residue was purified by silica gel column chlomatography (hexane:AcOEt=2:1) to afford the titled compound (32 mg) as a white solid.

¹H NMR (DMSO-d₆) δ: 9.95 (br, 1H), 7.94-7.85 (m, 1H), 7.73 (d, J=8.7 Hz, 2H), 7.48-7.12 (m, 4H), 6.80 (d, J=8.7 Hz, 2H), 4.69 (br, 1H), 4.50 (s, 2H), 3.36-3.28 (m, 4H), 1.70-1.54 (m, 5H), 1.38-1.10 (m, 4H) ppm. MS (ESI): 388.04 (M+H)⁺, 386.05 (M−H)⁻ IR (KBr)νₘₐₓ: 3283, 2941, 1634, 1508, 1281, 1223, 1084 cm⁻¹ m.p. 174.3° C.

### Example 22-A

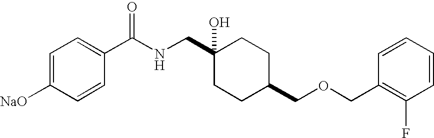

### N-[(trans-4-{[(2-Fluorobenzyl)oxy]methyl}-1-hydroxycyclohexyl)methyl]-4-hydroxybenzamide sodium salt

This compound was prepared with N-[(trans-4-{[(2-fluorobenzyl)oxy]methyl}-1-hydroxycyclohexyl)methyl]-4-hydroxybenzamide by a procedure similar to that in Example 1-A.

¹H NMR (DMSO-d₆) δ: 8.17 (br, 1H), 7.60-7.10 (m, 6H), 6.15 (d, J=8.4 Hz, 2H), 4.50 (s, 2H), 3.36-3.20 (m, 4H), 1.72-0.98 (m, 9H) ppm. MS (ESI): 388.05 (ES+), 386.03 (ES−) IR (KBr)νₘₐₓ: 3288, 2926, 1632, 1456, 1281 cm⁻¹

### Example 23

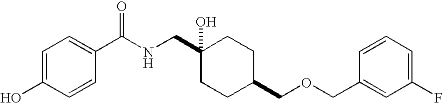

### N-[(trans-4-{[(3-Fluorobenzyl)oxy]methyl}-1-hydroxycyclohexyl)methyl]-4-hydroxybenzamide

This compound was prepared with 3-fluorobenzylbromide by a procedure similar to that in Example 22.

¹H NMR (DMSO-d₆) δ: 9.95 (br, 1H), 7.93-7.85 (m, 1H), 7.73 (d, J=8.6 Hz, 2H), 7.45-7.34 (m, 1H), 7.20-7.04 (m, 3H), 6.80 (d, J=8.7 Hz, 2H), 4.69 (br, 1H), 4.47 (s, 2H), 3.37-3.27 (m, 4H), 1.73-1.55 (m, 5H), 1.38-1.12 (m, 4H) ppm. MS (ESI): 388.04 (M+H)⁺, 386.05 (M−H)⁻ IR (KBr)νₘₐₓ: 3240, 2941, 1626, 1508, 1277, 1117 cm⁻¹ m.p. 167.6° C.

### Example 24

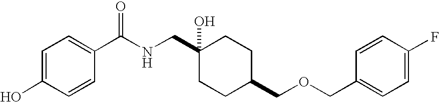

### N-[(trans-4-{[(4-Fluorobenzyl)oxy]methyl}-1-hydroxycyclohexyl)methyl]-4-hydroxybenzamide

This compound was prepared with 4-fluorobenzylbromide by a procedure similar to that in Example 22.

¹H NMR (DMSO-d₆) δ: 9.96 (br, 1H), 7.93-7.85 (m, 1H), 7.72 (d, J=8.8 Hz, 2H), 7.39-7.31 (m, 2H), 7.20-7.12 (m, 2H), 6.79 (d, J=8.8 Hz, 2H), 4.69 (br, 1H), 4.43 (s, 2H), 3.50-3.25 (m, 4H), 1.70-1.52 (m, 5H), 1.35-1.10 (m, 4H) ppm. MS (ESI): 388.14 (M+H)⁺, 386.12 (M−H)⁻ IR (KBr)νₘₐₓ: 3281, 2934, 1624, 1508, 1279, 1225, 1101 cm⁻¹ m.p. 167.6° C.

### Example 25

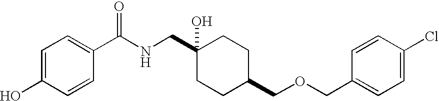

### N-[(trans-4-{[(4-Chlorobenzyl)oxy]methyl}-1-hydroxycyclohexyl)methyl]-4-hydroxybenzamide

This compound was prepared with 4-chlorobenzylbromide by a procedure similar to that in Example 22.

¹H NMR (DMSO-d₆) δ: 7.92-7.83 (m, 1H), 7.72 (d, J=8.4 Hz, 2H), 7.44-7.30 (m, 4H), 6.78 (d, J=8.2 Hz, 2H), 4.70 (br, 1H), 4.44 (s, 2H), 3.40-3.25 (m, 4H), 1.70-1.50 (m, 5H), 1.38-1.06 (m, 4H) ppm. (—OH was not observed) MS (ESI): 404.13 (M+H)⁺, 402.04 (M−H)⁻ IR (KBr)νₘₐₓ: 3221, 2934, 1630, 1508, 1277, 1111 cm⁻¹ m.p. 164.1° C.

### Example 26

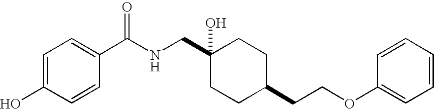

### 4-Hydroxy-N-{[trans-1-hydroxy-4-(2-phenoxyethyl)cyclohexyl]methyl}benzamide

DIAD (0.35 mL, 1.8 mmol) was added to a mixure of N-{[trans-1-hydroxy-4-(2-hydroxyethyl)cyclohexyl]methyl}-4-(methoxymethoxy)benzamide (0.41 g, 1.2 mmol), phenol (0.17 g, 1.8 mmol) and triphenylphosphine (0.47 g, 1.8 mmol) in THF (5.0 mL) at 0° C. and the mixture was stirred at room temperature for 16 hours. The mixture was diluted with CH₂Cl₂ and was washed with 2N aq. NaOH and water. The organic layer was dried over MgSO₄ and evaporated. The residue was purified by silica gel column chromatography (hexane-AcOEt 5:2 to 1:1) to afford N-{[trans-1-hydroxy-4-(2-phenoxyethyl)cyclohexyl]methyl}-4-(methoxymethoxy)benzamide. N-{[trans-1-Hydroxy-4-(2-phenoxyethyl)cyclohexyl]methyl}-4-(methoxymethoxy)benzamide was dissolved in 10% HCl-MeOH (12 mL) and the mixture was stirred at 50° C. for 30 min. After evaporation, the residue was purified by silica gel column chromatography (CH₂Cl₂:MeOH=30:1) to give the titled compound (0.25 g).

¹H NMR (DMSO-d₆) δ: 9.97 (br, 1H), 7.89 (t, J=5.8 Hz, 1H), 7.74 (d, J=8.7 Hz, 2H), 7.32-7.22 (m, 2H), 6.96-6.76 (m, 5H), 4.67 (br, 1H), 3.98 (t, J=6.4 Hz, 2H), 3.40-3.32 (m, 2H), 1.74-1.10 (m, 11H) ppm. MS (ESI): 370.12 (M+H)⁺, 368.11 (M−H)⁻ IR (KBr)νₘₐₓ: 3231, 2928, 1626, 1508, 1283, 1246, 1119 cm⁻¹ m.p. 168.7° C.

### Example 27

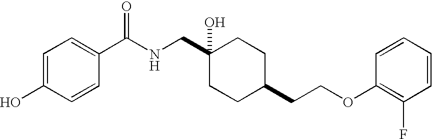

### N-({trans-4-[2-(2-Fluorophenoxy)ethyl]-1-hydroxycyclohexyl}methyl)-4-hydroxybenzamide

This compound was prepared with 2-fluorophenol by a procedure similar to that in Example 26.

¹H NMR (DMSO-d₆) δ: 9.96 (br, 1H), 7.89 (t, J=6.1 Hz, 1H), 7.74 (d, J=8.7 Hz, 2H), 7.24-7.16 (m, 3H), 6.96-6.76 (m, 3H), 4.67 (br, 1H), 4.06 (t, J=6.6 Hz, 2H), 3.40-3.32 (m, 2H), 1.74-1.10 (m, 11H) ppm. MS (ESI): 388.13 (M+H)⁺, 386.12 (M−H)⁻ IR (KBr)νₘₐₓ: 3233, 2928, 1632, 1508, 1281, 1113 cm⁻¹ m.p. 178.9° C.

### Example 28

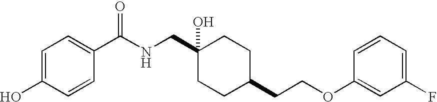

### N-({trans-4-[2-(3-Fluorophenoxy)ethyl]-1-hydroxycyclohexyl}methyl)-4-hydroxybenzamide

This compound was prepared with 3-fluorophenol by a procedure similar to that in Example 26.

¹H NMR (DMSO-d₆) δ: 9.97 (br, 1H), 7.89 (t, J=5.8 Hz, 1H), 7.74 (d, J=8.7 Hz, 2H), 7.35-7.23 (m, 1H), 6.85-6.69 (m, 5H), 4.67 (br, 1H), 4.00 (t, J=6.6 Hz, 2H), 3.40-3.32 (m, 2H), 1.74-1.10 (m, 11H) ppm. MS (ESI): 388.11 (M+H)⁺, 386.13 (M−H)⁻ IR (KBr)νₘₐₓ: 3238, 2930, 1624, 1508, 1281, 1134 cm⁻¹ m.p. 134.5° C.

### Example 29

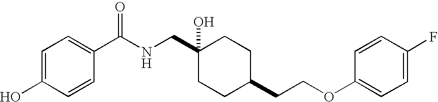

### N-({trans-4-[2-(4-Fluorophenoxy)ethyl]-1-hydroxycyclohexyl}methyl)-4-hydroxybenzamide

This compound was prepared with 4-fluorophenol by a procedure similar to that in Example 26.

¹H NMR (DMSO-d₆) δ: 9.96 (br, 1H), 7.89 (t, J=5.6 Hz, 1H), 7.74 (d, J=8.7 Hz, 2H), 7.14-7.04 (m, 2H), 6.98-6.88 (m, 2H), 6.80 (d, J=8.7 Hz, 2H), 4.67 (br, 1H), 3.96 (t, J=6.4 Hz, 2H), 3.40-3.32 (m, 2H), 1.74-1.10 (m, 11H) ppm. MS (ES1): 388.13 (M+H)⁺, 386.11 (M−H)⁻ IR (KBr)νₘₐₓ: 3285, 2937, 1634, 1508, 1209, 1026 cm⁻¹ m.p. 177.5° C.

### Example 30

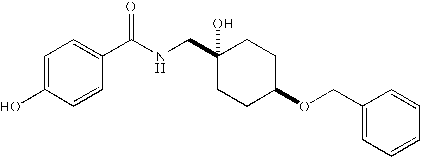

### N-{[trans-4-(Benzyloxy)-1-hydroxycyclohexyl]methyl}-4-hydroxybenzamide

A mixture of N-{[4-(benzyloxy)-1-hydroxycyclohexyl]methyl}-4-{[2-(trimethylsilyl)ethoxy]methoxy}benzamide (0.49 g, 1.0 mmol) and TBAF (1.0 M in THF, 5.0 mL) was refluxed for 16 hours. The mixture was diluted with AcOEt and was washed with sat. aq. NH₄Cl. The organic layer was dried over MgSO₄ and evaporated. The residue was purified by silica gel column chromatography (CH₂Cl₂:MeOH=20:1) and HPLC (DAICEL CHIRALCEL OJ, hexane:EtOH=7:3) to give the titled compound (80 mg).

1H NMR (DMSO-d₆) δ: 10.01 (br, 1H), 8.05-7.97 (m, 1H), 7.73 (d, J=8.6 Hz, 2H), 7.32-7.20 (m, 5H), 6.79 (d, J=8.6 Hz, 2H), 4.51 (br, 1H), 4.43 (s, 2H), 3.56-3.49 (m, 1H), 3.26 (d, J=6.0 Hz, 2H), 1.80-1.55 (m, 6H), 1.35-1.20 (m, 2H) ppm. MS (ESI): 356.18 (M+H)⁺, 354.17 (M−H)⁻ IR (KBr)νₘₐₓ: 3134, 2934, 1607, 1558, 1508, 1279, 1065 cm⁻¹

### Example 31

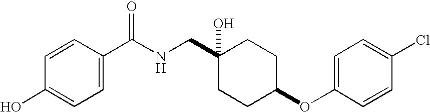

### N-{[trans-4-(4-Chlorophenoxy)-1-hydroxycyclohexyl]methyl}-4-hydroxybenzamide and

### Example 32

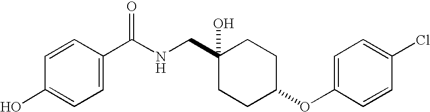

### N-{[cis-4-(4-Chlorophenoxy)-1-hydroxycyclohexyl]methyl}-4-hydroxybenzamide

A mixture of 4-hydroxybenzoic acid (0.55 g, 4.0 mmol), 1-(aminomethyl)-4-(4-chlorophenoxy)cyclohexanol (1.0 g, 4.0 mmol), EDCI (0.92 mg, 4.8 mmol) and HOBt.H₂O (0.74 g, 4.8 mmol) in DMF (40 mL) was stirred at room temperature for 16 hours. The mixture was diluted with AcOEt and was washed with sat. aq. NaHCO₃ and water. The organic layer was dried over MgSO₄ and evaporated. The residue was purified by silica gel column chromatography (hexane:AcOEt=1:3) to give the mixture of titled compounds (1.1 g). The mixture was separated by HPLC (DAICEL CHIRALPAK AD, hexane:EtOH:Et₂NH=85:15:0.1) to give Example 31 (0.32 g) and Example 32 (0.22 g).

### Data for Example 31:

¹H NMR (DMSO-d₆) δ: 9.96 (br, 1H), 8.02 (t, J=5.9 Hz, 1H), 7.73 (d, J=8.7 Hz, 2H), 7.29 (d, J=9.0 Hz, 2H), 6.96 (d, J=8.9 Hz, 2H), 6.80 (d, J=8.7 Hz, 2H), 4.62-4.46 (m, 2H), 3.30 (d, J=5.9 Hz, 2H), 1.92-1.30 (m, 8H) ppm. MS (ESI): 375.9 (M+H)⁺, 373.9 (M−H)⁻ IR (KBr)νₘₐₓ: 3234, 2949, 1632, 1508, 1491, 1283, 1238 cm⁻¹ m.p. 199.0° C.

### Data for Example 32:

¹H NMR (DMSO-d₆) δ: 9.96 (br, 1H), 8.03 (t, J=5.8 Hz, 1H), 7.74 (d, J=8.7 Hz, 2H), 7.28 (d, J=9.0 Hz, 2H), 6.96 (d, J=8.9 Hz, 2H), 6.80 (d, J=8.7 Hz, 2H), 4.55 (br, 1H), 4.35-4.20 (m, 1H), 3.26 (d, J=6.1 Hz, 2H), 1.88-1.36 (m, 8H) ppm. MS (ESI): 375.9 (M+H)⁺, 373.9 (M−H)⁻ IR (KBr)νₘₐₓ: 3240, 2949, 1632, 1508, 1491, 1281, 1240 cm⁻¹ m.p. 196.6° C.

### Example 33

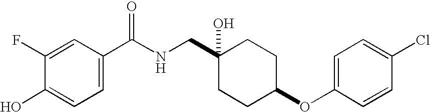

### N-{[trans-4-(4-Chlorophenoxy)-1-hydroxycyclohexyl]methyl}-3-fluoro-4-hydroxybenzamide and

### Example 34

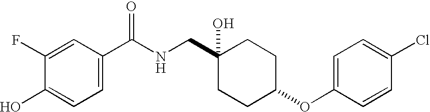

### N-{[cis-4-(4-Chlorophenoxy)-1-hydroxycyclohexyl]methyl}-3-fluoro-4-hydroxybenzamide

These compounds were prepared with 3-fluoro-4-hydroxybenzoic by a procedure similar to that in Example 31 and 32.

### Data for Example 33:

¹H NMR (DMSO-d₆) δ: 8.12 (t, J=5.9 Hz, 1H), 7.71-7.52 (m, 2H), 7.28 (d, J=8.9 Hz, 2H), 7.02-6.90 (m, 3H), 4.56-4.42 (m, 2H), 3.40-3.20 (m, 2H), 1.92-1.50 (m, 6H), 1.43-1.26 (m, 2H) ppm. (—OH was not observed) MS (ESI): 394.05 (M+H)⁺, 392.04 (M−H)⁻ IR (KBr)νₘₐₓ: 3350, 1957, 1639, 1512, 1310, 1238 cm⁻¹ m.p. 168.5° C.

### Data for Example 34:

¹H NMR (DMSO-d₆) δ: 8.11 (t, J=5.9 Hz, 1H), 7.72-7.54 (m, 2H), 7.28 (d, J=8.9 Hz, 2H), 7.01-6.90 (m, 3H), 4.34-4.20 (m, 1H), 3.26 (d, J=6.1 Hz, 2H), 1.86-1.36 (m, 8H) ppm. (OH was not observed) MS (ESI): 394.07 (M+H)⁺, 392.05 (M−H)⁻ IR (KBr)νₘₐₓ: 3319, 2941, 1618, 1512, 1489, 1300, 1242 cm⁻¹ m.p. 168.1° C.

### Examples 35-38

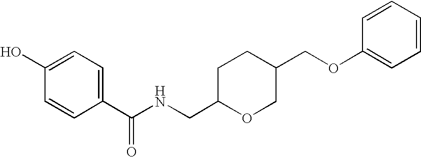

### (+)-4-Hydroxy-N-{[5S-(phenoxymethyl)tetrahydro-2H-pyran-2S-yl]methyl}benzamide

### (−)-4-Hydroxy-N-{[5R-(phenoxymethyl)tetrahydro-2H-pyran-2R-yl]methyl}benzamide

### (+)-4-Hydroxy-N-{[5R*-(phenoxymethyl)tetrahydro-2H-pyran-2S*-yl]methyl}benzamide

### (−)-4-Hydroxy-N-{[5S*-(phenoxymethyl)tetrahydro-2H-pyran-2R*-yl]methyl}benzamide

4-(Methoxymethoxy)-N-{[5-phenoxymethyl]tetrahydro-2H-pyran-2-yl}methyl}benzamide (678 mg, 1.76 mmol) was dissolved in 10˜20% HCl-MeOH (5 mL) and stirred at room temperature for 2 hours. To this mixture were added H₂O (50 mL) and AcOEt (50 mL). The aqueous layer was extracted with AcOEt (50 mL) and the combined organic layers were washed with sat. aq. NaHCO₃ (50 mL) and brine (50 mL), dried over Na₂SO₄, and concentrated in vacuo. The crude product was purified by silica gel column chromatography to give the mixture of the titled compounds (0.55 g, 92%). 4 stereoisomers were separated by Chiral column (Chiralpak AD-H, 20 mm I.D.×250 mm (No. ADH0CJ-DE003), DAICEL) using n-Hexane:2-Propanol:Et₂NH=90:10:0.1 as an eluent (Flow rate: 10 mL/min).

### Data for Example 35:

Sticky colorless solid, 99% ee, cis isomer, retention time 33 min

¹H NMR (300 MHz, DMSO) δ: 8.26-8.22 (m, 1H), 7.73-7.69 (m, 2H), 7.31-7.25 (m, 2H), 6.97-6.90 (m, 3H), 6.80-6.75 (m, 2H), 4.17-4.11 (m, 1H), 4.10-3.90 (m, 2H), 3.56-3.44 (m, 2H), 3.29-3.19 (m, 2H), 1.95 (bs, 1H), 1.87-1.67 (m, 2H), 1.50-1.30 (m, 2H) ppm. (—OH was not observed) MS (ESI): 342.1 (M+H)⁺, 340.1 (M−H)⁻ [α]^{D}=+12.00 (c=0.10, MeOH, 26° C.)

### Data for Example 36

Sticky colorless solid, 99% ee, cis isomer, retention time 36 min

¹H NMR (300 MHz, DMSO) δ: 8.25-8.22 (m, 1H), 7.72-7.69 (m, 2H), 7.31-7.26 (m, 2H), 6.97-6.90 (m, 3H), 6.78-6.76 (m, 2H), 4.16-4.11 (m, 1H), 4.04-3.90 (m, 2H), 3.55-3.47 (m, 2H), 3.27-3.23 (m, 2H), 1.95 (bs, 1H), 1.86-1.69 (m, 2H), 1.49-1.23 (m, 2H) ppm. (OH was not observed) MS (ESI): 342.1 (M+H)⁺, 340.1 (M−H)⁻ [α]^{D}=−20.00 (c=0.04, MeOH, 26° C.)

### Data for Example 37

Re-crystalized from IPA/IPE; white solid, >99% ee, trans, retention time 47 min

¹H NMR (300 MHz, CDCl₃) δ: 7.71-7.61 (m, 2H), 7.31-7.25 (m, 2H), 6.70-6.85 (m, 5H), 6.53 (bs, 1H), 6.22 (bs, 1H), 4.23-4.18 (m, 1H), 3.85-3.69 (m, 3H), 3.52-3.46 (m, 1H), 3.30-3.21 (m, 2H), 2.15-2.11 (m, 1H), 1.98-1.95 (m, 1H), 0.78-1.74 (m, 1H), 1.48-1.36 (m, 2H) ppm. MS (ESI): 342.1 (M+H)⁺, 340.1 (M−H)⁻ [α]^{D}=+28.9 (c=0.18, MeOH, 26° C.) mp=152.1° C. IR (KBr)=3355.9, 2935.5, 1635.5, 1508.2, 1226.6, 1074.3 cm⁻¹

### Data for Example 38

Recrystallized from IPA/IPE; white solid; 99% ee, trans, retention time 51 min

¹H NMR (300 MHz, CDCl₃) δ: 7.70-7.67 (m, 2H), 7.30-7.27 (m, 2H), 6.97-6.85 (m, 5H), 6.60-6.35 (m, 2H), 4.23-4.19 (m, 1H), 3.85-3.70 (m, 3H), 3.50-3.46 (m, 1H), 3.30-3.21 (m, 2H), 2.12 (bs, 1H), 1.98-1.95 (m, 1H), 1.77-1.73 (m, 1H), 1.52-1.36 (m, 2H) ppm. MS (ESI): 342.1 (M+H)⁺, 340.1 (M−H)⁻ [α]^{D}=−25.3 (c=0.19, MeOH, 26° C.) mp=152.4° C. IR (KBr)=3355.9, 2935.5, 1635.5, 1508.2, 1226.6, 1074.3 cm⁻¹

### Example 39-42

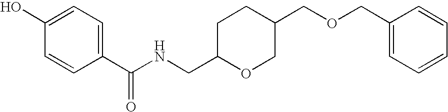

### 4-Hydroxy-N-{[5S-(benzyloxymethyl)tetrahydro-2H-pyran-2S-yl]methyl}benzamide

### 4-Hydroxy-N-{[5R-(benzyloxymethyl)tetrahydro-2H-pyran-2R-yl]methyl}benzamide

### 4-Hydroxy-N-{[5R*-(benzyloxymethyl)tetrahydro-2H-pyran-2S*-yl]methyl}benzamide

### 4-Hydroxy-N-{[5S*-(benzyloxymethyl)tetrahydro-2H-pyran-2R*-yl]methyl}benzamide

4-(benzyloxymethoxy)-N-{[5-phenoxymethyl]tetrahydro-2H-pyran-2-yl}methyl}benzamide (1.6 g, 4.0 mmol) was dissolved in 10˜20% HCl-MeOH (10 mL) and stirred at room temperature for 2 h. To the mixture were added H₂O (50 mL) and AcOEt (50 mL). The aqueous layer was extracted with AcOEt (50 mL) and the combined organic layers were washed with sat. NaHCO₃ (50 mL), brine (50 mL), dried over Na₂SO₄, and concentrated in vacuo. The crude product was purified by silica gel column chromatography to give the mixture of the titled compounds (1.20 g, 83%). 4 stereoisomers were separated by Chiral column (Chiralpak AD-H, 20 mm I.D.×250 mm (No. ADH0CJ-DE003), DAICEL) using n-Hexane/2-Propanol/Et₂NH=85:15:0.1 as an eluent (10 mL/min).

### Data for Example 39

colorless amorphous, >99% ee, cis isomer, retention time 18 min

¹H NMR (300 MHz, DMSO) δ: 8.25-8.21 (m, 1H), 7.72-7.69 (m, 2H), 7.38-7.25 (m, 5H), 6.80-6.76 (m, 2H), 4.50 (d, J=12.3 Hz, 1H), 4.45 (d, J=12.3 Hz, 1H), 3.83-3.79 (m, 1H), 3.61-3.56 (m, 1H), 3.48-3.43 (m, 3H), 3.28-3.15 (m, 2H), 1.77-1.59 (m, 3H), 1.44-1.41 (m, 1H), 1.32-1.19 (m, 1H) ppm. (OH was not observed) MS (ESI): 356.1 (M+H)⁺, 354.1 (M−H)⁻.

### Data for Example 40

colorless amorphous; >99% ee, cis isomer; retention time 21 min

¹H NMR (300 MHz, DMSO) δ: 8.25-8.21 (m, 1H), 7.72-7.69 (m, 2H), 7.38-7.25 (m, 5H), 6.78-6.76 (m, 2H), 4.50 (d, J=12.0 Hz, 1H), 4.45 (d, J=12.0 Hz, 1H), 3.83-3.79 (m, 1H), 3.61-3.56 (m, 1H), 3.48-3.43 (m, 3H), 3.28-3.17 (m, 2H), 1.77-1.59 (m, 3H), 1.45-1.41 (m, 1H), 1.32-1.19 (m, 1H) ppm. (OH was not observed) MS (ESI): 356.1 (M+H)⁺, 354.1 (M−H)⁻.

### Data for Example 41

colorless amorphous, >99% ee, trans isomer, retention time 34 min

¹H NMR (300 MHz, DMSO) δ: 8.26-8.22 (m, 1H), 7.71 (d, J=8.4 Hz, 2H), 7.37-7.25 (m, 5H), 6.77 (d, J=8.4 Hz, 2H), 4.45 (d, J=12.6 Hz, 1H), 4.40 (d, J=12.6 Hz, 1H), 3.97-3.94 (m, 1H), 3.27-3.21 (m, 5H), 3.10-3.03 (m, 1H), 1.79 (bs, 2H), 1.67-1.63 (m, 1H), 1.18 (bs, 2H) ppm. (OH was not observed) MS (ESI): 356.1 (M+H)⁺, 354.1 (M−H)⁻

### Data for Example 42

colorless amorphous; 98% ee, trans isomer; retention time 38 min

¹H NMR (300 MHz, DMSO) δ: 8.32-8.22 (m, 1H), 7.72 (d, J=8.7 Hz, 2H), 7.38-7.24 (m, 5H), 6.77 (d, J=8.7 Hz, 2H), 4.45 (d,J=12.9 Hz, 1H), 4.41 (d, J=12.9 Hz, 1H), 3.97-3.94 (m, 1H), 3.27-3.17 (m, 5H), 3.10-3.03 (m, 1H), 1.79 (bs, 2H), 1.67-1.64 (m, 1H), 1.18 (bs, 2H) ppm. (—OH was not observed) MS (ESI): 356.1 (M+H)⁺, 354.1 (M−H)⁻.

### Examples 43-46

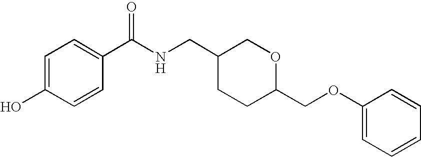

### (−)-4-Hydroxy-N-{[(3R,6S)-6-(phenoxymethyl)tetrahydro-2H-pyran-3-yl]methyl}benzamide

### (+)-4-Hydroxy-N-{[(3S,6R)-6-(phenoxymethyl)tetrahydro-2H-pyran-3-yl]methyl}benzamide

### (+)-4-Hydroxy-N-{[(3R,6R)-6-(phenoxymethyl)tetrahydro-2H-pyran-3-yl]methyl}benzamide

### (−)-4-Hydroxy-N-{[(3S,6S)-6-(phenoxymethyl)tetrahydro-2H-pyran-3-yl]methyl}benzamide

A mixture of to {[6-(phenoxymethyl)tetrahydro-2H-pyran-3-yl]methyl}amine (0.13 g), 4-hydroxybenzoic acid (83 mg, 0.60 mmol), HOBt.H₂O (0.11 g, 0.72 mmol) and EDCI (0.14 g, 0.72 mmol) in DMF (3.0 mL) was stirred at room temperature for 16 hours. The mixture was diluted with AcOEt and was washed with sat. aq. NaHCO₃ and water, dried over MgSO₄ and evaporated. The residue was dissolved with MeOH (3 mL) and 2N aq. NaOH (3 mL). The mixture was stirred for 2 hours and neutralized with 2 N aq. HCl (3 mL). The whole was extracted with AcOEt. The extract was washed with sat. aq. NaHCO₃ and water, dried over MgSO₄, and evaporated. The residue was purified by prep. TLC (hexane:AcOEt=1:2) to give the mixture of titled compounds. 4 stereoisomers were separated by chiral HPLC (DAICEL Chiralpak AD-H, hexane/EtOH/Et₂NH=85/15/0.1).

### Data for Example 43

colorless amorphous, >99% ee, cis isomer, retention time 25 min

¹H NMR (CDCl₃) δ: 7.67 (d, J=8.6 Hz, 2H), 7.31-7.24 (m, 3H), 6.98-6.82 (m, 4H), 6.47-6.37 (m, 1H), 5.89 (br, 1H), 4.05-3.48 (m, 7H), 2.04-1.50 (m, 5H) ppm. MS (ESI): 342.12 (M+H)⁺ IR (KBr) 3250, 2934, 2860, 1607, 1587, 1508, 1454, 1242 cm⁻¹ Isomer 1: [α]_{D}=−7.2 (c=0.25, MeOH)

### Data for Example 44

colorless amorphous, >99% ee, cis isomer, retention time 27 min

¹H NMR (CDCl₃) δ: 7.67 (d, J=8.6 Hz, 2H), 7.31-7.24 (m, 3H), 6.98-6.82 (m, 4H), 6.47-6.37 (m, 1H), 5.73 (br, 1H), 4.05-3.48 (m, 7H), 2.04-1.50 (m, 5H) ppm. MS (ESI): 342.13 (M+H)⁺ IR (KBr) 3250, 2934, 2860, 1607, 1587, 1508, 1454, 1242 cm⁻¹ Isomer 2: [α]_{D}=+8.8 (c=0.25, MeOH)

### Data for Example 45

colorless amorphous, >99% ee, trans isomer, retention time 59 min

¹H NMR (DMSO-d₆) δ: 9.94 (br, 1H), 8.25-8.15 (m, 1H), 7.67 (d, J=8.7 Hz, 2H), 7.31-7.23 (m, 2H), 6.96-6.88 (m, 3H), 6.78 (d, J=8.7 Hz, 2H), 3.96-3.86 (m, 3H), 3.65-3.50 (m, 1H), 3.16-3.00 (m, 3H), 1.93-1.65 (m, 3H), 1.45-1.10 (m, 2H) ppm. MS (ESI): 342.14 (M+H)⁺, 340.12 (M−H)⁻. Isomer 3: [α]_{D}=+2.4 (c=0.25, MeOH)

### Data for Example 46

colorless amorphous, >99% ee, trans isomer, retention time 71 min

¹H NMR (DMSO-d₆) δ: 9.93 (br, 1H), 8.25-8.15 (m, 1H), 7.67 (d, J=8.7 Hz, 2H), 7.31-7.23 (m, 2H), 6.96-6.88 (m, 3H), 6.78 (d, J=8.7 Hz, 2H), 3.96-3.86 (m, 3H), 3.65-3.50 (m, 1H), 3.16-3.00 (m, 3H), 1.93-1.65 (m, 3H), 1.45-1.10 (m, 2H) ppm. MS (ESI): 342.13 (M+H)⁺, 340.10 (M−H)⁻. Isomer 3: [α]_{D}=−3.4 (c=0.50, MeOH)

### Example 47

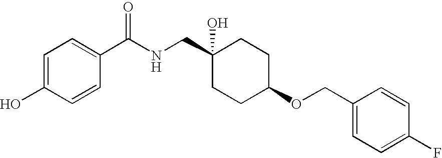

### N-({trans-4-[(4-Fluorobenzyl)oxy]-1-hydroxycyclohexyl}methyl)-4-hydroxybenzamide

To a solution of 4-{[({4-[(4-fluorobenzyl)oxy]-1-hydroxycyclohexyl}methyl)amino]carbonyl}phenyl acetate (4.0 g, 9.6 mmol) in MeOH (20 ml) and THF (20 ml) was added 2N—NaOH aq. (9.6 ml) at 0°0 C. and the mixture was stirred at the same temperature for 2 hr. The reaction mixture was adjusted to pH 4.0 with 2N—HCl aq. The solvent was removed in vacuo. The residue was extracted with ethyl acetate (50 ml×3). The combined organic layer was dried over Na₂SO₄, and concentrated in vacuo. The residue was purified by column chromatography on silica gel (dichloromethane:methanol=20:1 as eluent) and HPLC to afford the titled compound as a white solid (248 mg, 7%).

¹H NMR (DMSO-d₆) δ: 9.97 (br, 1H), 8.02-7.98 (m, 1H), 7.74-7.71 (m, 2H), 7.35-7.30 (m, 2H), 7.12-7.06 (m, 2H), 6.81-6.78 (m, 2H), 4.50 (br, 1H), 4.41 (s, 2H), 3.52 (br, 1H), 3.26 (d, J=6.0 Hz, 2H), 1.79-1.58 (m, 6H), 1.32-1.26 (m, 2H) ppm. MS (ESI): 374.12 (M+H)⁺, 372.12 (M−H)⁻ IR (KBr)νₘₐₓ: 2932, 1703, 1508, 1227, 1084, 826 cm⁻¹ Anal. Calcd. for C₂₁H₂₄NO₄F: C, 67.54; H, 6.48; N, 3.75. Found: C, 67.43; H, 6.47; N, 3.70. m.p. 122.1° C., 160.9° C.

### Example 48

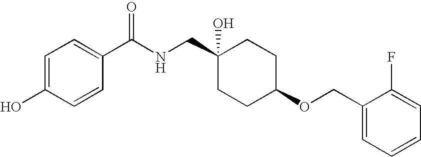

### N-({trans-4-[(2-Fluorobenzyl)oxy]-1-hydroxycyclohexyl}methyl)-4-hydroxybenzamide

This compound was prepared with 4-{[({4-[(2-fluorobenzyl)oxy]-1-hydroxycyclohexyl}methyl)amino]carbonyl}phenyl acetate by a procedure similar to that in Example 47 as a white solid.

¹H NMR (DMSO-d₆) δ: 9.96 (br, 1H), 8.01-7.97 (m, 1H), 7.73-7.70 (m, 2H), 7.44-7.29 (m, 2H), 7.18-7.10 (m, 2H), 6.80-6.77 (m, 2H), 4.50-4.48 (m, 3H), 3.55 (br, 1H), 3.26 (d, J=5.9 Hz, 2H), 1.80-1.57 (m, 6H), 1.31-1.27 (m, 2H) ppm. MS (ESI): 374.08 (M+H)⁺, 372.04 (M−H)⁻ IR (KBr)νₘₐₓ: 3142, 1607, 1234, 1067, 763 cm⁻¹ Anal. Calcd. for C₂₁H₂₄NO₄F: C, 67.54; H, 6.48; N, 3.75. Found: C, 67.32; H, 6.58; N, 3.78. m.p. 162.9° C., 179.9° C.

### Example 49

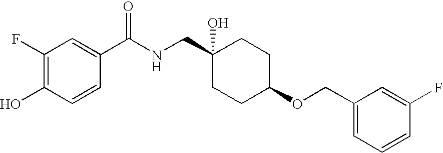

### 3-Fluoro-N-({trans-4-[(3-fluorobenzyl)oxy-1-hydroxycyclohexyl]methyl}-4-hydroxybenzamide

This compound was prepared with 4-hydroxy-3-fluorobenzoic acid and 1-(aminomethyl)-4-[(3-fluorobenzyl)oxy]cyclohexanol by a procedure similar to that in Example 8 as a white solid.

¹H NMR (DMSO-d₆) δ: 8.09-8.07 (m, 1H), 7.69-7.68 (m, 1H), 7.57-7.54 (m, 1H), 7.37-7.29 (m, 1H), 7.15-7.04 (m, 3H), 7.00-6.93 (m, 1H), 4.46 (s, 2H), 3.53 (br, 1H), 3.28-3.26 (m, 2H), 1.81-1.58 (m, 6H), 1.32-1.27 (m, 2H) ppm. [PhOH and OH proton were not observed.] MS (ESI): 392.05 (M+H)⁺, 390.03 (M−H)⁻ IR (KBr.)νₘₐₓ: 2934, 1589, 1110, 785 cm⁻¹ Anal. Calcd. for C₂₁H₂₃NO₄F₂: C, 64.44; H, 5.92; N, 3.58. Found: C, 64.12; H, 5.95; N, 3.61. m.p. 138.2° C.

### Example 50

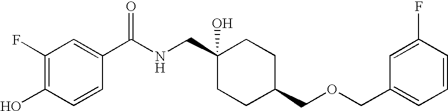

### 3-Fluoro-N-[(trans-4-{[(3-fluorobenzyl)oxy]methyl}-1-1-hydroxycyclohexyl)methyl]-hydroxybenzamide

This compound was prepared with 4-hydroxy-3-fluorobenzoic acid and trans-1-(aminomethyl)-4-{[(3-fluorobenzyl)oxy]methyl}cyclohexanol by a procedure similar to that in Example 8 as a white solid.

¹H NMR (DMSO-d₆) δ: 10.48 (br, 1H), 8.04-8.00 (m, 1H), 7.71-7.66 (m, 1H), 7.59-7.55 (m, 1H), 7.43-7.35 (m, 1H), 7.17-7.06 (m, 3H), 7.01-6.96 (m, 1H), 4.62 (br, 1H), 4.47 (s, 2H), 3.34 (m, 2H), 3.29 (d, J=6.0 Hz, 2H), 1.64-1.60 (m, 5H), 1.33-1.16 (m, 4H) ppm. MS (ESI): 406.07 (M+H)⁺, 404.09 (M−H)⁻ IR (KBr)νₘₐₓ: 3179, 1638, 1516, 1298, 1094 cm⁻¹ Anal. Calcd. for C₂₂H₂₅NO₄F₂: C, 65.17; H, 6.22; N, 3.45. Found: C, 65.14; H, 6.24; N, 3.47. m.p. 132.4° C.

### Example 51

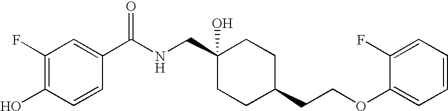

### 3-Fluoro-N-({trans-4-[2-(2-fluorophenoxy)ethyl]-1-hydroxycyclohexyl}methyl)-4-hydroxybenzamide

This compound was prepared with 4-hydroxy-3-fluorobenzoic acid and trans-1-(aminomethyl)-4-[2-(2-fluorophenyl)ethyl[cyclohexanol by a procedure similar to that in Example 8 as a white solid.

¹H NMR (DMSO-d₆) δ: 10.46 (br, 1H), 8.04-8.02 (m, 1H), 7.72-7.71 (m, 1H), 7.67-7.57 (m, 1H), 7.22-6.90 (m, 5H), 4.59 (br, 1H), 4.09-4.04 (m, 2H), 3.37 (m, 2H), 1.68-1.16 (m, 11H) ppm. MS (ESI): 406.05(M+H)⁺, 404.02(M−H)⁻ IR (KBr)νₘₐₓ: 3217,2928, 1634, 1508, 1285, 1113 cm⁻¹ Anal. Calcd. for C₂₂H₂₅NO₄F₂: C, 65.17; H, 6.22; N, 3.45. Found: C, 64.98; H, 6.18; N, 3.46; m.p. 184.7° C.

### Example 52 and 53

### Cis- and Trans-N-{[4-(4-chlorophenoxy)cyclohexyl]methyl}-3-fluoro-4-hydroxybenzamide

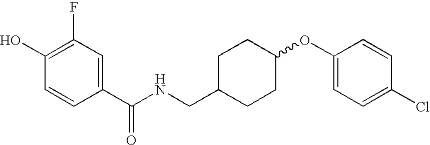

A mixture of 4-(azidomethyl)cyclohexyl 4-chlorophenyl ether (3.0 g, 11 mmol) and 10% Pd/C (0.3 g) in MeOH (50 mL) was stirred under H₂ atmosphere at rt. After 14 h, the mixture was filtered through a pad of celite and washed with MeOH (50 mL) and concentrated in vacuo. The residue (0.76 g out of 2.5 g, ˜3.4 mmol) was dissolved in DMF (20 mL) and to this were added 3-fluoro-4-hydroxybenzoic acid (0.5 g, 3.2 mmol), WSC (0.73 g, 3.8 mmol), HOBt (0.58 g, 3.8 mmol) and Et₃N (0.90 mL, 6.4 mmol) at rt. After 18 h, the reaction mixture was quenched by addition of sat. aq. NaHCO₃ (50 mL) and diluted with AcOEt (50 mL). The aqueous layer was extracted with AcOEt (50 mL×2) and the combined organic layer was washed with H₂O (50 mL×2) and brine (50 mL), dried over MgSO₄, filtered and concentrated. The residue was dissolved in MeOH (15 mL) and to this solution was added 2N NaOH (10 mL) and the mixture was stirred at rt. After 2 h, to this was added sat. aq. NaHCO₃ (50 mL) and extracted with AcOEt (100 mL). The aqueous layer was extracted with AcOEt (50 mL) and the combined organic layer was washed with brine (50 mL), dried over Na₂SO₄, filtered and concentrated in vacuo. The residue was purified by silica gel column chromatography (hexane:AcOEt=2:1˜1.5:1) and HPLC saparation to give 3-fluoro-4-hydroxy-N-[(4-phenoxycyclohexyl)methyl]benxamide (94.7 mg, 8% over 2 steps) and N-{[4-(4-chlorophenoxy)cyclohexyl]methyl}-3-fluoro-4-hydroxybenzamide (80.7 mg, 6% over 2 steps).

The cis/trans separation of N-{[4-(4-chlorophenoxy)cyclohexyl]methyl}-3-fluoro-4-hydroxybenzamide was carried out by chiral column (Chiralcel OJ, 20 mm I.D.×250 mm (No. 53-03-20910), DAICEL) using n-hexane:EtOH:Et₂NH=79:21:0.1 as an eluent (Flow rate=7 mL/min) at 40° C.

### Example 52

white solid., 99% de, trans isomer, retention time 24 min.

¹H NMR (300 MHz, CDCl₃) δ: 7.59 (dd, J=11.1, 2.1 Hz, 1H), 7.46-7.42 (m, 1H), 7.24-7.18 (m, 2H), 7.04 (t, J=8.4 Hz, 1H), 6.84-6.79 (m, 2H), 6.08 (bs, 1H), 4.11 (tt, J=10.8, 4.2 Hz, 1H), 3.34 (t, J=6.3 Hz, 2H), 2.19-2.15 (m, 2H), 1.94-1.90 (m, 2H), 1.71-1.59 (m, 1H), 1.50-1.36 (m, 2H), 1.29-1.07 (m, 2H) ppm. (OH was not observed.) MS (ESI): 378.07 (M+H)⁺, 376.08 (M−H)⁻

### Example 53

white solid, 98% de, cis isomer, retention time 28 min.

¹H NMR (300 MHz, CDCl₃) δ: 7.57 (dd, J=11.1, 2.1 Hz, 1H), 7.44-7.41 (m, 1H), 7.24-7.19 (m, 2H), 7.02 (t, J=8.4 Hz, 1H), 6.85-6.80 (m, 2H), 6.12 (bs, 1H), 4.49 (bs, 1H), 3.35 (t, J=7.5 Hz, 2H), 2.06-2.02 (m, 2H), 1.72-1.28 (m, 7H) ppm. (OH was not observed.) MS (ESI): 378.10 (M+H)⁺, 376.07 (M−H)⁻

### Example 54

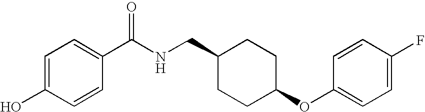

### N-{[cis-4-(4-Fluorophenoxy)cyclohexyl]methyl}-4-hydroxybenzamide

This compound was prepared with N-{[cis-4-(4-fluorophenoxy)cyclohexyl]methyl}-4-(methoxymethoxy)benzamide by a procedure similar to that in Example 6 as a white solid. ¹H NMR (DMSO-d₆) δ: 9.94 (br, 1H), 8.22-8.18 (m, 1H), 7.70 (d, J=8.7 Hz, 2H), 7.12-7.06 (m, 2H), 6.98-6.93 (m, 2H), 6.78 (d, J=8.7 Hz, 2H), 4.49 (m, 1H), 3.15-3.11 (m, 2H), 1.87-1.84 (m, 2H), 1.65-1.49 (m, 5H), 1.35-1.26 (m, 2H) ppm. MS (ESI): 344.20 (M+H)⁺, 342.19 (M−H)⁻ IR (KBr.) νₘₐₓ: 3283, 2934, 1632, 1502, 1202 cm⁻¹ Anal. Calcd. for C₂₀H₂₂NO₃F: C, 69.95; H, 6.46; N, 4.08. Found: C, 70.10; H, 6.46; N, 4.10. m.p. 170.5° C.

### Example 55

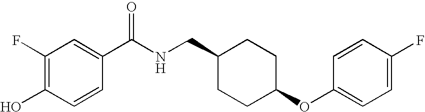

### 3-Fluoro-N-{[cis-4-(4-fluorophenoxy)cyclohexyl]methyl}-4-hydroxybenzamide

This compound was prepared with 3-fluoro-4-hydroxybenzoic acid and {[4-(4-fluorophenoxy)cyclohexyl]methyl}amine by a procedure similar to that in Example 8 as a white solid.

¹H NMR (DMSO-d₆) δ: 8.29 (m, 1H), 7.64-7.52 (m, 2H), 7.12-7.06 (m, 2H), 6.98-6.93 (m, 4H), 4.50 (br, 1H), 3.16-3.11 (m, 2H), 1.87-1.26 (m, 9H) ppm. MS (ESI): 362.13 (M+H)⁺, 360.08 (M−H)⁻ IR (KBr)νₘₐₓ: 1498, 1436, 833, 760 cm⁻¹. Anal. Calcd. for C₂₀H₂₁NO₃F₂: C, 66.47; H, 5.86; N, 3.88. Found: C, 66.35; H, 5.83; N, 3.90. m.p. 149.5° C.

### Example 56

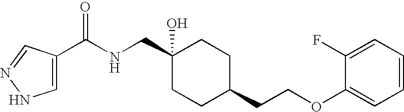

### N-({trans-4-[2-(2-Fluorophenoxy)ethyl]-1-hydroxycyclohexyl}methyl)-1H-pyrazole-4-carboxamide

This compound was prepared with 1H-pyrazole-4-carboxylic acid and trans-1-(aminomethyl)-4-[2-(2-fluorophenyl)ethyl[cyclohexanol by a procedure similar to that in Example 8 as a white solid.

¹H NMR (DMSO-d₆) δ: 13.11 (br, 1H), 8.08 (br, 2H), 7.82-7.78 (m, 1H), 7.23-7.08 (m, 3H), 6.95-6.87 (m, 1H), 4.59 (br, 1H), 4.09-4.04 (m, 2H), 3.31 (m, 2H), 1.68-1.16 (m, 11H) ppm. MS (ESI): 362.18 (M+H)⁺ IR (KBr)νₘₐₓ: 3173, 2924, 1636, 1508, 1283, 746 cm⁻¹ Anal. Calcd. for C₁₉H₂₄N₃O₃F: C, 63.14; H, 6.69; N, 11.63. Found: C, 62.99; H, 6.63; N, 11.61. m.p. 175.1° C.

### Example 57

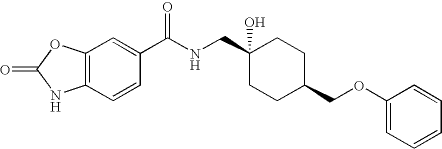

### N-{[trans-1-Hydroxy-4-(phenoxymethyl)cyclohexyl]methyl}-2-oxo-2,3-dihydro-1,3-benzoxazole-6-carboxamide

This compound was prepared with 2-oxo-2,3-dihydro-1,3-benzoxazole-6-carboxylic acid by a procedure similar to that in Example 8 as a white solid.

¹H NMR (DMSO-d₆) δ: 11.92 (br, 1H), 8.20-8.11 (m, 1H), 7.81 (s, 1H), 7.78-7.70 (m, 1H), 7.30-7.21 (m, 2H), 7.15 (d, J=8.1 Hz, 1H), 6.96-6.86 (m, 3H), 4.65 (s, 1H), 3.82 (d, J=6.1 Hz, 2H), 3.45-3.35 (m, 2H), 1.83-1.60 (m, 5H), 1.40-1.20 (m, 4H) ppm. MS (ESI): 397.22 (M+H)⁺, 395.21 (M−H)⁻ IR (KBr)νₘₐₓ: 2934, 1763, 1601, 1495, 1240, 754 cm⁻¹

### Example 58

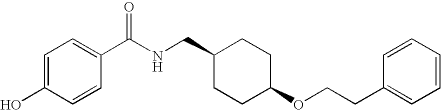

### 4-Hydroxy-N-{[cis-4-(2-phenylethoxy)cyclohexyl]methyl}benzamide

This compound was prepared with {[cis-4-(2-Phenylethoxy)cyclohexyl]methyl}amine by a procedure similar to that in Example 8 as a white solid.

1H NMR (DMSO-d₆) δ: 9.92 (br, 1H), 8.20-8.11 (m, 1H), 7.70 (d, J=8.7 Hz, 2H), 7.32-7.14 (m, 5H), 6.78 (d, J=8.7 Hz, 2H), 3.60-3.40 (m, 3H), 3.10-3.00 (m, 2H), 2.85-2.75 (m, 2H), 1.80-1.10 (m, 9H) ppm. IR (KBr)νₘₐₓ: 2922, 1541, 1277, 1238, 1175, 754 cm⁻¹

### Example 59

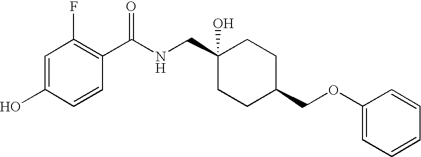

### 2-Fluoro-4-hydroxy-N-{[trans-1-hydroxy-4-(phenoxymethyl)cyclohexyl]methyl}benzamide

This compound was prepared with 2-fluoro-4-hydroxybenzoic acid by a procedure similar to that in Example 8 as a white solid.

¹H NMR (DMSO-d₆) δ: 10.47 (br, 1H), 7.76-7.45 (m, 2H), 7.31-7.21 (m, 2H), 6.96-6.85 (m, 3H), 6.72-6.55 (m, 2H), 4.68 (s, 1H), 3.82 (d, J=6.2 Hz, 2H), 3.45-3.35 (m, 2H), 1.83-1.60 (m, 5H), 1.40-1.20 (m, 4H) ppm. MS (ESI): 374.23 (M+H)⁺, 372.24 (M−H)⁻ IR (KBr)νₘₐₓ: 3200, 2938, 1495, 1227, 847, 768 cm⁻¹

### Example 60

### N-({trans-4-[(Benzyloxy)methyl]-1-hydroxycyclohexyl}methyl)-3-fluoro-4-hydoxybenzamide

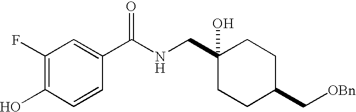

This compound was prepared with 3-fluoro-4-hydroxybenzoic acid and trans-1-(aminomethyl)-4-[(benzyloxy)methyl]cyclohexanol hydrochloride by a procedure similar to that in Example 8.

¹H NMR (300 MHz, DMSO) δ: 8.01 (m, 1H), 7.70-7.55 (m, 2H), 7.37-7.24 (m, 5H), 6.98 (t, J=8.61 Hz, 1H), 4.44 (s, 2H), 3.29-3.27 (m, 4H), 1.63-1.60 (m, 5H), 1.32-1.15 (m, 4H) ppm. (OH was not observed.) MS (ESI): 386.16 (M−H)⁻ mp=162.5° C. IR (KBr)νₘₐₓ: 3355.9, 2945.1, 1635.5, 1517.9, 1299.9, 1093.6 cm⁻¹. Anal. Calcd for C₂₂H₂₆NO₄F: C, 68.20; H, 6.76; N, 3.62; O, 16.52; F, 4.90. Found: C, 68.12; H, 6.93; N, 3.63.

### Example 61

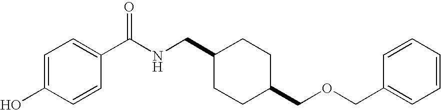

### N-({cis-4-[(Benzyloxy)methyl]cyclohexyl}methyl)-4-hydroxybenzamide

This compound was prepared with ({cis-4-[(benzyloxy)methyl]cyclohexyl}methyl)amine by a procedure similar to that in Example 8.

¹H NMR (CDCl₃) δ: 7.65 (d, J=8.7 Hz, 2H), 7.36-7.24 (m, 5H), 6.86 (d, J=8.7 Hz, 2H), 6.48 (s, 1H), 6.10-6.00 (m, 1H), 4.50 (s, 2H), 3.44-3.35 (m, 4H), 1.95-1.35 (m, 10H) ppm. MS (ESI): 354.23 (M+H)⁺, 352.23 (M−H)⁻

### Example 62

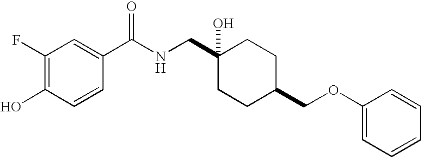

### 3-Fluoro-4-hydroxy-N-{[trans-1-hydroxy-4-(phenoxymethyl)cyclohexyl]methyl}benzamide

This compound was prepared with 3-fluoro-4-hydroxybenzoic acid by a procedure similar to that in Example 8.

¹H NMR (DMSO-d₆) δ: 10.46 (br, 1H), 8.04 (t, J=5.9 Hz, 1H), 7.73-7.55 (m, 2H), 7.30-7.22 (m, 2H), 7.02-6.88 (m, 4H), 4.63 (br, 1H), 3.82 (d, J=6.0 Hz, 2H), 3.37 (d, J=6.0 Hz, 2H), 1.86-1.58 (m, 5H), 1.40-1.20 (m, 4H) ppm. MS (ESI): 374.04 (M+H)⁺, 372.03 (M−H)⁻ IR (KBr)νₘₐₓ: 3296, 2934, 1499, 1242 cm⁻¹ m.p. 183.5° C.

### Example 63

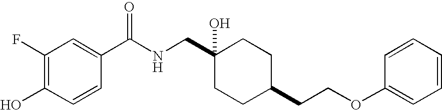

### 3-Fluoro-4-hydroxy-N-{[trans-1-hydroxy-4-(2-phenoxyethyl)cyclohexyl]methyl}benzamide

This compound was prepared with 3-fluoro-4-hydroxybenzoic acid and-trans-1-(aminomethyl)-4-(2-phenoxyethyl)cyclohexanol hydrochloride by a procedure similar to that in Example 8.

¹H NMR (DMSO-d₆) δ: 10.45 (br, 1H), 8.01 (t, J=5.9 Hz, 1H), 7.74-7.54 (m, 2H), 7.32-7.22 (m, 2H), 7.03-6.86 (m, 4H), 4.59 (br, 1H), 3.98 (t, J=6.4 Hz, 2H), 3.36 (d, J=6.4 Hz, 2H), 1.74-1.10 (m, 11H) ppm. MS (ESI): 388.14 (M+H)⁺, 386.16 (M−H)⁻ IR (KBr)νₘₐₓ: 3227, 2956, 1520, 1302 cm⁻¹ m.p. 164.0° C.

### Example 64

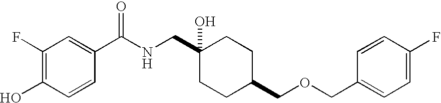

### 3-Fluoro-N-[(trans-4-{[(4-fluorobenzyl)oxy]methyl}-1-hydroxycyclohexyl)methyl]-4-hydroxybenzamide

This compound was prepared with 3-fluoro-4-hydroxybenzoic acid and trans-1-(aminomethyl)-4-{[(4-fluorobenzyl)oxy]methyl}cyclohexanol hydrochloride by a procedure similar to that in Example 8.

¹H NMR (DMSO-d₆) δ: 10.45 (br, 1H), 8.01 (t, J=5.9 Hz, 1H), 7.74-7.54 (m, 2H), 7.40-7.30 (m, 2H), 7.22-7.11 (m, 2H), 7.03-6.94 (m, 1H), 4.61 (br, 1H), 4.43 (s, 2H), 3.38-3.24 (m, 4H), 1.70-1.50 (m, 5H), 1.38-1.06 (m, 4H) ppm. MS (ESI): 406.12 (M+H)⁺, 404.13 (M−H)⁻ IR (KBr)νₘₐₓ: 3288, 2941, 1639, 1508, 1298 cm⁻¹ m.p. 155.9° C.

### Example 65

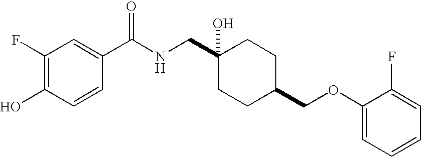

### 3-Fluoro-N-({trans-4-[(2-fluorophenoxy)methyl]-1-hydroxycyclohexyl}methyl)-4-hydroxybenzamide

This compound was prepared with 3-fluoro-4-hydroxybenzoic acid and trans-1-(aminomethyl)-4-[(2-fluorophenoxy)methyl]cyclohexanol hydrochloride by a procedure similar to that in Example 8.

¹H NMR (DMSO-d₆) δ: 10.46 (br, 1H), 8.03 (t, J=5.5 Hz, 1H), 7.74-7.54 (m, 2H), 7.24-6.88 (m, 5H), 4.63 (br, 1H), 3.90 (d, J=6.3 Hz, 2H), 3.42-3.32 (m, 2H), 1.95-1.55 (m, 5H), 1.42-1.22 (m, 4H) ppm. MS (ESI): 392.16 (M+H)⁺, 390.10 (M−H)⁻ IR (KBr)νₘₐₓ: 2926, 1562, 1508, 1307, 1250 cm⁻¹ m.p. 194.6° C.

### Example 66

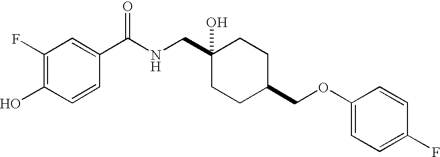

### 3-Fluoro-N-({trans-4-[(4-fluorophenoxy)methyl]-1-hydroxycyclohexyl}methyl)-4-hydroxybenzamide

This compound was prepared with 3-fluoro-4-hydroxybenzoic and trans-1-(aminomethyl)-4-[(4-fluorophenoxy)methyl]cyclohexanol hydrochloride by a procedure similar to that in Example 8.

¹H NMR (DMSO-d₆) δ: 10.47 (br, 1H), 8.15-7.95 (m, 1H), 7.78-7.53 (m, 2H), 7.20-6.87 (m, 5H), 4.64 (br, 1H), 3.80 (d, J=5.9 Hz, 2H), 3.50-3.25 (m, 2H), 1.89-1.55 (m, 5H), 1.45-1.18 (m, 4H) ppm. MS (ESI): 392.12 (M+H)⁺, 390.10 (M−H)⁻ IR (KBr)νₘₐₓ: 3288, 2926, 1628, 1508, 1299 cm⁻¹ m.p. 181.6° C.

### Example 67

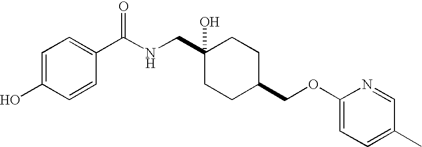

### 4-Hydroxy-N-[(trans-1-hydroxy-4-{[(5-methylpyridin-2-yl)oxy]methyl}cyclohexyl)methyl]benzamide

This compound was prepared with N-[(trans-1-hydroxy-4-{[(5-methylpyridin-2-yl)oxy]methyl}cyclohexyl)methyl]-4-(methoxymethoxy)benzamide by a procedure similar to that in Example 6.

¹H NMR (DMSO-d₆) δ: 9.98 (br, 1H), 8.06-7.85 (m, 2H), 7.73 (d, J=8.7 Hz, 2H), 7.55-7.47 (m, 1H), 6.80 (d, J=8.7 Hz, 2H), 6.69 (d, J=8.4 Hz, 1H), 4.71 (br, 1H), 4.08 (d, J=6.4 Hz, 2H), 3.45-3.30 (m, 2H), 2.19 (s, 3H), 1.86-1.52 (m, 5H), 1.43-1.13 (m, 4H) ppm. MS (ESI): 371.10 (M+H)⁺, 369.08 (M−H)⁻ IR (KBr)νₘₐₓ: 3358, 2934, 1570, 1512, 1277 cm⁻¹ m.p. 196.2° C.

### Example 68

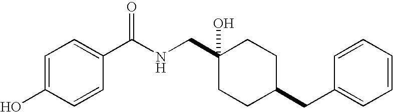

### N-[(trans-4-Benzyl-1-hydroxycyclohexyl)methyl]-4-hydroxybenzamide

A mixture of N-[(4-benzylidene-1-hydroxycyclohexyl)methyl]-4-(benzyloxy)benzamide (42 mg) and 20% Pd(OH)₂—C (10 mg) in MeOH (5 mL) was hydrogenated at 4 atm for 10 hours. The mixture was filtered through a pad of celite and the filtrate was evaporated. The residue was purified by silica gel column chromatography (hexane:AcOEt=1:2), followed by HPLC (DAICEL CHIRALCEL OJ-H, hexane:EtOH:Et₂NH=85:15:0.1) to give the titled compound (29 mg) as a white solid.

¹H NMR (DMSO-d₆) δ: 9.97 (br, 1H), 7.95-7.85 (m, 1H), 7.74 (d, J=8.6 Hz, 2H), 7.34-7.12 (m, 5H), 6.80 (d, J=8.6 Hz, 2H), 4.65 (s, 1H), 3.45-3.30 (m, 2H), 1.77-1.04 (m, 11H) ppm. MS (ESI): 340.20 (M+H)⁺, 338.21 (M−H)⁻ IR (KBr)νₘₐₓ: 3165, 2925, 1541, 1508, 1285 cm⁻¹

### Example 69

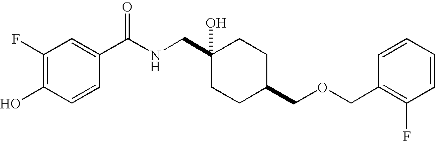

### 3-Fluoro-N-[(trans-4-{[(2-fluorobenzyl)oxy]methyl}-1-hydroxycyclohexyl)methyl]-4-hydroxybenzamide

This compound was prepared with 3-fluoro-4-hydroxybenzoic and trans-1-(aminomethyl)-4-{[(2-fluorobenzyl)oxy]methyl}cyclohexanol by a procedure similar to that in Example 8.

¹H-NMR (DMSO-d₆) δ: 8.02 (dd, J=5.9, 5.7 Hz, 1H), 7.69 (dd, J=12.5, 2 Hz, 1H), 7.57 (dd, J=8.4, 1.5 Hz, 1H), 7.44 (ddd, J=7.5, 7.5, 1.6 Hz, 1H), 7.40-7.32 (m, 1H), 7.23-7.14 (m, 2H), 6.99 (t, J=8.6 Hz, 1H), 4.62 (br, 1H), 4.50 (s, 2H) 3.25-3.45 (m, 4H), 1.66-1.57 (m, 5H), 1.34-1.10 (m, 4H) ppm. (OH was not observed.)

### Example 70

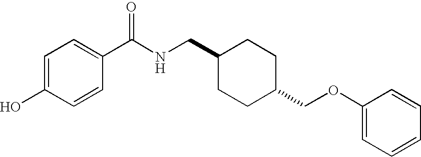

### 4-Hydroxy-N-{[trans-4-(phenoxymethyl)cyclohexyl]methyl}benzamide

This compound was prepared with {[4-(phenoxymethyl)cyclohexyl]methyl}amine hydrochloride by a procedure similar to that in Example 8.

¹H NMR (CDCl₃) δ: 7.67 (d, J=8.6 Hz, 2H), 7.30-7.24 (m, 2H), 6.96-6.84 (m, 5H), 6.17-6.08 (m, 1H), 3.76 (d, J=6.2 Hz, 2H), 3.36-2.98 (m, 2H), 2.03-0.98 (m, 10H) ppm. (OH was not observed.) MS (ESI): 340.17 (ES+), 338.15 (ES−)

### Example 71

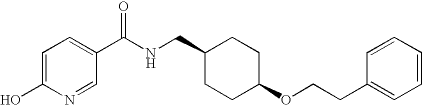

### 6-Hydroxy-N-{[cis-4-(2-phenethoxy)cyclohexyl]methyl}nicotinamide

This compound was prepared with 6-hydroxynicotinic acid and {[cis-4-(2-phenylethoxy)cyclohexyl]methyl}amine by a procedure similar to that in Example 8 as a white solid.

¹H NMR (DMSO-d₆) δ: 11.94 (br, 1H), 8.19-8.15 (m, 1H), 7.98-7.97 (m, 1H), 7.87-7.83 (m, 1H), 7.30-7.18 (m, 5H), 6.35-6.32 (m, 1H), 3.54 (t, J=6.9 Hz, 2H), 3.47 (br, 1H), 3.05-3.00 (m, 2H), 2.79 (t, J=6.9 Hz, 2H), 1.70-1.15 (m, 9H) ppm. MS (ESI): 355.19 (M+H)⁺, 353.21 (M−H)⁻ IR (KBr)νₘₐₓ: 3314, 3057, 2928, 2864, 1713, 1605, 1553, 1310, 1090 cm⁻¹ Anal. Calcd. for C₂₁H₂₆N₂O₃F: C, 71.16; H, 7.39; N, 7.90. Found: C, 71.15; H, 7.40; N, 7.90. m.p. 181.8° C.

### Example 72

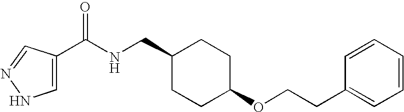

### N-{[cis-4-(2-Phenylethoxy)cyclohexyl]methyl}-1H-pyrazole-4-carboxamide

This compound was prepared with and 1H-pyrazole-4-carboxylic acid {[cis-4-(2-phenylethoxy)cyclohexyl]methyl}amine by a procedure similar to that in Example 8 as a white solid.

¹H NMR (DMSO-d₆) δ: 13.07 (br, 1H), 8.15 (br, 1H), 8.00-7.87 (m, 2H), 7.30-7.18 (m, 5H), 3.54 (t, J=6.9 Hz, 2H), 3.47 (br, 1H), 3.04-3.00 (m, 2H), 2.81-2.76 (m, 2H), 1.75-1.71 (m, 2H), 1.52-1.16 (m, 7H) ppm. MS (ESI): 328.25 (M+H)⁺, 326.19 (M−H)⁻ IR (KBr)νₘₐₓ: 2853, 1248, 1090 cm⁻¹ Anal. Calcd. for C₁₉H₂₅N₃O₂: C, 69.70; H, 7.70; N, 12.83. Found: C, 69.64; H, 7.66; N, 12.67. m.p. 145.0° C.

### Example 73

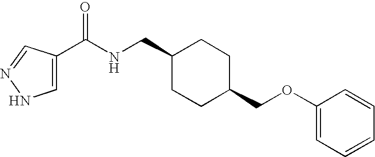

### N-{[cis-4-(Phenoxymethyl)cyclohexyl]methyl}-1H-pyrazole-4-carboxamide

This compound was prepared with 1H-pyrazole-4-carboxylic acid (50 mg, 0.4 mmol) and ({[cis-4-(phenoxymethyl)cyclohexyl]methyl}amine hydrochloride (171 mg, 0.7 mmol) by a procedure similar to that in Example 8 as a white solid (25 mg, 18%).

¹H NMR (DMSO-d₆) δ: 13.08 (brs, 1H), 8.13-7.90 (m, 3H), 7.30-7.25 (m, 2H), 6.95-6.88 (m, 3H), 3.87 (d, J=6.8 Hz, 2H), 3.20-3.15 (m, 2H), 1.90-1.41 (m, 10H) ppm. MS (ESI): 314.21 (M+H)⁺, 312.15 (M−H)⁻ IR (KBr)νₘₐₓ: 3317, 2926, 1626, 1570, 1246, 1036 cm⁻¹ Anal. Calcd. for C₁₈H₂₃N₃O₂: C, 68.98; H, 7.40; N, 13.41. Found: C, 68.68; H, 7.40; N, 13.35. m.p.: 150.1° C.

### Example 74

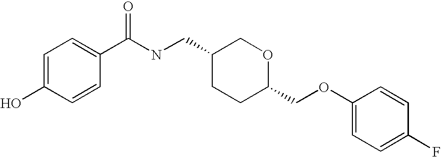

### N-({(3R,6S)-6-[(4-Fluorophenoxy)methyl]tetrahydro-2H-pyran-3-yl}methyl)-4-hydroxybenzamide

N-({(3R,6S)-6-[(4-fluorophenoxy)methyl]tetrahydro-2H-pyran-3-yl}methyl)-4-hydroxybenzamide was prepared with ({(3R,6S)-6-[(4-Fluorophenoxy)methyl]tetrahydro-2H-pyran-3-yl}methyl)amine by a procedure similar to that in Example 8. Cis stereoisomer was separated by Chiral column (Chiralpak AD-H, 20 mm I.D.×250 mm, DAICEL) using n-Hexane/2:Propanol:Et₂NH=85:15:0.1 as an eluent (10 mL/min).

colorless amorphous, >99% ee, cis isomer, retention time 24 min

¹H NMR (CDCl₃) δ: 7.66 (d, J=8.6 Hz, 2H), 7.01-6.80 (m, 6H), 6.50-6.25 (m, 2H), 4.08-3.46 (m, 7H), 2.05-1.50 (m, 5H) ppm. MS (ESI): 360.14 (M+H)⁺, 358.15 (M−H)⁻ IR (KBr)νₘₐₓ: 3350, 2936, 1609, 1508, 1452, 1207 cm⁻¹ [α]_{D}=−6.5 (c=0.4, MeOH)

### Example 75 and 76

N-({(2R*,5R*)-5-[(4-Fluorophenoxy)methyl]tetrahydro-2H-pyran-2-yl}methyl)-4-hydroxybenzamide was prepared with ({(2S*,5S*)-5-[(4-fluorophenoxy)methyl]tetrahydro-2H-pyran-2-yl}methyl)amine by a procedure similar to that in Example 8. The enantimers were separated by Chiral column (Chiralpak OJ-H, 20 mm I.D.×250 mm, DAICEL) using n-Hexane:Ethanol:Et₂NH=85:15:0.1 as an eluent (10 mL/min).

### Example 75

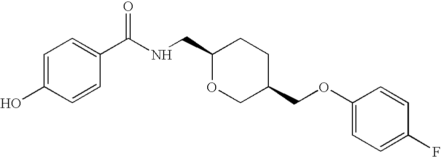

### N-({(2R,5R)-5-[(4-Fluorophenoxy)methyl]tetrahydro-2H-pyran-2-yl}methyl)-4-hydroxybenzamide

>99% ee, retention time 25 min.

¹H NMR (DMSO) δ: 9.96 (bs, 1H), 8.24 (t, J=5.5 Hz, 1H), 7.72 (d, J=8.7 Hz, 2H), 7.14-7.05 (m 2H), 7.01-6.90 (m, 2H), 6.78 (d, J=8.7 Hz, 2H), 4.14-3.75 (m, 3H), 3.53-3.16 (m, 4H), 2.00-1.65 (m, 3H), 1.50-1.30 (m, 2H) ppm. MS (ESI): 360.14 (M+H)⁺, 358.15 (M−H)⁻ IR (KBr)νₘₐₓ: 3350, 2936, 1609, 1508, 1452, 1207 cm⁻¹ [α]_{D}=−10 (c=0.4, MeOH)

### Example 76

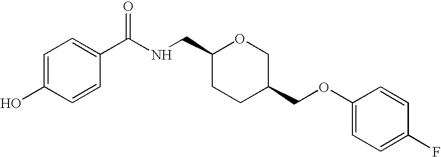

### N-({(2S,5S)-5-[(4-Fluorophenoxy)methyl]tetrahydro-2H-pyran-2-yl}methyl)-4-hydroxybenzamide

>99% ee, retention time 31 min.

¹H NMR (DMSO) δ: 9.96 (bs, 1H), 8.24 (t, J=5.5 Hz, 1H), 7.72 (d, J=8.7 Hz, 2H), 7.14-7.05 (m 2H), 7.01-6.90 (m, 2H), 6.78 (d, J=8.7 Hz, 2H), 4.14-3.75 (m, 3H), 3.53-3.16 (m, 4H), 2.00-1.65 (m, 3H), 1.50-1.30 (m, 2H) ppm. MS (ESI): 360.14 (M+H)⁺, 358.15 (M−H)⁻ IR (KBr)νₘₐₓ: 3350, 2936, 1609, 1508, 1452, 1207 cm⁻¹ [α]_{D}=+5 (c=0.4, MeOH)

### Example 77

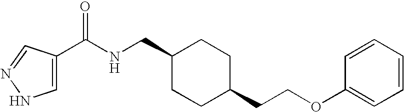

### N-{[cis-4-(2-Phenoxyethyl)cyclohexyl]methyl}-1H-pyrazole-4-carboxamide

This compound was prepared with 1H-pyrazole-4-carboxylic acid (57 mg, 0.5 mmol) and {[cis-4-(2-phenoxyethyl)cyclohexyl]methyl}amine hydrochloride (118 mg, 0.5 mmol) by a procedure similar to that in Example 8 as a white solid (50 mg, 30%).

¹H NMR (DMSO-d₆) δ: 13.08 (brs, 1H), 8.12-7.91 (m, 3H), 7.30-7.24 (m 2H), 6.93-6.88 (m, 3H), 4.01-3.96 (m, 2H), 3.19-3.14 (m, 2H), 1.69-1.42 (m, 12H) ppm. MS (ESI): 328.24 (M+H)⁺, 326.20 (M−H)⁻ IR (KBr)νₘₐₓ: 2924, 1636, 1246, 756 cm⁻¹ Anal. Calcd. for C₁₉H₂₅N₃O₂: C, 69.70; H, 7.70; N, 12.83. Found: C, 69.34; H, 7.60; N, 12.72. m.p.: 155.7° C.

### Example 78

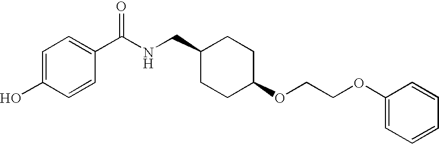

### 4-Hydroxy-N-{cis-4-(2-phenoxyethoxy)cyclohexyl}methyl}benzamide

This compound was prepared with 4-(methoxymethoxy)-N-{[cis-4-(2-phenoxyethoxy)cyclohexyl]methyl}benzamide (81 mg, 0.2 mmol) by a procedure similar to that in Example 6 as colorless amorphous (64 mg, 88%).

¹H NMR (DMSO-d₆) δ: 9.94 (brs, 1H), 8.20-8.16 (m, 1H), 7.71-7.68 (m, 2H), 7.30-7.25 (m, 2H), 6.95-6.90 (m, 3H), 6.79-6.76 (m, 2H), 4.09-4.06 (m, 2H), 3.70-3.67 (m, 2H), 3.57-3.52 (m, 1H), 3.10-3.06 (m, 2H), 1.75-1.26 (m, 9H) ppm.

### Example 79

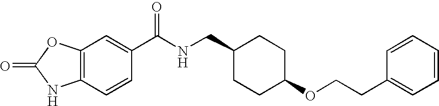

### 2-Oxo-N-{[cis-4-(2-phenylethoxy)cyclohexyl]methyl}-2,3-dihydro-1,3-benzoxazole-6-carboxamide

This compound was prepared with 2-oxo-2,3-dihydro-1,3-benzoxazole-6-carboxylic acid and {[cis-4-(2-phenylethoxy)cyclohexyl]methyl}amine hydrochloride by a procedure similar to that in Example 8 as a white solid.

¹H NMR (DMSO) δ: 8.40 (t, J=5.5 Hz, 1H), 7.82-7.68 (m, 2H), 7.35-7.10 (m, 6H), 3.54 (t, J=7.0 Hz, 2H), 3.51-3.43 (m, 1H), 3.08 (t, J=6.2 Hz, 2H), 2.79 (t, J=7.0 Hz, 2H), 1.82-1.11 (m, 9H) ppm. (NH was not observed.)

### Example 80

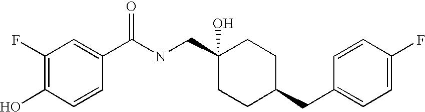

### 3-Fluoro-N-{[trans-4-(4-fluorobenzyl)-1-hydroxycyclohexyl]methyl}-4-hydroxybenzamide

This compound was prepared with 3-fluoro-4-hydroxybenzoic acid and trans-1-(aminomethyl)-4-(4-fluorobenzyl)cyclohexanol hydrochloride by a procedure similar to that in Example 8 as a white solid.

¹H NMR (DMSO-d₆) δ: 10.48 (br, 1H), 8.02 (t, J=5.9 Hz, 1H), 7.75-7.53 (m, 2H), 7.24-6.94 (m, 5H), 4.59 (br, 1H), 3.40-3.30 (m, 2H), 2.55-2.45 (m, 2H), 1.70-1.05 (m, 9H) ppm. MS (ESI): 376.17 (M+H)⁺, 374.22 (M−H)⁻ IR (KBr)νₘₐₓ: 3422, 2930, 1643, 1508, 1308, 1223 cm⁻¹

### Example 81

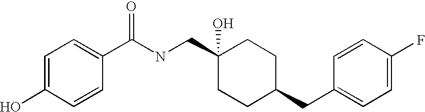

### N-{[trans-4-(4-Fluorobenzyl)-1-hydroxycyclohexyl]methyl}-4-hydroxybenzamide

This compound was prepared with trans-1-(aminomethyl)-4-(4-fluorobenzyl)cyclohexanol hydrochloride by a procedure similar to that in Example 8 as a white solid.

¹H NMR (DMSO-d₆) δ: 9.98 (br, 1H), 7.90 (t, J=5.7 Hz, 1H), 7.73 (d, J=8.6 Hz, 2H), 7.25-7.02 (m, 4H), 6.80 (d, J=8.6 Hz, 2H), 4.66 (br, 1H), 3.40-3.30 (m, 2H), 2.55-2.45 (m, 2H), 1.70-1.05 (m, 9H) ppm. MS (ESI): 358.18 (M+H)⁺, 356.21 (M−H)⁻ IR (KBr)νₘₐₓ: 3319, 2934, 1608, 1508, 1450, 1221 cm⁻¹

### Example 82

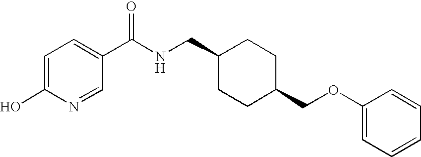

### 6-Hydroxy-N-{[cis-4-(phenoxymethyl)cyclohexyl]methyl}nicotinamide

This compound was prepared with 6-hydroxynicotinic acid (80 mg, 0.6 mmol) and {[cis-4-(phenoxymethyl)cyclohexyl]methyl}amine hydrochloride (147 mg, 0.6 mmol) by a procedure similar to that in Example 8 as a white solid (110 mg, 56%).

1H NMR (DMSO-d₆) δ: 11.94 (brs, 1H), 8.22-8.18 (m, 1H), 7.99-7.98 (m, 11H), 7.89-7.85 (m, 1H), 7.31-7.26 (m, 2H), 6.95-6.88 (m, 3H), 6.36-6.32 (m, 1H), 3.87 (d, J=8.1 Hz, 2H), 3.21-3.16 (m, 2H), 1.90-1.40 (m, 10H) ppm. MS (ESI): 341.17 (M+H)⁺, 339.19 (M−H)⁻ IR (KBr)νₘₐₓ: 3339, 2926, 1638, 1545, 1246, 1036 cm⁻¹ Anal. Calcd. for C₂₀H₂₄N₂O₃: C, 70.56; H, 7.11; N, 8.23. Found: C, 70.36; H 7.15; N, 8.31 m.p.: 189.8° C.

### Example 83

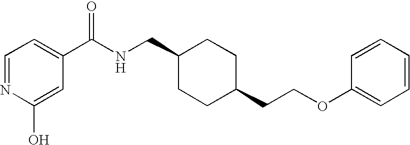

### 2-Hydroxy-N-{[cis-4-(2-phenoxyethyl)cyclohexyl]methyl}isonicotinamide

This compound was prepared with 2-hydroxyisonicotinic acid (63 mg, 0.5 mmol) (Tetrahedron Lett. 1988, 29, 4389) and {[cis-4-(2-phenoxyethyl)cyclohexyl]methyl}amine hydrochloride (105 mg, 0.5 mmol) by a procedure similar to that in Example 8 as a white solid (30 mg, 19%).

¹H NMR (DMSO-d₆) δ: 11.78 (brs, 1H), 8.58-8.56 (m, 1H), 7.45-7.43 (m, 1H), 7.30-7.25 (m, 2H), 6.94-6.88 (m, 3H), 6.71-6.70 (m, 1H), 6.47-6.44 (m, 1H), 4.00-3.96 (m, 2H), 3.20-3.16 (m, 2H), 1.71-1.39 (m, 12H) ppm. MS (ESI): 355.11 (M+H)⁺, 353.17 (M−H)⁻ IR (KBr)νₘₐₓ: 2920, 1639, 1244, 756 cm⁻¹ Anal. Calcd. for C₂₁H₂₆N₂O₃.0.1H₂O: C, 70.80.; H, 7.41; N, 7.86. Found: C, 70.73; H, 7.17; N, 7.78. m.p.: 199.9° C.

### Example 84

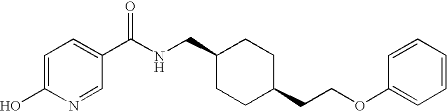

### 6-Hydroxy-N-{[cis-4-(2-phenoxyethyl)cyclohexyl]methyl}nicotinamide

This compound was prepared with 6-hydroxynicotinic acid (63 mg, 0.5 mmol) and {[cis-4-(2-phenoxyethyl)cyclohexyl]methyl}amine hydrochloride (105 mg, 0.5 mmol) by a procedure similar to that in Example 8 as a white solid (77 mg, 48%).

¹H NMR (DMSO-d₆) δ: 11.93 (brs, 1H), 8.21-8.17 (m, 1H), 7.98-7.97 (m, 1H), 7.88-7.84 (m, 1H), 7.30-7.25 (m, 2H), 6.93-6.88 (m, 3H), 6.35-6.32 (m, 1H), 4.00-3.96 (m, 2H), 3.19-3.15 (m, 2H), 1.69-1.36 (m, 12H) ppm. MS (ESI): 355.20 (M+H)⁺, 353.27 (M−H)⁻ IR (KBr)νₘₐₓ: 3329, 2920, 1614, 1246 cm⁻¹ Anal. Calcd. for C₂₁H₂₆N₂O₃: C, 71.16; H, 7.39; N, 7.90. Found: C, 70.80; H, 7.30; N, 7.93. m.p.: 167.6° C.

### Example 85

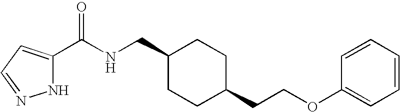

### N-{[cis-4-(2-Phenoxyethyl)cyclohexyl]methyl}-1H-pyrazole-5-carboxamide

This compound was prepared with 1H-pyrazole-5-carboxylic acid (50 mg, 0.5 mmol) and {[cis-4-(2-phenoxyethyl)cyclohexyl]methyl}amine hydrochloride (105 mg, 0.5 mmol) by a procedure similar to that in Example 8 as a white solid (44 mg, 30%).

¹H NMR (DMSO-d₆) δ: 13.19 (brs, 1H), 817-8.00 (m, 0.5H), 7.86-7.73 (m, 0.5H), 7.30-7.24 (m, 2H), 6.93-6.88 (m, 3H), 6.70-6.55 (m, 1H), 4.00-3.96 (m, 2H), 3.22-3.17 (m, 2H), 1.70-1.69 (m, 4H), 1.45-1.40 (m, 8H) ppm. (NH proton was not observed.] MS (ESI): 328.22 (M+H)⁺, 326.25 (M−H)⁻ IR (KBr)νₘₐₓ: 3144, 2922, 1634, 1556, 1250, 758 cm⁻¹ Anal. Calcd. for C₁₉H₂₅N₃O₂: C, 69.70; H, 7.70; N, 12.83. Found: C, 69.63; H, 7.50; N, 12.71. m.p.: 130.5° C.

### Example 86

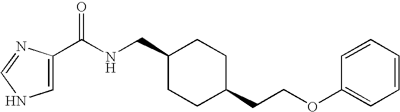

### N-{[cis-4-(2-Phenoxyethyl)cyclohexyl]methyl}-1H-imidazole-4-carboxamide

This compound was prepared with 1H-imidazole-4-carboxylic acid (35 mg, 0.3 mmol) and {[cis-4-(2-phenoxyethyl)cyclohexyl]methyl}amine hydrochloride (73 mg, 0.3 mmol) by a procedure similar to that in Example 8 as a white solid (50 mg, 48%).

¹H NMR (DMSO-d₆) δ: 12.43 (brs, 1H), 7.85-7.80 (m, 1H), 7.772-7.67 (m, 1H), 7.60-7.55 (m, 1H), 7.30-7.25 (m, 2H), 6.94-6.88 (m, 3H), 4.00-3.96 (m, 2H), 3.21-3.16 (m, 2H), 1.71-1.69 (m, 4H), 1.49-1.40 (m, 8H) ppm. MS (ESI): 328.25 (M+H)⁺, 326.29 (M−H)⁻ IR (KBr)νₘₐₓ: 3323, 2922, 1638, 1560, 1248, 754 cm⁻¹ Anal. Calcd. for C₁₉H₂₅N₃O₂: C, 69.70; H, 7.70; N, 12.83. Found: C, 69.57; H, 7.89; N, 12.83. m.p.: 169.6° C.

### Example 87

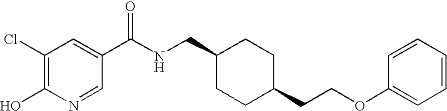

### 5-Chloro-6-hydroxy-N-{[cis-4-(2-phenoxyethyl)cyclohexyl]methyl}nicotinamide

This compound was prepared with 5-chloro-6-hydroxynicotinic acid (69 mg, 0.4 mmol) and {[cis-4-(2-phenoxyethyl)cyclohexyl]methyl}amine hydrochloride (93 mg, 0.4 mmol) by a procedure similar to that in Example 8 as a white solid (46 mg, 30%).

¹H NMR (DMSO-d₆) δ: 12.52 (brs, 1H), 8.29-8.25 (m, 1H), 8.17-8.16 (m, 1H), 8.01-8.00 (m, 1H), 7.30-7.24 (m, 2H), 6.94-6.88 (m, 3H), 4.00-3.96 (m, 2H), 3.20-3.15 (m, 2H), 1.78-1.63 (m, 4H), 1.49-1.35 (m, 8H) ppm. MS (ESI): 389.22 (M+H)⁺, 387.31 (M−H)⁻ IR (KBr)νₘₐₓ: 3325, 2920, 1665, 1533, 1244 cm⁻¹ Anal. Calcd. for C₂₁H₂₅N₃O₃Cl: C, 64.86; H, 6.48; N, 7.20. Found: C, 64.63; H, 6.64; N, 7.06.

### Example 88

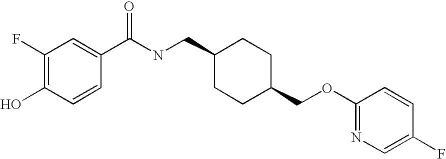

### 3-Fluoro-N-[(cis-4{[(5-fluoropyridin-2-yl)oxy]methyl}cyclohexyl)methyl]-4-hydroxybenzamide

This compound was prepared with 3-fluoro-4-hydroxybenzoic acid and [(cis-4-{[(5-fluoropyridin-2-yl)oxy]methyl}cyclohexyl)methyl]amine by a procedure similar to that in Example 8 as a white solid.

¹H NMR (CDCl₃) δ: 7.98 (d, J=3.0 Hz, 1H), 7.60-7.29 (m, 3H), 7.02 (t, J=8.6 Hz, 1H), 6.70 (dd, J=3.6, 9.1 Hz, 1H), 6.36-6.18 (m, 1H), 4.15 (d, J=7.1 Hz, 2H), 3.37 (t, J=6.6 Hz, 2H), 2.10-1.30 (m, 10H) ppm. (OH was not observed.) MS (ESI): 377.17 (M+H)⁺, 375.26 (M−H)⁻

### Example 89

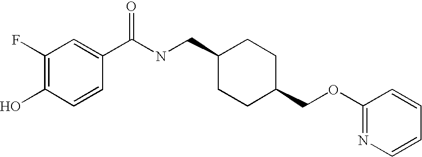

### 3-Fluoro-4-hydroxy-N-({cis-4-[(pyridin-2-yloxy)methyl]cyclohexyl}methyl)benzamide

This compound was prepared with 3-fluoro-4-hydroxybenzoic acid and ({cis-4-[(pyridin-2-yloxy)methyl]cyclohexyl}methyl)amine by a procedure similar to that in Example 8 as a white solid.

¹H NMR (CDCl₃) δ: 8.19-8.12 (m, 1H), 7.63-7.40 (m, 3H), 7.03 (t, J=8.4 Hz, 1H), 6.91-6.84 (m, 1H), 6.74 (d, J=8.2 Hz, 1H), 6.36-6.18 (m, 1H), 4.18 (d, J=7.3 Hz, 2H), 3.33 (t, J=6.4 Hz, 2H), 2.12-1.26 (m, 10H) ppm. (OH was not observed.) MS (ESI): 359.17 (M+H)⁺, 357.23 (M−H)⁻

### Example 90

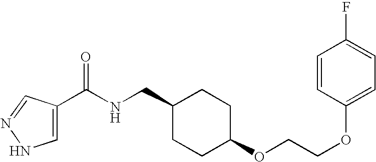

### N-({cis-4-[2-(4-Fluorophenoxy)ethoxy]cyclohexyl}methyl)-1H-pyrazole-4-carboxamide

This compound was prepared with 1H-pyrazole-4-carboxylic acid and ({4-[2-(4-fluorophenoxy)ethoxy]cyclohexyl}methyl)amine by a procedure similar to that in Example 8.

¹H NMR (CDCl₃) δ: 7.94 (s, 2H), 7.77-6.81 (m, 4H), 6.08 (t, J=5.9 Hz, 2H), 4.12-4.05 (m, 2H), 3.77-3.70 (m, 2H), 3.65-3.59 (m, 1H), 3.32-3.24 (m, 2H), 1.98-1.83 (m, 2H), 1.72-1.35 (m, 7H) (m, 8H) ppm. (NH was not observed.)

### Example 91

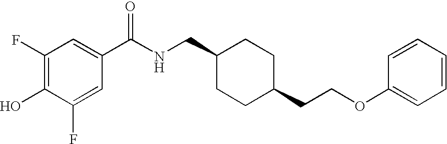

### 3,5-Difluoro-4-hydroxy-N-{[cis-4-(2-phenoxyethyl)cyclohexyl]methyl}benzamide

This compound was prepared with 3,5-difluoro-4-hydroxybenzoic acid (93 mg, 0.5 mmol) (J. Fluorine. Chem. 2000, 102, 169) and {[cis-4-(2-phenoxyethyl)cyclohexyl]methyl}amine hydrochloride (125 mg, 0.5 mmol) by a procedure similar to that in Example 8 as colorless amorphous (74 mg, 35%).

¹H NMR (DMSO-d₆) δ: 10.84 (brs, 1H), 8.41-8.36 (m, 1H), 7.58-7.51 (m, 2H), 7.30-7.24 (m, 2H), 6.93-6.88 (m, 3H), 4.01-3.96 (m, 2H), 3.23-3.18 (m, 2H), 1.82-1.63 (m, 4H), 1.53-1.32 (m, 8H) ppm. MS (ESI): 390.20 (M+H)⁺, 388.24 (M−H)⁻ Anal. Calcd. for C₂₂H₂₅NO₃F₂.0.1H₂O: C, 67.54; H, 6.49; N, 3.58. Found: C, 67.33; H, 6.57; N, 3.59.

### Example 92

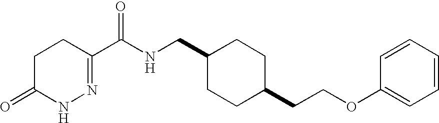

### 6-Oxo-N-{[cis-4-(2-phenoxyethyl)cyclohexyl]methyl}-1,4,5,6-tetrahydropyridazine-3-carboxamide

This compound was prepared with 6-oxo-1,4,5,6-tetrahydropyridazine-3-carboxylic acid and {[cis-4-(2-phenoxyethyl)cyclohexyl]methyl}amine hydrochloride by a procedure similar to that in Example 8 as colorless amorphous.

¹H NMR (DMSO-d₆) δ: 11.05 (s, 1H), 8.12-8.02 (m, 1H), 7.33-7.20 (m, 2H), 6.98-6.86 (m, 3H), 4.04-3.93 (m, 2H), 3.12 (d, J=6.9 Hz, 2H), 2.72 (d, J=8.4 Hz, 2H), 2.38 (d, J=8.6 Hz, 2H), 1.80-1.25 (m, 12H) ppm. MS (ESI): 356.33 (M−H)⁻

### Example 93

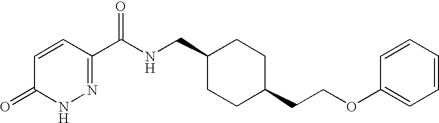

### 6-Oxo-N-{[cis-4-(2-phenoxyethyl)cyclohexyl]methyl}-1,6-dihydropyridazine-3-carboxamide

This compound was prepared with 6-oxo-1,6-dihydropyridazine-3-carboxylic acid (70 mg, 0.5 mmol) (Chem. Pharm. Bull. 1994, 42, 371) and {[cis-4-(2-phenoxyethyl)cyclohexyl]methyl}amine hydrochloride (135 mg, 0.5 mmol) by a procedure similar to that in Example 8 as a white solid (108 mg, 61%).

¹H NMR (DMSO-d₆) δ: 13.39 (brs, 1H), 8.45-8.40 (m, 1H), 7.82 (d, J=10.8 Hz, 1H), 7.30-7.24 (m, 2H), 6.97-6.88 (m, 4H), 4.00-3.96 (m, 2H), 3.23-3.18 (m, 2H), 1.81-1.63 (m, 4H), 1.49-1.33 (m, 8H) ppm. MS (ESI): 354.34 (M+H)⁺ IR (KBr)νₘₐₓ: 3379, 2852, 1657, 1533, 1250, 1007 cm⁻¹ Anal. Calcd. for C₂₀H₂₅N₃O₃.0.1CH₂Cl₂: C, 66.34; H, 6.98; N, 11.55. Found: C, 66.38; H, 7.07; N; 11.25. m.p.: 177.5° C.

### Example 94

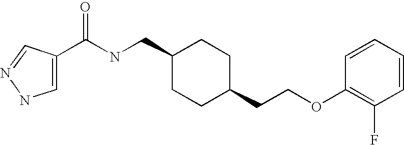

### N-({cis-4-[2-(2-Fluorophenoxy)ethyl]cyclohexyl}methyl)-1H-pyrazole-4-carboxamide

This compound was prepared with 1H-pyrazole-4-carboxylic acid and ({cis-4-[2-(2-fluorophenoxy)ethyl]cyclohexyl}methyl)amine hydrochloride by a procedure similar to that in Example 8.

¹H NMR (DMSO-d₆) δ: 13.08 (br, 1H), 8.29-7.76 (m, 3H), 7.26-6.86 (m, 4H), 4.13-4.02 (m, 2H), 3.23-3.10 (m, 2H), 1.82-1.28 (m, 12H) ppm. MS (ESI): 346.21 (M+H)⁺, 344.24 (M−H)⁻ IR (KBr)νₘₐₓ: 3361, 2926, 1630, 1579, 1504, 1259, 1201 cm⁻¹

### Example 95

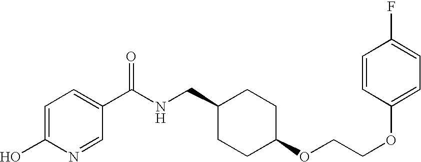

### N-({cis-4-[2-(4-Fluorophenoxy)ethoxy]cyclohexyl{methyl)-6-hydroxynicotinamide

This compound was prepared with 6-hydroxynicotinic acid and ({4-[2-(4-fluorophenoxy)ethoxy]cyclohexyl}methyl)amine by a procedure similar to that in Example 8.

¹H NMR (DMSO) δ: 11.94 (bs, 1H), 8.19 (t, J=5.8 Hz, 1H), 7.98 (d, J=2.6 Hz, 1H), 7.86 (dd, J=2.6, 9.6 Hz, 1H), 7.16-6.92 (m, 4H), 6.34 (d, J=9.6 Hz, 1H), 4.09-4.03 (m, 2H), 3.70-3.63 (m, 2H), 3.58-3.50 (m, 1H), 3.10-3.01 (m, 2H), 1.84-1.70 (m, 2H), 1.63-1.16 (m, 7H) ppm.

### Example 96

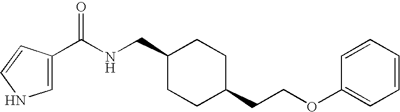

### N-{[cis-4-(2-Phenoxyethyl)cyclohexyl]methyl}-1H-pyrrole-3-carboxamide

This compound was prepared with 1H-pyrrole-3-carboxylic acid (44 mg, 0.4 mmol) and {[cis-4-(2-phenoxyethyl)cyclohexyl]methyl}amine hydrochloride (108 mg, 0.4 mmol) by a procedure similar to that in Example 8 as a yellow solid (33 mg, 25%).

¹H NMR (DMSO-d₆) δ: 11.07 (brs, 1H), 7.69-7.65 (m, 1H), 7.30-7.24 (m, 3H), 6.94-6.88 (m, 3H), 6.73-6.71 (m, 1H), 6.47-6.44 (m, 1H), 4.01-3.96 (m, 2H), 3.16-3.12 (m, 2H), 1.74-1.64 (m, 4H), 1.49-1.34 (m, 8H) ppm. MS (ESI): 327.26 (M+H)⁺, 325.28 (M−H)⁻ IR (KBr)νₘₐₓ: 3204, 2924, 1609, 1568, 1246, 754 cm⁻¹ Anal. Calcd. for C20H26N2O2: C, 73.59.; H, 8.03; N, 8.58. Found: C, 73.24; H, 7.93; N; 8.34. m.p.: 121.0° C.

### Example 97

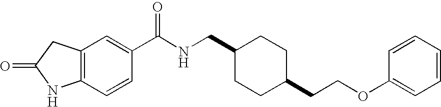

### 2-Oxo-N-{[cis-4-(2-phenoxyethyl)cyclohexyl]methyl}indoline-5-carboxyamide

This compound was prepared with 2-oxoindoline-5-carboxylic acid and {[cis-4-(2-phenoxyethyl)cyclohexyl]methyl}amine hydrochloride by a procedure similar to that in Example 8.

¹H NMR (DMSO-d₆) δ: 10.60 (br, 1H), 8.32-8.21 (m, 1H), 7.77-7.68 (m, 2H), 7.34-7.22 (m, 2H), 7.00-6.80 (m, 4H), 4.06-3.93 (m, 2H), 3.53 (s, 2H), 3.27-3.16 (m, 2H), 1.85-1.29 (m, 12H) ppm. MS (ESI): 393.32 (M+H)⁺, 391.37 (M−H)⁻ IR (KBr)νₘₐₓ: 3374, 2920, 1686, 1618, 1489, 1292, 1244 cm⁻¹

### Example 98

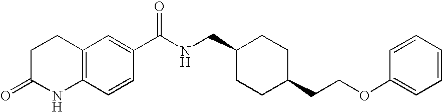

### 2-Oxo-N-{[cis-4-(2-phenoxyethyl)cyclohexyl]methyl}-1,2,3,4-tetrahydroquinoline-6-carboxamide

This compound was prepared with 2-oxo-1,2,3,4-tetrahydroquinoline-6-carboxylic acid (77 mg, 0.4 mmol) (Chem. Pharm. Bull. 1986, 34, 682) and {[cis-4-(2-phenoxyethyl)cyclohexyl]methyl}amine hydrochloride (108 mg, 0.4 mmol) by a procedure similar to that in Example 8 as a yellow solid (36 mg, 2%).

¹H NMR (DMSO-d₆) δ: 10.28 (brs, 1H), 8.29-8.24 (m, 1H), 7.69-7.63 (m, 2H), 7.30-7.24 (m, 2H), 6.94-6.85 (m, 4H), 4.01-3.97 (m, 2H), 3.23-3.18 (m, 2H), 2.94-2.88 (m, 2H), 2.47-2.45 (m, 2H), 1.78-1.66 (m, 4H), 1.52-1.36 (m, 8H) ppm. MS (ESI): 407.03 (M+H)⁺, 405.11 (M−H)⁻ Anal. Calcd. for C₂₅H₃₀N₂O₃.0.6H₂O: C, 71.95.; H, 7.54; N, 6.71. Found: C, 71.84; H, 7.47; N, 6.49.

### Example 99

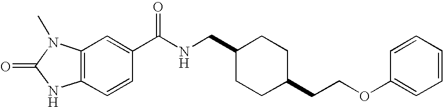

### 3-Methyl-2-oxo-N-{[cis-4-(2-phenoxyethyl)cyclohexyl]methyl}-2,3-dihydro-1H-benzimidazole-5-carboxamide

This compound was prepared with 3-methyl-2-oxo-2,3-dihydro-1H-benzimidazole-5-carboxylic acid and {[cis-4-(2-phenoxyethyl)cyclohexyl]methyl}amine hydrochloride by a procedure similar to that in Example 8.

¹H NMR (CDCl₃) δ: 9.32 (br, 1H), 7.58-7.54 (m, 1H), 7.45-7.39 (m, 1H), 7.33-7.24 (m, 2H), 7.09 (d, J=8.2 Hz, 1H), 6.98-6.86 (m, 3H), 6.18-6.08 (m, 1H), 4.04-3.94 (m, 2H), 3.53-3.40 (m, 5H), 1.94-1.38 (m, 12H) ppm. MS (ESI): 407.99 (M+H)⁺, 406.07 (M−H)⁻

### Example 100

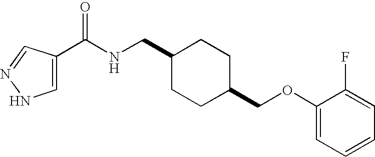

### N-({cis-4-[(2-Fluorophenoxy)methyl]cyclohexyl}methyl)-1H-pyrazole-4-carboxamide

This compound was prepared with 1H-pyrazole-4-carboxylic acid (56 mg, 0.5 mmol) and ({cis-4-[(2-fluorophenoxy)methyl]cyclohexyl}methyl)amine hydrochloride (137 mg, 0.5 mmol) by a procedure similar to that in Example 8 as a white solid (90 mg, 55%).

¹H NMR (DMSO-d₆) δ: 13.08 (brs, 1H), 8.11-7.97 (m, 3H), 7.23-7.09 (m, 3H), 6.95-6.89 (m, 1H), 3.96 (d, 2H, J=5.4 Hz), 3.20-3.15 (m, 2H), 1.93-1.48 (m, 10H) ppm. MS (ESI): 332.14 (M+H)⁺, 330.16 (M−H)⁻ Anal. Calcd. for C₁₈H₂₂N₃O₂F: C, 65.24; H, 6.69; N, 12.68. Found: C, 65.19; H, 6.54; N, 12.64. m.p.: 139.8° C.

### Example 101

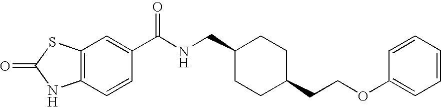

### 2-Oxo-N-{[cis-4-(2-phenoxyethyl)cyclohexyl]methyl}-2,3-dihydro-1,3-benzothiazole-6-carboxamid

This compound was prepared with 2-oxo-2,3-dihydro-1,3-benzothiazole-6-carboxylic acid (20 mg, 0.1 mmol) (Chem. Pharm. Bull. 1988, 36, 2253) and {[cis-4-(2-phenoxyethyl)cyclohexyl]methyl}amine hydrochloride (30 mg, 0.1 mmol) by a procedure similar to that in Example 8 as a white solid (28 mg, 67%).

¹H NMR (DMSO-d₆) δ: 8.40-8.36 (m, 1H), 8.05-8.04 (m, 1H), 7.80-7.76 (m, 1H), 7.30-7.24 (m, 2H), 7.17-7.14 (m, 1H), 6.94-6.88 (m, 3H), 4.01-3.96 (m, 2H), 3.25-3.20 (m, 2H), 1.72-1.37 (m, 12H) ppm. (NH was not observed.] MS (ESI): 411.04 (M+H)⁺, 409.10 (M−H)⁻ Anal. Calcd. for C₂₃H₂₆N₂O₃S : C, 67.29.; H, 6.38; N, 6.82. Found: C, 67.27; H, 6.42; N, 6.81. m.p.: 159.9° C., 175.7° C.

### Example 102

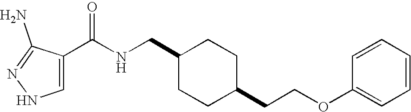

### 3-Amino-N-{[cis-4-(2-phenoxyethyl)cyclohexyl]methyl)-1H-pyrazole-4-carboxamide

This compound was prepared with 3-amino-1H-pyrazole-4-carboxylic acid (64 mg, 0.5 mmol) and {[cis-4-(2-phenoxyethyl)cyclohexyl]methyl}amine hydrochloride (135 mg, 0.5 mmol) by a procedure similar to that in Example 8 as a white solid (81 mg, 47%).

¹H NMR (DMSO-d₆) δ: 11.82-11.69 (m, 1H), 7.96-7.65 (m, 2H), 7.30-7.25 (m, 2H), 6.93-6.88 (m, 3H), 5.92-5.80 (m, 1H), 5.34-5.22 (m, 1H), 4.00-3.96 (m, 2H), 3.15-3.10 (m, 2H), 1.75-1.62 (m, 4H), 1.52-1.32 (m, 8H) ppm. MS (ESI): 343.17 (M+H)⁺, 341.17 (M−H)⁻ Anal. Calcd. for C₁₉H₂₆N₄O₂.0.2H₂O: C, 65.95.; H, 7.69; N, 16.19. Found: C, 65.87; H, 7.82; N, 16.01. m.p.: 129.3° C.

### Example 103

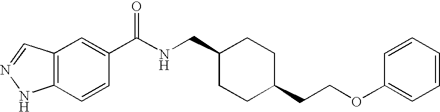

### N-{[cis-4-(2-Phenoxyethyl)cyclohexyl]methyl}-1H-indazole-5-carboxamide

This compound was prepared with 1H-indazole-5-carboxylic acid (65 mg, 0.4 mmol) (Helv. Chim. Acta. 1976, 59, 2618) and {[cis-4-(2-phenoxyethyl)cyclohexyl]methyl}amine hydrochloride (108 mg, 0.4 mmol) by a procedure similar to that in Example 8 as a white solid (37 mg, 25%).

¹H NMR (DMSO-d₆) δ: 13.25 (brs, 1H), 8.46-8.41 (m, 1H), 8.32 (s, 1H), 8.20 (s, 1H), 7.86-7.83 (m, 1H), 7.58-7.54 (m, 1H), 7.30-7.25 (m, 2H), 6.94-6.88 (m, 3H), 4.01-3.97 (m, 2H), 3.28-3.23 (m, 2H), 1.84-1.63 (m, 4H), 1.54-1.35 (m, 8H) ppm. MS (ESI): 378.12 (M+H)⁺, 376.16 (M−H)⁻ Anal. Calcd. for C₂₃H₂₇N₃O₂: C, 73.18.; H, 7.21; N, 11.13. Found: C, 72.80; H, 7.18; N, 11.08. m.p.: 144.5° C.

### Example 104

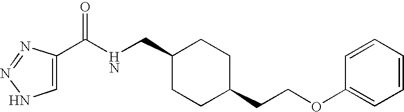

### N-{[cis-4-(2-Phenoxyethyl)cyclohexyl]methyl}-1H-1,2,3-triazole-4-carboxamide

This compound was prepared with 1H-1,2,3-triazole-4-carboxylic acid (45 mg, 0.4 mmol) (J. Amer. Chem. Soc. 1954, 76, 4931) and {[cis-4-(2-phenoxyethyl)cyclohexyl]methyl}amine hydrochloride (108 mg, 0.4 mmol) by a procedure similar to that in Example 8 as a white solid (24 mg, 19%).

¹H NMR (DMSO-d₆) δ: 8.45-8.30 (m, 2H), 7.30-7.23 (m, 2H), 6.94-6.88 (m, 3H), 4.01-3.96 (m, 2H), 3.25-3.20 (m, 2H), 1.71-1.42 (m, 12H) ppm. [NH proton was not observed.] MS (ESI): 329.10 (M+H)⁺, 327.12 (M−H)⁻ Anal. Calcd. for C₁₈H₂₄N₄O₂: C, 65.83.; H, 7.37; N, 17.06. Found: C, 65.45; H, 7.08; N, 17.10. m.p.: 141.2° C.

### Example 105

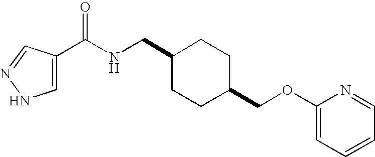

### N-({cis-4-[(Pyridin-2-yloxy)methyl]cyclohexyl}methyl)-1H-pyrazole-4-carboxamide

This compound was prepared with 1H-pyrazole-4-carboxylic acid and ({cis-4-[(pyridin-2-yloxy)methyl]cyclohexyl}methyl)amine by a procedure similar to that in Example 8.

¹H NMR (CDCl₃) δ: 13.07 (br, 1H), 8.21-7.84 (m, 4H), 7.69 (ddd, J=8.4, 7.0, 2.1 Hz, 1H), 6.95 (m, 1H), 6.80 (d, J=8.2 Hz, 1H), 4.18 (d, J=7.1 Hz, 2H), 3.17 (m, 2H), 1.91 (br, 1H), 1.73 (br, 1H), 1.60-1.31(m, 8H) ppm. MS (ESI): 315.09 (M+H)⁺, 313.10 (M−H)⁻ IR (KBr)νₘₐₓ: 3358, 2849, 1631, 1475, 1435, 1246, 1022, 777 cm⁻¹

### Example 106

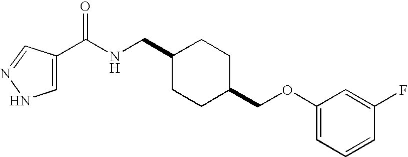

### N-({cis-4-[(3-Fluorophenoxy)methyl]cyclohexyl}methyl)-1H-pyrazole-4-carboxamide

This compound was prepared with 1H-pyrazole-4-carboxylic acid and ({[cis-4-(3-fluorophenoxymethyl)cyclohexyl]methyl}amine hydrochloride by a procedure similar to that in Example 8.

¹H NMR (DMSO-d₆) δ: 13.08 (brs, 1H), 8.00-7.97 (m, 3H), 7.33-7.26 (m, 1H), 6.85-6.71 (m, 3H), 3.90 (d, J=6.8 Hz, 2H), 3.16 (t, J=6.6 Hz, 2H), 1.96-1.85 (m, 1H), 1.79-1.67 (m, 1H), 1.58-1.32 (m, 8H) ppm. MS (ESI): 332.12 (M+H)⁺, 338.15 (M−H)⁻ IR (KBr)νₘₐₓ: 3348, 2920, 1625, 1577, 1490, 1284, 1134, 1041 cm⁻¹

### Example 107

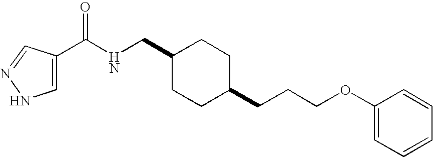

### N-{[cis-4-(3-Phenoxypropyl)cyclohexyl]methyl}-1H-pyrazole-4-carboxamide

This compound was prepared with 1H-pyrazole-4-carboxylic acid and {[cis-4-(3-phenoxypropyl)cyclohexyl]methyl}amine by a procedure similar to that in Example 8.

¹H NMR (DMSO-d₆) δ: 13.09 (s, 1H), 8.15-7.90 (m, 3H), 7.33-7.22 (m, 2H), 6.96-6.87 (m, 3H), 3.94 (t, J=6.4 Hz, 2H), 3.16 (t, J=6.4 Hz, 2H), 1.80-1.63 (m, 4H), 1.53-1.25 (m, 10H) ppm. MS (ESI): 342.09 (M+H)⁺, 340.12 (M−H)⁻ IR (KBr)νₘₐₓ: 3310, 2924, 1626, 1604, 1566, 1539, 1499, 1246, 752, 691 cm⁻¹

### Example 108

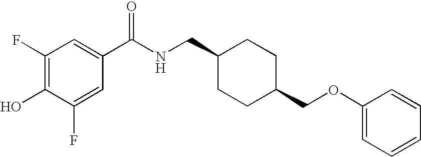

### 3,5-Difluoro-4-hydroxy-N-{[cis-4-(phenoxymethyl)cyclohexyl]methyl}benzamide

This compound was prepared with 3,5-difluoro-4-hydroxybenzoic acid (87 mg, 0.5 mmol) and {[cis-4-(phenoxymethyl)cyclohexyl]methyl}amine hydrochloride (128 mg, 0.5 mmol) by a procedure similar to that in Example 8 as colorless amorphous (49 mg, 26%).

¹H NMR (CDCl₃) δ: 7.37-7.25 (m, 5H), 6.96-6.88 (m, 3H), 6.00 (brs, 1H), 3.88-3.85 (m, 2H), 3.44-3.39 (m, 2H), 2.03-1.47 (m, 10H) ppm. MS (ESI): 376.05 (M+H)⁺, 374.06 (M−H)³¹ Anal. Calcd. for C₂₁H₂₃NO₃F₂.0.2H₂O: C, 66.55; H, 6.22; N, 3.70. Found: C, 66.34; H, 6.20; N, 3.65.

### Example 109

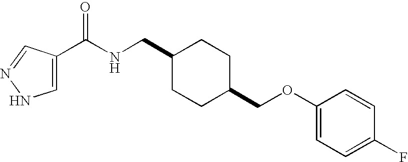

### N-({cis-4-[(4-Fluorophenoxy)methyl]cyclohexyl}methyl)-1H-pyrazole-4-carboxamide

This compound was prepared with 1H-pyrazole-4-carboxylic acid (22 mg, 0.2 mmol) and ({cis-4-[(4-fluorophenoxy)methyl]cyclohexyl}methyl)amine hydrochloride (55 mg, 0.2 mmol) by a procedure similar to that in Example 8 as a white solid (35 mg, 54%).

¹H NMR (DMSO-d₆) δ: 13.08 (brs, 1H), 8.14 (brs, 1H), 8.00-7.96 (m, 1H), 7.88 (brs, 1H), 7.13-7.07 (m, 2H), 6.98-6.93 (m, 2H), 3.87-3.84 (m, 2H), 3.20-3.15 (m, 2H), 1.88-1.40 (m, 10H) ppm. MS (ESI): 332.10 (M+H)⁺, 330.10 (M−H)⁻ Anal. Calcd. for C₁₈H₂₂N₃O₂F: C, 65.24; H, 6.69; N, 12.68. Found: C, 65.05; H, 6.65; N, 12.67. m.p.: 147.1° C.

### Example 110

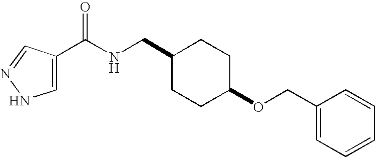

### N-{[cis-4-(Benzyloxy)cyclohexyl]methyl}-1H-pyrazole-4-carboxamide

This compound was prepared with 1H-pyrazole-4-carboxylic acid and {[cis-4-(benzyloxy)cyclohexyl]methyl}amine by a procedure similar to that in Example 8.

¹H NMR (DMSO-d₆) δ: 13.08 (s, 1H), 8.23-7.80 (m, 3H), 7.40-7.20 (m, 5H), 4.45 (s, 2H), 3.63-3.53 (m, 1H), 3.08 (t, J=6.4 Hz, 2H), 1.90-1.77 (m, 2H), 1.65-1.20 (m, 7H) ppm. MS (ESI): 314.08 (M+H)⁺, 312.07 (M−H)⁻ IR (KBr)νₘₐₓ: 3335, 3126, 2928, 1630, 1580, 1537, 1246, 1065, 735, 696 cm⁻¹

### Example 111

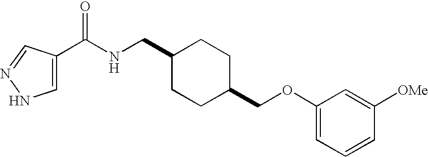

### N-({cis-4-[(3-Methoxyphenoxy)methyl]cyclohexyl}methyl)-1H-pyrazole-4-carboxamide

This compound was prepared with 1H-pyrazole-4-carboxylic acid and ({cis-4-[(3-methoxyphenoxy)methyl]cyclohexyl}methyl)amine by a procedure similar to that in Example 8.

¹H NMR (DMSO-d₆) δ: 13.07 (brs, 1H), 8.20-8.10 (m, 1H), 8.03-7.95 (m, 1H), 7.92-7.84 (m, 1H), 7.16 (m, 1H), 6.56-6.46 (m, 3H), 3.86 (d, J=6.8 Hz, 2H), 3.73 (s, 3H), 3.18 (t, J=6.8 Hz, 2H), 1.97-1.82 (m, 1H), 1.80-1.65 (m, 1H), 1.59-1.30 (m, 8H) ppm. MS (ESI): 344.18 (M+H)⁺, 342.25 (M−H)⁻

### Example 112

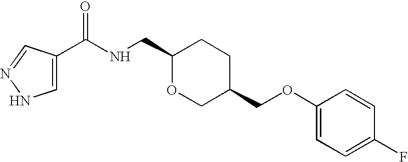

### N-({(2R,5R)-5-[(4-Fluorophenoxy)methyl]tetrahydro-2H-pyran-2-yl}methyl)-1H-pyrazole-4-carboxamide

This compound was prepared with 1H-pyrazole-4-carboxylic acid by a procedure similar to that in Example 75.

¹H NMR (DMSO) δ: 13.08 (bs, 1H), 8.24-7.83 (m, 3H), 7.17-6.92 (m, 4H), 4.17-3.87(m, 3H), 3.58-3.11 (m, 4H), 2.03-1.26 (m, 5H) ppm.

### Example 113 and 114

4 stereoisomers were prepared with 1H-pyrazole-4-carboxylic acid and ({5-[2-(4-fluorophenoxy)ethyl]tetrahydro-2H-pyran-2-yl}methyl)amine by a procedure similar to that in Example 8.

4 stereoisomers were separated by Chiral column (Chiralpak AD-H, 20 mm I.D.×250 mm (No. ADH0CJ-DE003), DAICEL) using n-Hexane:2-Propanol:Et₂NH=85:15:0.1 as an eluent (10 mL/min).

### Example 113

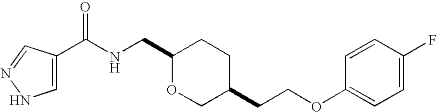

### N-({(2R,5S)-5-[2-(4-Fluorophenoxy)ethyl]tetrahydro-2H-pyran-2-yl}methyl)-1H-pyrazole-4-carboxamide

Retention time 29 min-32 min

¹H NMR (DMSO-d) δ: 13.28 (br, 1H), 8.15-7.93 (m, 3H), 7.10 (t, J=8.8 Hz, 2H), 6.94 (dd, J=9.0, 4.6 Hz, 2H), 3.99 (t, J=6.3 Hz, 2H), 3.73 (m, 1H), 3.55-3.10 (m, 4H), 1.96-1.65 (m, 5H), 1.50-1.35 (m, 2H)ppm. MS (ESI): 348.16 (M+H)⁺, 346.17 (M−H)⁻

### Example 114

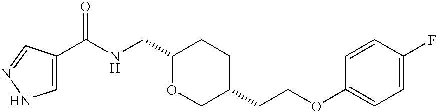

### N-({(2S,5R)-5-[2-(4-fluorophenoxy)ethyl]tetrahydro-2H-pyran-2-yl}methyl)-1H-pyrazole-4-carboxamide

Retention time 39 min-43 min.

¹H NMR (DMSO-d) δ: 13.09 (br, 1H), 8.18-7.96 (m, 3H), 7.10 (t, J=8.9 Hz, 2H), 6.94 (dd, J=9.2, 4.4 Hz, 2H), 3.99 (t, J=6.4 Hz, 2H), 3.73 (m, 1H), 3.54-3.13 (m, 4H), 1.96-1.61 (m, 5H), 1.50-1.35 (m, 2H)ppm. MS (ESI): 348.09 (M+H)⁺, 346.11 (M−H)⁻

### Example 115

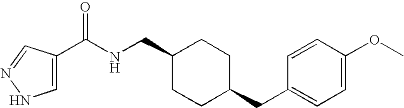

### N-{[cis-4-(4-Methoxybenzyl)cyclohexyl]methy}-1H-pyrazole-4-carboxamide

This compound was prepared with 1H-pyrazole-4-carboxylic acid and {[cis-4-(4-methoxybenzyl)cyclohexy]methyl}amine by a procedure similar to that in Example 8.

¹H NMR (DMSO) δ: 13.07 (bs, 1H), 8.19-7.83 (m, 3H), 7.07 (d, J=8.6 Hz, 2H), 6.82 (d, J=8.6 Hz, 2H), 3.71 (s, 3H), 3.23-3.11 (m, 2H), 2.50-2.43 (m, 2H), 1.78-1.20 (m, 9H) ppm.

### Examples 116 and 116(2)

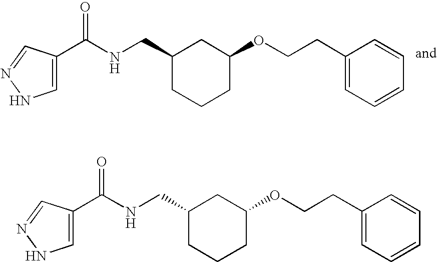

### N-{[(1R,3S)-3-(2-Phenylethoxy)cyclohexyl]methyl}-1H-pyrazole-4-carboxamide and N-{[(1S,3R)-3-(2-Phenylethoxy)cyclohexyl]methyl}-1H-pyrazole-4-carboxamide

N-{[cis-3-(2-phenylethoxy)cyclohexyl]methyl}-1H-pyrazole-4-carboxamide (0.11 g, 0.34 mmol) was prepared with 1H-pyrazole-4-carboxylic acid and {[(cis-3-(2-Phenylethoxy)cyclohexyl)methyl]amine by a procedure similar to that in Example 8, and separated by chiral column (Chiralcel OJ-H, 20 mm I.D.×250 mm (No. OJH0CJ-DH004), DAICEL) using n-Hexane/EtOH/Et₂NH=93/7/0.1 as an eluent (Flow rate: 10 mL/min) to give the titled compounds.

### Example 116

First peak: (32 mg) retention time 39.8 min, >99% ee.

¹H NMR (DMSO-d₆) δ: 13.08 (s, 1H), 8.15-7.95 (m, 3H), 7.32-7.12 (m, 5H), 3.61 (t, J=7.1 Hz, 2H), 3.28-2.96 (m, 3H), 2.76 (t, J=7.1 Hz, 2H), 2.08-1.90 (m, 2H), 1.78-1.40 (m, 3H), 1.27-0.70 (m, 4H) ppm. MS (ESI): 328.15 (M+H)⁺, 326.23 (M−H)⁻

### Example 116(2)

Second peak: (27 mg) retention time 45.3 min, >99% ee

¹H NMR data was identical with that of example 116. MS (ESI): 328.15 (M+H)⁺, 326.23 (M−H)⁻

### Example 117

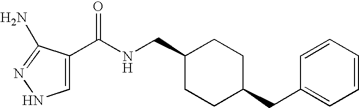

### 3-Amino-N-[(cis-4-benzylcyclohexyl)methyl]-1H-pyrazole-4-carboxamide

This compound was prepared with 3-amino-1H-pyrazole-4-carboxylic acid (38 mg, 0.3 mmol) and [(cis-4-benzylcyclohexyl)methyl]amine (61 mg, 0.3 mmol) by a procedure similar to that in Example 8 as a white solid (8.8 mg, 9%).

¹H NMR (DMSO-d₆) δ: 11.80-11.69 (m, 1H), 7.93-7.64 (m, 2H), 7.30-7.15 (m, 5H), 5.86-5.27 (m, 2H), 3.16-3.12 (m, 2H), 2.56-2.53 (m, 2H), 1.67-1.40 (m, 10H) ppm. MS (ESI): 313.23 (M+H)⁺, 311.13 (M−H)⁻

### Example 118

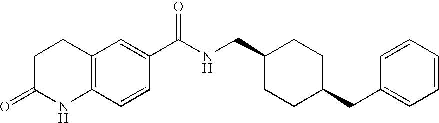

### N-[(cis-4-Benzylcyclohexyl)methyl]-2-oxo-1,2,3,4-tetrahydroquinoline-6-carboxamide

This compound was prepared with 2-oxo-1,2,3,4-tetrahydroquinoline-6-carboxylic acid (57 mg, 0.3 mmol) and [(cis-4-benzylcyclohexyl)methyl]amine (61 mg, 0.3 mmol) by a procedure similar to that in Example 8 as a white solid (35 mg, 31%).

¹H NMR (DMSO-d₆) δ: 10.27 (brs, 1H), 8.28-8.23 (m, 1H), 7.69-7.63 (m, 2H), 7.30-7.15 (m, 5H), 6.88-6.85 (m, 1H), 3.25-3.20 (m, 2H), 2.94-2.88 (m, 2H), 2.56-2.45 (m, 4H), 1.73-1.29 (m, 10H) ppm. MS (ESI): 377.21 (M+H)⁺, 375.20 (M−H)⁻ Anal. Calcd. for C₂₄H₂₈N₂O₂.0.1H₂O: C, 76.20; H, 7.51; N, 7.41. Found: C, 76.11; H, 7.57; N, 7.31. m.p. 188.5° C.

### Example 119

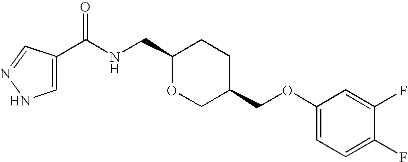

### N-({(2R,5R)-5-[(3,4-Difluorophenoxy)methyl]tetrahydro-2H-pyran-2-yl}methyl)-1H-pyrazole-4-carboxamide

To a solution of tert-butyl {[(5S)-5-(hydroxymethyl)tetrahydro-2H-pyran-2-yl]methyl}carbamate (300 mg, 1.22 mmol) and 3,4-difluorophenol (397.7 mg, 3.06 mmol) in THF (4.9 mL), were added PPh₃ (737.7 mg, 2.81 mmol) and DIAD (0.56 mL, 2.32 mmol) at 0° C. The mixture was irradiated by microwave at 180° C. for 5 min. Then the mixture was cooled to room temprature and was diluted AcOEt. The oganic layer was washed with 2N NaOH aq. and brine. The organic layer was dried over Na₂SO₄, was filtered and evaporated. The crude product was purified by silica gel column chromatography (hexane:AcOEt=50:1-20:1) to give tert-butyl ({(2R,5R)-5-[(3,4-difluorophenoxy)methyl]tetrahydro-2H-pyran-2-yl}methyl)carbamate (55.5 mg, 0.155 mmol) This was dissolved in HCl-MeOH (1 mL) and the mixture was stirred at 40° C. for 2 hr. The mixture was evaporated to give the crude amine. The amine was dissolved in DMF (2 mL) and were added 1H-pyrazole-4-carboxylic acid (17.4 mg, 0.155 mmol), Et₃N (0.064 mL, 0.466 mmol), HOBt (28.5 mg, 0.186 mmol) and WSC (35.6 mg, 0.186 mmol) at 0° C. The mixture was stirred at room temperature overnight. 2N NaOH aq was added to the mixture and the mixture was stirred at room temperature for 1 hr. The mixture was extracted with AcOEt and the organic layer was washed with brine. The organic layer was dried over Na₂SO₄, was filtered and evaporated. The crude product was purified by silica gel column chromatography (CH₂Cl₂:MeOH=20:1) to give the titled compound.

¹H NMR (DMSO-d) δ: 13.08 (br, 1H), 8.17-7.92 (m, 3H), 7.35-7.25 (m, 1H), 7.13-7.05 (m, 1H), 6.84-6.75 (m, 1H), 4.14 (t, J=9.1 Hz, 1H), 4.03-3.87 (m, 2H), 3.58-3.11 (m, 4H), 1.94 (br, 1H), 1.88-1.64 (m, 2H), 1.53-1.29 (m, 2H) ppm. MS (ESI): 352.20 (M+H)⁺, 350.15 (M−H)⁻

### Example 120 and Example 121

To a suspension of LiAlH₄ (119.7 mg, 3.,15 mmol) in THF (10 mL) the solution of 2-(azidomethyl)-5-[(4-chlorophenoxy)methyl]tetrahydro-2H-pyran (444.3 mg, 1.58 mmol) in THF (6 mL) was added at 0° C. Then the mixture was stirred at 0° C. for 1.25 hr. The reaction was quenched by Na₂SO₄.10H₂O (1.6 g, 4.97 mmol) and KF (200 mg, 3.44 mmol). The mixture was stirred at room temperature for 1 hr. The mixture was filtered through a pad of celite and the filtrate was evaporated to give the crude compound.

To a solution of the crude compound in DMF (5 mL), were added 1H-pyrazole-4-carboxylic acid (177.1 mg, 1.58 mmol), HOBt (290.4 mg, 1.90 mmol) and WSC (363.5 mg, 1.90 mmol) at 0° C. The mixture was stirred at room temperature overnight. 2N NaOH aq was added to the mixture and the mixture was stirred at room temperature for 1 hr. The mixture was extracted with AcOEt and the organic layer was washed with brine. The organic layer was dried over Na₂SO₄, was filtered and evaporated. The crude product was purified by silica gel column chromatography (CH₂Cl₂:MeOH=20:1) to give the mixture of 4 stereoisomers.

4 stereoisomers were separated by Chiral column (Chiralcel OJ-H, 20 mm I.D.×250 mm (No. OJH0CJ-DH004), DAICEL) using n-Hexane:EtOH:Et2NH=88:12:0.1 as an eluent (18.9 mL/min).

### Example 120

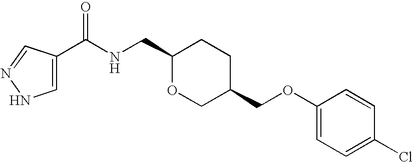

### N-({(2R,5R)-5-[(4-Chlorophenoxy)methyl]tetrahydro-2H-pyran-2-yl}methyl)-1H-pyrazole-4-carboxamide

Retention time 12 min-20 min (13 min)

¹H NMR (DMSO-d) δ: 13.10 (br, 1H), 8.23-7.83 (m, 3H), 7.33 (d, J=9.0 Hz, 2H), 6.99 (d, J=8.8 Hz, 2H), 4.14 (t, J=9.0 Hz, 1H), 4.05-3.86 (m, 2H), 3.58-3.12 (m, 4H), 1.95 (br, 1H), 1.89-1.66 (m, 2H), 1.53-1.20 (m, 2H)ppm. MS (ESI): 350.05 (M+H)⁺, 348.06 (M−H)⁻

### Example 121

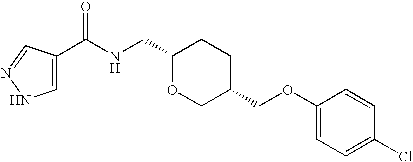

### N-({(2S,5S)-5-[(4-Chlorophenoxy)methyl]tetrahydro-2H-pyran-2-yl}methyl)-1H-pyrazole-4-carboxamide

Retention time 20 min-24 min (22 min)

¹H NMR (DMSO-d) δ: 13.09 (br, 1H), 8.20-7.85 (m, 3H), 7.32-7.28 (m, 2H), 7.04-6.94 (m, 2H), 4.14 (t, J=8.7 Hz, 1H), 4.05-3.86 (m, 2H), 3.60-3.10 (m, 4H), 1.95 (br, 1H), 1.86-1.64 (m, 2H), 1.53-1.20 (m, 2H)ppm. MS (ESI): 350.04 (M+H)⁺, 348.06 (M−H)⁻

### Example 122

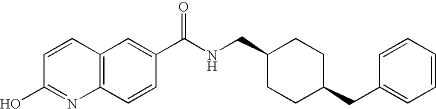

### N-[(cis-4-Benzylcyclohexyl)methyl]-2-hydroxyquinoline-6-carboxamide

This compound was prepared with 2-hydroxyquinoline-6-carboxylic acid (38 mg, 0.2 mmol) and [(cis-4-benzylcyclohexyl)methyl]amine (53 mg, 0.2 mmol) by a procedure similar to that in Example 8 as a white solid (26 mg, 34%).

¹H NMR (DMSO-d₆) δ: 11.93 (brs, 1H), 8.46-8.42 (m, 1H), 8.18-8.17 (m, 1H), 7.97-7.94 (m, 2H), 7.33-7.25 (m, 3H), 7.18-7.16 (m, 3H), 6.57-6.53 (m, 1H), 3.29-3.24 (m, 2H), 2.58-2.55 (m, 2H), 1.83-1.64 (m, 2H), 1.44-1.30 (m, 8H) ppm. MS (ESI): 375.06 (M+H)⁺, 373.08 (M−H)⁻ Anal. Calcd. for C₂₄H₂₆N₂O₂.0.4H₂O: C, 75.52; H, 7.08; N, 7.34. Found: C, 75.18; H, 6.94; N, 7.09. m.p.: 236.7° C.

### Example 123

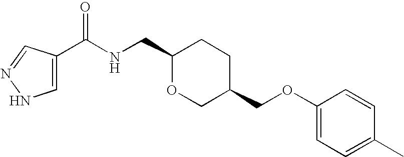

### N-({(2R,5R)-5-[(4-Methylphenoxy)methyl]tetrahydro-2H-pyran-2-yl}methyl)-1H-pyrazole-4-carboxamide

This compound was prepared with p-cresol by a procedure similar to that in Example 119.

Cis and trans isomers were separated by Chiral column (Chiralcel OJ-H, 20 mm I.D.×250 mm (No. OJH0CJ-DH004), DAICEL) using 5 min-7 min (5 min)

¹H NMR (DMSO-d) δ: 13.10 (br, 1H), 8.21-7.86 (m, 3H), 7.08 (d, J=8.1 Hz, 2H), 6.84 (d, J=8.6 Hz, 2H), 4.10 (t, J=9.0 Hz, 1H), 3.99-3.87 (m, 2H), 3.57-3.12 (m, 4H), 2.23 (s, 3H), 1.98-1.65 (m, 3H), 1.52-1.28 (m, 2H) ppm. MS (ESI): 330.10 (M+H)⁺, 28.12 (M−H)⁻

### Example 124

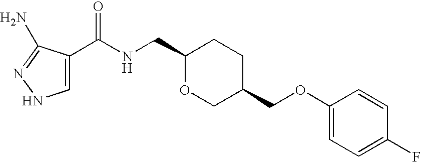

### 3-Amino-N-({(2R,5R)-5-[(4-fluorophenoxy)methyl]tetrahydro-2H-pyran-2-yl}methyl)-1H-pyrazole-4-carboxamide

3-Amino-N-({(2R*15R*)-5-[(4-fluorophenoxy)methyl]tetrahydro-2H-pyran-2-yl}methyl)-1H-pyrazole-4-carboxamide was prepared with 3-amino-1H-pyrazole-4-carboxylic acid (127 mg, 1.0 mmol) and ({(2R*, 5R*)-5-[(4-fluorophenoxy)methyl]tetrahydro-2H-pyran-2-yl}methyl)amine (239 mg, 1.0 mmol) by a procedure similar to that in Example 8, and separated by Chiral column (Chiralpak OJ-H, 20 mm I.D.×250 mm (No. OJH0CJ-DH004), DAICEL) using n-Hexane/Ethanol/Et₂NH=85/15/0.1 as an eluent (10 mL/min) to afford the titled compound (50 mg, 14%).

¹H NMR (DMSO-d₆) δ: 11.73 (brs, 1H), 7.83-7.67 (m, 2H), 7.14-7.07 (m, 2H), 6.99-6.94 (m, 2H), 5.85 (brs, 1H), 4.15-4.08 (m, 1H), 3.99-3.90 (m, 2H), 3.55-3.50 (m, 1H), 3.47-3.37 (m, 1H), 3.27-3.09 (m, 2H), 1.94-1.72 (m, 3H), 1.43-1.24 (m, 2H) ppm. (1H was not observed.) MS (ESI): 349.15 (M+H)⁺, 347.14 (M−H)⁻ Anal. Calcd. for C₁₇H₂₁FN₄O₃.0.2H₂O: C, 58.01; H. 6.13; N, 15.92. Found: C, 58.03; H, 6.34; N, 15.60.

### Example 125

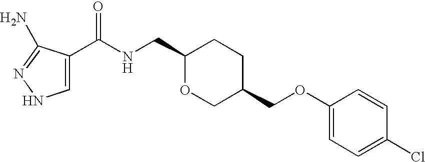

### 3-Amino-N-({(2R,5R)-5-[(4-chlorophenoxy)methyl]tetrahydro-2H-pyran-2-yl}methyl)-1H-pyrazole-4-carboxamide

This compound was prepared with 4-chlorophenol and 3-amino-1H-pyrazole-4-carboxylic acid by a procedure similar to that in Example 119.

¹H NMR (DMSO-d₆) δ: 13.70 (s, 1H), 7.82-7.65 (m, 2H), 7.30 (d, J=8.9 Hz, 2H), 6.98 (d, J=8.9 Hz, 2H), 4.20-3.85 (m, 3H), 3.60-3.05 (m, 4H), 2.00-1.62 (m, 3H), 1.50-1.05 (m, 2H) ppm. (—NH₂ was not observed.) MS (ESI): 365.07 (M+H)⁺, 363.09 (M−H)⁺⁻

### Example 126

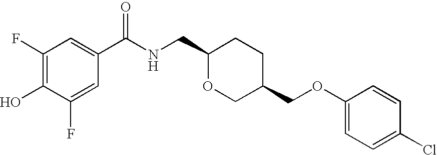

### N-({(2R,5R)-5-[(4-Chlorophenoxy)methyl]tetrahydro-2H-pyran-2-yl}methyl)-3,5-difluoro-4-hydroxybenzamide

This compound was prepared with 4-chlorophenol and 3,5-difluoro-4-hydroxybenzoic acid by a procedure similar to that in Example 119.

¹H NMR (DMSO-d₆) δ: 8.52-8.42 (m, 1H), 7.63-7.46 (m, 2H), 7.30 (d, J=8.8 Hz, 2H), 6.98 (d, J=8.8 Hz, 2H), 4.18-4.07 (m, 1H), 4.02-3.85 (m, 2H), 3.57-3.20 (m, 4H), 2.00-1.63 (m, 3H), 1.53-1.15 (m, 2H) ppm. (—OH was not observed.) MS (ESI): 412.13 (M+H)⁺, 410.13 (M−H)⁺⁻

### Example 127

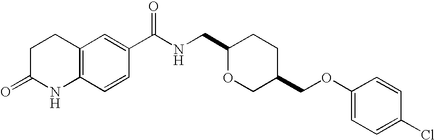

### N-({(2R,5R)-5-[(4-Chlorophenoxy)methyl]tetrahydro-2H-pyran-2-yl}methyl)-2-oxo-1,2,3,4-tetrahydroquinoline-6-carboxamide

This compound was prepared with 4-chlorophenol and 2-oxo-1,2,3,4-tetrahydroquinoline-6-carboxylic acid by a procedure similar to that in Example 119 as a white solid.

¹H NMR (CDCl₃) δ: 8.48 (s, 1H), 7.64 (s, 1H), 7.59-7.57 (m, 1H), 7.27-7.20 (m, 2H), 6.85-6.79 (m, 3H), 6.55-6.52 (m, 1H), 4.17-4.11 (m, 2H), 3.99-3.93 (m, 1H), 3.84-3.76 (m, 1H), 3.68-3.63 (m, 1H), 3.61-3.53 (m, 1H), 3.27-3.18 (m, 1H), 3.02-2.96 (m, 2H), 2.69-2.64 (m, 2H), 2.11-1.76 (m, 3H), 1.64-1.40 (m, 1H), 1.36-1.22 (m, 1H) ppm. MS (ESI): 429 (M+H)⁺

### Example 128

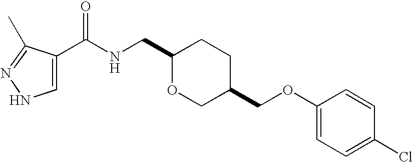

### N-({(2R,5R)-5-[(4-Chlorophenoxy)methyl]tetrahydro-2H-pyran-2-yl}methyl)-3-methyl-1H-pyrazole-4-carboxamide

This compound was prepared with 4-chlorophenol and 1H-3-methylpyrazole-4-carboxylic acid by a procedure similar to that in Example 119.

¹H NMR (CDCl₃) δ: 7.86 (s, 1H), 7.21 (d, J=8.8 Hz, 2H), 6.96-6.87 (m, 1H), 6.83 (d, J=8.8 Hz, 2H), 4.18-4.10 (m, 2H), 3.97-3.92 (m, 1H), 3.83-3.67 (m, 3H), 3.21-3.16 (m, 1H), 2.50 (s, 3H), 2.13-2.03 (m, 1H) 1.98-1.76 (m, 2H), 1.62-1.39 (m, 1H), 1.33-1.22 (m, 1H) ppm. (1H was not observed.) MS (ESI): 364 (M+H)⁺

### Example 129

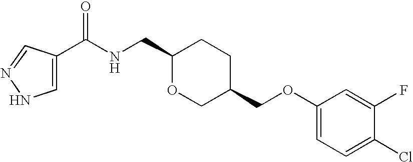

### N-({(2R,5R)-5-[(4-Chloro-3-fluorophenoxy)methyl]tetrahydro-2H-pyran-2-yl}methyl)-1H-pyrazole-4-carboxamide

This compound was prepared with 4-chloro-3-fluorophenol and 1H-pyrazole-4-carboxylic acid by a procedure similar to that in Example 119.

¹H NMR (DMSO-d₆) δ: 13.09 (brs, 1H), 8.12-8.03 (m, 3H), 7.46 (t, J=8.1 Hz, 1H), 7.14-7.09 (m, 1H), 6.88-6.84 (m, 1H), 4.18 (t, J=8.1 Hz, 1H), 4.04-3.98 (m, 1H), 3.93-3.89 (m, 1H), 3.55-3.50 (m, 1H), 3.47-3.40 (m, 1H), 3.29-3.13 (m, 2H), 1.96-1.70 (m, 3H), 1.50-1.36 (m, 2H) ppm. MS (ESI): 368.03 (M+H)⁺, 366.03 (M−H)⁻

### Example 130

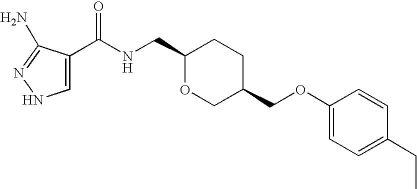

### 3-Amino-N-({(2R,5R)-5-[(4-ethylphenoxy)methyl]tetrahydro-2H-pyran-2-yl}methyl)-1H-pyrazole-4-carboxamide

This compound was prepared with 4-ethylphenol and 3-amino-1H-pyrazole-4-carboxylic acid by a procedure similar to that in Example 119.

¹H NMR (CDCl₃) 5: 7.55 (s, 1H), 7.11-7.08 (m, 2H), 6.85-6.81 (m, 2H), 6.81-6.72 (m, 1H), 5.58-5.31 (m, 2H), 4.17-4.07 (m, 2H), 3.98-3.93 (m, 1H), 3.72-3.49 (m, 3H), 3.14-3.05 (m, 1H), 2.57 (d, J=7.5 Hz, 2H), 2.07-1.79 (m, 3H), 1.62-1.35 (m, 2H), 1.19 (t, J=7.5 Hz, 3H) ppm. (NH was not observed.) MS (ESI): 359 (M+H)⁺

### Example 131

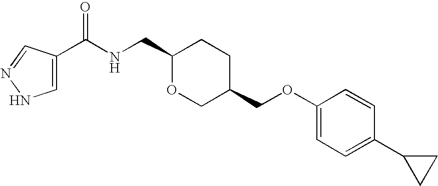

### N-({(2R,5R)-5-[(4-Cyclopropylphenoxy)methyl]tetrahydro-2H-pyran-2-yl}methyl)-1H-pyrazole-4-carboxamide

This compound was prepared with 4-cyclopropylphenol by a procedure similar to that in Example 119 as colorless oil.

¹H NMR (DMSO-d₆) δ: 13.09 (brs, 1H), 8.10-8.03 (m, 3H), 7.00-6.96 (m, 2H), 6.85-6.81 (m, 2H), 4.12-4.06 (m, 1H), 3.97-3.89 (m, 2H), 3.55-3.49 (m, 1H), 3.48-3.40 (m, 1H), 3.28-3.18 (m, 2H), 1.93-1.67 (m, 4H), 1.44-1.34 (m, 2H), 0.90-0.83 (m, 2H), 0.59-0.53 (m, 2H) ppm. MS (ESI): 356.14 (M+H)⁺, 354.16 (M−H)⁻ Anal. Calcd. for C₂₀H₂₅N₃O₃.0.3H₂O: C, 66.57; H, 7.15; N, 11.65. Found: C, 66.41; H, 7.17; N, 11.49.

### Example 132

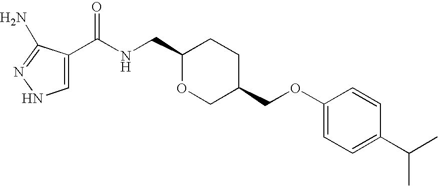

### 3-Amino-N-({(2R,5R)-5-[(4-isopropylphenoxy)methyl]tetrahydro-2H-pyran-2-yl}methyl)-1H-pyrazole-4-carboxamide

This compound was prepared with 4-isopropylphenol and 3-amino-1H-pyrazole-4-carboxylic acid by a procedure similar to that in Example 119 as colorless oil.

¹H NMR (DMSO-d₆) δ: 11.76 (brs, 1H), 7.86-7.71 (m, 2H), 7.14 (d, J=8.1 Hz, 2H), 6.87 (d, J=8.1 Hz, 2H), 4.13-4.07 (m, 1H), 3.99-3.89 (m, 2H), 3.55-3.50 (m, 2H), 3.30-3.08 (m, 2H), 2.87-2.77 (m, 1H), 1.93-1.67 (m, 3H), 1.48-1.36 (m, 2H), 1.18-1.15 (m, 6H) ppm. (NH₂ were not observed.) MS (ESI): 373.21 (M+H)⁺, 371.21 (M−H)⁻

### Example 133

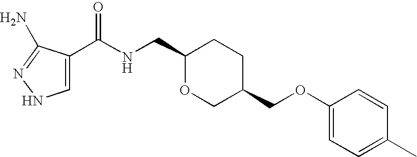

### 3-Amino-N-({(2R,5R)-5-[(4-methylphenoxy)methyl]tetrahydro-2H-pyran-2-yl}methyl)-1H-pyrazole-4-carboxamide

This compound was prepared with p-cresol and 3-amino-1H-pyrazole-4-carboxylic acid by a procedure similar to that in Example 119.

¹H NMR (DMSO-d) δ: 11.73 (br, 1H), 8.20-7.50 (m, 2H), 7.08 (d, J=8.3 Hz, 2H), 6.85 (d, J=8.4 Hz, 2H), 6.10-5.20 (m, 2H), 4.10 (t, J=8.9 Hz, 1H), 3.97-3.89 (m, 2H), 3.53 (dd, J=11.5, 2.5 Hz, 1H), 3.45-3.38 (m, 1H), 3.28-3.22 (m, 1H), 3.18-3.12 (m, 1H), 2.24 (s, 3H), 1.97-1.91 (m, 1H), 1.87-1.81 (m, 1H), 1.77-1.68 (m, 1H), 1.48-1.43 (m, 1H), 1.39-1.31 (m, 1H) ppm. MS (ESI): 345.23 (M+H)⁺, 343.22 (M−H)⁻

### Example 134

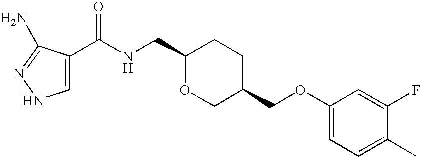

### 3-Amino-N-({(2R,5R)-5-[(3-fluoro-4-methylphenoxy)methyl]tetrahydro-2H-pyran-2-yl}methyl)-1H-pyrazole-4-carboxamide

This compound was prepared with 3-fluoro-4-cresol and 3-amino-1H-pyrazole-4-carboxylic acid by a procedure similar to that in Example 119.

¹H NMR (DMSO-d) δ: 11.70 (br, 1H), 8.10-7.50 (m, 2H), 7.15 (d, J=8.8 Hz, 1H), 6.78 (dd, J=11.9, 2.4 Hz, 1H), 6.71 (dd, J=8.4,2.4 Hz, 1H), 6.20-5.05 (m, 2H), 4.12 (t, J=9.1 Hz, 1H), 3.96 (dd, J=9.4, 6.7 Hz, 1H), 3.90 (d, J=11.6 Hz, 1H), 3.51 (dd, J=11.6, 2.6 Hz, 1H), 3.44-3.38 (m, 1H), 3.28-3.20 (m, 1H), 3.18-3.10 (m, 1H), 2.14 (s, 3H), 1.97-1.69 (m, 1H), 1.84-1.77 (m, 1H), 1.76-1.67 (m, 1H), 1.48-1.42 (m, 1H), 1.40-1.30 (m, 1H) ppm. MS (ESI): 363.18 (M+H)⁺, 361.13 (M−H)⁻

### Example 135

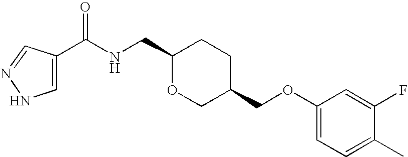

### N-({(2R,5R)-5-[(3-Fluoro-4-methylphenoxy)methyl]tetrahydro-2H-pyran-2-yl}methyl)-1H-pyrazole-4-carboxamide

This compound was prepared with 3-fluoro-4-cresol by a procedure similar to that in Example 119.

¹H NMR (DMSO-d) δ: 13.08 (br, 1H), 8.20-7.85 (m, 3H), 7.15 (t, J=8.8 Hz, 1H), 6.78 (dd, J=11.9, 2.4Hz, 1H), 6.70 (dd, J=8.4,2.4 Hz, 1H), 4.12 (t, J=9.0 Hz, 1H), 3.96 (dd, J=9.4, 6.7 Hz, 1H), 3.90 (d, J=11.6 Hz, 1H), 3.52 (dd, J=11.6, 2.6 Hz, 1H), 3.47-3.41 (m, 1H), 3.37-3.25 (m, 1H), 3.22-3.15 (m, 1H), 2.14 (s, 3H), 1.96-1.90 (m, 1H), 1.84-1.78 (m, 1H), 1.76-1.68 (m, 1H), 1.48-1.43 (m, 1H), 1.42-1.33 (m, 1H) ppm. MS (ESI): 348.21 (M+H)⁺, 346.15 (M−H)⁻

### Example 136

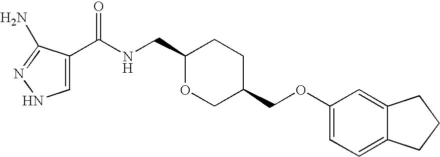

### 3-Amino-N-({(2R,5R)-5-[(2,3-dihydro-1H-inden-5-yloxy)methyl]tetrahydro-2H-pyran-2-yl}methyl)-1H-pyrazole-4-carboxamide

This compound was prepared with indan-5-ol and 3-amino-1H-pyrazole-4-carboxylic acid by a procedure similar to that in Example 119.

¹H NMR (DMSO-d) δ: 11.81 (br, 1H), 8.20-7.50 (m, 2H), 7.08 (d, J=8.1 Hz, 1H), 6.84-6.80 (m, 1H), 6.69 (dd, J=8.1, 2.2 Hz, 1H), 6.20-5.00 (m, 2H), 4.09 (t, J=8.9 Hz, 1H), 3.97-3.86 (m, 2H), 3.51 (dd, J=11.6, 2.6Hz, 1H), 3.44-3.37 (m, 1H), 3.27-3.19 (m, 1H), 3.18-3.10 (m, 1H), 2.81 (t, J=7.4 Hz, 2H), 2.76 (t, J=7.3 Hz, 2H), 2.03-1.96 (m, 2H), 1.95-1.89 (m, 1H), 1.85-1.78 (m, 1H), 1.76-1.64 (m, 1H), 1.48-1.41 (m, 1H), 1.39-1.29 (m, 1H) ppm. MS (ESI): 371.12 (M+H)⁺, 369.14 (M−H)⁻

### Example 137

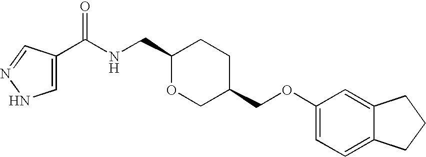

### N-({(2R,5R)-5-[(2,3-Dihydro-1H-inden-5-yloxy)methyl]tetrahydro-2H-pyran-2-yl}methyl)-1H-pyrazole-4-carboxamide

This compound was prepared with indan-5-ol by a procedure similar to that in Example 119.

¹H NMR (DMSO-d) δ: 13.08 (br, 1H), 8.20-7.85 (m, 3H), 7.08 (d, J=8.2 Hz, 1H), 6.82 (s, 1H), 6.89 (dd, J=8.2, 2.2 Hz, 1H), 4.09 (t, J=8.9 Hz, 1H), 3.97-3.88 (m, 2H), 3.52 (dd, J=11.6, 2.6 Hz, 1H), 3.47-3.40 (m, 1H), 3.38-3.24 (m, 1H), 3.23-3.14 (m, 1H), 2.81 (t, J=7.4 Hz, 2H), 2.76 (t, J=7.3 Hz, 2H), 2.04-1.90 (m, 3H), 1.85-1.78 (m, 1H), 1.75-1.65 (m, 1H), 1.49-1.42 (m, 1H), 1.40-1.31 (m, 1H) ppm. MS (ESI): 356.21 (M+H)⁺, 354.12 (M−H)⁻
Preparation 1

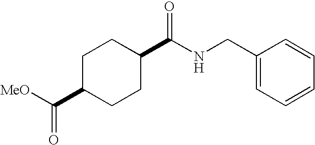

### cis-Methyl-4-[(benzylamino)carbonyl]cyclohexanecarboxylate

A mixture of cis-4-(methoxycarbonyl)cyclohexanecarboxylic acid (35 g, 0.19 mol) (J. Am. Chem. Soc. 1956, 78, 4000-4002.), benzyl amine (22 g, 0.21 mol), EDCI (40 g, 0.21 mol) and HOBt-H₂O (5.7 g, 37 mmol) in DMF (380 mL) was stirred at room temperature for 16 hours. The mixture was concentrated under reduced pressure and diluted with AcOEt. The organic layer was washed with 2 N aq. HCl, sat. aq. NaHCO₃ and water, dried over MgSO₄, and concentrated in vacuum to give the titled compound. (51 g)

¹H NMR (CDCl₃) δ: 7.38-7.22 (m, 5H), 5.78 (br, 1H), 4.44 (d, J=5.8 Hz, 2H), 3.69 (s, 3H), 2.63-2.54 (m, 1H), 2.30-2.05 (m, 3H), 1.80-1.50 (m, 6H) ppm.
Preparation 2

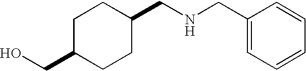

### {cis-4-[(Benzylamino)methyl]cyclohexyl}methanol

A solution of cis-Methyl 4-[(benzylamino)carbonyl]cyclohexanecarboxylate (51 g, 0.19 mol) in THF (200 mL) was added dropwise to a suspension of LiAlH₄ (21 g, 0.56 mol) in THF (1.0 L) at 0° C. and the mixture was refluxed for 16 hours. The reaction mixture was added dropwise to a suspension of Na₂SO₄.10H₂O (excess) in CH₂Cl₂ at 0° C. and the mixture was stirred at room temperature for 3 hours. The white suspension was filtered and the filtrate was concentrated in vacuum. The residue was dissolved with CH₂Cl₂ and filtered through cotton. The filtrate was evaporated to give the titled compound (40 g, 0.17 mol).

¹H NMR (CDCl₃) δ: 7.35-7.20 (m, 5H), 3.78 (s, 2H), 3.52 (d, J=6.9 Hz, 2H), 2.55 (d, J=7.1 Hz, 2H), 1.90-1.30 (m, 10H) ppm. (OH and NH were not observed.)
Preparation 3

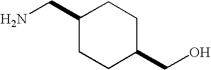

### [cis-4-(Aminomethyl)cyclohexyl]methanol

A mixture of {cis-4-[(benzylamino)methyl]cyclohexyl}methanol (35 g, 0.15 mol) and 20% w/w Pd(OH)₂—C (3.0 g) in MeOH (300 ml) was hydrogenated at 4 atm for 10 h. The mixture was filtered through a pad of celite and the filtrate was evaporated to give the titled compound (22 g).

¹H NMR (CDCl₃) δ: 3.52 (d, J=6.9 Hz, 2H), 2.61 (d, J=6.1 Hz, 2H), 1.80-1.30 (m, 10H) ppm. (OH and NH₂ were not observed.)
Preparation 4

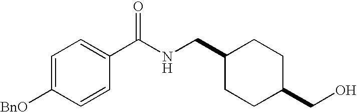

### 4-(Benzyloxy)-N-{[cis-4-(hydroxymethyl)cyclohexyl]methyl}benzamide

To a mixture of [cis-4-(aminomethyl)cyclohexyl]methanol (2.8 g, 20 mmol), triethylamine (3.3 mL, 24 mmol) and DMAP (0.24 g, 2.0 mmol) in CH₂Cl₂, TBSCI (3.3 g, 22 mmol) was added at 0° C. and the mixture was stirred at room temperature for 16 hours. To the mixture, sat. aq. NaHCO₃ was added and the whole was extracted with CH₂Cl₂. The extract was dried over MgSO₄ and evaporated to afford {[cis-4-({[tert-butyl(dimethyl)silyl]oxy}methyl)cyclohexyl]methyl}amine. A mixture of {[cis-4-({[tert-butyl(dimethyl)silyl]oxy}methyl)cyclohexyl]methyl}amine, 4-(benzyloxy)benzoic acid (4.6 g, 20 mmol), EDCI (4.2 g, 22 mmol) and HOBt-H₂O (3.4 g, 22 mmol) in DMF (40 mL) was stirred at room temperature for 16 hours. The mixture was diluted with AcOEt and washed with sat. aq. NaHCO₃ and water, dried over MgSO₄, and evaporated to afford 4-(benzyloxy)-N-{[cis-4-({[tert-butyl(dimethyl)silyl]oxy}methyl)cyclohexyl]methyl}benzamide. A mixture of 4-(benzyloxy)-N-{[cis-4-({[tert-butyl(dimethyl)silyl]oxy}methyl)cyclohexyl]methyl}benzamide and TBAF (1.0 M in THF, 30 mL) was stirred at room temperature for 4 hours. The mixture was diluted with AcOEt and was washed with 2 N aq. HCl and water, dried over MgSO₄ and evaporated. The residue was purified by silica gel colomn chromatography (hexane-AcOEt 1:3) to give the titled compound (1.9 g).

¹H NMR (CDCl₃) δ: 7.72 (d, J=8.9 Hz, 2H), 7.46-7.30 (m, 5 H), 7.00 (d, J=8.9 Hz, 2H), 6.05-5.95 (m, 1H), 5.11 (s, 2H), 3.56 (dd, J=5.6, 6.8 Hz, 2H), 3.40 (dd, J=5.9, 7.4 Hz, 2 H), 1.90-1.40 (m, 10H) ppm. (OH was not observed.)
Preparation 5

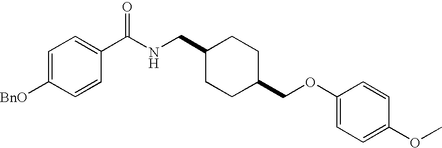

### 4-(Benzyloxy)-N-({cis-4-[(4-methoxyphenoxy)methyl]cyclohexyl}methyl)benzamide

Cyanomethylenetributylphosphorane (80 mg, 0.30 mmol) was added to a mixture of 4-(benzyloxy)-N-{[cis-4-(hydroxymethyl)cyclohexyl]methyl}benzamide (71 mg, 0.2 mmol), 4-methoxyphenol (37 mg, 0.30 mmol) in benzene (1.0 mL). The mixture was refluxed for 1hour and purified by silica gel column chlomatography (hexane-AcOEt 4:1) to give the titled compound (84 mg).

¹H NMR (CDCl₃) δ: 7.72 (d, J=8.8 Hz, 2H), 7.46-7.30 (m, 5H), 7.00 (d, J=8.8 Hz, 2H), 6.83 (s, 4H), 6.10-6.00 (m, 1H), 5.11 (s, 2H), 3.82 (d, J=7.0 Hz, 2H), 3.77 (s, 3H), 3.41 (dd, J=6.2,7.1 Hz, 2H), 2.06-1.40 (m, 10H) ppm.
Preparation 6

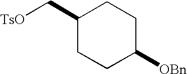

### [cis-4-(Benzyloxy)cyclohexyl]methyl 4-methylbenzenesulfonate

Triflic acid (1.3 mL) was added to a mixture of (4-hydroxycyclohexyl)methyl 4-methylbenzenesulfonate (21 g, 75 mmol) (J. Org. Chem. 1970, 35, 2386-2390.) and benzyl 2,2,2-trichloroacetimidate (38 g, 0.15 mol) in CH₂Cl₂ at 0° C. and the mixture was stirred at room temperature for 16 hours. To the mixture, sat. aq. NaHCO₃ was added and the mixture was extracted with CH₂Cl₂. The extract was dried over MgSO₄ and evaporated. The residue was purified by silica gel column chiomatography (hexane-AcOEt 10:1) to give the titled compound (13 g).

¹H NMR (CDCl₃) δ: 7.78 (d, J=8.4 Hz, 2H), 7.40-7.10 (m, 7 H), 4.46 (s, 2H), 3.86 (d, J=6.9 Hz, 2H), 3.65-3.58 (m, 1H), 2.45 (s, 3H), 1.98-1.24 (m, 9H) ppm.
Preparation 7

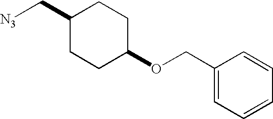

### ({[cis-4-(Azidomethyl)cyclohexyl]oxy}methyl)benzene

A mixture of [cis-4-(benzyloxy)cyclohexyl]methyl 4-methylbenzenesulfonate (14 g, 37 mmol) and sodium azide (12 g, 0.19 mol) in DMF (150 mL) was stirred at 85° C. for 3 hours. The mixture was diluted with AcOEt and washed with water. The organic layer was dried over MgSO₄ and evaporated. The residue was purified by silica gel column chlomatography (hexane:AcOEt=20:1) to give the titled compound (6.9 g).

¹H NMR (CDCl₃) δ: 7.36-7.22 (m, 5H), 4.50 (s, 2 H), 3.68-3.61 (m, 1H), 3.16 (d, J=6.6 Hz, 2H), 2.04-1.86 (m, 2H), 1.70-1.36 (m, 7H) ppm.
Preparation 8

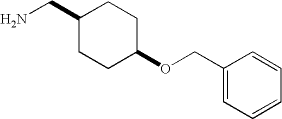

### [cis-4-(Benzyloxy)cyclohexyl]methylamine

A solution of ({[cis-4-(azidomethyl)cyclohexyl]oxy}methyl)benzene (6.9 g, 28 mmol) in THF (20 mL) was added dropwise to a suspension of LiAlH₄ (1.6 g, 42 mmol) in THF (140 mL) at 0° C. and the mixture was stirred at room temperature for 1 hour. The mixture was quenched with Na₂SO₄.10H₂O (excess) and the white suspension was filtered. The filtrate was evaporated to give the titled compound (5.7 g).

¹H NMR (CDCl₃) δ: 7.40-7.22 (m, 5H), 4.50 (s, 2 H), 3.66-3.60 (m, 1H), 2.58 (d, J=5.7 Hz, 2H), 2.00-1.30 (m, 9H) ppm. (NH₂ was not observed.)
Preparation 9

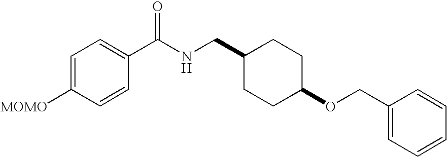

### N-{[cis-4-(Benzyloxy)cyclohexyl]methyl}-4-(methoxymethoxy)benzamide

A mixture of 4-(methoxymethoxy)benzoic acid (2.6 g, 14 mmol), [cis-4-(benzyloxy)cyclohexyl]methylamine (3.0 g, 14 mmol), EDCI (3.2 g, 17 mmol) and HOBt.H₂O (0.43 g, 2.8 mmol) in DMF (70 mL) was stirred at room temperature for 16 hours. The mixture was diluted with AcOEt and was washed with sat. aq. NaHCO₃ and water, dried over MgSO₄ and evaporated. The residue was crystallized from CH₂Cl₂-diisopropylether to give the titled compound (4.2 g) as a white solid.

¹H NMR (CDCl₃) δ: 7.72 (d, J=8.8 Hz, 2H), 7.38-7.23 (m, 5H), 7.06 (d, J=8.8 Hz, 2H), 6.18-6.08 (m, 1H), 5.21 (s, 2H), 4.50 (s, 2H), 3.68-3.62 (m, 1H), 3.48 (s, 3H), 3.34 (t, J=6.4 Hz, 2H), 2.04-1.90 (m, 2H), 1.75-1.40 (m, 7H) ppm.
Preparation 10

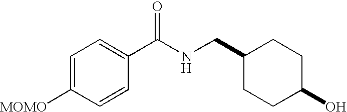

### N-[(cis-4-Hydroxycyclohexyl)methyl]-4-(methoxymethoxy)benzamide

A mixture of N-{[cis-4-(benzyloxy)cyclohexyl]methyl}-4-(methoxymethoxy)benzamide (4.0 g, 10 mmol) and 20% Pd(OH)₂—C (0.50 g) in EtOH (200 mL) was hydrogenated under hydrogene atomsphere at 4 atm at room temperature for 8 h. The mixture was filtered by celite and evaporated. The titled compound (2.9 g) was afforded by crystallization from CH₂Cl₂-diisopropylether as a white solid.

¹H NMR (CDCl₃) δ: 7.73 (d, J=8.2 Hz, 2H), 7.06 (d, J=9.0 Hz, 2H), 6.20-6.10 (m, 1H), 5.22 (s, 2H), 4.05-3.98 (m, 1H), 3.48 (s, 3H), 3.35 (t, J=6.6 Hz, 2H), 1.84-1.36 (m, 9H) ppm. (OH was not observed.)
Preparation 11

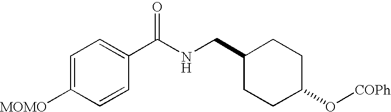

### trans-4-({[4-(Methoxymethoxy)benzoyl]amino}methyl)cyclohexyl benzoate

A mixture of N-[(cis-4-hydroxycyclohexyl)methyl]-4-(methoxymethoxy)benzamide (0.58 g, 2.0 mmol), benzoic acid (0.37 g, 3.0 mmol) and cyanomethylenetributylphosphorane (0.80 g, 3.0 mmol) in benzene (10 mL) was refluxed for 4 hours. The residue was purified by silica gel column chlomatography (hexane:AcOEt=3:1) to give the titled compound (0.28 g).

¹H NMR (CDCl₃) δ: 8.06-8.00 (m, 2H), 7.75 (d, J=8.8 Hz, 2H), 7.50-7.40 (m, 3H), 7.07 (d, J=8.8 Hz, 2H), 6.32-6.20 (m, 1H), 5.22 (s, 2H), 5.00-4.87 (m, 1H), 3.48 (s, 3H), 3.34 (t, J=6.4 Hz, 2H), 2.20-2.10 (m, 2H), 1.98-1.88 (m, 2H), 1.78-1.42 (m, 3H), 1.30-1.12 (m, 2H) ppm.
Preparation 12

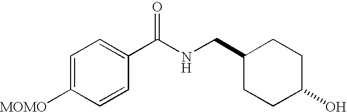

### N-[(trans-4-Hydroxycyclohexyl)methyl]-4-(methoxymethoxy)benzamide

A mixture of trans-4-({[4-(methoxymethoxy)benzoyl]amino}methyl)cyclohexyl benzoate (0.24 g, 0.61 mmol), 2N aq. NaOH (3 mL), MeOH (3 mL) and THF (3 mL) was stirred at room temperature for 1 hour. The mixture was diluted with water and was extracted with CH₂Cl₂. The extract was dried over MgSO₄ and evaporated to give the titled compound (0.17 g).

¹H NMR (CDCl₃) δ: 7.72 (d, J=8.8 Hz, 2H), 7.07 (d, J=8.6 Hz, 2H), 6.15-6.00 (br, 1H), 5.22 (s, 2H), 3.65-3.50 (m, 1H), 3.48 (s, 3H), 3.31 (t, J=6.6 Hz, 2H), 2.13-1.95 (m, 2H), 1.91-1.80 (m, 2H), 1.65-1.00 (m, 5H) ppm. (OH was not observed.)
Preparation 13

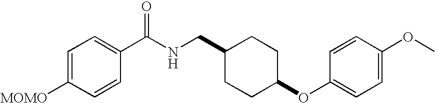

### 4-(Methoxymethoxy)-N-{[cis-4-(4-methoxyphenoxy)cyclohexyl]methyl}benzamide

A mixture of N-[(trans-4-hydroxycyclohexyl)methyl]-4-(methoxymethoxy)benzamide (30 mg, 0.10 mmol), 4-methoxyphenol (19 mg, 0.15 mmol) and cyanomethylenetributylphosphorane (40 mg, 0.15 mmol) in benzene (0.5 mL) was refluxed for 4 hours. The mixture was purified by silica gel column chlomatography (hexane:AcOEt=3:1) to give the titled compound (11 mg).

¹H NMR (CDCl₃) δ: 7.72 (d, J=8.9 Hz, 2H), 7.07 (d, J=8.7 Hz, 2H), 6.90-6.80 (m, 4H), 6.20-6.05 (m, 1H), 5.22 (s, 2H), 4.46-4.38 (m, 1H), 3.77 (s, 3H), 3.48 (s, 3H), 3.36 (t, J=6.4 Hz, 2H), 2.15-1.40 (m, 9H) ppm.
Preparation 14

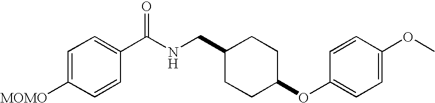

### N-{[cis-4-(4-Chlorophenoxy)cyclohexyl]methyl}-4-(methoxymethoxy)benzamide

This compound was prepared with 4-chlorophenol by a procedure similar to that in Preparation 13.

¹H NMR (CDCl₃) δ: 7.72 (d, J=8.8 Hz, 2H), 7.21 (d, J=9.2 Hz, 2H), 7.07 (d, J=8.8 Hz, 2H), 6.83 (d, J=9.0 Hz, 2H), 6.20-6.10 (m, 1 H), 5.22 (s, 2H), 4.53-4.46 (m, 1H), 3.48 (s, 3H), 3.36 (t, J=6.4 Hz, 2H), 2.10-2.00 (m, 2H) 1.80-1.40 (m, 7H) ppm.

Preparation 15

### 1-(Aminomethyl)-4-(phenoxymethyl)cyclohexanol hydrochloride

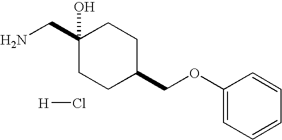

4-(Phenoxymethyl)cyclohexanone (5.0 g, 24 mmol) (Tetrahedron 1969, 25, 2159-2192.) was added to a mixture of trimethylsilylcyanide (3.5 mL, 26 mmol) and zinc iodide (0.38 g, 1.2 mmol) in toluene (48 mL) at −78° C. The mixture was stirred at 0° C. for 4 hours. The mixture was added dropwise to a suspension of LiAlH₄ (1.8 g) in TIF (100 mL) at 0° C. and the mixture was stirred at room temperature for 2 hours. The mixture was quenched with excess of Na₂SO₄.10H₂O and stirred for 4 hours. After filtration, the filtrate was concentrated to give 1-(aminomethyl)-4-(phenoxymethyl)cyclohexanol. 4N HCl in AcOEt (7 mL) was added to a solution of 1-(aminomethyl)-4-(phenoxymethyl)cyclohexanol in EtOH (30 mL) and the mixture was concentrated. The residue was crystallized from MeOH (15 mL) to give 1-(aminomethyl)-4-(phenoxymethyl)cyclohexanol hydrochloride (4.9 g).

hu 1H NMR (DMSO-d₆) δ: 7.93 (br, 3H), 7.32-7.24 (m, 2 H), 6.96-6.88 (m, 3H), 5.08 (br, 1H), 3.83 (d, J=6.1 Hz, 2H), 2.83 (s, 2H), 1.85-1.70 (m, 5H), 1.50-1.12 (m, 4H) ppm.
Preparation 16

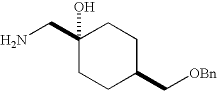

### trans-1-(Aminomethyl)-4-[(benzyloxy)methyl]cyclohexanol hydrochloride

4-[(Benzyloxy)methyl]cyclohexanone (7.5 g, 34 mmol) (J. Med. Chem. 1993, 36, 654-670) was added to a mixture of ZnI₂ (0.54 g, 1.7 mmol) and TMSCN (4.8 mL, 36 mmol) in toluene (34 mL) at −78° C. and the mixture was stirred at −78° C. for 3 hours. The mixture was dropwised to a suspension of LiAlH₄ (2.6 g, 68 mmol) in THF (136 mL) at 0° C. and the mixture was stirred at room temperature for 2 hours. The mixture was quenched with Na₂SO₄.10H₂O (excess) and stirred for 4 hours. After filtration, the filtrate was evaporated. The residue was dissolved with ethanol and 4N HCl in AcOEt (10 mL) was added at 0° C. The sovent was removed in vacuum. The residue was crystallized from ethanol to afford the titled compound (6.1 g) as a white solid.

¹H NMR (DMSO-d₆) δ: 7.93 (br, 3H), 7.38-7.24 (m, 5H), 5.07 (br, 1H), 4.45 (s, 2H), 3.28 (d, J=6.0 Hz, 2H), 2.79 (s, 2H), 1.75-1.00 (m, 9H) ppm.
Preparation 17

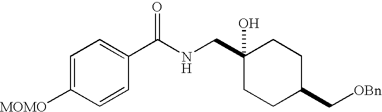

### N-({trans-4-[(Benzyloxy)methyl]-1-hydroxycyclohexyl}methyl)-4-(methoxymethoxy)benzamide

A mixture of 4-(methoxymethoxy)benzoic acid (4.0 g, 22 mmol), trans-1-(aminomethyl)-4-[(benzyloxy)methyl]cyclohexanol hydrochloride (6.1 g, 21 mmol), Et₃N (5.9 mL, 42 mmol), EDCI (4.8 g, 25 mmol) and HOBt.H₂O (0.64 g, 4.2 mmol) in DMF (60 mL) was stirred at room temperature for 16 hours. The mixture was diluted with AcOEt and washed with sat. aq. NaHCO₃ and water, dried over MgSO₄, and evaporated. The residue was crystallized from CH₂Cl₂-hexane to afford the titled compound (7.2 g) as a white solid.

¹H NMR (CDCl₃) δ: 7.75 (d, J=8.9 Hz, 2H), 7.96-7.26 (m, 5H), 7.07 (d, J=8.9 Hz, 2H), 6.56-6.46 (m, 1H), 5.22 (s, 2H), 4.49 (s, 2H), 3.57 (d, J=5.9 Hz, 2H), 3.48 (s, 3H), 3.33 (d, J=6.4 Hz, 2H), 2.41 (s, 1H), 1.90-1.10 (m, 9H) ppm.
Preparation 18

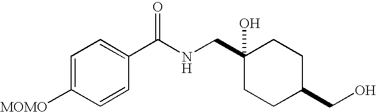

### N-{[trans-1-Hydroxy-4-(hydroxymethyl)cyclohexyl]methyl}-4-(methoxymethoxy)benzamide

A mixture of N-({trans-4-[(benzyloxy)methyl]-1-hydroxycyclohexyl}methyl)-4-(methoxymethoxy)benzamide (6.5 g, 16 mmol) and 20% Pd(OH)₂—C (0.5 g) in EtOH (160 mL) was hydrogenated under 4 atm at room temperature for 4 hours and at 60° C. for 4 hours. The mixture was filtered through a pad of celite and the filtrate was evaporated. The residue was purified by silica gel column chromatography (CH₂Cl₂:MeOH=12:1) to give the titled compound (4.5 g) as a white solid.

¹H NMR (DMSO-d₆) δ: 8.02 (t, J=5.9 Hz, 1H), 7.84 (d, J=8.6 Hz, 2H), 7.07 (d, J=8.8 Hz, 2H), 5.25 (s, 2H), 4.64 (s, 1H), 4.40 (t, J=5.1 Hz, 1H), 3.39-3.30 (m, 5H), 3.24 (d, J=5.9 Hz, 2H), 1.68-1.55 (m, 4H), 1.44-1.02 (m, 5H) ppm.
Preparation 19

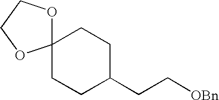

### 8-[2-(Benzyloxy)ethyl]-1,4-dioxaspiro[4.5]decane

To a solution of 2-(1,4-dioxaspiro[4.5]dec-8-yl)ethanol (2.0 g, 11 mmol) (J. Am. Chem. Soc. 1991, 113, 8016-8024.) in DMF (20 mL), NaH (60%, 0.48 g, 12 mmol) was added at 0° C. and the mixture was stirred at room temperature for 3 hours. The mixture was quenched with water and the whole was extracted with AcOEt. The organic layer was washed with water, dried over MgSO₄, and evaporated. The residue was purified by silica gel column chromatography (hexane:AcOEt=10:1) to give the titled compound (2.2 g).

¹H NMR (CDCl₃) δ: 7.40-7.22 (m, 5H), 4.50 (s, 2H), 3.93 (s, 4H), 3.50 (t, J=6.4 Hz, 2H), 1.80-1.16 (m, 11H) ppm.

Preparation 20

### 4-[2-(Benzyloxy)ethyl]cyclohexanone

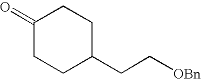

A mixture of 8-[2-(benzyloxy)ethyl]-1,4-dioxaspiro[4.5]decane (2.2 g, 8.0 mmol) and 2N aq. HCl (40 mL) in THF was stirred at 50° C. for 3 hours. The mixture was extracted with AcOEt and the extract was washed with sat. aq. NaHCO₃ and water, dried over MgSO₄ and evaporated to give 4-[2-(benzyloxy)ethyl]cyclohexanone (1.9 g).

¹H NMR (CDCl₃) δ: 7.40-7.25 (m, 5H), 4.52 (s, 2H), 3.54 (t, J=6.3 Hz, 2H), 2.45-1.30 (m, 11H) ppm.

Preparation 21

### trans-1-(Aminomethyl)-4-[2-(benzyloxy)ethyl]cyclohexanol hydrochloride

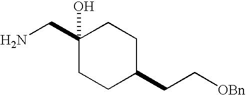

A solution of 4-[2-(benzyloxy)ethyl]cyclohexanone (1.9 g) in toluene (16 mL) was added to a mixture of ZnI₂ (0.13 g, 0.40 mmol) and TMSCN (1.2 mL, 8.8 mmol) in toluene (10 mL), at −78° C. and the mixture was stirred at −78° C. for 2 hours. The mixture was dropwised to a suspension of LiAlH₄ (0.61 g, 16 mmol) in THF (40 mL) at 0° C. and stirred at room temperature for 1 hour. The mixture was quenched with Na₂SO₄.10H₂O (excess) and KF (excess). After filtration, the filtrate was evaporated. The residue was dissolved with ethanol and 4N HCl in AcOEt (3 mL) was added at 0° C. The sovent was removed in vacuum. The residue was crystallized from ethanol to afford the titled compound (1.5 g) as a white solid.

¹H NMR (DMSO-d₆) δ: 7.89 (br, 3H), 7.40-7.24 (m, 5H), 5.00 (br, 1H), 4.44 (s, 2H), 3.44 (t, J=6.3 Hz, 2H), 2.79 (s, 2H), 1.75-1.00 (m, 11H) ppm.
Preparation 22

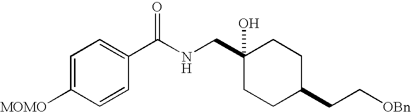

### N-({trans-4-[2-(Benzyloxy)ethyl]-1-hydroxycyclohexyl}methyl)-4-(methoxymethoxy)benzamide

This compound was prepared with trans-1-(aminomethyl)-4-[2-(benzyloxy)ethyl]cyclohexanol hydrochloride by a procedure similar to that in Preparation 17.

¹H NMR (CDCl₃) δ: 7.75 (d, J=8.8 Hz, 2H), 7.38-7.24 (m, 5H), 7.06 (d, J=8.8 Hz, 2H), 6.55 (t, J=5.5 Hz, 1H), 5.20 (s, 2H), 4.49 (s, 2H), 3.58-3.45 (m, 7H), 2.42 (br, 1H), 1.86-1.05 (m, 11H) ppm.
Preparation 23

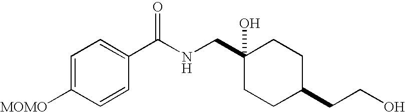

### N-{[trans-1-Hydroxy-4-(2-hydroxyethyl)cyclohexyl]methyl}-4-(methoxymethoxy)benzamide

A mixture of N-({trans-4-[2-(benzyloxy)ethyl]-1-hydroxycyclohexyl}methyl)-4-(methoxymethoxy)benzamide (1.4 g, 3.2 mmol) and 20% Pd(OH)₂—C (0.50 g) in EtOH (60 mL) was hydrogenated under 4 atm at 8 hours. The mixture was filtered through a pad of celite and the filtrate was evaporated. The residue was purified by silica gel column chlomatography (hexane-AcOEt 1:2 to AcOEt only) to give the titled compound (1.0 g).

¹H NMR (CDCl₃) δ: 7.76 (d, J=8.9 Hz, 2H), 7.06 (d, J=9.0 Hz, 2H), 6.65-6.53 (m, 1H), 5.21 (s, 2H), 3.73-3.64 (m, 2H), 3.58 (d, J=5.9 Hz, 2H), 3.48 (s, 3H), 2.43 (br, 1H), 1.88-1.10 (m, 11H) ppm.
Preparation 24

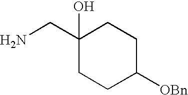

### 1-(Aminomethyl)-4-(benzyloxy)cyclohexanol hydrochloride

4-(Benzyloxy)cyclohexanone (19 g, 94 mmol) (J. Org. Chem. 1982, 47, 3881-3886.) was added dropwise to a mixture of ZnI₂ (1.5 g, 4.7 mmol) and TMSCN (13 mL, 98 mmol) in toluene (100 mL) at 0° C. and the mixture was stirred at room temperature for 2 hours. The mixture was dropwised to a suspension of LiAlH₄ (8.5 g, 98 mmol) in THF (400 mL) at 0° C. and the mixture was stirred at room temperature for 2 h. The mixture was quenched with Na₂SO₄.10H₂O (excess) and stirred for 4 hours. After filtration, the filtrate was evaporated. The residue was dissolved with ethanol and 4N HCl in AcOEt (25 mL) was added at 0° C. The sovent was removed in vacuum. The residue was crystallized from ethanol to afford the titled compound (6.1 g) as a white solid.

¹H NMR (DMSO-d₆) δ: 8.00 (br, 3H), 7.40-7.20 (m, 5H), 4.91 (br, 1H), 4.82-4.44 (m, 2H), 3.60-3.24 (m, 1H), 2.80-2.65 (m, 2H), 1.85-1.20 (m, 8H) ppm.

Preparation 25

### Ethyl 4-{[2-(trimethylsilyl)ethoxy]methoxy}benzoate

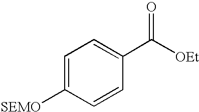

To a mixture of ethyl 4-hydroxybenzoate (4.3 g, 26 mmol) and i-Pr₂NEt (5.4 mL, 31 mmol) in CH₂Cl₂ (52 mL), SEMCl (5.0 mL, 28 mmol) was added at 0° C. and the mixture was stirred at room temperature for 72 hours. The mixture was diluted with CH₂Cl₂.The whole was washed with sat. aq. NH₄CI, dried over MgSO₄, and evaporated to give ethyl 4-{[2-(trimethylsilyl)ethoxy]methoxy}benzoate (9.0 g).

¹H NMR (CDCl₃) δ: 7.99 (d, J=9.1 Hz, 2H), 7.05 (d, J=8.9 Hz, 2H), 5.27 (s, 2H), 4.35 (q, J=7.3 Hz, 2H), 3.79-3.71 (m, 2H), 1.38 (t, J=7.1 Hz, 3H), 0.99-0.89 (m, 2H), −0.01 (s, 9H) ppm.

Preparation 26

### 4-{2-(Trimethylsilyl)ethoxylmethoxy}benzoic acid

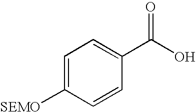

A mixture of ethyl 4-{[2-(trimethylsilyl)ethoxy]methoxy}benzoate (9.0 g) and 8N aq. KOH (20 mL) in EtOH (50 mL) was stirred at room temperature for 6 hours. The mixture was acidified with c.HCl at 0° C. The precipitate was filtered and washed with water to give the titled compound (6.7 g) as a white crystal.

¹H NMR (CDCl₃) δ: 8.07 (d, J=8.9 Hz, 2H), 7.09 (d, J=9.0 Hz, 2H), 5.29 (s, 2H), 3.81-3.72 (m, 2H), 1.00-0.92 (m, 2H), 0.00 (s, 9H) ppm.
Preparation 27

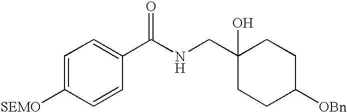

### N-{[4-(Benzyloxy)-1-hydroxycyclohexyl]methyl}-4-{[2-(trimethylsilyl)ethoxy]methoxy}benzamide

A mixture of 4-{[2-(trimethylsilyl)ethoxy]methoxy}benzoic acid (4.0 g, 15 mmol), 1-(aminomethyl)-4-(benzyloxy)cyclohexanol hydrochloride (4.1 g, 15 mmol), Et₃N (4.2 mL, 30 mmol), EDCI (3.5 g, 18 mmol) and HOBt.H₂O (0.46 g, 3.0 mmol) in DMF (45 mL) was stirred at room temperature for 16 hours. The mixture was diluted with AcOEt. The whole was washed with sat. aq. NaHCO₃ and water, dried over MgSO₄ and evaporated to give the titled compound (7.8 g) as a white solid.

¹H NMR (CDCl₃) δ: 7.75 (d, J=8.6 Hz, 2H), 7.38-7.22 (m, 5H), 7.07 (d, J=8.6 Hz, 2H), 6.57-6.45 (m, 1H), 5.26 (s, 2H), 4.58-4.50 (m, 2H), 3.82-3.34 (m, 5H), 2.00-1.30 (m, 8H), 1.00-0.91 (m, 2H), 0.00 (s, 9H) ppm.
Preparation 28

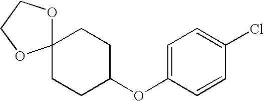

### 8-(4-Chlorophenoxy)-1,4-dioxaspiro[4.5]decane

DIAD (12 mL, 60 mmol) was added dropwise to a mixture of 1,4-dioxaspiro[4.5]decan-8-ol (6.3 g, 40 mmol) (J. Chem. Soc., Perkin Trans. 1, 2002, 2251-2255.), 4-chlorophenol (7.7 g, 60 mmol) and triphenylphosphine (16 g, 60 mmol) in THF (200 mL) at 0° C. and the mixture was stirred at room temperature for 16 hours. After evaporation, the residue was treated with 2N aq. NaOH and the whole was extracted with CH₂Cl₂. The extract was dried over MgSO₄ and evaporated. The residue was purified by silica gel column chlomatography (hexane:AcOEt=20:1 to 5:1) the titled compound (6.8 g).

¹H NMR (CDCl₃) δ: 7.22 (d, J=9.2 Hz, 2H), 6.84 (d, J=9.0 Hz, 2H), 4.40-4.32 (m, 1H), 3.98-3.94 (m, 4H), 1.96-1.84 (m, 6H), 1.68-1.55 (m, 2H) ppm.

Preparation 29

### 4-(4-Chlorophenoxy)cyclohexanone

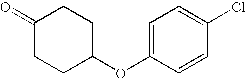

A mixture of 8-(4-chlorophenoxy)-1,4-dioxaspiro[4.5]decane (6.8 g, 25 mmol) and 2N aq. HCl (50 mL) in acetone (80 mL) was refluxed for 3 hours. The mixture was diluted with AcOEt. The whole was washed with sat. aq. NaCl and sat. aq. NaHCO₃, dried over MgSO₄ and evaporated to give 4-(4-chlorophenoxy)cyclohexanone (5.6 g).

¹H NMR (CDCl₃) δ: 7.26 (d, J=9.0 Hz, 2H), 6.90 (d, J=9.0 Hz, 2H), 4.70-4.62 (m, 1H), 2.74-2.60 (m, 2H), 2.39-2.00 (m, 6H) ppm.

Preparation 30

### 1-(Aminomethyl)-4-(4-chlorophenoxy)cyclohexanol

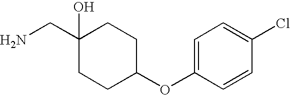

4-(4-Chlorophenoxy)cyclohexanone (5.6 g) was added to a mixture of ZnI₂ (80 mg, 0.25 mmol) and TMSCN (2.8 g, 28 mmol) in benzene (10 mL) at 0° C. and the mixture was stirred at room temperature for 30 min. The mixture was added dropwise to a suspension of LiAlH₄ (2.3 g, 60 mmol) in ether (80 mL) at 0° C. and the mixture was stirred at room temperature for 2 hours. The mixture was quenched with Na₂SO₄10H₂O (excess) and the white suspension was filtered. After the filtrate was evapotated to give the titled compound (6.8 g) as cis-trans mixture.

¹H NMR (CDCl₃) δ: 7.26-7.18 (m, 2H), 6.87-6.80 (m, 2H), 4.55-4.10 (m, 1H), 2.66-2.62 (m, 2H), 2.16-1.20 (m, 8H) ppm. (OH and NH2 were not observed)
Preparation 31

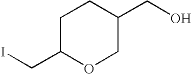

### 2-[(Iodomethyl)tetrahydro-2H-pyran-5-yl]methanol

To a suspension of I₂ (16 g, 63.5 mmol) and NaHCO₃ (5.3 g, 64 mmol) in ether (70 mL) and H₂O (33 mL) was added a solution of 2-(hydroxymethyl)-5-hexene-1-ol (5.5 g, 42 mmol) in ether (40 mL) at 0° C. The mixture was stirred at room temperture for 8 hours. Then the reaction was quenched by addition of sat. aq. Na₂S₂O₃ at 0° C. The aqueous layer was extracted with ether (50 mL×2) and the combined organic layers were washed with brine (50 mL), dried over Na₂SO₄, filtered and concentrated in vacuo. The crude product was purified by silica gel column chromatography (hexane:AcOEt=1.2:1) to give the titled compound (8.2 g, 75%) as yellow oil.

¹H NMR (300 MHz, CDCl₃, cis/trans mixture) δ: 4.18-3.15 (m, 7H), 1.92-1.29 (m, 5H) ppm. (OH was not observed.)

¹³C NMR (75 MHz, CDCl₃, trans-major isomer) δ: 76.9, 71.2, 64.5, 38.3, 31.0, 26.2, 9.5 ppm.

¹³C NMR (75 MHz, CDCl₃, cis-minor isomer) δ: 76.8, 68.7, 62.9, 35.5, 27.5, 24.0, 10.0 ppm.

MS (ESI): 257.0 (M+H)⁺
Preparation 32

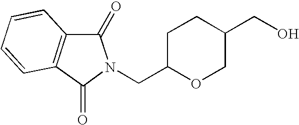

### N-{[5-(Hydroxymethyl)tetrahydro-2H-pyran-2-yl]methyl}-phthalimide

To a solution of 2-[(iodomethyl)tetrahydro-5-2H-pyran-5-yl]methanol (1.8 g, 6.9 mmol) in DMF (45 mL) was added potassium phthalimide (1.8 g, 9.7 mmol) at rt and the mixture was stirred at 90° C. After 5 hours the mixture was cooled to rt and to this mixture was added H₂O (50 mL). The whole was extracted with AcOEt (100 mL×2). The organic layers were washed with H₂O (50 mL×2), brine (50 mL), dried over Na₂SO₄, filtered and concentrated in vacuo. The crude product was purified by silica gel column chromatography (hexane:AcOEt=1:1.2) to give the titled compound (1.3 g, 68%) as a white solid

¹³C NMR (75 MHz, CDCl₃) δ: 168.3 (major/minor), 133.9 (major or minor), 133.8 (major or minor), 131.9 (major/minor), 123.2 (major/minor), 74.9 (major), 74.6 (minor), 70.9 (major), 67.9 (minor), 64.5 (major), 62.5 (minor), 42.5 (major), 42.1 (minor), 38.3 (major), 35.7 (minor), 28.8 (major), 26.0 (major), 25.2 (minor), 23.6 (minor) ppm. MS (ESI): 276.1 (M+H)⁺
Preparation 33

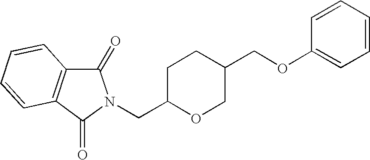

### N-{[5-(Phenoxymethyl)tetrahydro-2H-pyran-2-yl]methyl}-phthalimnide

To a mixture of N-{[5-(hydroxymethyl)tetrahydro-2H-pyran2-yl]methyl}-phthalide (1.2 g, 4.2 mmol), phenol (0.47 g, 5.0 mmol) and PPh₃ in THF (20 mL) was added DEAD (40% in toluene, 2.7 g, 6.3 mmol) at 0° C. and the mixture was stirred at room temperture for 15 hours. Then the reaction mixture was quenched by addition of H₂O (50 mL) and diluted with AcOEt (50 mL). The aqueous layer was extracted with AcOEt (50 mL) and the combined organic layers were washed with brine (50 mL), dried over Na₂SO₄, filtered and concentrated in vacuo. The crude product was purified by silica gel column chromatography (hexane:AcOEt=8:1˜4:1) to give the titled compound (0.67 g, 45%).

¹H NMR (300 MHz, CDCl₃, cis/trans mixture) δ: 7.88-7.70 (m, 4H), 7.31-7.23 (m, 2H), 6.96-6.82 (m, 3H), 4.16-3.57 (m, 6H), 3.22-3.15 (m, 1H), 2.15-1.73 (m, 2H), 1.52-1.26 (m, 3H) ppm.
Preparation 34

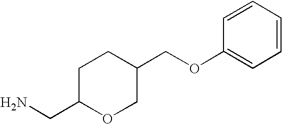

### N-{[5-(Phenoxymethyl)tetrahydro-2H-pyran-2-yl]methyl}amine

To a suspension of N-{[5-(phemoxymethyl)tetrahydro-2H-pyran-2-yl]methyl}-phthalimide (0.67 g, 1.90 mmol) in EtOH (10 mL) was added hydrazine hydrate (0.14 g, 2.9 mmol) and the mixture was refluxed for 3 hours. After evaporation 10% aq. NaOH (50 mL) was added and the mixture was stirred for 30 min. Then the aqueous layer was extracted with CHCl₃ (30 mL×3). The combined organic layers were dried over Na₂SO₄, filtered and concentrated in vacuo to give the titled compound (0.44 g, crude).

¹H NMR (300 MHz, CDCl₃, cis/trans mixture) δ: 7.31-7.24 (m, 2H), 6.96-6.86 (m, 3H), 4.23-3.99 (m, 2H), 3.84-3.63 (m, 2H), 3.31-3.21 (m, 1H), 2.74-2.64 (m, 2H), 2.17-1.21 (m, 5H) ppm. (NH₂ was not observed.) MS (ESI): 222.1 (M+H)⁺
Preparation 35

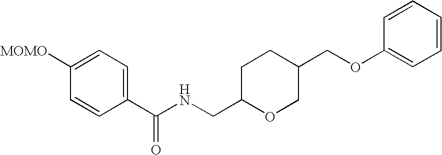

### 4-(Methoxymethoxy)-N-{[5-phenoxymethyl]tetrahydro-2H-pyran-2-yl}methyl}benzamide

This compound was prepared with {[5-(phenoxymethyl)tetrahydro-2H-pyran-2-yl]methyl}amine by a procedure as a white solid similar to that in Preparation 9.

¹H NMR (300 MHz, CDCl₃, cis/trans mixture) δ: 7.77-7.72 (m, 2H), 7.31-7.25 (m, 2H), 7.08-6.86 (m, 5H), 5.22 (s, 2H), 4.22-3.98 (m, 3H), 3.85-3.63 (m, 3H), 3.48 (s, 3H), 3.43-3.18 (m, 2H), 2.17-1.36 (m, 5H) ppm. MS (ESI): 386.17 (M+H)⁺
Preparation 36

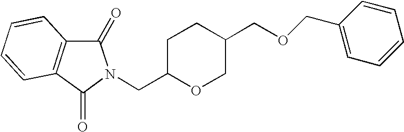

### N-{[5-(Benzyloxymethyl)tetrahydro-2H-pyran-2-yl]methyl}-phthalinmide

To a solution of N-{[5-(hydroxymethyl)tetrahydro-2H-pyran2-yl]methyl}-phthalimide (1.3 g, 4.7 mmol) in CH₂Cl₂ (20 mL) were added Ag₂O (2.2 g, 9.4 mmol) and BnBr (0.84 mL, 7.1 mmol) at rt. After 50 hours, the mixture was filtered through a pad of celite and the filtrate was concentrated in vacuo. The crude product was purified by silica gel column chromatography (hexane-AcOEt 5:1) to give the titled compound (1.6 g, 92%) as a white solid.

¹³C NMR (75 MHz, DMSO) δ: 168.4 (major), 168.3 (minor), 138.4 (minor), 138.3 (major), 133.8 (major/minor). 132.0 (major/minor), 128.3 (major/minor), 127.5 (minor), 127.4 (major), 127.3 (major/minor), 123.2 (major/minor), 74.9 (major), 74.5 (minor), 73.1 (minor), 72.8 (major), 71.9 (major), 71.2 (major), 70.2 (minor), 68.3 (minor), 42.6 (major), 42.1 (minor), 36.0 (major), 33.9 (minor), 28.9 (major), 26.4 (major), 25.2 (minor), 23.7 (minor) ppm.

Preparation 37

### {[5-(Benzyloxymethyl)tetrahydro-2H-pyran-2-yl]methyl}amine

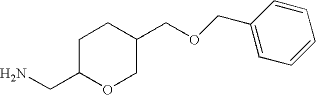

This compound was prepared with N-{[5-(benzyloxymethyl)tetrahydro-2H-pyran-2-yl]methyl}-phthalimide by a procedure similar to that in Preparation 34.

¹H NMR (300 MHz, CDCl₃, cis/trans mixture) δ: 7.37-7.28 (m, 5H), 4.60-4.43 (m, 2H), 4.14-3.97 (m, 1H), 3.74-3.50 (m, 1H), 3.33-3.14 (m, 3H), 2.73-2.66 (m, 2H), 1.99-1.17 (m, 5H) ppm. (NH2 was not observed.) MS (ESI): 236.1 (M+H)⁺.

Preparation 38

### 4-(Benzyloxymethoxy)-N-{[5-phenoxymethyl]tetrahydro-2H-pyran-2-yl}methyl}benzamide

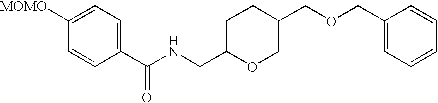

This compound was prepared with {[5-(benzyloxymethyl)tetrahydro-2H-pyran-2-yl]methyl}amine by a procedure similar to that in Preparation 9.

¹H NMR (300 MHz, CDCl₃, cis/trans mixture) δ: 7.76-7.71 (m, 2H), 7.28-7.37 (m, 5H), 7.04-7.09 (m, 2H), 4.60-4.43 (m, 2H), 3.83-3.75 (m, 1H), 3.48 (s, 3H), 3.31-3.14 (m, 3H), 1.97-1.31 (m, 5H) ppm. MS (ESI): 400.2 (M+H)⁺.

Preparation 39

### 2-(Benzyloxymethyl)-hex-5-en-1-ol

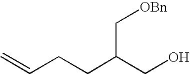

To a solution of 2-(hydroxymethyl)-hex-5-en1-ol in CH₂Cl₂ were added BnBr (3.8 mL, 44 mmol) and Ag₂O (10 g, 44 mmol) at rt for 10 hours. Then the mixture was filtered through a pad of celite and the filtrate was ceoncentrated in vacuo. The crude product was purified by silica gel column chromatography (hexane-AcOEt 7:1) to give the titled compound (5.0 g, 78%) as colorless oil.

¹H NMR (300 MHz, CDCl₃) δ: 7.38-8.29 (m, 5H), 5.86-5.71 (m, 1H), 5.04-4.94-7.24 (m, 2H), 4.55 (d, J=12.2 Hz, 1H), 4.49 (d, J=12.2 Hz, 1H), 3.45-3.77 (m, 4H), 2.12-2.07 (m, 2H), 1.93-1.85 (m, 1H), 1.50-1.32 (m, 2H) ppm.

Preparation 40

### 2-Benzyloxymethyl-4-oxiran-2-ylbutan-1-ol

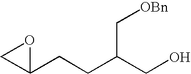

To a solution of 2-(benzyloxymethyl)-hex-5-en-1-ol in CH₂Cl₂ were added NaHCO₃ and meta-chlor-prbenzoic acid (mCPBA) at 0° C. After 7 hours, the reaction was quenched by addition of sat.a. NaHCO₃ (50 mL). The aqueous layer was extracted with CH₂Cl₂ and the combined organic layers were washed with brine (50 mL), dried over MgSO₄, filtered and concentrated in vacuo. The crude product was purified by silica gel column chromatography (hexane-AcOEt 3:1˜1:1) to give the titled compound (2.6 g, 60%) as pale yellow oil

¹H NMR (300 MHz, CDCl₃) δ: 7.28-7.38 (m, 5H), 4.54 (d, J=12.0 Hz, 1H), 4.50 (d, J=12.0 Hz, 1H), 3.76-3.46 (m, 4 H), 2.92-2.87 (m, 1H), 2.75 (dt, J=0.7, 4.9 Hz, 1H), 2.47 (ddd, J=0.9, 2.7, 4.9 Hz, 1H), 1.70-1.94 (m, 1H), 1.69-1.35 (m, 4H) ppm. MS (ESI): 237.1 (M+H)⁺.

Preparation 41

### {5-[(Benzyloxy)methyl]tetrahydro-2H-pyran-2-yl}methanol

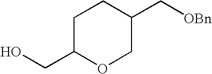

To a solution of 2-benzyloxymethyl-4-oxiran-2-ylbutan-1-ol in CH₂Cl₂ was added BF₃.OEt₂ at −78° C. and the mixture was warmed to 0° C. After 4 hours, the reacton was quenched by addition of H₂O (50 mL). The aqueous layer was extracted with CH₂Cl₂ (50 mL) and the combined organic layers were washed with brinie (50 mL), dried over MgSO₄, filtered and concentrated in vacuo. The crude product was purified by silica gel column chromatography (hexane-AcOEt 3:1) to give the titled product (1.5 g including unknown impurity) ¹H NMR (300 MHz, CDCl₃, cis/trans mixture) δ: 7.37-7.09 (m, 5H), 4.60-4.42 (m, 4 H) 3.89-4.16 (m, 2H), 3.70-3.17 (m, 5H), 2.00-1.18 (m, 5H) ppm. (OH was not observed.)

Preparation 42

### 5-[(Benzyloxy)methyl]-2-(phenoxymethyl)tetrahydro-2H-pyran

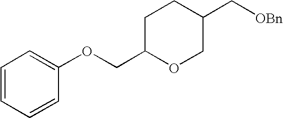

To a mixture of {5-[(benzyloxy)methyl]tetrahydro-2H-pyran-2-yl}methanol (1.5 g, 6.2 mmol), phenol (0.7 g, 7.4 mmol) and PPh₃ (2.0 g, 7.4 mmol) in THF (25 mL) was added DEAD (diethylazodicarboxylate) (40% in toluene, 4.0 g) at 0° C. and the mixture was stirred at rt for 14 hours. Then the reaction was quenched by addition of H₂O (50 mL) and extracted with AcOEt (50 mL×2). The combined organic layers were washed with brine (50 mL), dried over MgSO₄, filtered and concentrated in vacuo. The crude product was purified by silica gel column chromatography (hexane:AcOEt=12:1) to give the titled product (0.30 g, 15%)

¹H NMR (300 MHz, CDCl₃, cis/trans mixture) δ: 7.37-7.24 (m, 7H), 6.96-6.90 (m, 3H), 4.63-4.42 (m, 2H), 4.20-3.54 (m, 5H), 3.35-3.22 (m, 2H), 2.02-1.72 (m, 3H), 1.55-1.23 (m, 2H) ppm. MS (ESI): 313.2 (M+H)⁺.

Preparation 43

### [6-(Phenoxymethyl)tetrahydro-2H-pyran-3-yl}methanol

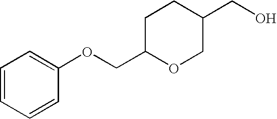

To a mixture of 5-[(benzyloxy)methyl]-2-(phenoxymethyl)tetrahydro-2H-pyran (0.3 g, 0.95 mmol) and Pd(OH)₂/C (20 wt.% Pd on carbon, 0.15 g) in THF (5 mL) was added 10˜20% HCl-MeOH (0.5 mL). The mixture was stirred under H₂ atmosphere (4 atm) at rt for 5 hours. Then the mixture was filtered through a pad of celite and the filtrate was concentrated in vacuo. The crude product was purified by silica gel column chromatography (hexane:AcOEt=2:1) to give the titled compound (0.16 g, 77%)

¹H NMR (300 MHz, CDCl₃, cis-trans mixture) δ: 7.31-7.25 (m, 2H), 6.97-6.90 (m, 3H), 4.21-3.23 (m, 7H), 1.98-1.27 (m, 5H) ppm. (OH was not observed) MS (ESI): 223.0 (M+H)⁺.

Preparation 44

### [6-(Phenoxymethyl)tetrahydro-2H-pyran-3-yl]methyl methanesulfonate

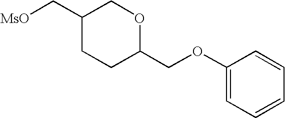

Methanesulfonyl chloride (68 μL, 0.88 mmol) was added to a mixture of [6-(phenoxymethyl)tetrahydro-2H-pyran-3-yl}methanol (0.16 g, 0.73 mmol) and triethylamine (0.20 mL, 1.5 mmol) in CH₂Cl₂ at 0° C. and the mixture was stirred at 0° C. for 2 hours. The mixture was treated with sat. aq. NaHCO₃ and was extracted with CH₂Cl₂ The extract was dried over MgSO₄ and evaporated to give [6-(phenoxymethyl)tetrahydro-2H-pyran-3-yl]methyl methanesulfonate (0.20 g).

¹H NMR (CDCl₃) δ: 7.32-7.24 (m, 2H), 7.00-6.88 (m, 3H), 4.50-3.24 (m, 7H), 3.04-3.01 (m, 3H), 2.20-1.30 (m, 5H) ppm.

Preparation 45

### 5-(Azidomethyl)-2-(phenoxymethyl)tetrahydro-2H-pyran

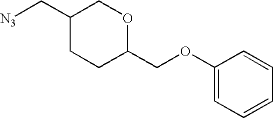

[6-(Phenoxymethyl)tetrahydro-2H-pyran-3-yl]methyl methanesulfonate (0.20 g) was dissolved with DMF (3.5 mL). To the solution, NaN₃ (0.22 g, 3.4 mmol) was added and the mixture was stirred 100° C. for 3 hours. After cooling to room temperature, the mixture was diluted with ether and washed with water. The organic layer was dried over MgSO₄ and evaporated to give 5-(azidomethyl)-2-(phenoxymethyl)tetrahydro-2H-pyran (0.16 g).

¹H NMR (CDCl₃) δ: 7.32-7.24 (m, 2H), 7.00-6.88 (m, 3H), 4.20-3.08 (m, 7H), 2.08-1.20 (m, 5H) ppm.

Preparation 46

### {[6-(Phenoxymethyl)tetrahydro-2H-pyran-3-yl]methyl}amine

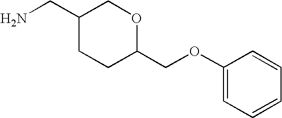

A solution of 5-(azidomethyl)-2-(phenoxymethyl)tetrahydro-2H-pyran (0.16 g) in THF (1.0 mL) was added to a suspension of LiAlH₄ (0.64 mmol) in THF (2.0 mL) at 0° C. and the mixture was stirred at room temperature for 30 min. The mixture was quenched with Na₂SO₄.10H₂O (excess) and KF (excess). After stirring for 4 hours, the suspension was filtered and evaporated to give {[6-(phenoxymethyl)tetrahydro-2H-pyran-3-yl]methyl}amine (0.13 g).

¹H NMR (CDCl₃) δ: 7.32-7.24 (m, 2H), 7.00-6.88 (m, 3H), 4.20-2.53 (m, 7H), 2.08-1.10 (m, 5H) ppm.
Preparation 47

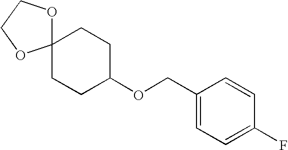

### 8-[(4-Fluorobenzyl)oxy]-1,4-dioxaspiro[4.5]decane

NaH (880 mg, 22 mmol; 60%) was washed with n-hexane (5 ml×2) and the powder was dried in vacuo. To the flask was added THF (5 ml) and cooled to 0° C. To the suspension was added a solution of 1,4-dioxaspiro[4.5]decan-8-ol (3.2 g, 20 mmol) in THF (15 ml) and the reaction mixture was stirred at room temperature for 30 min. To the mixture was added a solution of 1-(bromomethyl)-4-fluorobenzene (4.5 g, 24 mmol) in THF (5 ml) at 0° C., stirred at room temperature for 17 hr. To the reaction mixture was added NaH (400 mg, 10 mmol; 60%) and the mixture was refluxed for 6 hr. Sat. aq. NaHCO₃ (20 ml) was poured into the reaction mixture and the whole was extracted with ethyl acetate (50 ml×3). The combined organic layer was dried over Na₂SO₄, concentrated in vacuo. The residue was purified by column chromatography on silica gel (n-hexane: ethyl acetate=10:1 as eluent) to afford the titled compound as yellow oil (5.0 g, 94%).

¹H NMR (CDCl₃) δ: 7.33-7.27 (m, 2H), 7.04-6.97 (m, 2H), 4.48 (s, 2H), 3.98-3.89 (m, 4H), 3.54-3.48 (m, 1H), 1.89-1.71 (m, 6H), 1.60-1.50 (m, 2H) ppm.
Preparation 48

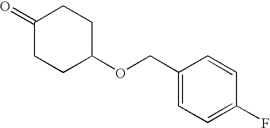

### 4-[(4-Fluorobenzyl)oxy]cyclohexanone

This compound was prepared with 8-[(4-fluorobenzyl)oxy]-1,4-dioxaspiro[4.5]decane by a procedure similar to that in Preparation 20 as yellow oil.

¹H NMR (CDCl₃) δ: 7.36-7.31 (m, 2H), 7.08-7.02 (m, 2H), 4.56 (s, 2H), 3.83-3.81 (m, 1H), 2.67-2.56 (m, 2H), 2.33-1.98 (m, 6H) ppm.
Preparation 49

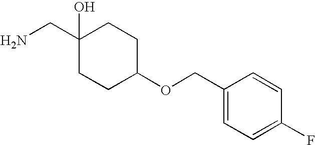

### 1-(Aminomethyl)-4-[(4-fluorobenzyl)oxy]cyclohexanol

This compound was prepared with 4-[(4-fluorobenzyl)oxy]cyclohexanone by a procedure similar to that in Preparation 30 as brown oil.

¹H NMR (CDCl₃, cis/trans mixture) δ: 7.34-7.16 (m, 2H), 7.05-6.99 (m, 2H), 4.53 (s, 1.4H), 4.47 (s, 0.6H), 3.63-3.61 (m, 0.3H), 3.38-3.29 (m, 0.7H), 2.64 (s, 0.6H), 2.58 (s, 1.4H), 1.90-1.19 (m, 8H) ppm. (OH and NH₂ were not observed.) MS (ESI): 254.10 (M+H)⁺
Preparation 50

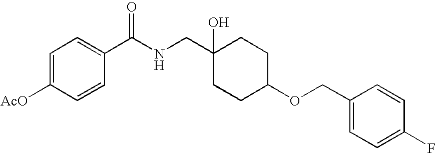

### 4-{[({4-[(4-Fluorobenzyl)oxy]-1-hydroxycyclohexyl}methyl)amino]carbonyl}phenyl acetate

This compound was prepared with 4-acetoxybenzoic acid and 1-(aminomethyl)-4-[(4-fluorobenzyl)oxy]cyclohexanol by a procedure similar to that in Preparation 9 as a white solid.

¹H NMR (CDCl₃, cis/trans mixture) δ: 7.83-7.80 (m, 2H), 7.32-7.29 (m, 2H), 7.18-7.15 (m, 2H), 7.08-6.99 (m, 2H), 6.61 (br, 1H), 4.51-4.46 (m, 2H), 3.52-3.46 (m, 2H), 3.41-3.39 (m, 1H), 2.35-2.28 (m, 3H), 1.85-1.68 (m, 6H), 1.49-1.41 (m, 2H) ppm. (OH was not observed.) MS (ESI): 416.03 (M+H)⁺, 414.03 (M−H)⁻

Preparation 51

### 8-[(2-Fluorobenzyl)oxy]-1,4-dioxaspiro[4.5]decane

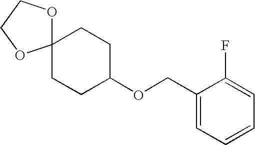

This compound was prepared with 2-fluorobenzyl bromide by a procedure similar to that in Preparation 47.

¹H NMR (CDCl₃) δ: 7.49-7.43 (m, 1H), 7.30-7.20 (m, 1H), 7.16-6.97 (m, 2H), 4.59 (s, 2H), 3.97-3.91 (m, 4H), 3.59-3.50 (m, 1H), 1.94-1.73 (m, 6H), 1.63-1.48 (m, 2H) ppm.
Preparation 52

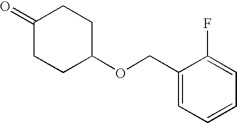

### 4-[(2-Fluorobenzyl)oxy]cyclohexanone

This compound was prepared with 8-[(2-fluorobenzyl)oxy]-1,4-dioxaspiro[4.5]decane by a procedure similar to that in Preparation 20 as colorless oil.

¹H NMR (CDCl₃) δ: 7.49-7.43 (m, 1H), 7.33-7.25 (m, 1H), 7.18-7.03 (m, 2H), 4.66 (s, 2H), 3.88-3.83 (m, 1H), 2.68-2.58 (m, 2H), 2.32-1.92 (m, 6H) ppm.
Preparation 53

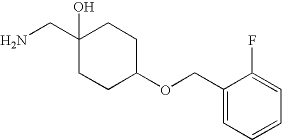

### 1-(Aminomethyl)-4-[(2-fluorobenzyl)oxy]cyclohexanol

This compound was prepared with 4-[(2-fluorobenzyl)oxy]cyclohexanone by a procedure similar to that in Preparation 30 as yellow oil.

¹H NMR (CDCl₃, cis/trans mixture) δ: 7.49-7.42 (m, 1H), 7.28-6.99 (m, 3H), 4.63 (s, 1.4H), 4.56 (s, 0.6H), 3.66-3.65 (m, 0.4H), 3.41-3.32 (m, 0.6H), 2.64-2.63 (m, 0.6H), 2.58-2.57 (m, 1.4H), 1.92-1.20 (m, 8H) ppm. [OH and NH₂ proton were not observed.] MS (ESI): 254.07 (M+H)⁺
Preparation 54

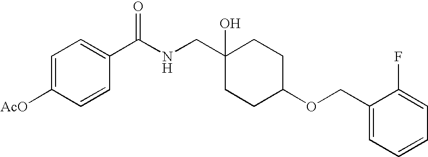

### 4-{[({4-[(2-Fluorobenzyl)oxy]-1-hydroxycyclohexyl}methyl)armino]carbonyl}phenyl acetate

This compound was prepared with 4-acetoxybenzoic acid and 1-(aminomethyl)-4-[(2-fluorobenzyl)oxy]cyclohexanol by a procedure similar to that in Preparation 9 as a white solid.

¹H NMR (CDCl₃, cis/trans mixture) δ: 7.83-7.80 (m, 2H), 7.46-7.41 (m, 1H), 7.29-6.99 (m, 6H), 6.55 (br, 1H), 4.62 (s, 1.5H), 4.56 (s, 0.5H), 3.52-3.44 (m, 3H), 2.32 (s, 3H), 1.88-1.66 (m, 6H), 1.51-1.43 (m, 2H) ppm. MS (ESI): 416.06 (M+H)⁺
Preparation 55

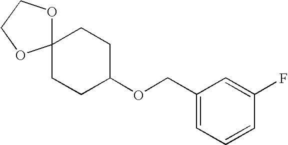

### 8-[(3-Fluorobenzyl)oxy]-1,4-dioxaspiro[4.5]decane

This compound was prepared with 3-fluorobenzyl bromide by a procedure similar to that in Preparation 47 as yellow oil.

¹H NMR (CDCl₃) δ: 7.33-7.25 (m, 1H), 7.11-7.05 (m, 2H), 6.98-6.91 (m, 1H), 4.52 (s, 2H), 3.99-3.91 (m, 4H), 3.55-3.48 (m, 1H), 1.90-1.75 (m, 6H), 1.64-1.50 (m, 2H) ppm.
Preparation 56

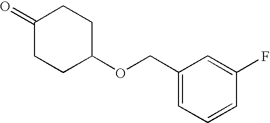

### 4-[(3-Fluorobenzyl)oxy]cyclohexanone

This compound was prepared with 8-[(3-fluorobenzyl)oxy]-1,4-dioxaspiro[4.5]decane by a procedure similar to that in Preparation 20 as yellow oil.

¹H NMR (CDCl₃) δ: 7.36-7.26 (m, 1H), 7.14-7.08 (m, 2H), 7.01-6.95 (m, 1H), 4.60 (s, 2H), 3.83-3.81 (m, 1H), 2.69-2.57 (m, 2H), 2.32-2.13 (m, 4H), 2.05-1.98 (m, 2H) ppm.
Preparation 57

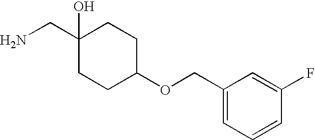

### 1-(Aminomethyl)-4-[(3-fluorobenzyl)oxy]cyclohexanol

This compound was prepared with 4-[(3-fluorobenzyl)oxy]cyclohexanone by a procedure similar to that in Preparation 30 as yellow oil.

¹H NMR (CDCl₃, cis/trans mixture) δ: 7.32-7.24 (m, 1H), 7.11-7.06 (m, 2H), 6.97-6.92 (m, 1H), 4.55-4.49 (m, 2H), 3.76-3.31 (m, 1H), 2.64-2.56 (m, 2H), 2.02-1.13 (m, 8H) ppm. (OH and NH₂ were not observed.) MS (ESI): 254.11 (M+H)⁺
Preparation 58

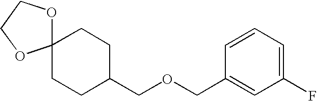

### 8-{[(3-Fluorobenzyl)oxy]methyl}-1,4-dioxaspiro[4.51]decane

This compound was prepared with 3-fluorobenzyl bromide and 1,4-dioxaspiro[4.5]dec-8-ylmethanol by a procedure similar to that in Preparation 47 as colorless oil.

¹H NMR (CDCl₃) δ: 7.33-7.26 (m, 1H), 7.10-6.93 (m, 3H), 4.49 (s, 2H), 3.98-3.90 (m, 4H), 3.31 (d, J=6.6 Hz, 2H), 1.85-1.50 (m, 7H), 1.35-1.21 (m, 2H) ppm.
Preparation 59

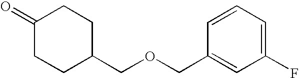

### 4-{[(3-Fluorobenzyl)oxy]methyl}cyclohexanone

This compound was prepared with 8-{[(3-fluorobenzyl)oxy]methyl}-1,4-dioxaspiro[4.5]decane by a procedure similar to that in Preparation 20 as yellow oil.

¹H NMR (CDCl₃) δ: 7.35-7.27 (m, 1H), 7.10-6.95 (m, 3H), 4.52 (s, 2H), 3.40 (d, J=6.1 Hz, 2H), 2.43-2.28 (m, 4H), 2.17-2.06 (m, 3H), 1.55-1.44 (m, 2) ppm.
Preparation 60

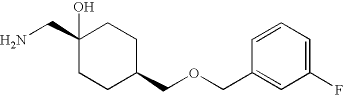

### trans-1-(Aminomethyl)-4-{[(3-fluorobenzyl)oxy]methyl}cyclohexanol

This compound was prepared with 4-{[(3-fluorobenzyl)oxy]methyl}cyclohexanone by a procedure similar to that in Preparation 30 as yellow oil.

¹H NMR (DMSO-d₆) δ: 7.43-7.40 (m, 1H), 7.16-7.07 (m, 3H), 4.46 (s, 2H), 3.28 (d, J=5.9 Hz, 2H), 2.46-2.35 (m, 2H), 1.64-1.61 (m, 4H), 1.27-1.01 (m, 5H) ppm. [NH₂ and OH proton were not observed.] MS (ESI): 268.18 (M+H)⁺
Preparation 61

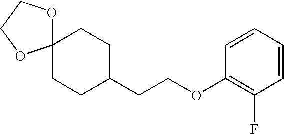

### 8-[2-(2-Fluorophenoxy)ethyl]1,4-dioxaspiro[4.5]decane

This compound was prepared with 2-(1,4-dioxaspiro[4.5]dec-8-yl)ethanol and 2-fluorophenol by a procedure similar to that in Preparation 42 as colorless oil.

¹H NMR (CDCl₃) δ: 7.10-6.84 (m, 4H), 4.09-4.05 (m, 2H), 3.94 (s, 4H), 1.82-1.74 (m, 6H), 1.68-1.51 (m, 3H), 1.38-1.24 (m, 2H) ppm.
Preparation 62

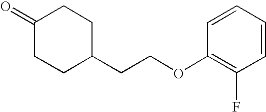

### 4-[2-(2-Fluorophenoxy)ethyl]cyclohexanone

This compound was prepared with 8-[2-(2-fluorophenoxy)ethyl]1,4-dioxaspiro[4.5]decane by a procedure similar to that in Preparation 20 as a white solid.

¹H NMR (CDCl₃) δ: 7.12-6.91 (m, 4H), 4.16-4.05 (m, 2H), 2.41-2.36 (m, 4H), 2.18-2.05 (m, 3H), 1.88-1.76 (m, 2H), 1.56-1.42 (m, 2H) ppm.
Preparation 63

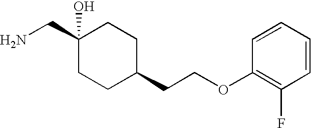

### trans-1-(Aminomethyl)-4-[2-(2-fluorophenyl)ethyl]cyclohexanol

This compound was prepared with 4-[2-(2-fluorophenyl)ethyl]cyclohexanone by a procedure similar to that in Preparation 30 as a white solid.

¹H NMR (DMSO-d₆) δ: 7.28-6.86 (m, 4H), 4.15-3.97 (m, 2H), 2.48-2.37 (m, 2H), 1.81-0.94 (m, 11H) ppm. [NH₂ and OH proton were not observed.] MS (ESI): 268.11 (M+H)⁺
Preparation 64

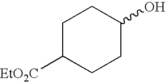

### Ethyl 4-hydroxycyclohexanecarboxylate

To a solution of ethyl 4-oxocyclohexanecarboxylate (13.5 g, 79 mmol) in MeOH (150 mL) at 0° C. was added NaBH₄ (5.3 g, 140 mmol) and the mixture was stirred at rt for 3 h. Then the reaction was quenched by addition of H₂O (50 mL) and extracted with AcOEt (150 mL×1, 50 mL×2). The combined organic layer was washed with H₂O (50 mL), dried over Na₂SO₄, filtered and concentrated in vacuo. The residue was purified by silica gel column chromatography (hexane-AcOEt 1.5:1-1:1) to give the titled compound (12 g, 88%, cis/trans=3:7) as clear oil.

¹H NMR (300MHz, CDCl₃, cis/trans mixture) δ: 4.17-4.08 (m, 2H), 3.90 (bs, 0.3H), 3.68-3.57 (m, 0.7H), 2.42-1.28 (m, 9H), 1.27-1.22 (m, 3H) ppm. (OH was not observed.)
Preparation 65

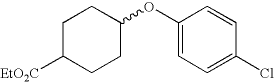

### Ethyl 4-(4-chlorophenoxy)cycohexanecarboxylate

To a solution of ethyl 4-hydroxycyclohexanecarboxylate (3.1 g, 18 mmol) in toluene (50 mL) were added PPh₃ (5.2 g, 20 mmol) and p-chlorophenol (2.6 g, 20 mmol). To the mixture was added DEAD (40% in toluene, 9.4 g, 21 mmol) at 0° C. and the mixture was stirred at rt for 7 h. The reaction was quenched by addition H₂O (100 mL) and diluted with AcOEt (100 mL). The aqueous layer was extracted with AcOEt (50 mL) and the combined organic layer was washed with brine (50 mL), dried over MgSO₄, filtered and concentrated in vacuo. The residue was purified by silica gel column chromatography (hexane:AcOEt=12:1) to give the titled compound (3.5 g, 68%).

¹H NMR (300MHz, CDCl₃, cis/trans mixture) δ: 7.24-7.19 (m, 2H), 6.86-6.79 (m, 2H) 4.47-4.40 (m, 1H), 4.18-4.09 (m, 2H), 2.44-1.41 (m, 9H), 1.28-1.24 (m, 3H) ppm.
Preparation 66

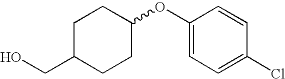

### [4-(4-Chlorophenoxy)cyclohexyl]methanol

To a suspension of lithium aluminum hydride (1.4 g, 37 mmol) in Et₂O (30 mL) was added a solution of ethyl 4-(4-chlorophenoxy)cycohexanecarboxylate (3.5 g, 12 mmol) in Et₂O (30 mL) at 0° C. and the mixture was stirred at rt. After 2 h, the reaction was quenched by addition of H₂O (1.4 mL), 15% NaOH (1.4 mL) and H₂O (4.2 mL). The mixture was diluted with AcOEt (50 mL) and stirred for 1 h. Then the mixture was filtered and concentrated in vacuo to give the titled compound (2.9 g).

¹H NMR (300MHz, CDCl₃, cisitrans mixture) δ: 7.24-7.19 (m, 2H), 6.90-6.79 (m, 2H) 4.49-4.05 (m, 1H), 3.53-3.47 (m, 2H), 2.20-1.02 (m, 9H) ppm. (OH was not observed.)

Preparation 67

### 4-(Azidomethyl)cyclohexyl 4-chlorophenyl ether

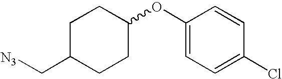

To a solution of [4-(4-chlorophenoxy)cyclohexyl]methanol (2.9 g, crude from above procedure) and Et₃N (3.5 mL, 25 mmol) in CH₂Cl₂ (100 mL) was added MsCl (1.2 mL, 15 mmol) at 0° C. After 1.5 h, the reaction mixture was quenched by addition of sat. aq. NaHCO₃ (50 mL). The aqueous layer was extracted with CH₂Cl₂ (30 mL×2) and the combined organic layer was washed with brine (30 mL), dried over Na₂SO₄, filtered and comcentrated in vacuo. The residue was dissolved in DMF (60 mL) and to this solution was added NaN₃ (1.6 g, 25 mmol) and stirred at 80° C. for 3 h. Then the reaction was quenched by addedtion of sat. aq. NaHCO₃ (30 mL) and extracted with AcOEt (100 mL). The aqueous layer was extracted with AcOEt (50 mL×2) and the combined organic layer was extracted with H₂O (50 mL×2), brine (50 mL), dried over Na₂SO₄, filtered and concentrated in vacuo. The residue was purified by silica gel column chromatography (hexane:AcOEt=1:15) to give the titled compound (3.0 g, 91% over 3 steps).

¹H NMR (300MHz, CDCl₃, cis/trans mixture) δ: 7.29-7.18 (m, 2H), 6.86-6.79 (m, 2H) 4.50-4.04 (m, 1H), 3.19 (d, J=6.42 Hz, 2H), 2.18-1.10 (m, 9H).
Preparation 68

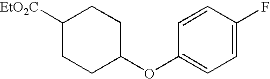

### Ethyl 4-(4-fluorophenoxy)cyclohexanecarboxylate

This compound was prepared with 4-fluorophenol by a procedure similar to that in Preparation 65 as colorless oil.

¹H NMR (CDCl₃, cis/trans mixture) δ: 6.98-6.92 (m, 2H), 6.87-6.83 (m, 2H), 4.38-4.11 (m, 3H), 2.42-2.28 (m, 1H), 2.18-1.90 (m, 4H), 1.76-1.39 (m, 4H), 1.29-1.23 (m, 3H) ppm.
Preparation 69

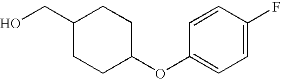

### [4-(4-Fluorophenoxy)cyclohexyl]methanol

This compound was prepared with ethyl 4-(4-fluorophenoxy)cyclohexanecarboxylate by a procedure similar to that in Preparation 66.

¹H NMR (CDCl₃, cis/trans mixture) δ: 6.99-6.81 (m, 4H), 4.45-3.70 (m, 1H), 3.54-3.48 (m, 2H), 2.19-1.37 (m, 9H) ppm. (OH was not observed.)
Preparation 70

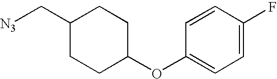

### 1-}[4-(Azidomethyl)cyclohexyl]oxy}-4-fluorobenzene

This compound was prepared with [4-(4-fluorophenoxy)cyclohexyl]methanol by a procedure similar to that in Preparation 67.

¹H NMR (CDCl₃, cis/trans mixture) δ: 6.99-6.81 (m, 4H), 4.45 (br, 1H), 3.21-3.18 (m, 2H), 2.19-1.41 (m, 9H) ppm.
Preparation 71

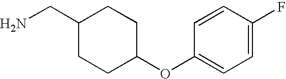

### {[4-(4-Fluorophenoxy)cyclohexyl]methyl}amine

To a solution of 1-{[4-(azidomethyl)cyclohexyl]oxy}-4-fluorobenzene (5.6 g, 21 mmol) in MeOH (50 ml) was added 10% Pd—C (0.5 mg) and the whole mixture was stirred at room temperature for 5 hr under hydrogen atmosphere. The reaction mixture was filtered through a pad of celite and the filtrate was concentrated in vacuo to afford the titled compound as colorless oil (4.4 g).

¹H NMR (DMSO-d₆, cis/trans mixture) δ: 8.05 (br, 2H), 7.14-7.06 (m, 2H), 6.98-6.93 (m, 2H), 4.52 (br, 0.8H), 4.19 (br, 0.2H), 2.81 (d, J=6.6 Hz, 0.4H), 2.69 (d, J=6.8 Hz, 1.6H), 2.05-1.08 (m, 9H) ppm. MS (ESI): 224.11 (M+H)⁺
Preparation 72

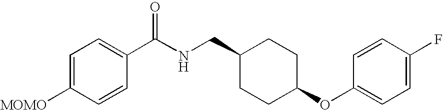

### N-{[cis-4-(4-Fluorophenoxy)cyclohexyl]methyl}-4-(methoxymethoxy)benzamide

This compound was prepared with {[4-(4-fluorophenoxy)cyclohexyl]methyl}amine by a procedure similar to that in Preparation 9 as a white solid.

¹H NMR (DMSO-d₆) δ: 8.34 (m, 1H), 7.83-7.80 (m, 2H), 7.12-7.04 (m, 4H), 6.98-6.93 (m, 2H), 5.24 (s, 2H), 4.50 (br, 1H), 3.38 (s, 3H), 3.18-3.13 (m, 2H), 1.88-1.31 (m, 9H) ppm.
Preparation 73

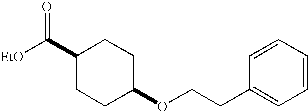

### Ethyl cis-4-(2-phenylethoxy)cyclohexanecarboxylate

Iodotrimethylsilane (0.36 mL, 2.5 mmol) was added to a mixture of ethyl 4-oxocyclohexanecarboxylate (8.5 g, 50 mmol) and dimethyl(2-phenylethoxy)silane (9.9 g, 55 mmol) (Synlett 2002, 313-315.) in CH₂Cl₂ (50 mL) at 0° C. and the mixture was stirred at room temperature for 16 hours. The mixture was quenched with water and extracted with AcOEt (200 mL). The extract was washed with sat. aq. NaHCO₃ (50 mL), dried over MgSO₄ and concentrated. The residue was purified by silica gel column chromatography (hexane-AcOEt 15:1) to give the titled compound (2.7 g).

¹H NMR (CDCl₃) δ: 7.35-7.15 (m, 5H), 4.13 (q, J=7.1 Hz, 2H), 3.60 (t, J=7.3 Hz, 2H), 3.52-3.40 (m, 1H), 2.87 (t, J=7.3 Hz, 2H), 2.42-2.24 (m, 1H), 1.97-1.40 (m, 8H), 1.25 (t, J=7.1 Hz, 3H) ppm.
Preparation 74

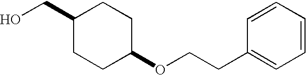

This compound was prepared ethyl cis-4-(2-phenylethoxy)cyclohexanecarboxylate by a procedure similar to that in Preparation 66.

¹H NMR (CDCl₃) δ: 7.40-7.12 (m, 5H), 3.81-3.39 (m, 5H), 2.87 (t, J=7.1 Hz, 2H), 1.95-1.15 (m, 9H) ppm. (OH was not observed.)
Preparation 75

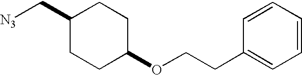

### cis-4-(Azidomethyl)cyclohexyl 2-phenylethyl ether

This compound was prepared [cis-4-(2-phenylethoxy)cyclohexyl]methanol by a procedure similar to that in Preparation 67.

¹H NMR (CDCl₃) δ: 7.37-7.12 (m, 5H), 3.66-3.48 (m, 3H), 3.00 (d, J=6.8 Hz, 2H), 2.87 (t, J=7.1 Hz, 2H), 1.96-1.17 (m, 9H) ppm.
Preparation 76

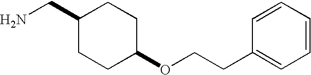

### {[cis-4-(2-Phenylethoxy)cyclohexyl]methyl}amine

This compound was prepared cis-4-(azidomethyl)cyclohexyl 2-phenylethyl ether by a procedure similar to that in Preparation 71.

¹H NMR (CDCl₃) δ: 7.40-7.14 (m, 5H), 3.72-3.46 (m, 3H), 2.87 (t, J=7.3 Hz, 2H), 2.53 (d, J=5.4 Hz, 2H), 2.00-1.15 (m, 9H) ppm. (NH₂ was not observed.)
Preparation 77

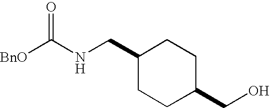

### Benzyl {[cis-4-(hydroxymethyl)cyclohexyl]methyl}carbamate

Benzylchloroformate (15 mL, 0.11 mol) was added dropwise to a mixture of [cis-4-(aminomethyl)cyclohexyl]methanol (14 g, 0.10 mol) and diisopropylethylamine (21 mL, 0.12 mol) in CH₂Cl₂ (200 mL) at 0° C. and the mixture was stirred at room temperature for 2 hours. The mixture was diluted with CH₂Cl₂ (200 mL) and washed with sat. aq. NH₄Cl. The organic layer was dried over MgSO₄ and concentrated in vacuum. The residue was purified by silica gel column chromatography (hexane-AcOEt 1:1 to 1:2) to give the titled compound (14 g).

¹H NMR (CDCl₃) δ: 7.38-7.26 (m, 5H), 5.10 (s, 2H), 4.80-4.70 (m, 1H), 3.58-3.50 (m, 2H), 3.20-3.10 (m, 2H), 1.75-1.20 (m, 10H) ppm. (OH was not observed.)
Preparation 78

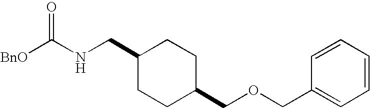

### Benzyl ({cis-4-[(benzyloxy)methyl]cyclohexyl}methyl)carbamate

NaH (88 mg, 2.2 mmol) was added to a solution of benzyl {[cis-4-(hydroxymethyl)cyclohexyl]methyl}carbamate (0.55 g, 2.0 mmol) in THF (4.0 mL) at 0° C. and the mixture was stirred at room temperature for 30 min. Benzylbromide (0.29 mL, 2.4 mmol) was added to the mixture at 0° C. The mixture was sitrred at room temperature for 7 hours. The mixture was quenched with sat. aq. NH₄Cl and extracted with CH₂Cl₂. The extract was dried over MgSO₄ and concentrated in vacuum. The residue was purified by silica gel column chromatography (hexane-AcOEt 8:1) to give the titled compound (0.27 g).

¹H NMR (CDCl₃) δ: 7.40-7.24 (m, 10H), 5.09 (s, 2H), 4.80-4.65 (m, 1H), 4.49 (s, 2H), 3.36 (d, J=7.1 Hz, 2H), 3.20-3.08 (m, 2H), 1.90-1.22 (m, 10H) ppm.
Preparation 79

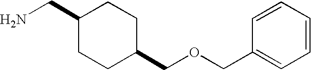

### ({cis-4-[(Benzyloxy)methyl]cyclohexyl}methyl)amine

A mixture of benzyl ({cis-4-[(benzyloxy)methyl]cyclohexyl}methyl)carbamate (0.27 g, 0.73 mmol) and KOH (0.21 g, 3.7 mmol) in EtOH (0.40 mL) was refluxed for 3 hours. The mixture was acidified with 2N aq. HCl (20 mL) and the aqueous layer was washed with AcOEt. The aqueous layer was alkalized with 2N aq. NaOH (21 mL) and extracted with CH₂Cl₂. The extract was dried over MgSO₄ and concentrated to give the titled compound (0.10 g).

¹H NMR (CDCl₃) δ: 7.40-7.25 (m, 5H), 4.50 (s, 2H), 3.37 (d, J=7.1 Hz, 2H), 2.60 (d, J=6.6 Hz, 2H), 1.94-1.80 (m, 1H), 1.64-1.22 (m, 9H) ppm. (NH₂ was not observed.)
Preparation 80

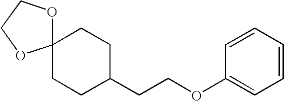

### 8-(2-Phenoxyethyl)-1,4-dioxaspiro[4.5]decane

This compound was prepared with 2-(1,4-dioxaspiro[4.5]dec-8-yl)ethanol by a procedure similar to that in Preparation 42.

¹H NMR (CDCl₃) δ: 7.31-7.24 (m, 2H), 6.96-6.86 (m, 3H), 3.99 (t, J=6.4 Hz, 2H), 3.95 (s, 4H), 1.84-1.22 (m, 11H) ppm.
Preparation 81

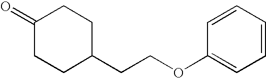

### 4-(2-Phenoxyethyl)cyclohexanone

This compound was prepared with 8-(2-phenoxyethyl)-1,4-dioxaspiro[4.5]decane by a procedure similar to that in Preparation 20.

¹H NMR (CDCl₃) δ: 7.34-7.25 (m, 2H), 7.00-6.88 (m, 3H), 4.05 (t, J=6.3 Hz, 2H), 2.45-1.40 (m, 11H) ppm.
Preparation 82

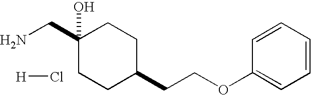

### trans-1-(Aminomethyl)-4-(2-phenoxyethyl)cyclohexanol hydrochloride

This compound was prepared with 4-(2-phenoxyethyl)cyclohexanone by a procedure similar to that in Preparation 16.

¹H NMR (DMSO-d₆) δ: 7.75 (br, 3H), 7.32-7.23 (m, 2H), 6.97-6.88 (m, 3H), 4.99 (br, 1H), 3.98 (t, J=6.4 Hz, 2H), 2.82 (s, 2H), 1.78-1.00 (m, 11H) ppm.
Preparation 83

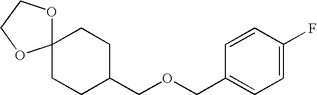

### 8-{[(4-Fluorobenzyl)oxy]methyl}-1,4-dioxaspiro[4.5]decane

This compound was prepared with 4-fluorobenzyl bromide and 1,4-dioxaspiro[4.5]dec-8-ylmethanol by a procedure similar to that in Preparation.

¹H NMR (CDCl₃) δ: 7.40-7.24 (m, 2H), 7.10-6.98 (m, 2H), 4.45 (s, 2H), 3.94 (s, 4H), 3.30 (d, J=6.6 Hz, 2H), 1.86-1.20 (m, 9H) ppm.
Preparation 84

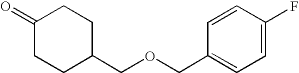

### 4-{[(4-Fluorobenzyl)oxy]methyl}cyclohexanone

This compound was prepared with 8-{[(4-fluorobenzyl)oxy]methyl}-1,4-dioxaspiro[4.5]decane by a procedure similar to that in Preparation 20.

¹H NMR (CDCl₃) δ: 7.35-7.25 (m, 2H), 7.10-7.00 (m, 2H), 4.48 (s, 2H), 3.38 (d, J=6.1 Hz, 2H), 2.48-2.00 (m, 7H), 1.56-1.36 (m, 2H) ppm.
Preparation 85

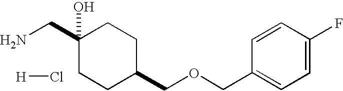

### trans-1-(Aminomethyl)-4-{[(4-fluorobenzyl)oxy]methyl}cyclohexanol hydrochloride

This compound was prepared with 4-{[(4-fluorobenzyl)oxy]methyl}cyclohexanone by a procedure similar to that in Preparation 16.

¹H NMR (DMSO-d₆) δ: 7.86 (br, 3H), 7.39-7.31 (m, 2H), 7.22-7.13 (m, 2H), 5.04 (br, 1H), 4.42 (s, 2H), 3.28 (d, J=6.2 Hz, 2H), 2.79 (s, 2H), 1.75-1.00 (m, 9H) ppm.
Preparation 86

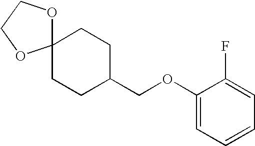

### 8-[(2-Fluorophenoxy)methyl]-1,4-dioxaspiro[4.5]decane

This compound was prepared with 2-(1,4-dioxaspiro[4.5]dec-8-yl)methanol and 2-fluorophenol by a procedure similar to that in Preparation 42.

¹H NMR (CDCl₃) δ: 7.12-6.80 (m, 4H), 3.96 (s, 4H), 3.86 (d, J=6.3 Hz, 2H), 2.00-1.28 (m, 9H) ppm.
Preparation 87

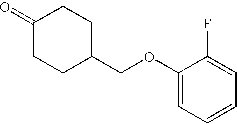

### 4-[(2-Fluorophenoxy)methyl]cyclohexanone

This compound was prepared with 8-[(2-fluorophenoxy)methyl]-1,4-dioxaspiro[4.5]decane by a procedure similar to that in Preparation 20.

¹H NMR (CDCl₃) δ: 7.13-6.80 (m, 4H), 3.94 (d, J=6.2 Hz, 2H), 2.50-2.16 (m, 7H), 1.68-1.50 (m, 2H) ppm.
Preparation 88

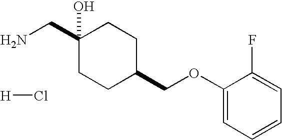

### trans-1-(Aminomethyl)-4-[(2-fluorophenoxy)methyl]cyclobexanol hydrochloride

This compound was prepared with 4-[(2-fluorophenoxy)methyl]cyclohexanone by a procedure similar to that in Preparation 16.

¹H NMR (DMSO-d₆) 5: 7.87 (br, 3H), 7.24-7.07 (m, 3H), 6.96-6.88 (m, 1H), 5.08 (s, 1H), 3.92 (d, J=6.4 Hz, 2H), 2.83 (s, 2H), 1.90-1.12 (m, 9H) ppm.
Preparation 89

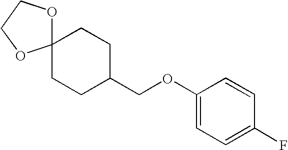

### 8-[(4-Fluorophenoxy)methl]-1,4-dioxaspiro[4.5]decane

This compound was prepared with 2-(1,4-dioxaspiro[4.5]dec-8-yl)methanol and 4-fluorophenol by a procedure similar to that in Preparation 42.

¹H NMR (CDCl₃) δ: 7.05-6.70 (m, 4H), 3.96 (s, 4H), 3.75 (d, J=6.2 Hz, 2H), 1.96-1.20 (m, 9H) ppm.
Preparation 90

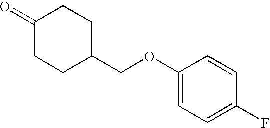

### 4-[(4-Fluorophenoxy)methyl]cyclohexanone

This compound was prepared with 8-[(4-fluorophenoxy)methyl]-1,4-dioxaspiro[4.5]decane by a procedure similar to that in Preparation 20.

¹H NMR (CDCl₃) δ: 7.12-6.72 (m, 4H), 3.84 (d, J=5.9 Hz, 2H), 2.58-2.12 (m, 7H), 1.70-1.48 (m, 2H) ppm.
Preparation 91

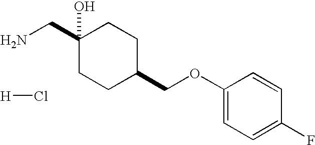

### trans-1-(Aminomethyl)-4-[(4-fluorophenoxy)methyl]cyclohexanol hydrochloride

This compound was prepared with 4-[(4-fluorophenoxy)methyl]cyclohexanone by a procedure similar to that in Preparation 16.

¹H NMR (DMSO-d₆) δ: 7.83 (br, 3H), 7.18-7.05 (m, 2H), 7.00-6.88 (m, 2H), 5.07 (s, 1H), 3.81 (d, J=6.2 Hz, 2H), 2.83 (s, 2H), 1.87-1.10 (m, 9H) ppm.
Preparation 92

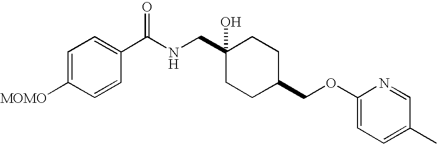

### N-[(trans-1-Hydroxy-4-{[(5-methylpyridin-2-yl)oxy]methyl}cyclohexyl)methyl]-4-(methoxymethoxy)benzamide

A mixture of N-{[trans-1-hydroxy-4-(hydroxymethyl)cyclohexyl]methyl}-4-(methoxymethoxy)benzamide (0.16 g, 0.50 mmol) and NaH (60%, 24 mg, 0.60 mmol) was stirred at room temperature for 30 min. 2-Fluoro-5-methylpyridine (78 mg, 0.70 mmol) was added to the mixture. The mixture was stirred at 200° C. for 10 min with microwave irradiation, and quenched with NaHCO₃. The whole was extracted with AcOEt. The extract was washed with water, dried over MgSO₄ and concentrated. The residue was purified by silica gel column chromatography (hexane:AcOEt=4:5) to give the titled compound (97 mg).

¹H NMR (CDCl₃) δ: 7.90-7.80 (m, 1H), 7.76 (d, J=8.9 Hz, 2H), 7.43-7.33 (m, 1H), 7.07 (d, J=8.9 Hz, 2H), 6.64 (d, J=8.2 Hz, 1H), 6.58-6.47 (m, 1H), 5.22 (s, 2H), 4.13 (d, J=6.3 Hz, 2H), 3.60 (d, J=5.9 Hz, 2H), 3.48 (s, 3H), 2.57 (br, 1H), 2.24 (s, 3H), 2.00-1.77 (m, 5H), 1.60-1.21 (m, 4H) ppm.
Preparation 93

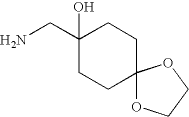

### 8-(Aminomethyl)-1,4-dioxaspiro[4.5]decan-8-ol

This compound was prepared with 8-oxo-1,4-dioxaspiro[4.5]decane by a procedure similar to that in Preparation 30.

¹H NMR (CDCl₃) δ: 3.98-3.90 (m, 4H), 2.97 (br, 1H), 2.02-1.45 (m, 8H) ppm. (NH₂ was not observed.)
Preparation 94

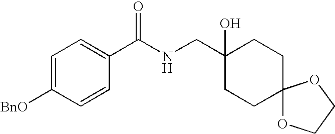

### 4-(Benzyloxy)-N-[(8-hydroxy-1,4-dioxaspiro[4.5]dec-8-yl)methyl]benzamide

This compound was prepared with 4-benzyloxybenzoic acid and 8-(aminomethyl)-1,4-dioxaspiro[4.5]decan-8-ol by a procedure similar to that in Preparation 9.

¹H NMR (CDCl₃) δ: 7.75 (d, J=8.9 Hz, 2H), 7.50-7.30 (m, 5H), 6.98 (d, J=8.9 Hz, 2H), 6.63-6.53 (m, 1H), 5.10 (s, 2H), 3.96-3.92 (m, 4H), 3.49 (d, J=5.9 Hz, 2H), 2.96 (br, 1H), 1.96-1.56 (m, 8H) ppm.
Preparation 95

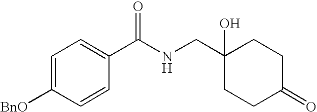

### 4-(Benzyloxy)-N-[(1-hydroxy-4-oxocyclohexyl)methyl]benzamide

This compound was prepared with 4-(benzyloxy)-N-[(8-hydroxy-1,4-dioxaspiro[4.5]dec-8-yl)methyl]benzamide by a procedure similar to that in Preparation 20.

¹H NMR (CDCl₃) δ: 7.76 (d, J=8.9 Hz, 2H), 7.46-7.30 (m, 5H), 7.02 (d, J=8.9 Hz, 2H), 6.60-6.50 (m, 1H), 5.12 (s, 2H), 3.86 (s, 1H), 3.37 (d, J=5.9 Hz, 2H), 2.84-2.68 (m, 2H), 2.36-2.24 (m, 2H), 2.14-2.00 (m, 2H), 1.85-1.70 (m, 2H) ppm.
Preparation 96

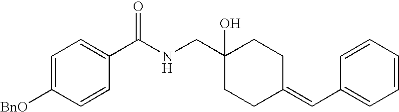

### N-[(4-Benzylidene-1-hydroxycyclohexyl)methyl]-4-(benzyloxy)benzamide

A mixture of 4-(benzyloxy)-N-[(1-hydroxy-4-oxocyclohexyl)methyl]benzamide (0.35 g, 1.0 mmol), diethyl benzylphosphonate (0.46 g, 2.0 mmol) and NaH (60%, 0.16 g, 4.0 mmol) in Dimethoxyethane (10 mL) was stirred at room temperature for 16 hours. The mixture was quenched with water and extracted with AcOEt. The extract was washed with sat. aq. NaCl, dried over MgSO₄ and concentrated. The residue was purified by silica gel column chromatography (hexane:AcOEt=3:2) to give the titled compound (42 mg).

¹H NMR (CDCl₃) δ: 7.87 (d, J=8.6 Hz, 2H), 7.50-7.13 (m, 1OH), 7.01 (d, J=8.6 Hz, 2H), 6.54-6.51 (m, 1H), 6.31 (s, 1H), 5.12 (s, 2H), 3.57-3.50 (m, 2H), 2.71-2.23 (m, 5H), 1.90-1.45 (m, 3H) ppm. (OH was not observed.)
Preparation 97

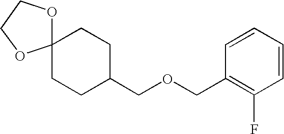

### 8-{[(2-Fluorobenzyl)oxy]methyl}-1,4-dioxaspiro[4.5]decane

This compound was prepared with 2-fluorobenzyl bromide and 1,4-dioxaspiro[4.5]dec-8-ylmethanol by a procedure similar to that in Preparation 47.

¹H NMR (CDCl₃) δ: 7.46-6.98 (m, 4H), 4.56 (s, 2H), 3.94 (s, 4H), 3.34 (d, J=6.6 Hz, 2H), 1.90-1.20 (m, 9H) ppm.
Preparation 98

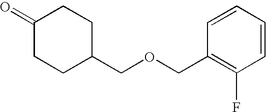

### 4-{[(2-Fluorobenzyl)oxy]methyl}cyclohexanone

This compound was prepared with 8-{[(2-fluorobenzyl)oxy]methyl}-1,4-dioxaspiro[4.5]decane by a procedure similar to that in Preparation 20.

¹H-NMR (CDCl₃) δ: 7.45-7.37 (m, 1H), 7.34-7.23 (m, 1H), 7.17-7.02 (m, 2H), 4.59 (s, 2H), 3.43 (d, J=6.0 Hz, 2H), 2.45-2.27 (m, 4H), 2.18-2.05 (m, 3H), 1.54-1.38 (m, 2H) ppm.
Preparation 99

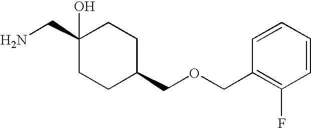

### trans-1-(Aminomethyl)-4-{[(2-fluorobenzyl)oxy]methyl}cyclohexanol

This compound was prepared with 4-{[(2-fluorobenzyl)oxy]methyl}cyclohexanone by a procedure similar to that in Preparation 30 as yellow oil.

¹H-NMR (CDCl₃) 6:7.89 (br, 2H), 7.38 (dd, J=7.5, 7.3 Hz, 1H), 7.27-7.22 (m, 1H), 7.12 (dd, J=7.5, 7.3 Hz, 1H), 7.02 (dd, J=9, 8.4 Hz, 1H), 4.53 (s, 2H), 3.31 (s, 2H), 3.15 (s, 2H), 1.90-1.45 (m, 7H), 1.20-0.98 (m, 2H) ppm. (OH was not observed.)
Preparation 100

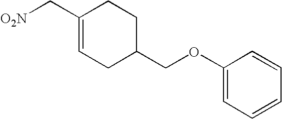

### {[4-(Nitromethyl)cyclohex-3-en-1-yl]methoxy}benzene

A mixture of 4-(phenoxymethyl)cyclohexanone (3.1 g, 15 mmol) and ethylene diamine (0.10 mL, 1.5 mmol) in nitromethane (60 mL) was refluxed for 6 hours. The mixture was concentrated and purified by silica gel column chromatography (hexane:AcOEt=10:1) to give the titled compound (3.2 g).

¹H NMR (CDCl₃) δ: 7.32-7.24 (m, 2H), 6.98-6.87 (m, 3H), 6.00-5.93 (m, 1H), 4.84 (s, 2H), 3.86 (d, J=6.2 Hz, 2H), 2.44-1.94 (m, 6H), 1.58-1.45 (m, 1H) ppm.
Preparation 101

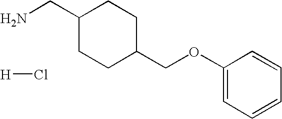

### {[4-(Phenoxymethyl)cyclohexyl]methyl}amine hydrochloride

NaBH₄ (2.2 g, 59 mmol) was added to a mixture of {[4-(nitromethyl)cyclohex-3-en-1-yl]methoxy}benzene (3.2 g, 13 mmol) and NiCl₂.6H₂O in MeOH (130 mL) and THF (65 mL) at 0° C. and the mixture was stirred for 2 hours. The mixture was absorbed to amine-gel (10 g) and evaporated. The residue was eluted with CH₂Cl₂-MeOH (10: 1). After evaporation, a mixture of the residue and 10% Pd—C (1.0 g) in EtOH (100 mL) was hydrogenated at 1 atm for 3 hours. The mixture was filtered through a pad of celite and the filtrate was concentrated. To a solution of the residue in AcOEt (20 mL), 4N HCl in AcOEt (3 mL) was added and collected with filtration to give the titled compound (1.9 g).

¹H NMR (DMSO-d₆) δ: 8.03 (br, 3H), 7.32-7.24 (m, 2H), 6.96-6.88 (m, 3H), 3.90-3.75 (m, 2H), 2.80-2.60 (m, 2H), 1.97-0.90 (m, 10H) ppm.
Preparation 102

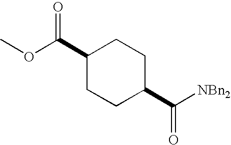

### Methyl cis-4-[(dibenzylamino)carbonyl]cyclohexanecarboxylate

This compound was prepared with dibenzylamine by a procedure similar to that in Preparation 1 as yellow oil.

¹H NMR (CDCl₃) δ: 7.40-7.23 (m, 6H), 7.18-7.13 (m, 4H), 4.57 (s, 2H), 4.47 (s, 2H), 3.72 (s, 3H), 2.63-2.54 (m, 2H), 2.30-2.23 (m, 2H), 1.92-1.78 (m, 2H), 1.71-1.64 (m, 2H), 1.57-1.44 (m, 2H) ppm.
Preparation 103

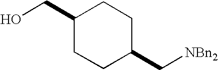

### {cis-4-[(Dibenzylamino)methyl]cyclohexyl}methanol

To a suspension of LiAlH₄ (2.1 g, 55 mmol) in THF (100 mL) was added a solution of methyl cis-4-[(dibenzylamino)carbonyl]cyclohexanecarboxylate (8.0 g, 22 mmol) in THF (100 mL) at 0° C., and the mixture was refluxed for 3 hr. The mixture was stirred at 70° C. for 16 hr. To the reaction mixture were added Na₂SO₄.10H₂O (20 g) and KF (2.0 g) and the mixture was stirred at room temperature for 1 hr. The mixture was filtered through a pad of celite and the filtrate was evaporated in vacuo to afford the titled compound as colorless oil (8.4 g).

¹H NMR (CDCl₃) δ: 7.37-7.21 (m, 10H), 3.77-3.66 (m, 1H), 3.50 (s, 4H), 3.34 (d, J=6.8 Hz, 2H), 2.27 (d, J=7.5 Hz, 2H), 1.95-1.31 (m, 8H), 1.06-0.94 (m, 2H) ppm.
Preparation 104

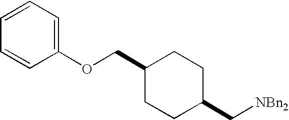

### Dibenzyl{[cis-4-(phenoxymethyl)cyclohexyl]methyl}amine

To a solution of {cis-4-[(dibenzylamino)methyl]cyclohexyl}methanol (3.0 g, 9.3 mmol) in toluene (30 mL) were added triphenylphosphine (2.7 g, 10 mmol) and phenol (1.0 g, 10 mmol). After cooling to 0C., to the mixture was added DIAD (2.0 mL, 10.2 mmol), and the mixture was stirred at room temperature for 3.5 hr. The solvent was removed in vacuo. The residue was purified by column chromatography on silica gel (hexane:ethyl acetate=10:1 as eluent) to afford the titled compound as a white solid (3.5 g, 94%).

¹H NMR (CDCl₃) δ: 7.38-7.19 (m, 12H), 6.94-6.83 (m, 3H), 3.67 (d, J=6.8 Hz, 2H), 3.52 (s, 4H), 2.30 (d, J=7.6 Hz, 2H), 1.87-1.18 (m, 10H) ppm.
Preparation 105

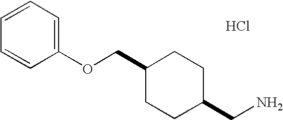

### {[cis-4-(Phenoxymethyl)cyclohexyl]methyl}amine hydrochloride

To a solution of dibenzyl{[cis-4-(phenoxymethyl)cyclohexyl]methyl}amine (3.5 g, 8.8 mmol) in MeOH (150 mL) was added 20% Pd(OH)₂—C (1.8 g) and the mixture was hydrogenated under 4 atm at 50° C. for 13 hr. To the reaction mixture was added 10% HCl-MeOH (10 mL) and the mixture was hydrogenated under 4 atm at 55° C. for 10 hr. The reaction mixture was filtered through a pad of celite and the filtrate was concentrated in vacuo. To a solution of the residue in MeOH (60 mL) were added 10% HCl-MeOH (10 mL) and 10% Pd—C (0.9 g). The mixture was hydrogenated under 4 atm at 55° C. for 15 hr. The reaction mixture was filtered through a pad of celite and the filtrate was concentrated in vacuo. To a mixture of the crude material in AcOEt (15 mL) was added 4N HCl-AcOEt (2.2 mL) and the mixture was stirred at room temperature for 4 hr. The precipitate was filtered, washed with AcOEt, and dried in vacuo to afford the titled compound as a white solid (820 mg, 37%).

¹H NMR (DMSO-d6) δ: 7.95-7.84 (m, 3H), 7.31-7.25 (m, 2H), 6.94-6.89 (m, 3H), 3.88 (d, J=7.0 Hz, 2H), 2.76 (d, J=7.1 Hz, 2H), 1.92-1.80 (m, 2H), 1.52-1.47 (m, 8H) ppm.
Preparation 106

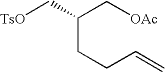

### (2S)-2-({[(4-Methylphenyl)sulfonyl]oxy}methyl)hex-5-en-1-yl acetate

To a mixture of (2R)-2-(hydroxymethyl)hex-5-en-1-yl acetate (7.9 g, 46 mmol) (Tetrahedron Asymmeery 1999, 10, 4057-4064.) and triethylamine (19 mL, 0.14 mol) in CH₂Cl₂ (90 mL), p-TsCl (13 g, 49 mmol) was added at 0° C. and the mixture was stirred at room temperature for 7 h. The mixture was washed with sat. aq. NaCl, dried over MgSO₄ and evaporated. The residue was purified by silica gel column chlomatography (hexane:AcOEt=8:1 to 6:1) to give the titled compound (13 g).

¹H NMR (CDCl₃) δ: 7.79 (d, J=8.3 Hz, 2H), 7.36 (d, J=8.3 Hz, 2H), 5.79-5.62 (m, 1H), 5.03-4.92 (m, 2H), 4.08-3.89 (m, 4H), 2.46 (s, 3H), 2.09-1.92 (m, 6H), 1.51-1.35 (m, 2H) ppm.
Preparation 107

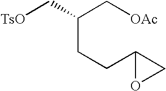

### (2S)-2-({[(4-Methylphenyl)sulfonyl]oxy}methyl)-4-oxiran-2-ylbutyl acetate

This compound was prepared with (2S)-2-({[(4-methylphenyl)sulfonyl]oxy}methyl)hex-5-en-1-yl acetate by a procedure similar to that in Preparation 40.

¹H NMR (CDCl₃) δ: 7.80 (d, J=8.2 Hz, 2H), 7.36 (d, J=7.9 Hz, 2H), 4.20-3.87 (m, 4H), 2.91-2.39 (m, 6H), 2.18-1.32 (m, 8H) ppm.
Preparation 108

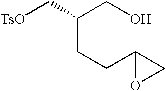

### (2S)-2-(Hydroxymethyl)-4-oxiran-2-ylbutyl 4-methylbenzenesulfonate

To a solution of (2S)-2-({[(4-methylphenyl)sulfonyl]oxy}methyl)-4-oxiran-2-ylbutyl acetate (14 g, 0.37 mmol) in MeOH (200 mL), K₂CO₃ (10 g, 74 mmol) was added and stirred at 0° C. for 30 min. The mixture was filtered and The filtrate was diluted with AcOEt. It was washed with water, dried over MgSO₄ and evaporated to give the titled compound (12 g).

¹H NMR (CDCl₃) δ: 7.80 (d, J=8.2 Hz, 2H), 7.36 (d, J=8.1 Hz, 2H), 4.19-3.99 (m, 2H), 3.71-3.53 (m, 2H), 2.91-2.82 (m, 1H), 2.78-2.71 (m, 1H), 2.54-2.42 (m, 4H), 2.13-1.12 (m, 5H) ppm. (OH was not observed.)
Preparation 109

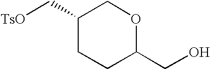

### [(3S)-6-(Hydroxymethyl)tetrahydro-2H-pyran-3-yl]methyl 4-methylbenzenesulfonate

To a solution of (2S)-2-(hydroxymethyl)-4-oxiran-2-ylbutyl 4-methylbenzenesulfonate (10 g, 31 mmol) in CH₂Cl₂ (100 mL), p-TsOH·H₂O (0.29 g, 1.5 mmol) was added at 0° C. and the mixture was stirred at 0° C. for 1 hour and at room temperature for 30 min. The mixture was washed with sat. aq. NaHCO₃, dried over MgSO₄ and evaporated. The residue was purified by silica gel column chlomatography (hexane:AcOEt=3:2 to 2:3) to give the titled compound (6.1 g).

¹H NMR (CDCl₃) δ: 7.87-7.72 (m, 2H), 7.44-7.31 (m, 2H), 4.24-3.05 (m, 7H), 2.46 (s, 3H), 2.10-1.13 (m, 5H) ppm. (OH was not observed.)
Preparation 110

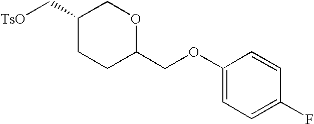

### {(3S)-6-[(4-Fluorophenoxy)methyl]tetrahydro-2H-pyran-3-yl}methyl 4-methylbenzenesulfonate

This compound was prepared with [(3S)-6-(hydroxymethyl)tetrahydro-2H-pyran-3-yl]methyl 4-methylbenzenesulfonate by a procedure similar to that in Preparation 106.

¹H NMR (CDCl₃) δ: 7.85-7.75 (m, 2H), 7.41-7.32 (m, 2H), 7.04-6.72 (m, 4H), 4.30-3.10 (m, 7H), 2.55-2.40 (m, 3H), 2.16-1.18 (m, 5H) ppm.
Preparation 111

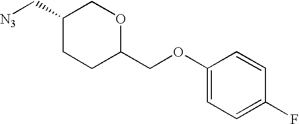

### (5R)-5-(Azidomethyl)-2-[(4-fluorophenoxy)methyl]tetrahydro-2H-pyran

This compound was prepared with {(3S)-6-[(4-fluorophenoxy)methyl]tetrahydro-2H-pyran-3-yl}methyl 4-methylbenzenesulfonate by a procedure similar to that in Preparation 7.

¹H NMR (CDCl₃) δ: 7.05-6.75 (m, 4H), 4.18-3.05 (m, 7H), 2.11-1.17 (m, 5H) ppm.
Preparation 112

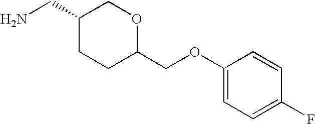

### ({(3R)-6-[(4-Fluorophenoxy)methyl]tetrahydro-2H-pyran-3-yl}methyl)amine

This compound was prepared with (5R)-5-(azidomethyl)-2-[(4-fluorophenoxy)methyl]tetrahydro-2H-pyran by a procedure similar to that in Preparation 8.

¹H NMR (CDCl₃) δ: 7.03-6.81 (m, 4H), 4.18-2.50 (m, 7H), 2.15-1.11 (m, 5H) ppm. (NH₂ was not observed.)
Preparation 113

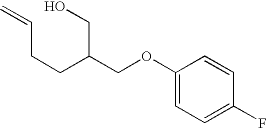

### 2-[(4-Fluorophenoxy)methyl]hex-5-en-1-ol

This compound was prepared with 2-but-3-en-1-ylpropane-1,3-diol by a procedure similar to that in Preparation 104.

¹H NMR (CDCl₃) δ: 7.05-6.85 (m, 4H), 5.90-5.73 (m, 1H), 5.10-4.95 (m, 2H), 4.05-3.90 (m, 2H), 3.83-3.68 (m, 2H), 2.25-1.98 (m, 3H), 1.93-1.78 (m, 1H), 1.67-1.48 (m, 2H). (OH was not observed.)
Preparation 114

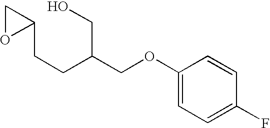

### 2-[(4-Fluorophenoxy)methyl]-4-oxiran-2-ylbutan-1-ol

This compound was prepared with 2-[(4-fluorophenoxy)methyl]hex-5-en-1-ol by a procedure similar to that in Preparation 40.

¹H NMR (CDCl₃) δ: 7.04-6.74 (m, 4H), 4.05-3.90 (m, 2H), 3.85-3.69 (m, 2H), 30.1-2.90 (m, 1H), 2.81-2.73 (m, 1H), 2.55-2.47 (m, 1H), 2.11-1.48 (m, 5H) ppm. (OH was not observed.)
Preparation 115

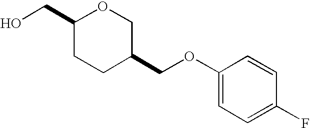

### {(2S*,5S*)-5-[(4-Fluorophenoxy)methyl]tetrahydro-2H-pyran-2-yl}methanol

This compound was prepared with 2-[(4-fluorophenoxy)methyl]-4-oxiran-2-ylbutan-1-ol by a procedure similar to that in Preparation 109.

¹H NMR (CDCl₃) δ: 7.03-6.75 (m, 4H), 4.15-4.05 (m, 2H), 4.00-3.91 (m, 1H), 3.70-3.45 (m, 4H), 2.05-1.75 (m, 3H), 1.55-1.34 (m, 2H) ppm. (OH was not observed.)
Preparation 116

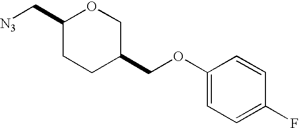

### (2S*,5S*)-2-Azidomethyl)-5-[(4-fluorophenoxy)methyl]tetrahydro-2H-pyran

This compound was prepared with {(2S*,5S*)-5-[(4-fluorophenoxy)methyl]tetrahydro-2H-pyran-2-yl}methanol by a procedure similar to that in Preparation 67.

¹H NMR (CDCl₃) δ: 7.05-6.75 (m, 4H), 4.18-3.95 (m, 3H), 3.70-3.46 (m, 2H), 3.34-3.15 (m, 2H), 2.05-1.75 (m, 3H), 1.55-1.40 (m, 2H) ppm.
Preparation 117

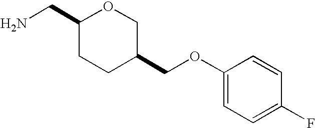

### ({(2S*,5S*)-5-[(4-Fluorophenoxy)methyl]tetrahydro-2H-pyran-2-yl}methyl)amine

This compound was prepared with (2S*,5S*)-2-(azidomethyl)-5-[(4-fluorophenoxy)methyl]tetrahydro-2H-pyran by a procedure similar to that in Preparation 8.

¹H NMR (CDCl₃) δ: 7.03-6.81 (m, 4H), 4.20-3.95 (m, 2H), 3.70-3.60 (m, 1H), 3.42-3.20 (m, 2H), 2.73-2.64 (m, 2H), 2.15-1.70 (m, 3H), 1.56-1.25 (m, 2H) ppm. (NH₂ was not observed.)
Preparation 118

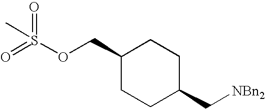

### {cis-4-[(Dibenzylamino)methyl]cyclohexyl}methyl methanesulfonate

To a solution of {cis-4-[(dibenzylamino)methyl]cyclohexyl}methanol (35 g, 108 mmol) in dichloromethane (200 mL) was added triethylamine (30 mL, 216 mmol). To the mixture was added methanesulfonyl chloride (10 mL, 130 mmol) at 0° C., and the mixture was stirred at 0° C. for 1 hr. To the mixture was added sat. aq. NaHCO₃ (250 mL), and the whole mixture was extracted with dichloromethane (100 mL×3). The combined organic layer was washed with brine (300 mL), dried over Na₂SO₄, concentrated in vacuo to afford the titled compound as yellow oil (46 g).

¹H NMR (CDCl₃) δ: 7.37-7.19 (m, 10H), 3.91 (d, J=7.1 Hz, 2H), 3.50 (s, 4H), 2.93 (s, 3H), 2.28 (d, J=7.6 Hz, 2H), 1.91-0.98 (m, 10 H) ppm.
Preparation 119

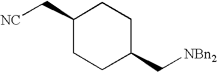

### {cis-4-[(Dibenzylamino)methyl]cyclohexyl}acetonitrile

To a solution of {cis-4-[(dibenzylamino)methyl]cyclohexyl}methyl methanesulfonate (46 g, 108 rnmol) in DMSO (200 mL) were added sodium cyanide (8.0 g, 162 mmol) and 15-crown-5 ether (11 mL, 54 mmol) and the reaction mixture was stirred at 60° C. for 19 hr. To the mixture was added H₂O (500 mL), and the whole mixture was extracted with AcOEt (200 mL×3). The combined organic layer was washed with H₂O (500 mL), dried over Na₂SO₄, concentrated in vacuo. The residue was purified by column chromatography on silica gel (hexane: ethyl acetate=10:1 as eluent) to afford the titled compound as a white solid (25 g, 68%).

¹H NMR (CDCl₃) δ: 7.37-7.20 (m, 10H), 3.50 (s, 4H), 2.28 (d, J=8.1 Hz, 2H), 2.10 (d, J=8.1 Hz, 2H), 1.81-1.78 (m, 2H), 1.58-1.36 (m, 6H), 1.08-1.00 (m, 2H) ppm.
Preparation 120

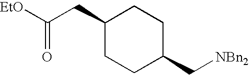

### Ethyl {cis-4-[(benzylamino)methyl]cyclohexyl}acetate

To a cold EtOH (125 mL) was added conc. H₂SO₄ (63 mL) at 0° C., and the mixture was stirred at 0° C. for 10 min. To the mixture of {cis-4-[(dibenzylamino)methyl]cyclohexyl}acetonitrile (25 g, 74 mmol) in EtOH (40 mL) was added the mixture of H₂SO₄ in EtOH solution at 0° C. The reaction mixture was refluxed for 4.5 hr. After evaporation, the residue was cooled at 0° C., H₂O (100 mL) was added. The mixture was basified with NaOH until pH 8. The whole mixture was extracted with AcOEt (100 mL×3). The combined organic layer was washed with H₂O (200 mL), dried over Na₂SO₄, concentrated in vacuo. The residue was purified by column chromatography on silica gel (hexane:ethyl acetate=40:1 as eluent) to afford the titled compound as colorless oil (16 g, 61%).

¹H NMR (CDCl₃) δ: 7.37-7.19 (m, 10H), 4.16-4.05 (m, 2H), 3.50 (s, 4H), 2.28-2.26 (m, 2H), 2.16-2.07 (m, 2H), 2.00-1.86 (m, 1H), 1.83-1.70 (m, 1H), 1.58-1.49 (m, 2H), 1.43-1.28 (m, 4H), 1.23 (t, J=8.1 Hz, 3H), 1.09-0.99 (m, 2H) ppm. MS (ESI): 380.26 (M+H)⁺
Preparation 121

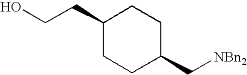

### 2-{cis-4-[(Dibenzylamino)methyl]cyclohexyl}ethanol

To a suspension of LiAlH4 (2.4 g, 63 mmol) in THF (150 mL) was added a solution of ethyl {cis-4-[(benzylamino)methyl]cyclohexyl}acetate (15.8 g, 42 mmol) in THF (200 mL) at 0° C., and the reaction mixture was stirred at 0° C. for 1 hr. To the reaction mixture were added Na₂SO₄.10H₂O (24 g) and KF (2.4 g) and the mixture was stirred at room temperature for 1 hr. The mixture was filtered through a pad of celite and the filtrate was evaporated in vacuo to afford the titled compound as yellow oil (15.9 g).

¹H NMR (CDCl₃) δ: 7.38-7.19 (m, 10H), 3.61-3.56 (m, 2H), 3.50 (s, 4H), 2.27 (d, J=7.3 Hz, 2H), 1.85-1.71 (m, 1H), 1.56-1.34 (m, 9H), 1.05-0.96 (m, 2H) ppm. —OH was no observed.]
Preparation 122

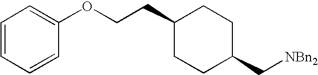

### Dibenzyl{[cis-4-(2-phenoxyethyl)cyclohexyl]methyl}amine

To a solution of 2-{cis-4-[(dibenzylamino)methyl]cyclohexyl}ethanol (3.5 g, 10 mmol) in toluene (33 mL) were added triphenylphosphine (3.0 g, 11 mmol) and phenol (1.1 g, 11 mmol). To the mixture was added diisopropylazodicarboxylate (2.2 mL, 11 mmol) at 0° C., the mixture was stirred at room temperature for 18 hr. The reaction mixture was concentrated in vacuo. The residue was purified by column chromatography on silica gel (hexane:ethyl acetate=40:1 as eluent) to afford the titled compound as colorless oil (4.0 g, 92%).

¹H NMR (CDCl₃) δ: 7.34-7.19 (m, 12H), 6.95-6.84 (m, 3H), 3.91-3.86 (m, 2H), 3.50 (s, 4H), 2.28 (d, J=8.1 Hz, 2H), 1.86-1.73 (m, 1H), 1.62-1.37 (m, 9H), 1.11-1.02 (m, 2H) ppm.
Preparation 123

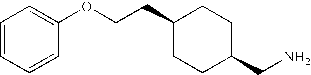

### {[cis-4-(2-Phenoxyethyl)cyclohexyl]methyl}amine hydrochloride

To a solution of dibenzyl{[cis-4-(2-phenoxyethyl)cyclohexyl]methyl}amine (8.4 g, 23 mmol) in MeOH (80 mL) was added THF (10 mL). To the mixture were added 10% Pd—C (0.8 g) and ammonium formate (3.3 g, 52 mmol), the reaction mixture was stirred at 62° C. for 1 hr. The reaction mixture was filtered through a pad of celite and the filtrate was concentrated in vacuo. To the residue was added H₂O (50 mL) and brine (50 mL). The mixture was extracted with CH₂Cl₂ (50 mL×3), dried over Na₂SO₄, concentrated in vacuo. To this residue was added AcOEt (30 mL), and the mixture was cooled to 0° C. To the mixture was added 4N HCl-AcOEt (5.5 mL). The reaction mixture was stirred at room temperature for 2 hr. The reaction mixture was concentrated in vacuo, filtered and washed with AcOEt. The solid was recrystallized from ⁱPrOH (60 mL) to afford the titled compound as a white solid (3.3 g, 52%).

¹H NMR (DMSO-d₆) δ: 8.06-7.78 (m, 3H), 7.31-7.25 (m, 2H), 6.94-6.89 (m, 3H), 4.00-3.96 (m, 2H), 2.75 (d, J=5.4 Hz, 2H), 1.82-1.65 (m, 4H), 1.55-1.31 (m, 8H) ppm.
Preparation 124

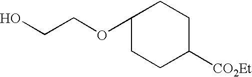

### Ethyl 4-(2-hydroxyethoxy)cyclohexanecarboxylate

To a solution of ethyl 1,4-dioxaspiro[4.5]decane-8-carboxylate (5.0 g, 23 mmol) in dichloromethane (80 mL) were added triethylsilane (4.1 mL, 26 mmol) and tert-butyldimethylsilyl trifluoromethanesulfonate (0.5 mL, 2.3 mmol) at 0° C. The reaction mixture was stirred at room temperature for 19 hr. To the mixture was added water (100 mL) and the whole mixture was extracted with dichloromethane (50 mL×3) and the combined organic layer was dried over Na₂SO₄, concentrated in vacuo. The residue was purified by column chromatography on silica gel (hexane:ethyl acetate=2:1˜1:1 as eluent) to afford the titled compound as yellow oil (1.0 g, 20%).

¹H NMR (CDCl₃, cis/trans mixture) δ: 4.21-4.08 (m, 2H), 3.72-3.50 (m, 4.5H), 3.32-3.22 (m, 0.5H), 2.40-1.22 (m, 13H) ppm.
Preparation 125

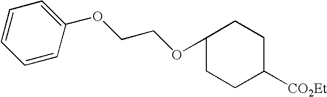

### Ethyl 4-(2-phenoxyethoxy)cyclohexanecarboxylate

To a solution of ethyl 4-(2-hydroxyethoxy)cyclohexanecarboxylate (1.0 g, 4.7 mmol) in toluene (15 mL) were added triphenylphosphine (1.3 g, 5.1 mmol), phenol (484 mg, 5.1 mmol) and diisopropyl azodicarboxylate (1.0 mL, 5.1 mmol) at 0C. The mixture was stirred at 0° C. for 1 hr, then warmed to room temperature for 2 hr. The reaction mixture was evaporated and the residue was purified by column chromatography on silica gel (hexane: ethyl acetate=δ: 1 as eluent) to afford the titled compound as yellow oil (761 mg, 56%).

¹H NMR (CDCl₃, cis/trans mixture) δ: 7.30-7.21 (m, 2H), 6.97-6.82 (m, 3H), 4.16-4.09 (m, 4H), 3.86-3.76 (m, 2H), 3.57 (m, 0.25H), 3.40-3.30 (m, 0.75H), 2.36-1.23 (m, 12H) ppm.
Preparation 126

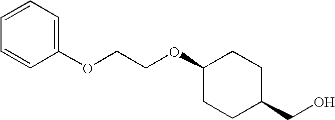

### [cis-4-(2-Phenoxyethoxy)cyclohexyl]methanol

To a suspension of LiAlH₄ (148 mg, 3.9 mmol) in THF (4 mL) was added a solution of ethyl 4-(2-phenoxyethoxy)cyclohexanecarboxylate (761 mg, 2.6 mmol) in THF (6 mL) at 0° C., and the mixture was stirred for 30 min. To the reaction mixture were added Na₂SO₄.10H₂O (1.4 g) and KF (0.2 g) and the whole mixture was stirred at room temperature for 0.5 hr. The mixture was filtered through a pad of celite and the filtrate was evaporated in vacuo. The residue was purified by column chromatography on silica gel (hexane:ethyl acetate=3:1 as eluent) to afford the titled compound as colorless oil (111 mg, 17%).

¹H NMR (CDCl₃) δ: 7.31-7.23 (m, 2H), 6.97-6.91 (m, 3H), 4.14-4.09 (m, 2H), 3.78-3.74 (m, 2H), 3.64-3.62 (m, 1H), 3.47 (d, J=6.1 Hz, 2H), 1.96-1.88 (m, 2H), 1.59-1.38 (m, 7H) ppm. (OH was not observed.)
Preparation 127

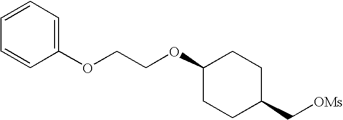

### [cis-4-(2-Phenoxyethoxy)cyclohexyl]methyl methanesulfonate

To a solution of [cis-4-(2-phenoxyethoxy)cyclohexyl]methanol in dichloromethane (1.5 mL) was added triethylamine (0.1 mL, 0.9 mmol). To the mixture was cooled at 0° C., a solution of methanesulfonyl chloride (60 mg, 0.5 mmol) in dichloromethane (1.5 mL) was added and the mixture was stirred at 0° C. for 0.5 hr. To the reaction mixture was added sat. aq. NaHCO₃ (10 mL) and the mixture was extracted with dichloromethane (15 mL×3). The combined organic layers were washed with brine (20 mL×1), dried over Na₂SO₄, concentrated in vacuo to afford the titled compound as yellow oil (126 mg).

¹H NMR (CDCl₃) δ: 7.32-7.25 (m, 2H), 6.97-6.91 (m, 3H), 4.14-4.09 (m, 2H), 4.03 (d, J=7.0 Hz, 2H), 3.77-3.74 (m, 2H), 3.66-3.64 (m, 1H), 2.98 (s, 3H), 1.96-1.35 (m, 9H) ppm.
Preparation 128

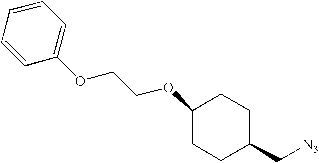

### (2-{[cis-4-(Azidomethyl)cyclohexyl]oxy}ethoxy)benzene

To a solution of [cis-4-(2-phenoxyethoxy)cyclohexyl]methyl methanesulfonate (126 mg, 0.4 mmol) in DMF (3 mL) was added sodium azide (50 mg, 0.8 mmol). The reaction mixture was stirred at 90° C. for 3 hr. To the mixture was added water (20 mL), and the mixture was extracted with ethyl acetate (20 mL×3). The combined organic layers were washed with brine (30 mL), dried over Na₂SO₄, concentrated in vacuo to afford the titled compound as yellow oil (93 mg).

¹H NMR (CDCl₃) δ: 7.31-7.25 (m, 2H), 6.97-6.91 (m, 3H), 4.14-4.10 (m, 2H), 3.78-3.74 (m, 2H), 3.64 (m, 1H), 3.13 (d, J=6.6 Hz, 2H), 1.94-1.89 (m, 2H), 1.52-1.35 (m, 7H) ppm.
Preparation 129

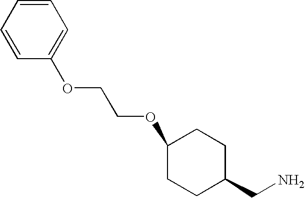

### {[cis-4-(2-Phenoxyethoxy)cyclohexyl]methyl}amine

To a solution of (2-{[cis-4-(azidomethyl)cyclohexyl]oxy}ethoxy)benzene (93 mg, 0.3 mmol) in methanol (3 mL) was added 10% Pd—C (9 mg) and stirred at room temperature for 1 hr under H₂ (1 atm). The mixture was flltered through a pad of celite and the filtrate was concentrated in vacuo to afford the titled compound as yellow oil (78 mg).

¹H NMR (CDCl₃) δ: 7.31-7.25 (m, 2H), 6.97-6.91 (m, 3H), 4.14-4.10 (m, 2H), 3.78-3.74 (m, 2H), 3.63-3.62 (m, 1H), 2.56-2.54 (m, 2H), 1.93-1.87 (m, 2H), 1.50-1.25 (m, 7H) ppm. [NH₂ proton was not observed.]
Preparation 130

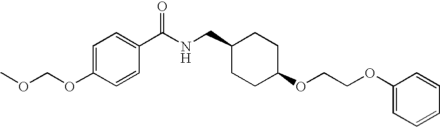

### 4-(Methoxymethoyx)-N-{[cis-4-(2-phenoxyethoxy)cyclohexyl]methyl}benzamide

This compound was prepared with {[cis-4-(2-phenoxyethoxy)cyclohexyl]methyl}amine (78 mg, 0.3 mmol) by a procedure similar to that in Preparation 17 as colorless oil (86 mg, 66%)

¹H NMR (CDCl₃) δ: 7.73-7.70 (m, 2H), 7.30-7.26 (m, 2H), 7.06-7.03 (m, 2H), 6.96-6.91 (m, 3H), 6.16-6.06 (m, 1H), 5.21 (s, 2H), 4.13-4.10 (m, 2H), 3.78-3.74 (m, 2H), 3.66-3.61 (m, 1H), 3.48 (s, 3H), 3.33-3.29 (m, 2H), 1.95-1.93 (m, 2H), 1.67-1.43 (m, 7H) ppm. MS (ESI): 414.24 (M+H)⁺
Preparation 131

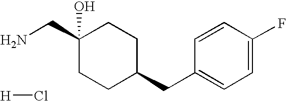

### trans-1-(Aminomethyl)-4-(4-fluorobenzyl)cyclohexanol hydrochloride

This compound was prepared with 4-fluorobenzylcyclohexanone (WO 2001028987) by a procedure similar to that in Preparation 16.

¹H NMR (DMSO-d₆) δ: 7.80 (br, 3H), 7.27-7.01 (m, 4H), 5.00 (br, 1H), 2.89 (s, 2H), 2.59-2.42 (m, 2H), 1.78-0.94 (m, 9H) ppm.
Preparation 132

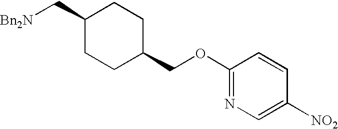

### N,N-Dibenzyl-1-(cis-4-{[(5-nitropyridin-2-yl)oxy]methyl}cyclohexyl)methanamine

To a solution of {cis-4-[(dibenzylamino)methyl]cyclohexyl}methanol (0.64 g, 2.0 mmol) in DMF, NaH (60%, 0.18 g, 4.4 mmol) was added at 0° C. and the mixture was stirred at room temperature for 1 hour. 2-Chloro-5-nitropyridine (0.96 g, 6.0 mmol) was added at 0° C., and the mixture was stirred at room temperature overnight. The mixture was quenched with water and diluted with AcOEt. The organic layer was washed with water, dried over MgSO₄ and evaporated. The residue was purified by silica gel column chlomatography (hexane-AcOEt 40:1) to give the titled compound (0.51 g).

¹H NMR (CDCl₃) δ: 9.04 (d, J=2.8 Hz, 1H), 8.33 (dd, J=2.8, 9.1 Hz, 1H), 7.47-7.15 (m, 10H), 6.75 (d, J=9.2 Hz, 1H), 4.16 (d, J=6.9 Hz, 2H), 3.52 (s, 4H), 2.30 (d, J=7.4 Hz, 2H), 2.02-1.09 (m, 10H) ppm.
Preparation 133

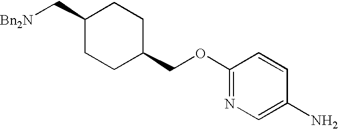

### 6-({cis-4-[(Dibenzylamino)methyl]cyclohexyl}methoxy)pyridin-3-amine

A mixture of N,N-dibenzyl-1-(cis-4-{[(5-nitropyridin-2-yl)oxy]methyl}cyclohexyl)methanamine (0.51 g, 1.1 mmol), Fe (0.31 g, 5.5 mmol), NH₄Cl (29 mg, 0.55 mmol) in EtOH (10 mL) and water (2 mL) was refluxed for 6 h. To the mixture, water (20 mL) was added and extracted with CH₂Cl₂. The extract was washed with water, dried over MgSO₄ and evaporated. The residue was purified by silica gel column chlomatography (hexane-AcOEt 7:3) to give the titled compound (0.44 g).

¹H NMR (CDCl₃) δ: 7.62 (d, J=3.0 Hz, 1H), 7.43-7.16 (m, 10H), 7.01 (dd, J=3.0, 8.7 Hz, 1H), 6.54 (d, J=8.7 Hz, 1H), 3.93 (d, J=7.1 Hz, 2H), 3.51 (s, 4H), 2.29 (d, J=7.4 Hz, 2H), 2.00-1.15 (m, 10H) ppm. (NH₂ was not observed.)
Preparation 134

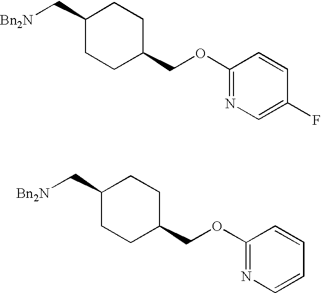

### N,N-Dibenzyl-1-(cis-4-{[(5-fluoropyridin-2-yl)oxy]methy}cyclohexyl)methanamine and N-benzyl-1-phenyl-N-({cis-4-[(pyridin-2-yloxy)methyl]cyclohexyl}methyl)methanamine

To a solution of 6-({cis-4-[(dibenzylanino)methyl]cyclohexyl}methoxy)pyridin-3-amine (0.22 g, 0.55 mmol) in EtOH, ethyl nitrite (15% in EtOH, 0.31 mL) was slowly added at 0° C. To the mixture, HBF₄ (42% in water, 0.23 mL) was slowly added at 0° C. and the mixture was stirred at 0° C. for 1 hour. To the mixture, cold ether was added and insoluble purple oil was washed with cold ether. A mixture of the oil and heptane (0.6 mL) was heated at 100° C. for 1 hour. To the mixture, water was added and extracted with CH₂Cl₂. The extract was dried over MgSO₄ and evaporated. The residue was purified by prep. TLC (hexane-AcOEt 10:1) to give the titled compounds (61 mg and 23 mg).

### N,N-Dibenzyl-1-(cis-4-{[(5-fluoropyridin-2-yl)oxy]methy}cyclohexyl)methanamine (61 mg)

¹H NMR (CDCl₃) δ: 7.95 (d, J=2.8 Hz, 1H), 7.43-7.16 (m, 11H), 6.64 (dd, J=3.6, 9.1 Hz, 1H), 3.98 (d, J=7.1 Hz, 2H), 3.51 (s, 4H), 2.29 (d, J=7.6 Hz, 2H), 2.00-1.13 (m, 10H) ppm. MS (ESI): 419.25 (M+H)⁺

### N-Benzyl-1-phenyl-N-({cis-4-[(pyridin-2-yloxy)methyl]cyclohexyl}methyl)methanamine (31 mg)

¹H NMR (CDCl₃) δ: 8.16-8.09 (m, 1H), 7.60-7.50 (m, 1H), 7.43-7.15 (m, 10H), 6.87-6.65 (m, 2H), 4.02 (d, J=6.9 Hz, 2H), 3.51 (s, 4H), 1.92 (d, J=7.4 Hz, 2H), 2.02-1.16 (m, 10H) ppm.
Preparation 135

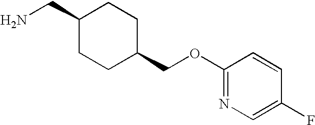

### [(cis-4-{[(5-Fluoropyridin-2-yl)oxy]methyl}cyclohexyl)methyl]amine

This compound was prepared with N,N-dibenzyl-1-(cis-4-{[(5-fluoropyridin-2-yl)oxy]methyl}cyclohexyl)methanamine by a procedure similar to that in Preparation 123.

¹H NMR (CDCl₃) δ: 8.02-7.94 (m, 1H), 7.40-7.26 (m, 1H), 6.75-6.65 (m, 1H), 4.16 (d, J=7.3 Hz, 2H), 2.63 (d, J=6.1 Hz, 2H), 2.12-1.20 (m, lOH) ppm. (NH₂ was not observed.)
Preparation 136

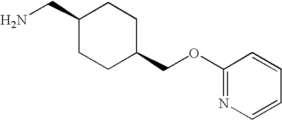

### ({cis-4-[(Pyridin-2-yloxy)methyl]cyclohexyl}methyl)amine

This compound was prepared with N-benzyl-1phenyl-N-({cis-4-[(pyridin-2-yloxy)methyl]cyclohexyl}methyl)methanamine by a procedure similar to that in Preparation 123.

¹H NMR (CDCl₃) δ: 8.20-8.11 (m, 1H), 7.62-7.51 (m, 1H), 6.91-6.70 (m, 2H), 4.20 (d, J=7.3 Hz, 2H), 2.62 (d, J=6.1 Hz, 2H), 2.12-1.20 (m, lOH) ppm. (NH₂ was not observed.)
Preparation 137

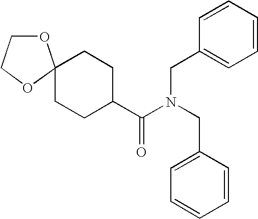

### N,N-Dibenzyl-1,4-dioxaspiro[4.5]decane-8-carboxamide

This compound was prepared with 1,4-dioxaspiro[4.5]decane-8-carboxylic acid and dibenzylamine by a procedure similar to that in Preparation 1.

¹H NMR (CDCl₃) δ: 7.40-7.23 (m, 10H), 4.59 (s, 2H), 4.46 (s, 2H), 3.95-3.93 (m, 4H), 2.61-2.51 (m, 1H), 2.10-1.95 (m, 2H), 1.85-1.77 (m, 4H), 1.53-1.42 (m, 2H) ppm.
Preparation 138

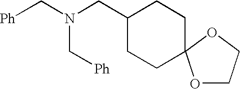

### N,N-Dibenzyl-1-(1,4-dioxaspiro[4.5]dec-8-yl)methanamine

This compound was prepared with N,N-dibenzyl-1,4-dioxaspiro[4.5]decane-8-carboxamide by a procedure similar to that in Preparation 2.

¹H NMR (CDCl₃) δ: 7.39-7.18 (m, 10H), 3.96-3.86 (m, 4H), 3.79-3.71 (m, 1H), 3.51 (s, 4H), 2.24 (d, J=7.2 Hz, 2H), 1.92-1.80 (m, 3H), 1.75-1.40 (m, 4H), 1.14-0.98 (m, 2H) ppm.
Preparation 139

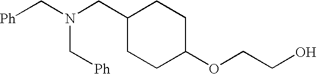

### 2-({4-[(Dibenzylamino)methyl]acclohexyl}oxy)ethanol

To a solution of N,N-dibenzyl-1-(1,4-dioxaspiro[4.5]dec-8-yl)methanamine (2.8 g, 7.9 mmol) in dichloromethane (70 mL) was added diisobutylaluminium hydride (0.93 M in hexane, 15 mL, 16 mmol) at 0° C. under nitrogen and the mixture was stirred for 3 hours. Na₂SO₄.10H₂O (12 g) and KF (2 g) were added to the mixture and the resulting mixture was stirred for 2 hours at room temperature. After filtration, the filtrate was evaporated to affors the titled compound. (2.6 g)

¹H NMR (CDCl₃) δ: 7.42-7.16 (m, 10H), 3.75-3.50 (m, 2H), 3.59-3.06 (m, 7H), 2.35-2.10 (m, 2H), 2.11-0.63 (m, 9H) ppm. (OH was not observed.)
Preparation 140

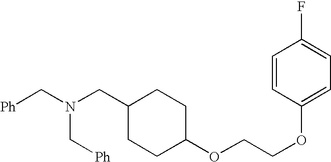

### N,N-Dibenzyl-1-{4-[2-(4-fluorophenoxy)ethoxy]cyclohexyl}methanamine

This compound was prepared with 2-({4-[(dibenzylamino)methyl]cyclohexyl}oxy)ethanol by a procedure similar to that in Preparation 33.

¹H NMR (CDCl₃) δ: 7.39-7.17 (m, 10H), 7.02-6.77 (m, 4H), 4.17-4.00 (m, 2H), 3.85-3.60 (m, 2H), 3.58-3.49 (m, 5H), 2.26-2.16 (m, 2H), 1.81-1.11 (m, 9H) ppm.
Preparation 141

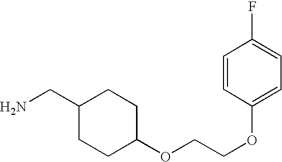

### ({4-[2-(4-Fluorophenoxy)ethoxy]cyclohexyl}methyl)amine

This compound was prepared with N,N-dibenzyl-1-{4-[2-(4-fluorophenoxy)ethoxy]cyclohexyl}methanamine by a procedure similar to that in Preparation 123.

¹H NMR (CDCl₃) 5: 7.03-6.82 (m, 4H), 4.12-4.02 (m, 2H), 3.84-3.70 (m, 2H), 3.66-3.21 (m, 1H), 2.62-2.51 (m, 2H), 2.17-0.84 (m, 9H) ppm. (NH₂ was not observed.)
Preparation 142

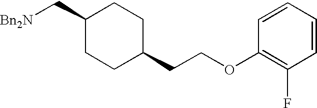

### N,N-Dibenzyl-1-{cis-4-[2-(2-fluorophenoxy)ethyl]cyclohexyl}methanamine

This compound was prepared with 2-{cis-4-[(dibenzylamino)methyl]cyclohexyl}ethanol and 2-fluorophenol by a procedure similar to that in Preparation 33.

¹H NMR (CDCl₃) δ: 7.45-6.81 (m, 14H), 4.00-3.91 (m, 2H), 3.51 (s, 4H), 2.28 (d, J=7.6 Hz, 2H), 1.93-0.94 (m, 12H) ppm.
Preparation 143

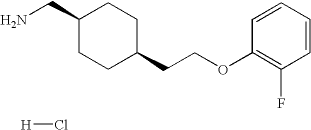

### ({cis-4-[2-(2-Fluorophenoxy)ethyl]cyclohexyl}methyl)amine hydrochloride

This compound was prepared with N,N-dibenzyl-1-{cis-4-[2-(2-fluorophenoxy)ethyl]cyclohexyl}methanamine by a procedure similar to that in Preparation 123.

¹H NMR (DMSO-d₆) δ: 8.04 (br, 3H), 7.26-7.06 (m, 3H), 7.00-6.87 (m, 1H), 6.06 (t, J=6.1 Hz, 2H), 2.74 (d, J=7.3 Hz, 2H), 1.88-1.29 (m, 12H) ppm.
Preparation 144

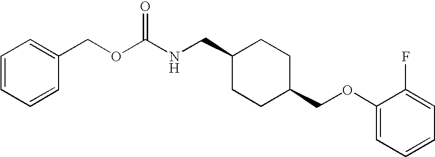

### Benzyl ({cis-4-[(2-fluorophenoxy)methyl]cyclohexyl}methyl)carbamate

This compound was prepared with benzyl {[cis-4-(hydroxymethyl)cyclohexyl]methyl}carbamate and 2-fluorophenol by a procedure similar to that in Preparation 33.

¹H NMR (CDCl3) δ: 7.44-7.22 (m, 5H), 7.14-6.81 (m, 4H), 5.10 (s, 2H), 4.85-4.68 (m, 1H), 3.96-3.82 (m, 2H), 3.24-3.11 (m, 2H), 2.14-1.19 (m, 10H) ppm.
Preparation 145

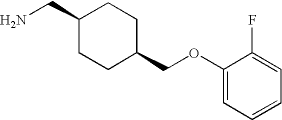

### ({cis-4-[(2-Fluorophenoxy)methyl]cyclohexyl}methyl)amine

This compound was prepared with benzyl ({cis-4-[(2-fluorophenoxy)methyl]cyclohexyl}methyl)carbamate by a procedure similar to that in Preparation 79.

MS (ESI): 237.10 (M+H)⁺
Preparation 146

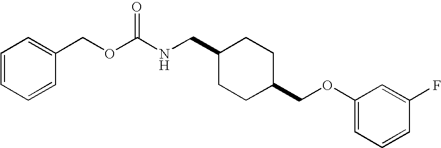

### Benzyl ({cis-4-[(3-fluorophenoxy)methyl]cyclohexyl}methyl)carbamate

This compound was prepared with benzyl {[cis-4-(hydroxymethyl)cyclohexyl]methyl}carbamate and 3-fluorophenol by a procedure similar to that in Preparation 33.

¹H NMR (CDCl₃) δ: 7.37-7.30 (m, 5H), 7.26-7.19 (m, 1H), 6.68-6.58 (m, 3H), 5.10 (s, 2H), 4.80-4.72 (m, 1H), 3.84-3.81 (m, 2H), 3.20-3.15 (m, 2H), 2.13-1.85 (m, 1H), 1.84-1.30 (m, 9H) ppm. MS (ESI): 372.01 (M+H)⁺
Preparation 147

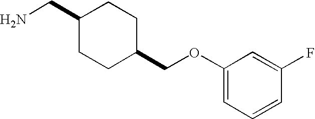

### ({cis-4-[(3-Fluorophenoxy)methyl]cyclohexyl}methyl)amine

This compound was prepared with benzyl ({cis-4-[(3-fluorophenoxy)methyl]cyclohexyl}methyl)carbamate by a procedure similar to that in Preparation 79.

¹H NMR (CDCl₃) δ: 7.25-7.13 (m, 1H), 6.88-6.41 (m, 3H), 3.85-3.81 (m, 2H), 2.48-2.85 (m, 2H), 2.29-1.87 (m, 1H), 1.84-1.02 (m, 9H) ppm. (NH₂ was not observed.) MS (ESI): 238.15 (M+H)⁺
Preparation 148

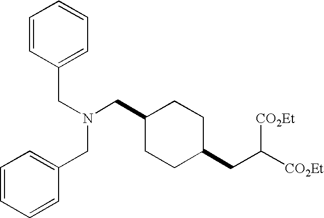

### Diethyl ({cis-4-[(dibenzylamino)methyl]cyclohexyl}methyl)malonate

To a stirred mixture of diethyl malonate (1.1 g, 7.1 mmol) in DMF (15 mL) was added sodium hydride (0.28 g, 7.1 mmol) at 0° C. After stirring for 15 minutes, {cis-4-[(dibenzylamino)methyl]cyclohexyl}methyl methanesulfonate (2.7 g, 6.7 mmol) in DMF (5 mL) was added, and the mixture was heated at 120° C. for 1 day. The mixture was cooled, quenched with sat. aq. NaHCO₃, and extracted with AcOEt. The organic layer was washed with H₂O and brine, dried over MgSO₄, and concentrated. The residue was purified by silica gel column chromatography (hexane:AcOEt=7:1) to give the titled compound (0.38 g).

¹H NMR (CDCl₃) δ: 7.42-7.17 (m, 1 OH), 4.17 (q, J=7.2 Hz, 4H) 3.50 (s, 4H), 3.31 (t, J=7.7 Hz, 1H), 2.27 (d, J=7.5 Hz, 2H), 1.95-1.27 (m, 10H), 1.25 (t, J=7.2 Hz, 6H), 1.10-0.90 (m, 2H) ppm. MS (ESI): 466.14 (M+H)⁺
Preparation 149

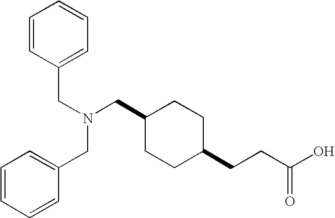

### 3-{cis-4-[(Dibenzylamino)methyl]cyclohexyl}propanoic acid

A mixture of diethyl ({cis-4-[(dibenzylamino)methyl]cyclohexyl}methyl)malonate (0.38 g, 0.82 mmol) and 2 N aq. HCl (4 mL) in acetic acid (4 mL) was stirred at 120° C. for 3 days. The mixture was concentrated, and dried in vacuum to give the titled compound (0.38 g). MS (ESI): 366.14 (M+H)⁺, 364.15 (M−H)⁻
Preparation 150

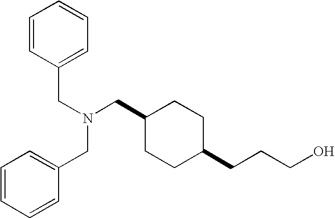

### 3-{cis-4-[(Dibenzylamino)methyl]cyclohexyl}propan-1-ol

This compound was prepared with 3-{cis-4-[(dibenzylamino)methyl]cyclohexyl}propanoic acid by a procedure similar to that in

Preparation 103.

¹H NMR (CDCl₃) δ: 7.43-7.17 (m, 10H), 3.67-3.50 (m, 2H), 3.50 (s, 4H), 2.27 (d, J=7.5 Hz, 2H), 1.95-0.70 (m, 14H) ppm. (OH was not observed.) MS (ESI): 352.14 (M+H)⁺
Preparation 151

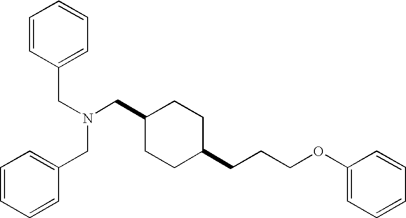

### N,N-Dibenzyl-1-[cis-4-(3-phenoxypropyl)cyclohexyl]methanamine

This compound was prepared with 3-{cis-4-[(dibenzylamino)methyl]cyclohexyl}propan-1-ol by a procedure similar to that in Preparation 33.

¹H NMR (CDCl₃) δ: 7.43-7.16 (m, 12H), 6.97-6.80 (m, 3H), 3.88 (t, J=6.8 Hz, 2H), 3.51 (s, 4H), 2.34-2.25 (m, 2H), 1.96-0.70 (m, 14H) ppm. MS (ESI): 428.19 (M+H)⁺
Preparation 152

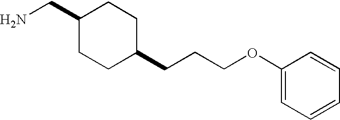

### {[cis-4-(3-Phenoxypropyl)cyclohexyl]methyl}amine

This compound was prepared with N,N-dibenzyl-1-[cis-4-(3-phenoxypropyl)cyclohexyl]methanamine by a procedure similar to that in Preparation 103.

MS (ESI): 248.17 (M+H)⁺
Preparation 153

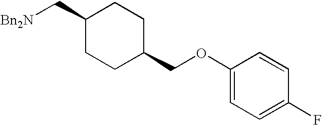

### Dibenzyl({cis-4-[(4-fluorophenoxy)methylcyclohexyl}methyl)amine

This compound was prepared with {cis-4-[(dibenzylamino)methyl]cyclohexyl}methanol (1.5 g, 4.6 mmol) and 4-fluorophenol (570 mg, 5.1 mmol) by a procedure similar to that in Preparation 104 as a white solid (1.9 g, quant.).

¹H NMR (CDCl₃) δ: 7.38-7.21 (m, 10H), 6.97-6.91 (m, 2H), 6.79-6.74 (m, 2H), 3.62 (d, J=6.8 Hz, 2H), 3.51 (s, 4H), 2.30 (d, J=7.3 Hz, 2H), 1.85-1.23 (m, 10H) ppm.
Preparation 154

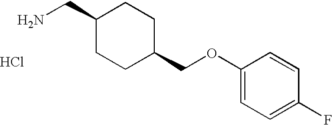

### ({cis-4-[(4-Fluorophenoxy)methyl]cyclohexy}methyl)amine hydrochloride

This compound was prepared with dibenzyl({cis-4-[(4-fluorophenoxy)methy]cyclohexyl}methyl)amine (1.9 g, 4.7 mmol) by a procedure similar to that in Preparation 105 as a white solid (543 mg, 43%).

¹H NMR (DMSO-d₆) δ: 7.96-7.79 (m, 2H), 7.14-7.08 (m, 2H), 6.97-6.92 (m, 2H), 3.86 (d, J=5.4 Hz, 2H), 2.76 (d, J=5.4 Hz, 2H), 1.91-1.79 (m, 2H), 1.51-1.46 (m, 8H) ppm.
Preparation 155

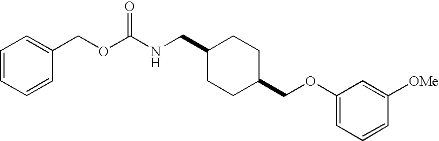

### Benzyl ({cis-4-[(3-methoxyphenoxy)methyl]cyclohexyl}methyl)carbamate

This compound was prepared with benzyl {[cis-4-(hydroxymethyl)cyclohexyl]methyl}carbamate and 3-methoxyphenol by a procedure similar to that in Preparation 33.

¹H NMR (CDCl₃) δ: 7.41-7.29 (m, 5H), 7.17 (t, J=8.1 Hz, 1H), 6.53-6.43 (m, 3H), 5.16 (s, 2H), 4.80-4.67 (1H, m), 3.81 (d, J=6.9 Hz, 2H), 3.79 (s, 3H), 3.17 (d, J=6.6 Hz, 2H), 2.21-1.90 (m, 1H), 1.78-1.30 (m, 9H) ppm. MS (ESI): 384.17 (M+H)⁺

Preparation 156

### ({cis-4-[(3-Methoxyphenoxy)methyl]cyclohexyl}methyl)amine

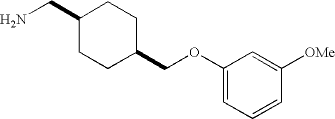

This compound was prepared with benzyl ({cis-4-[(3-methoxyphenoxy)methyl]cyclohexyl}methyl)carbamate by a procedure similar to that in Preparation 79.

¹H NMR (DMSO-d₆) δ: 7.16 (t, J=8.3 Hz, 1H), 6.56-6.42 (m, 3H), 3.84 (d, J=6.9 Hz, 2H), 3.72 (s, 3H), 2.47-2.45 (m, 2H), 1.97-1.82 (m, 1H), 1.61-1.27 (m, 9H) ppm. NH₂ was not observed. MS (ESI): 250.13 (M+H)⁺
Preparation 157

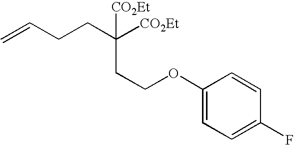

### Diethyl but-3-en-1-yl[2-(4-fluorophenoxy)ethyl]malonate

To a solution of diethyl but-3-en-1-ylmalonate (2.00 g, 9.3 mmol) in DMF was added NaH (448.0 mg, 11.2 mmol) at 0° C. The mixture was stirred at 0° C. for 30 min. Then 1-(2-bromoethoxy)-4-fluorobenzene was added and the mixture was stirred at room temperature for 10.5 hr. The reaction mixture was quenched with water and the mixture was extracted with AcOEt. The organic layer was dried over Na₂SO₄, filtered and evaporated. The crude product was purified by silica gel column chromatography (hexane:AcOEt=200:1 to 20:1) to give the titled compound (2.2 g).

¹H NMR (CDCl₃) δ: 7.00-6.90 (m, 2H), 6.80-6.76 (m, 2H), 5.85-5.67 (m, 1H), 5.07-4.96 (m, 2H), 4.30-4.20 (m, 4H), 4.05-3.95 (m, 2H), 2.45-2.40 (m, 2H), 2.15-1.92 (m, 4H), 1.29-1.15 (m, 6H) ppm.
Preparation 158

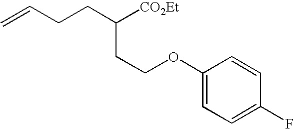

### Ethyl 2-[2-(4-fluorophenoxy)ethyl]hex-5-enoate

To a solution of diethyl but-3-en-1-yl[2-(4-fluorophenoxy)ethyl]malonate (2.21 g, 6.27 mmol) in DMSO (20 mL) and H₂O (0.11 mL), LiCl(798 mg, 18.8 mmol) was added and the mixture was stirred at 150° C. for 2 days. Then the mixture was cooled to room temperature and was poured into AcOEt/H₂O. The mixture was extracted twice with AcOEt and the combined organic layers were washed with brine. The organic layer was dried over Na₂SO₄, was filtered and evaporated. The crude product was purified by silica gel column chromatography (hexane:AcOEt=100:1-20:1) to give the titled compound (1.24 g, 4.22 mmol)

¹H NMR (CDCl₃) δ: 7.00-6.90 (m, 2H), 6.83-6.75 (m, 2H), 5.86-5.70 (m, 1H), 5.10-4.95 (m, 2H), 4.20-4.10 (m, 2H), 4.04-3.85 (m, 2H), 2.72-2.55 (m, 1H), 2.20-1.52 (m, 6H), 1.24 (t, J=6.8 Hz, 3H) ppm.
Preparation 159

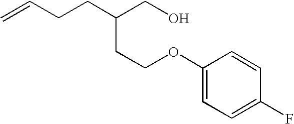

### 2-[2-(4-Fluorophenoxy)ethyl]hex-5-en-1-ol

To a suspension of LAH (167.7 mg, 4.42 mmol) in THF (30 mL) the solution of ethyl 2-[2-(4-fluorophenoxy)ethyl]hex-5-enoate (1.24 g, 4.42 mmol) in THF (15 mL) was added at 0° C. Then the mixture was stirred at room temperature for 5.5 hr. The reaction was quenched by Na₂SO₄10H₂O (2.0 g, 6.21 mmol) and KF (0.25 g, 43.0 mmol). The mixture was stirred at room temperature overnight. The mixture was filtered through a pad of celite and the filtrate was evaporated to give the titled compound (1.03 g).

¹H NMR (CDCl₃) δ: 7.00-6.94 (m, 2H), 6.86-6.81 (m, 2H), 5.89-5.70 (m, 1H), 5.10-4.90 (m, 2H), 4.10-3.95 (m, 2H), 3.70-3.55 (m, 2H), 2.20-2.05 (m, 2H), 1.90-1.72 (m, 4H), 1.60-1.40 (m, 1H) ppm. (—OH was not observed.)
Preparation 160

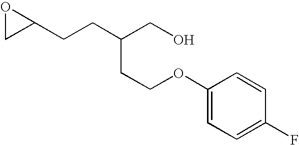

### 4-(4-Fluorophenoxy)-2-(2-oxiran-2-ylethyl)butan-1-ol

To a solution of 2-[2-(4-fluorophenoxy)ethyl]hex-5-en-1-ol (1.03 g, 4.32 mmol) in CH₂Cl₂ (40 mL), NaHCO₃ (942.9 mg, 11.2 mmol) and mCPBA (1.40 g, 8.11 mmol) was added at 0° C. and the mixture was stirred at 0° C. for 30 min. Then the mixture was allowed to warm to room temperature and was stirred overnight. The reaction was quenched by sat. Na₂S₂O₃aq and the mixture was stirred for 1 hr. The mixture was extracted 3 times with CH₂Cl₂ and the combined organic layers were washed with sat. NaHCO₃ and brine. The organic layer was dried over Na₂SO₄, filtered and evaporated to give the titled compound (1.11 g, 43.6 mmol).

¹H NMR (CDCl₃) δ: 7.01-6.94 (m, 2H), 6.90-6.80 (m, 2H), 4.15-3.96 (m, 2H), 3.70-3.58 (m, 2H), 2.96-2.90 (m, 1H), 2.80-2.74 (m, 1H), 2.53-2.47 (m, 1H), 1.90-1.78 (m, 4H), 1.70-1.45 (m, 3H) ppm. (OH was not observed.)
Preparation 161

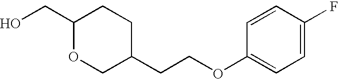

### {5-[2-(4-Fluorophenoxy)ethyl]tetrahydro-2H-pyran-2-yl}methanol

To a solution of 4-(4-fluorophenoxy)-2-(2-oxiran-2-ylethyl)butan-1-ol (1.11 g, 4.37 mmol) in CH₂Cl₂ (130 mL), p-TsOH.H₂O (12.4 mg, 0.066 mmol) was added. The mixture was stirred at room temperature for 4 hr. The reaction was quenched by sat. NaHCO₃aq, and the mixture was extracted with CH₂Cl₂. The organic layer was dried over Na₂SO₄, filtered and evaporated. The crude product was purified by silica gel column chromatography (hexane-AcOEt 9:1-3:2) to give the titled compound (898.6 mg).

¹H NMR (CDCl₃) δ: 7.05-6.90 (m, 2H), 6.86-6.77 (m, 2H), 4.10-3.80 (m, 3H), 3.70-3.10 (m, 4H), 2.10-1.70 (m, 3H), 1.60-1.20 (m, 4H) ppm. (—OH was not observed.)
Preparation 162

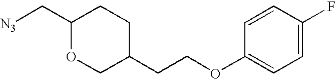

### 2-(Azidomethyl)-5-[2-(4-fluorophenoxy)ethyl]tetrahydro-2H-pyran

This compound was prepared with benzyl {5-[2-(4-fluorophenoxy)ethyl]tetrahydro-2H-pyran-2-yl}methanol by a procedure similar to that in Preparation 67.

¹H NMR (CDCl₃) δ: 7.00-6.93 (m, 2H), 6.85-6.79 (m, 2H), 4.10-3.85 (m, 3H), 3.70-2.89 (m, 4H), 2.10-1.70 (m, 3H), 1.60-1.10 (m, 4H) ppm.
Preparation 163

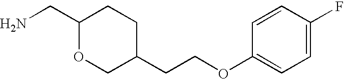

### {5-[2-(4-Fluorophenoxy)ethyl]tetrahydro-2H-pyran-2-yl}methyl)amine

This compound was prepared with 2-(azidomethyl)-5-[2-(4-fluorophenoxy)ethyl]tetrahydro-2H-pyran by a procedure similar to that in Preparation 8.

¹H NMR (CDCl₃) δ: 7.05-6.90 (m, 2H), 6.86-6.75 (m, 2H), 4.06-3.80 (m, 3H), 3.49(s, 2H), 2.75-2.68 (m, 2H), 2.15-1.10 (m, 7H) ppm MS (ESI): 254.17 (M+H)⁺
Preparation 164

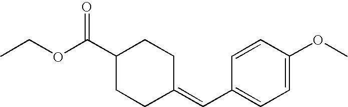

### Ethyl 4-(4-methoxybenzylidene)cyclohexanecarboxylate

A mixture of NaH (60%, 1.0 g, 25 mmol) and DMSO (20 mL) was stirred for 2 hours at 80° C. under nitrogen. After cooling to room temperature, Diethyl 4-methoxybenzylphosphate (5.2 g, 20 mmol) was added to the mixture. After 1 hour, to the mixture was added ethyl 4-oxocyclohexanecarboxylate (3.4 g, 20 mmol) and the reaction mixture was stirred for 3 hours at 60° C. The mixture was quenched with water and the whole was extracted with ethyl acetate. The organic layer was washed with water and brine, dried and evaporated. The residue was purified by silica gel chromatography (hexane:ethyl acetate=10:1) to afford the titled compound (0.39 g)

¹H NMR (CDCl₃) δ: 7.12 (d, J=8.7 Hz, 2H), 6.86 (d, J=8.7 Hz, 2H), 6.22 (s, 1H), 4.13 (q, J=7.1 Hz, 2H), 3.81 (s, 3H), 2.90-2.78 (m, 1H), 2.57-1.47 (m, 8H), 1.26 (t, J=7.1 Hz, 3H) ppm.
Preparation 165

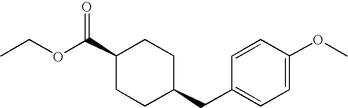

### Ethyl cis-4-(4-methoxybenzyl)cyclohexanecarboxylate

A mixture of ethyl 4-(4-methoxybenzylidene)cyclohexanecarboxylate (0.39 g, 1.4 mmol) and 10% Pd on C (40 mg) in methanol (20 mL) was stirred for 4 hours under hydrogen (4 kg/cm²). After filtration through a pad of celite, the filtrate was evaporated. The residue was purified by silica gel chromatography (hexane:ethyl acetate=12:1) to afford the titled compound (0.29 g)

¹H NMR (CDCl₃) δ: 7.06 (d, J=8.7 Hz, 2H), 6.82 (d, J=8.7 Hz, 2H), 4.15 (q, J=7.1 Hz, 2H), 3.79 (s, 3H), 2.57-2.43 (m, 3H), 2.10-1.92 (m, 2H), 1.69-1.17 (m, 10H) ppm.
Preparation 166

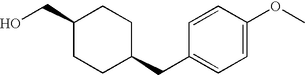

### [cis-4-(4-Methoxybenzyl)cyclohexyl]methanol

This compound was prepared with ethylcis-4-(4-methoxybenzyl)cyclohexanecarboxylate by a procedure similar to that in Preparation 121.

¹H NMR (CDCl₃) δ: 7.06 (d, J=8.7 Hz, 2H), 6.82 (d, J=8.7 Hz, 2H), 3.79 (s, 3H), 3.61-3.54 (m, 2H), 2.52 (d, J=7.6 Hz, 2H), 1.81-1.20 (m, 9H) ppm. (—OH was not observed.)
Preparation 167

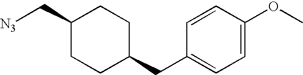

### 1-{[cis-4-(Azidomethyl)cyclohexyl]methyl}-4-methoxybenzene

This compound was prepared with [cis-4-(4-methoxybenzyl)cyclohexyl]methanol by a procedure similar to that in Preparation 67.

¹H NMR (CDCl₃) δ: 7.06 (d, J=8.6 Hz, 2H), 6.83 (d, J=8.6 Hz, 2H), 3.79 (s, 3H), 3.25 (d, J=7.3 Hz, 2H), 2.52 (d, J=7.6 Hz, 2H), 1.83-1.67 (m, 2H), 1.62-1.22 (m, 7H) ppm.
Preparation 168

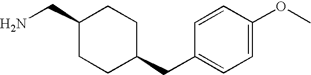

### {[cis-4-(4-Methoxybenzyl)cyclohexyl]methyl}amine

This compound was prepared with 1-{[cis-4-(azidomethyl)cyclohexyl]methyl}-4-methoxybenzene by a procedure similar to that in Preparation 8.

MS (ESI): 234.15 (M+H)⁺
Preparation 169

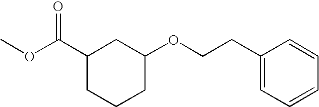

### Methyl 3-(2-phenylethoxy)cyclohexanecarboxyate

To a stirred mixture of methyl 3-oxocyclohexanecarboxylate (0.52 g, 3.3 mmol) (J. Am. Chem. Soc. 1987, 109, 3493-3494.) and phenethyl alcohol (0.48 mL, 4.0 mmol) in CH₂Cl₂ (5 mL) were added bismuth(III) chloride (0.53 g, 1.7 mmol) and triethylsilane (1.2 mL, 7.3 mmol) at room temparature. After stirring for 1 day at room temparature, the mixture was filtered over celite, and the filterate was concentrated. The residue was purified by silica gel colomn chromatography (hexane-AcOEt 10:1) to give the titled compound (0.77 g) as cis-trans (1:1) mixture.

¹H NMR (CDCl₃) δ: 7.35-7.15 (m, 5H), 3.77-3.60 (m, 2H), 3.67 (s, 3H), 3.35-3.15 (m, 0.5H), 2.87 (t, J=7.4 Hz, 2H), 2.73-2.58 (m, 0.5H), 2.37-1.10 (m, 9H) ppm.
Preparation 170

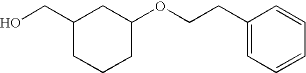

### [3-(2-Phenylethoxy)cyclohexyl]methanol

This compound was prepared as cis-trans (1:1) mixture with methyl 3-(2-phenylethoxy)cyclohexanecarboxylate by a procedure similar to that in preparation 121.

¹H NMR (CDCl₃) δ: 7.35-7.16 (m, 5H), 3.74-3.17 (m, 5H), 2.87 (t, J=7.3 Hz, 2H), 2.15-0.80 (m, 9H) ppm. (—OH was not observed.)
Preparation 171

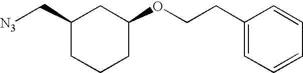

### cis-3-(Azidomethyl)cyclohexyl 2-phenylethyl ether

This compound was prepared with [3-(2-phenylethoxy)cyclohexyl]methanol by a procedure similar to that in preparation 67.

¹H NMR (CDCl₃) δ: 7.35-7.16 (m, 5H), 3.68 (t, J=7.3 Hz, 2H), 3.30-3.15 (m, 1H), 3.16 (d, J=6.6 Hz, 2H), 2.87 (t, J=7.3 Hz, 2H), 2.13-1.96 (m, 2H), 1.88-1.50 (m, 3H), 1.33-0.80 (m, 4H) ppm.
Preparation 172

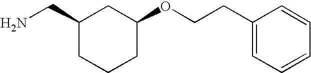

### {[(cis-3-(2-Phenylethoxy)cyclohexyl)methyl]amine

This compound was prepared with cis-3-(azidomethyl)cyclohexyl 2-phenylethyl ether by a procedure similar to that in preparation 8.

MS (ESI): 234.25 (M+H)⁺
Preparation 173

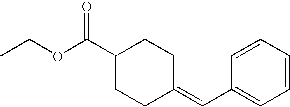

### Ethyl 4-(benzylidene)cyclohexanecarboxylate

This compound was prepared with diethyl 4-methoxybenzylphosphate by a procedure similar to that in Preparation 163.

¹H NMR (CDCl₃) δ: 7.37-7.12 (m, 5H), 6.29 (s, 1H), 4.13 (q, J=7.1 Hz, 2H), 2.92-2.79 (m, 1H), 2.59-2.36 (m, 2H), 2.32-2.16 (m, 1H), 2.14-1.91 (m, 2H), 1.77-1.46 (m, 3H), 1.26 (t, J=7.1 Hz, 3H) ppm.
Preparation 174

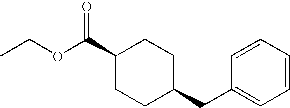

### Ethyl cis-4-benzylcyclohexanecarboxylate

This compound was prepared with ethyl 4-(benzylidene)cyclohexanecarboxylate by a procedure similar to that in Preparation 164.

¹H NMR (CDCl₃) δ: 7.34-7.06 (m, 5H), 4.15 (q, J=7.1 Hz, 2H), 2.60-2.42 (m, 3H), 2.10-1.88 (m, 2H), 1.75-1.13 (m, 10H) ppm.
Preparation 175

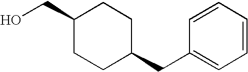

### (cis-4-Benzylcyclohexyl)methanol

This compound was prepared with ethyl cis-4-benzylcyclohexanecarboxylate by a procedure similar to that in Preparation 121.

¹H NMR (CDCl₃) δ: 7.36-7.07 (m, 5H), 3.79-3.49 (m, 3H), 2.64-2.49 (m, 2H), 1.93-1.18 (m, 10H) ppm. (—OH was not observed.)
Preparation 176

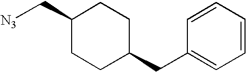

### 1-{[cis-4-(Azidomethyl)cyclohexyl]methyl}benzene

This compound was prepared with (cis-4-benzylcyclohexyl)methanol by a procedure similar to that in Preparation 67.

¹H NMR (CDCl₃) δ: 7.35-7.19 (m, 5H), 3.26 (d, J=7.3 Hz, 2H), 2.58 (d, J=7.6 Hz, 2H), 1.88-1.69 (m, 2H), 1.63-1.19 (m, 7H) ppm.
Preparation 177

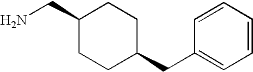

### {[cis-4-(4-Benzyl)cyclohexyl]methy}amine

This compound was prepared with 1-{[cis-4-(azidomethyl)cyclohexyl]methyl}benzene by a procedure similar to that in Preparation 8.

MS (ESI): 244.15 (M+H)⁺
Preparation 178

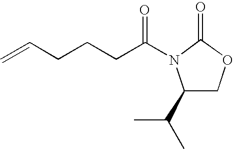

### (4R)-3-Hex-5-enoyl-4-isopropyl-1,3-oxazolidin-2-one

To a solution of hex-5-enoic acid (11.24 g, 87.0 mmol), (4R)-4-isopropyl-1,3-oxazolidin-2-one (12.91 g, 113.1 mmol) and DMAP (1.06 g, 8.70 mmol) in CH₂Cl₂, was added DCC (23.33 g, 113.1 mmol) at 0° C. and the mixture was stirred at 0° C. for 15 min. Then the mixture was stirred at room temperature overnight and was filtered through a pad of celite and the filtrate was washed with sat. NaHCO₃aq. The organic layer was dried over Na₂SO₄, filtered and evaporated. The crude product was purified by silica gel column chromatography (hexane-AcOEt 20:1-10:1) to give the titled compound (16.60 g, 73.7 mmol).

¹H NMR (CDCl₃) δ: 5.90-5.70 (m, 1H), 5.10-4.95 (m, 2H), 4.50-4.42 (m, 1H), 4.35-4.15 (m, 2H), 3.10-2.80 (m, 2H), 2.46-2.30 (m, 1H), 2.20-2.08 (m, 2H), 1.90-1.68 (m, 2H), 0.92 (d, J=7.1 Hz, 3H), 0.88 (d, J=6.9 Hz, 3H) ppm
Preparation 179

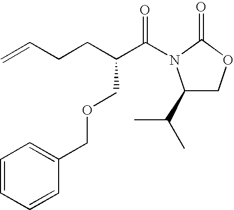

### (4R)-3-{(2S)-2-[(Benzyloxy)methyl]hex-5-enoyl}-4-isopropyl-1,3-oxazolidin-2-one

To a solution of (4R)-3-hex-5-enoyl-4-isopropyl-1,3-oxazolidin-2-one (16.60 g, 73.7 mmol) in CH₂Cl₂, was added TiCl₄ (8.89 mL, 81.1 mmol) at 0° C. and the mixture was stirred at 0° C. for 5 min. To the resulting slurry diisopropylethylamine (14.1 mL, 81.1 mmol) was added and the mixture was stirred at 0° C. for 1 hr. Benzyl chloromethyl ether (23.1 mL, 165.8 mmol) was added dropwise and the mixrure was allowed to warm to room temperature. The mixture was stirred at room temperature for 1.5 hr and quenched by careful addition of sat. NH₄Cl aq. The mixture was extracted twice with CH₂Cl₂ and the combined organic layers were dried over Na₂SO₄, filtered and evaporated. The crude product was purified by silica gel column chromatography (hexane:AcOEt=20:1 to 11:2) to give the titled compound (18.74 g, 54.3 mmol).

¹H NMR (CDCl₃) δ: 7.38-7.21 (m, 5H), 5.86-5.68 (m, 1H), 5.05-4.90 (m, 2H), 4.57-4.40 (m, 3H), 4.35-4.11 (m, 3H), 3.77-3.60 (m, 2H), 2.42-2.25 (m, 1H), 2.15-2.00 (m, 2H), 1.94-1.78(m, 1H), 1.65-1.50 (m, 1H), 0.88 (d, J=7.1 Hz, 3H), 0.78 (d, J=6.9 Hz, 3H) ppm.
Preparation 180

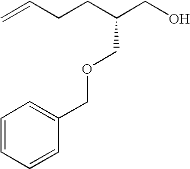

### (2R)-2-[(Benzyloxy)methyl]hex-5-en-1-ol

To a suspension of LAH (6.18 g, 162.9 mmol) in THF (250 mL) the solution of (4R)-3-{(2S)-2-[(benzyloxy)methyl]hex-5-enoyl}-4-isopropyl-1,3-oxazolidin-2-one (18.74 g, 54.3 mmol) in THF (50 mL) was added at 0° C. Then the mixture was stirred at 0° C. for 30 mn. The reaction was quenched by Na₂SO₄.10H₂O (26.24 g, 81.3 mmol) and KF (3.30 g, 56.7 mmol). The mixture was stirred at room temperature for 1 hr. The mixture was filtered through a pad of celite and the filtrate was evaporated to give the crude product. Then the crude product was purified by silica gel column chromatography (hexane:AcOEt=4:1) to give the titled compound (10.51 g, 47.7 mmol).

¹H NMR (CDCl₃) δ: 7.40-7.23 (m, 5H), 5.90-5.68 (m, 1H), 5.06-4.92 (m, 2H), 4.57-4.48 (m, 2H), 3.81-3.58 (m, 3H), 3.51-3.45 (m, 1H), 2.59-2.49 (m, 1H), 2.13-2.03 (m, 2H), 1.98-1.82 (m, 1H), 1.50-1.29 (m, 2H) ppm.
Preparation 181

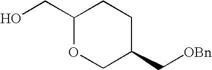

### {(5S)-5-[(Benzyloxy)methyl]tetrahydro-2H-pyran-2-yl}methanol

To a solution of (2R)-2-[(benzyloxy)methyl]hex-5-en-1-ol (10.51 g, 47.7 mmol) in CH₂Cl₂ (300 mL) were NaHCO₃ (16.03 g, 190.8 mmol) and mCPBA (23.85 g, 138.2 mmol) at 0° C. Then the mixture was stirred at room temperature for 2 days. The reaction was quenched with sat. Na₂S₂O₃ aq at 0° C. and the mixture was stirred at room temperature for 1.5 hr. The mixture was separated and the aqueous layer was extracted twice with CH₂Cl₂. The combined organic layers were washed with sat. NaHCO₃aq and brine. The organic layer was dried over MgSO₄ and filtered.

To the obtained solution, was added p-TsOH (907.3 mg, 4.77 mmol). The mixture was stirred at 55° C. for 1.75 hr. The mixture was cooled to room temperature. The reaction mixture was quenched by sat. NaHCO₃aq, and the mixture was extracted with CH₂Cl₂. The combined organic layers were dried over Na₂SO₄, and the crude product was purified by silica gel column chromatography (hexane:AcOEt=3:1 to 3:2) to give the titled compound (5.61 g, 23.7 mmol).

¹H NMR (CDCl₃) δ: 7.45-7.15 (m, 5H), 4.60-4.42 (m, 2H), 4.20-3.97 (m, 1H), 3.73-3.10 (m, 6H), 2.10-1.20 (m, 5H) ppm. (OH was not observed.)
Preparation 182

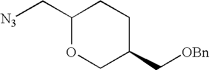

### (5S)-2-(Azidomethyl)-5-[(benzyloxy)methyl]tetrahydro-2H-pyran

This compound was prepared with {(5S)-5-[(benzyloxy)methyl]tetrahydro-2H-pyran-2-yl}methanol by a procedure similar to that in Preparation 67.

¹H NMR (CDCl₃) δ: 7.40-7.20 (m, 5H), 4.62-4.42 (m, 2H), 4.18-3.97 (m, 1H), 3.73-3.10 (m, 6H), 2.00-1.82 (m, 2H), 1.75-1.20 (m, 3H) ppm.
Preparation 183

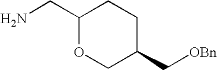

### ({(5S)-5-[(Benzyloxy)methyl]tetrahydro-2H-pyran-2-yl}methyl)amine

This compound was prepared with (5S)-2-(azidomethyl)-5-[(benzyloxy)methyl]tetrahydro-2H-pyran by a procedure similar to that in Preparation 8.

¹H NMR (CDCl₃) δ: 7.36-7.26 (m, 5H), 4.60-4.47 (m, 2H), 4.18-3.97 (m, 1H), 3.30-3.48 (m, 2H), 3.30-3.14 (m, 2H), 2.71-2.66 (m, 3H), 2.00-1.22 (m, 5H) ppm.
Preparation 184

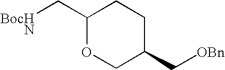

### tert-Butyl ({(5S)-5-[(benzyloxy)methyl]tetrahydro-2H-pyran-2-yl}methyl)carbamate

The reaction mixture of ({(5S)-5-[(benzyloxy)methyl]tetrahydro-2H-pyran-2-yl}methyl)amine (5.51 g, 21.1 mmol), Boc₂O (5.06 g, 23.2 mmiol) and Et₃N (8.82 mL, 63.3 mmol) in CH₂Cl₂ was stirred at room temperature overnight. The mixture was diluted with CH₂Cl₂ and was washed with sat. NaHCO₃aq and brine. The organic layer was dried over Na₂SO₄, filtered and evaporated. The crude product was purified by silica gel column chromatography (hexane-AcOEt 9:1-17:3) to give the titled compound (7.58 g, 22.6 mmol).

¹H NMR (CDCl₃) δ: 7.39-7.24 (m, 5H), 4.98-4.86 (m, 1H), 4.60-4.40 (m, 2H), 4.12-3.92 (m, 1H), 3.70-3.08 (m, 5H), 3.06-2.86 (m, 1H), 2.00-1.18 (m, 14H) ppm.
Preparation 185

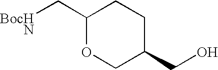

### tert-Butyl {[(5S)-5-(hydroxymethyl)tetrahydro-2H-pyran-2-yl]methy}carbamate

This compound was prepared with tert-butyl-({(5S)-5-[(benzyloxy)methyl]tetrahydro-2H-pyran-2-yl}methyl)carbamate by a procedure similar to that in Preparation 3.

¹H NMR (CDCl₃) δ: 4.95 (br, 1H), 4.12-4.00 (m, 1H), 3.90-3.65 (m, 1H), 3.60-3.10 (m, 4H), 3.08-2.92 (m, 1H), 1.93-1.13 (m, 14H) ppm. (OH was not observed.)
Preparation 186

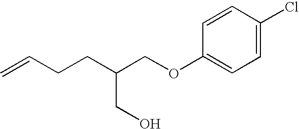

### 2-[(4-Chlorophenoxy)methyl]hex-5-en-1-ol

This compound was prepared with 2-but-3-en-1-ylpropane-1,3-diol and 4-chlorophenol by a procedure similar to that in Preparation 104.

¹H NMR (CDCl₃) δ: 7.26-7.17 (m, 2H), 6.87-6.74 (m, 2H), 5.92-5.72 (m, 1H), 5.09-4.97 (m, 2H), 4.04-3.94 (m, 2H), 3.87-3.65 (m, 2H), 2.20-2.00 (m, 3H), 1.65-1.45 (m, 2H) ppm. (OH was not observed.)
Preparation 187

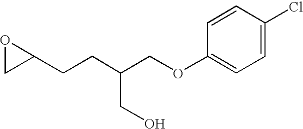

### 2-[(4-Chlorophenoxy)methyl]-4-oxiran-2-ylbutan-1-ol

This compound was prepared with 2-[(4-chlorophenoxy)methyl]hex-5-en-1-ol by a procedure similar to that in Preparation 40.

¹H NMR (CDCl₃) δ: 7.32-7.15 (m, 2H), 6.90-6.72 (m, 2H), 4.06-3.96 (m, 2H), 3.85-3.66 (m, 2H), 3.00-2.90 (m, 1H), 2.78 (t, J=4.5 Hz, 1H), 2.13-1.96 (m, 1H) 1.94-1.50 (m, 5H)ppm. (OH was not observed.)
Preparation 188

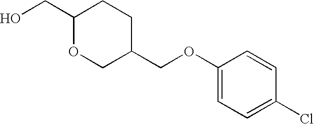

### {5-[(4-Chlorophenoxy)methyl]tetrahydro-2H-pyran-2-yl}methanol

This compound was prepared with 2-[(4-chlorophenoxy)methyl]-4-oxiran-2-ylbutan-1-ol by a procedure similar to that in Preparation 109.

¹H NMR (CDCl₃) 5: 7.30-7.19 (m, 2H), 6.90-6.76(m, 2H), 4.25-3.24 (m, 7H), 2.14-1.34 (m, 5H) ppm. (—OH was not observed.)
Preparation 189

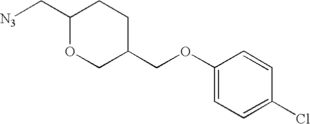

### 2-(Azidomethyl)-5-[(4-chlorophenoxy)methyl]tetrahydro-2H-pyran

This compound was prepared with {5-[(4-chlorophenoxy)methyl]tetrahydro-2H-pyran-2-yl}methanol by a procedure similar to that in Preparation 67.

¹H NMR (CDCl₃) δ: 7.40-7.17 (m, 2H), 6.95-6.72(m, 2H), 4.23-3.43 (m, 5H), 3.37-3.18 (m, 2H), 2.23-1.92 (m, 2H), 1.90-1.19 (m, 3H) ppm.

Experimental Example

NR2B Binding Assay and Human Dofetilide Binding Assay were conducted using the method described above. The results of these studies are summarized in Table 1.

Table 1. Results of NR2B Binding Assay and Human Dofetilide Binding Assay

| | | NR2B Binding | Dofetilide Bi |
|---|---|---|---|
| Compound | Structure | IC50 (nM) | IC50 (uM) |
| Example 9 | 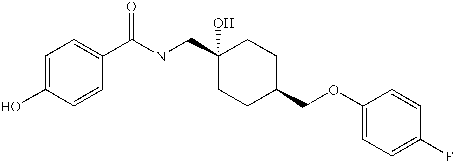 | 7.5 | 26.3 |
| Example 10 | 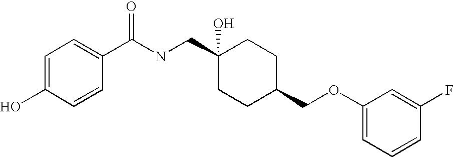 | 12.0 | 25.8 |
| Example 11 | 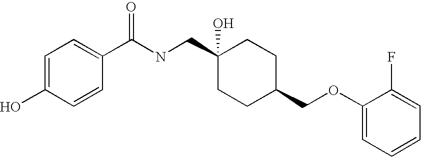 | 8.5 | >100 |
| Example 13 | 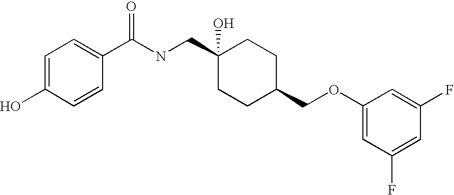 | 30.0 | 62.6 |
| Example 15 | 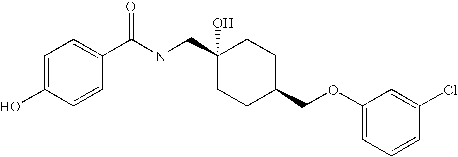 | 21.0 | 20.7 |
| Example 16 | 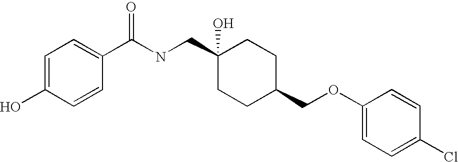 | 19.8 | >100 |
| Example 17 | 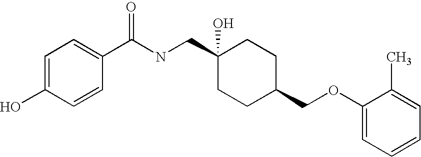 | 23.4 | >100 |
| Example 18 | 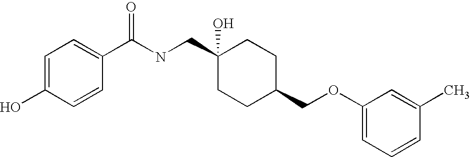 | 15.2 | >100 |
| Example 19 | 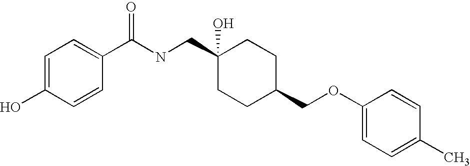 | 7.7 | >100 |
| Example 21 | 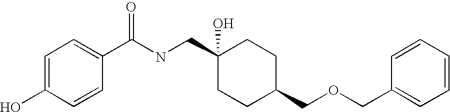 | 11.4 | >100 |
| Example 22 | 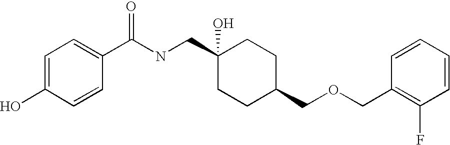 | 11.0. | >100 |
| Example 23 | 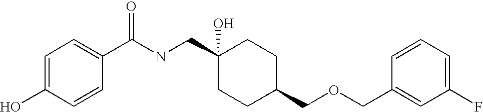 | 28.6 | >100 |
| Example 24 | 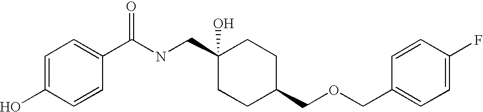 | 21.8 | 97.0 |
| Example 30 | 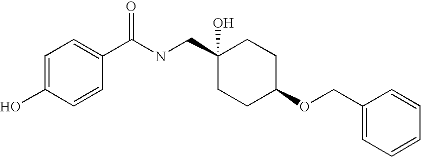 | 23.6 | >100 |
| Example 33 | 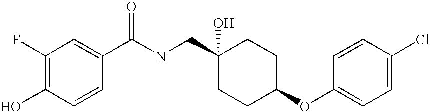 | 19.3 | 95.6 |
| Example 34 | 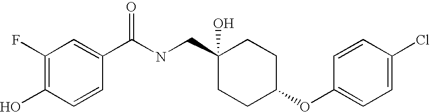 | 14.7 | >30 |
| Example 35 | 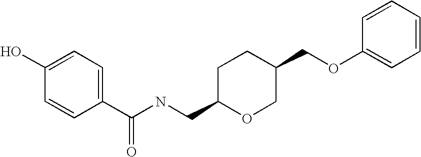 | 20.9 | 51.6 |
| Example 36 | 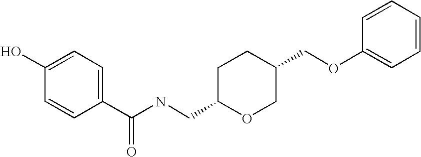 | 7.5 | 55.2 |
| Example 39 | 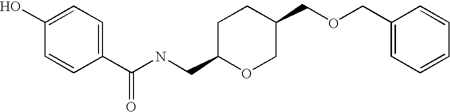 | 8.2 | >100 |
| Example 40 | 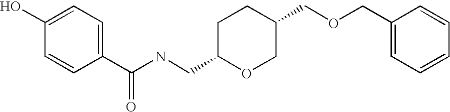 | 10.4 | >100 |
| Example 43 | 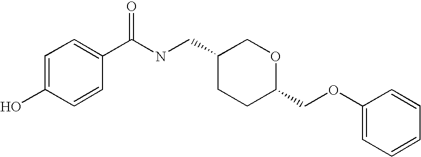 | 7.6 | >100 |
| Example 48 | 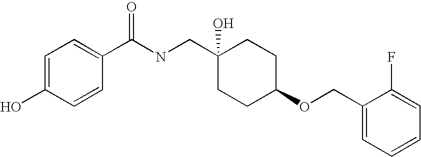 | 25.5 | >100 |
| Example 50 | 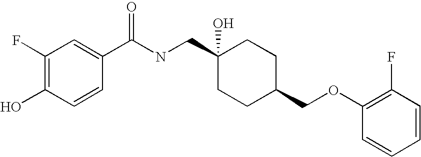 | 27.5 | >100 |
| Example 56 | 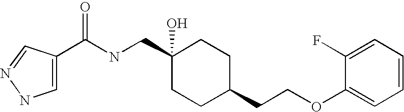 | 30.2 | >100 |
| Example 60 | 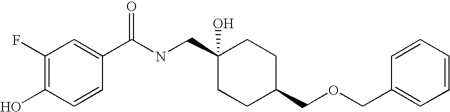 | 18.2 | >100 |
| Example 62 | 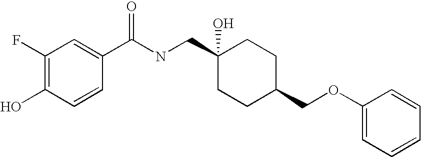 | 9.1 | >30 |
| Example 63 | 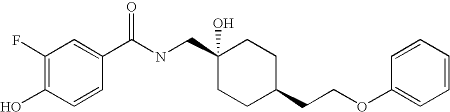 | 6.2 | >30 |
| Example 64 | 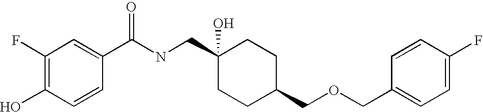 | 26.5 | >100 |
| Example 65 | 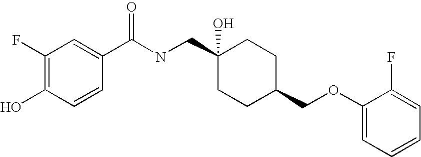 | 10.0 | >100 |
| Example 66 | 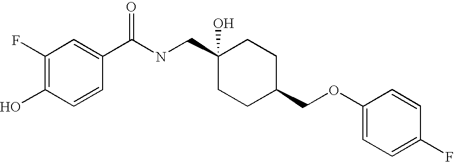 | 12.6 | >100 |
| Example 67 | 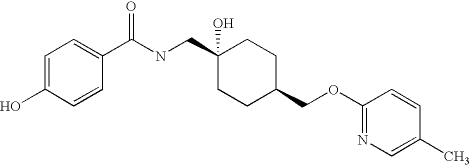 | 16.9 | >30 |
| Example 68 | 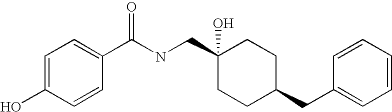 | 7.3 | >100 |
| Example 69 | 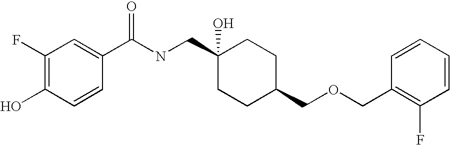 | 13.2 | >100 |
| Example 71 | 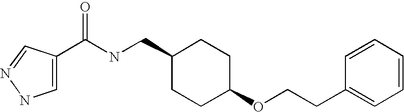 | 7.1 | >100 |
| Example 73 | 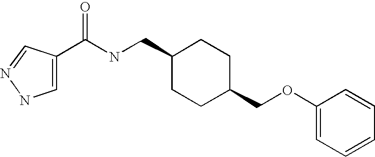 | 15.4 | >30 |
| Example 74 | 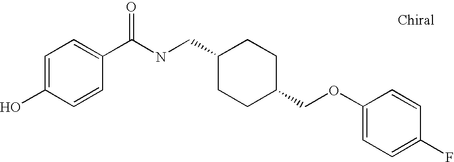 | 7.2 | 54.7 |
| Example 75 | 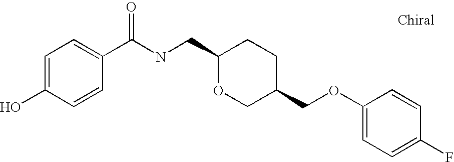 | 6.2 | 26.8 |
| Example 76 | 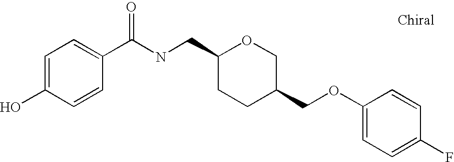 | 29.3 | 27.7 |
| Example 78 | 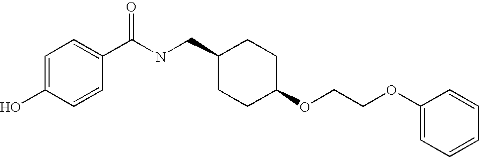 | 5.4 | 40.8 |
| Example 80 | 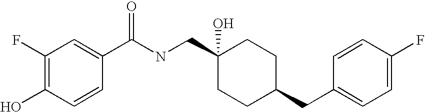 | 8.9 | >100 |
| Example 81 | 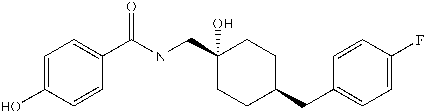 | 9.5 | 35.7 |
| Example 89 | 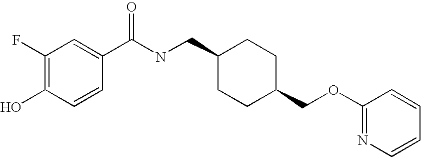 | 9.0 | >100 |
| Example 90 | 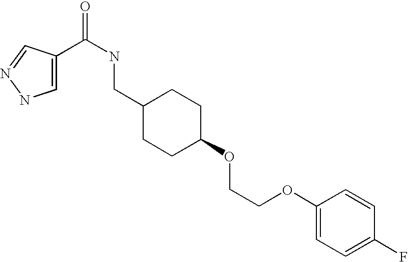 | 5.2 | >100 |
| Example 91 | 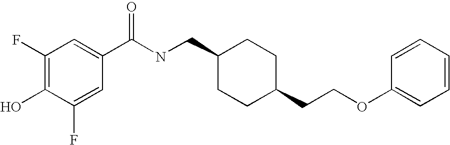 | 13.0 | >30 |
| Example 95 | 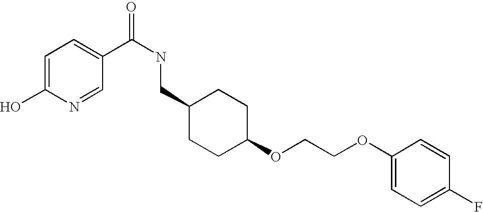 | 4.0 | >100 |
| Example 105 | 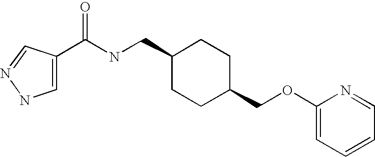 | 27.2 | >100 |
| Example 106 | 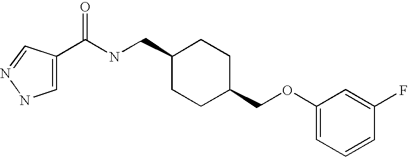 | 16.0 | 29.1 |
| Example 108 | 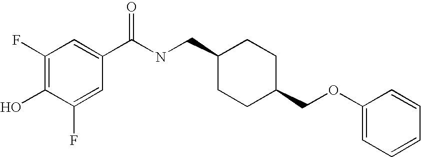 | 17.8 | >100 |
| Example 113 | 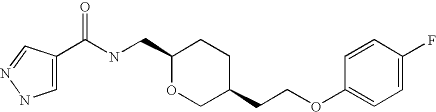 | 6.5 | >100 |
| Example 114 | 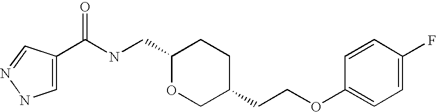 | 7.8 | 99.4 |
| Example 116 | 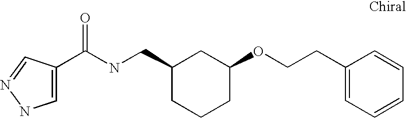 | 25.9 | >100 |
| Example 120 | 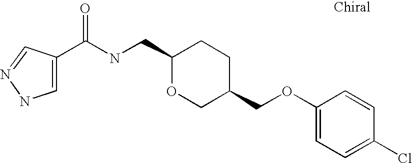 | 12.2 | >30 |
| Example 123 | 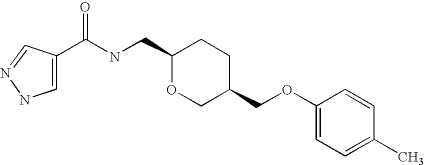 | 7.1 | >100 |
| Example 125 | 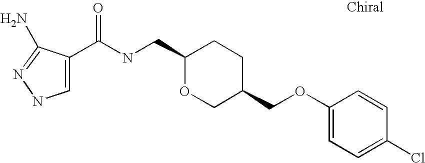 | 12.5 | >30 |
| Example 126 | 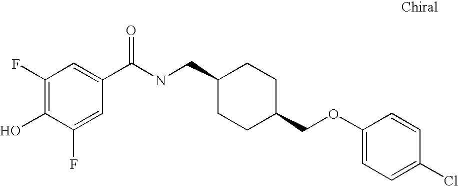 | 18.1 | >100 |
| Example 129 | 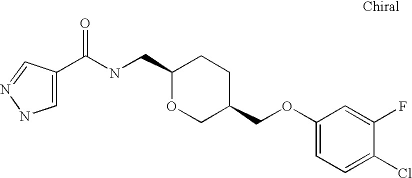 | 10.3 | >30 |

IC₅₀: the concentration of the individual compound required to reduce the amount of ligand by 50%.

1. A compound of the formula (I): 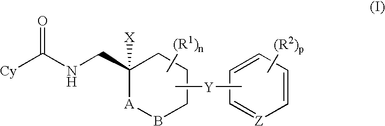
or a pharmaceutically acceptable salt or solvate thereof, wherein:
A and B independently represent CH₂ or O, with the proviso that A and B are not simultaneously O;
Cy represents one of the following 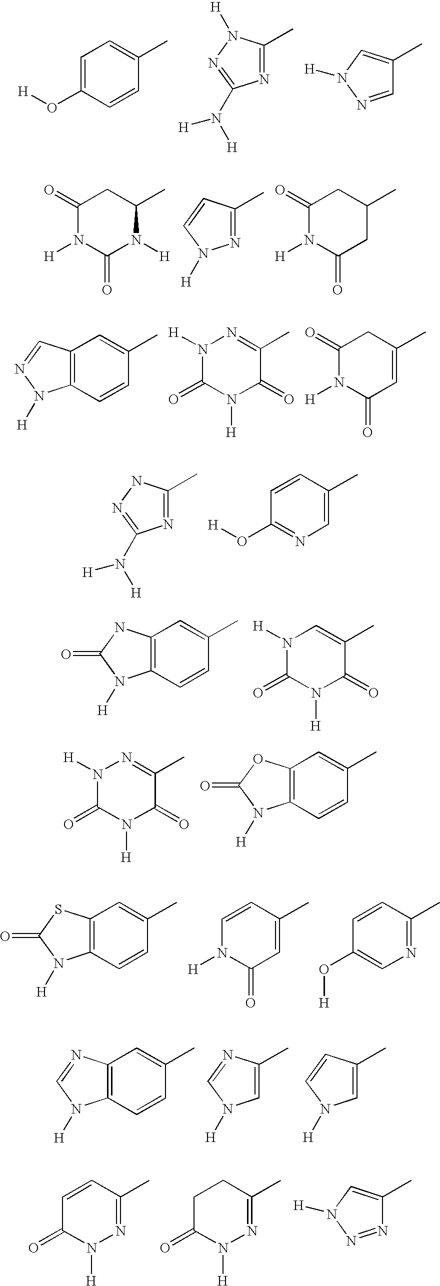 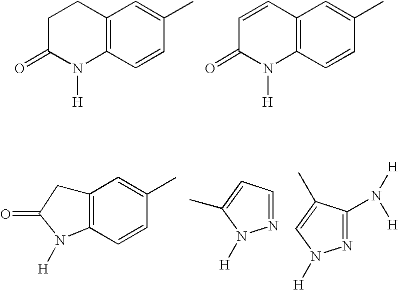
optionally substituted by one to three groups selected from hydroxy, halogen, C₁₋₆alkyl,
C₁₋₆alkoxy, C₁₋₆ haloalkyl, C₁₋₆alkylamino and amino;
R¹ and R² are independently selected from hydroxy, halogen, C₁₋₆alkyl, C₁₋₆alkoxy,
C₁₋₆haloalkyl and C₃₋₈ cycloalkyl;
n represents an integer from 0-4;
X is hydrogen, hydroxy, halogen or C₁₋₆ alkoxy;
Y is oxy, thio, a 1-4 membered alkylene, a 2-4 membered alkylene ether, 2-4 membered alkylene thioether or an oxyethyleneoxy group, optionally substituted by 1 to 4 groups independently selected from hydroxy, halogen, C₁₋₆alkyl, C₁₋₆alkoxy and C₁₋₆haloalkyl;
Z is CH or N; and
p represents an integer from 0-5 when Z is CH or 0-4 when Z is N, when p represents 2 or more, two of R²s may be taken together with the carbon atoms to which they are attached to form a 5-8 membered cycloalkyl ring.

2. A compound according to claim 1 wherein A and B represent carbon atoms.

3. A compound according to claim 1 wherein A represents O and B represents C.

4. A compound according to claim 1 wherein A represents C and B represents O.

5. A compound according to claim 1 where Cy is selected from optionally further substituted 4-hydroxyphenyl, 1H-pyrazol-4-yl, 2-oxo-2,3-dihydro-1,3-benzoxazole-6-yl and 2-hydroxy-5-pyridyl.

6. A compound according to claim 1 wherein Cy represents 4-hydroxyphenyl, optionally further substituted by fluoro or methyl.

7. A compound according to claim 1 wherein n represents 0.

8. A compound according to claim 1 wherein R² represents methoxy, chloro, fluoro or methyl.

9. A compound according to claim 1 wherein p represents 0-2.

10. A compound according to claim 1 wherein X is hydrogen or hydroxy.

11. A compound according to claim 1 wherein Y is selected from methylene, oxyethyleneoxy, oxymethylene, methyleneoxy, methyleneoxymethylene, ethyleneoxy, oxyethylene and oxy.

12. A compound according to claim 1 wherein Y is para located to and in a trans configuration to X.

13. A compound of formula (I) selected from the group consisting of:
4-Hydroxy-N-{[cis-4-(phenoxymethyl)cyclohexyl]methyl}benzamide;
4-Hydroxy-N-({cis-4-[(4-methoxyphenoxy)methyl]cyclohexyl}methyl)benzamide;
N-{[cis-4-(Benzyloxy)cyclohexyl]methyl}-4-hydroxybenzamide;
N-({cis-4-[(4-Chlorobenzyl)oxy]cyclohexyl}methyl)-4-hydroxybenzamide;
N-({cis-4-[(3-Chlorobenzyl)oxy]cyclohexyl}methyl)-4-hydroxybenzamide;
4-Hydroxy-N-{[cis-4-(4-methoxyphenoxy)cyclohexyl]methyl}benzamide;
N-{[cis-4-(4-Chlorophenoxy)cyclohexyl]methyl}-4-hydroxybenzamide;
4-Hydroxy-N-{[1-hydroxy-4-(phenoxymethyl)cyclohexyl]methyl}benzamide;
N-({trans-4-[(4-Fluorophenoxy)methyl]-1-hydroxycyclohexyl}methyl)-4-hydroxybenzamide;
N-({trans-4-[(3-Fluorophenoxy)methyl]-1-hydroxycyclohexyl}methyl)-4-hydroxybenzamide;
N-({trans-4-[(2-Fluorophenoxy)methyl]-1-hydroxycyclohexyl}methyl)-4-hydroxybenzamide;
N-({trans-4-[(2,6-Difluorophenoxy)methyl]-1-hydroxycyclohexyl}methyl)-4-hydroxybenzamide;
N-({trans-4-[(3,5-Difluorophenoxy)methyl]-1-hydroxycyclohexyl}methyl)-4-hydroxybenzamide;
N-({trans-4-[(3-Chlorophenoxy)methyl]-1-hydroxycyclohexyl}methyl)-4-hydroxybenzamide;
N-({trans-4-[(4-Chlorophenoxy)methyl]-1-hydroxycyclohexyl}methyl)-4-hydroxybenzamide;
4-Hydroxy-N-({trans-1-hydroxy-4-[(2-methylphenoxy)methyl]cyclohexyl}methyl)benzamide;
4-Hydroxy-N-({trans-1-hydroxy-4-[(3-methylphenoxy)methyl]cyclohexyl}methyl)benzamide;
4-Hydroxy-N-({trans-1-hydroxy-4-[(4-methylphenoxy)methyl]cyclohexyl}methyl)benzamide;
N-({trans-4-[(Benzyloxy)methyl]-1-hydroxycyclohexyl}methyl)-4-hydroxybenzamide;
N-[(trans-4-{[(2-Fluorobenzyl)oxy]methyl}-1-hydroxycyclohexyl)methyl]-4-hydroxybenzamide;
N-[(trans-4-{[(4-Fluorobenzyl)oxy]methyl}-1-hydroxycyclohexyl)methyl]-4-hydroxybenzamide;
4-Hydroxy-N-{[trans-1-hydroxy-4-(2-phenoxyethyl)cyclohexyl]methyl}benzamide;
N-({trans-4-[2-(2-Fluorophenoxy)ethyl]-1-hydroxycyclohexyl}methyl)-4-hydroxybenzamide;
N-({trans-4-[2-(3-Fluorophenoxy)ethyl]-1-hydroxycyclohexyl}methyl)-4-hydroxybenzamide;
N-({trans-4-[2-(4-Fluorophenoxy)ethyl]-1-hydroxycyclohexyl}methyl)-4-hydroxybenzamide;
N-{[trans-4-(Benzyloxy)-1-hydroxycyclohexyl]methyl}-4-hydroxybenzamide;
N-{[trans-4-(4-Chlorophenoxy)-1-hydroxycyclohexyl]methyl}-4-hydroxybenzamide;
N-{[cis-4-(4-Chlorophenoxy)-1-hydroxycyclohexyl]methyl}-4-hydroxybenzamide;
N-{[trans-4-(4-Chlorophenoxy)-1-hydroxycyclohexyl]methyl}-3-fluoro-4-hydroxybenzamide;
N-{[cis-4-(4-Chlorophenoxy)-1-hydroxycyclohexyl]methyl}-3-fluoro-4-hydroxybenzamide;
(+)-4-hydroxy-N-{[5S-(phenoxymethyl)tetrahydro-2H-pyran-2S-yl]methyl}benzamide;
(−)-4-hydroxy-N-{[5R-(phenoxymethyl)tetrahydro-2H-pyran-2R-yl]methyl}benzamide;
4-hydroxy-N-{[5S-(benzyloxymethyl)tetrahydro-2H-pyran-2S-yl]methyl}benzamide;
4-hydroxy-N-{[5R-(benzyloxymethyl)tetrahydro-2H-pyran-2R-yl]methyl}benzamide;
(−)-4-Hydroxy-N-{[(3R,6S)-6-(phenoxymethyl)tetrahydro-2H-pyran-3-yl]methyl}benzamide;
(+)-4-Hydroxy-N-{[(3S,6R)-6-(phenoxymethyl)tetrahydro-2H-pyran-3-yl]methyl}benzamide;
N-({trans-4-[(2-Fluorobenzyl)oxy]-1-hydroxycyclohexyl}methyl)-4-hydroxybenzamide;
3-Fluoro-N-({trans-4-[2-(2-fluorophenoxy)ethyl]-1-hydroxycyclohexyl}methyl)-4-hydroxybenzamide;
trans - N-{[4-(4-chlorophenoxy)cyclohexyl]methyl}-3-fluoro-4-hydroxybenzamide;
cis- N-{[4-(4-chlorophenoxy)cyclohexyl]methyl}-3-fiuoro-4-hydroxybenzamide;
N-{[cis-4-(4-Fluorophenoxy)cyclohexyl]methyl}-4-hydroxybenzamide;
3-Fluoro-N-{[cis-4-(4-fluorophenoxy)cyclohexyl]methyl}-4-hydroxybenzamide;
N-({trans-4-[2-(2-Fluorophenoxy)ethyl]-1-hydroxycyclohexyl}methyl)-1H-pyrazole-4-carboxamide;
4-hydroxy-N-{[cis-4-(2-phenylethoxy)cyclohexyl]methyl}benzamide;
2-fluoro-4-hydroxy-N-{[trans-1-hydroxy-4-(phenoxymethyl)cyclohexyl]methyl}benzamide;
N-({trans-4-[(benzyloxy)methyl]-1-hydroxycyclohexyl}methyl)-3-fluor6-4-hydoxybenzamide;
N-({cis-4-[(Benzyloxy)methyl]cyclohexyl}methyl)-4-hydroxybenzamide
3-Fluoro-4-hydroxy-N-{[trans-1-hydroxy-4-(phenoxymethyl)cyclohexyl]methyl}benzamide;
3-Fluoro-4-hydroxy-N-{[trans-1-hydroxy-4-(2-phenoxyethyl)cyclohexyl]methyl}benzamide;
3-Fluoro-N-[(trans-4-{[(4-fluorobenzyl)oxy]methyl}-1-hydroxycyclohexyl)methyl]-4-hydroxybenzamide;
3-Fluoro-N-({trans-4-[(2-fluorophenoxy)methyl]-1-hydroxycyclohexyl}methyl)-4-hydroxybenzamide;
3-Fluoro-N-({trans-4-[(4-fluorophenoxy)methyl]-1-hydroxycyclohexyl}methyl)-4-hydroxybenzamide;
4-Hydroxy-N-[(trans-1-hydroxy-4-{[(5-methylpyridin-2-yl)oxy]methyl}cyclohexyl)methyl]benzamide;
N-[(trans-4-Benzyl-1-hydroxycyclohexyl)methyl]-4-hydroxybenzamide;
3-fluoro-N-[(trans-4-{[(2-fluorobenzyl)oxy]methyl}-1-hydroxycyclohexyl)methyl]-4-hydroxybenzamide;
6-Hydroxy-N-{[cis-4-(2-phenethoxy)cyclohexyl]methyl}nicotinamide;
N-{[cis-4-(2-Phenylethoxy)cyclohexyl]methyl}-1H-pyrazole-4-carboxamide;
N-{[cis-4-(Phenoxymethyl)cyclohexyl]methyl}-1H-pyrazole-4-carboxamide;
N-{[cis-4-(2-Phenoxyethyl)cyclohexyl]methyl}-1H-pyrazole-4-carboxamide;
N-({cis-4-[(3-Fluorophenoxy)methyl]cyclohexyl}methyl)-1H-pyrazole-4-carboxamide;
N-({cis-4-[(4-Fluorophenoxy)methyl]cyclohexyl}methyl)-1H-pyrazole-4-carboxamide;
N-({(2R,5R)-5-[(4-Fluorophenoxy)methyl]tetrahydro-2H-pyran-2-yl}methyl)- H-pyrazole-4-carboxamide;
N-{[cis-4-(4-Methoxybenzyl)cyclohexyl]methyl}-1H-pyrazole-4-carboxamide;
3-Amino-N-[(cis-4-benzylcyclohexyl)methyl]-1H-pyrazole-4-carboxamide;
N-({(2R,5R)-5-[(4-Chlorophenoxy)methyl]tetrahydro-2H-pyran-2-yl}methyl)-1H-pyrazole-4-carboxamide;
3-Amino-N-({(2R,5R)-5-[(4-fluorophenoxy)methyl]tetrahydro-2H-pyran-2-yl}methyl)-1H-pyrazole-4-carboxamide;
3-Amino-N-({(2R5R)-5-[(4-chlorophenoxy) methyl]tetrahydro-2H-pyran-2-yl}methyl)-1H-pyrazole-4-carboxamide; and
3-Amino-N-({(2R,5R)-5-[(4-ethylphenoxy)methyl]tetrahydro-2H-pyran-2-yl}methyl)-1H-pyrazole-4-carboxamide;
or a pharmaceutically acceptable salt or solvate thereof.

14. A pharmaceutical composition including a compound of the formula (I) or a pharmaceutically acceptable salt or solvate thereof, as defined in claim 1, together with a pharmaceutically acceptable excipient.

15. A compound of the formula (I) or a pharmaceutically acceptable salt or solvate thereof, as defined in claim 1, for use as a medicament.

16. (canceled)

17. (canceled)

18. A method of treatment of a mammal, including a human being, to treat a disease for which an NMDA NR2B antagonist is indicated, including treating said mammal with an effective amount of a compound of the formula (I) or with a pharmaceutically acceptable salt, solvate or composition thereof, as defined in claim 1.

19. A method according to claim 18 wherein the disease is selected from pain, stroke, traumatic brain injury, Parkinson's disease, Alzheimer's disease, depression, anxiety and migraine.

20. A combination of a compound of the formula (I), as defined in claim 1, and another pharmacologically active agent.
